(19) United States
(12) Patent Application Publication
(10) Pub. No.: US 2023/0272221 A1
(43) Pub. Date: Aug. 31, 2023

(54) REAGENTS FOR LABELING BIOMOLECULES

(71) Applicant: Ultima Genomics, Inc., Newark, CA (US)

(72) Inventors: Linda G. LEE, Palo Alto, CA (US); Steven MENCHEN, Fremont, CA (US)

(21) Appl. No.: 18/111,220

(22) Filed: Feb. 17, 2023

Related U.S. Application Data
(63) Continuation of application No. PCT/US2021/046344, filed on Aug. 17, 2021.
(60) Provisional application No. 63/162,371, filed on Mar. 17, 2021.
(60) Provisional application No. 63/067,172, filed on Aug. 18, 2020.

(51) Int. Cl. C09B 49/04; C09B 11/24; C07H 19/20

(52) U.S. Cl. CPC C09B 49/04; C09B 11/24; C07H 19/20

(57) ABSTRACT

The present disclosure provides labeling reagents for labeling substrates such as nucleotides, proteins, antibodies, lipids, and cells. The labeling reagents provided herein may comprise fluorescent labels and semi-rigid linkers. Methods for nucleic acid sequencing using materials comprising such labeling reagents are also provided herein. Also provided are labeling reagents for labeling multiple substrates simultaneously using energy transfer.

### CROSS REFERENCE

This application is a continuation of International Application No. PCT/US2021/046344, filed on Aug. 17, 2021, which claims the benefit of U.S. Provisional Patent Application No. 63/067,172, filed on Aug. 18, 2020, and U.S. Provisional Patent Application No. 63/162,371, filed on Mar. 17, 2021, each of which is hereby incorporated by reference in its entirety.

### BACKGROUND

The detection, quantification, and sequencing of cells and biological molecules may be important for molecular biology and medical applications, such as diagnostics. Genetic testing may be useful for a number of diagnostic methods. For example, disorders that are caused by rare genetic alterations (e.g., sequence variants) or changes in epigenetic markers, such as cancer and partial or complete aneuploidy, may be detected or more accurately characterized with deoxyribonucleic acid (DNA) or ribonucleic acid (RNA) sequence information.

Nucleic acid sequencing is a process that can be used to provide sequence information for a nucleic acid sample. Such sequence information may be helpful in diagnosing and/or treating a subject with a condition. For example, the nucleic acid sequence of a subject may be used to identify, diagnose, and potentially develop treatments for genetic diseases. As another example, research into pathogens may lead to treatment of contagious diseases.

Nucleic acid sequencing may comprise the use of fluorescently labeled moieties. Such moieties may be labeled with organic fluorescent dyes. The sensitivity of a detection scheme can be improved by using dyes with both a high extinction coefficient and quantum yield, where the product of these characteristics may be termed the dye's “brightness.” Dye brightness may be attenuated by quenching phenomena, including quenching by biological materials, quenching by proximity to other dyes, and quenching by solvent. Other routes to brightness loss include photobleaching, reactivity to molecular oxygen, and chemical decomposition.

### SUMMARY

The present disclosure provides improved optical (e.g., fluorescent) labeling reagents and methods of nucleic acid processing comprising the use of optically (e.g., fluorescently) labeled moieties. The materials and methods provided herein may comprise the use of organic fluorescent dyes. The materials provided herein may allow for optimized molecular quenching to facilitate efficient nucleic acid processing and detection. Molecular quenching mechanisms can include photoinduced electron transfer, photoinduced hole transfer, Forster energy transfer, Dexter quenching, and the like. A general solution to many types of quenching requires physical separation of the dye from the quencher moiety, but existing solutions all have advantages and disadvantages in terms of ease of use, cost, solvent-dependence and polydispersity. Accordingly, the present disclosure recognizes the need for materials and methods that address these limitations and provides materials comprising improved linker moieties.

In an aspect, the present disclosure provides a fluorescent labeling reagent comprising: (a) a fluorescent dye moiety; and (b) a linker that is connected to said fluorescent dye moiety and configured to couple to a substrate for fluorescently labelling said substrate, wherein said linker comprises at least five non-proteinogenic amino acids.

In some embodiments, said fluorescent labeling reagent further comprises a second fluorescent dye, wherein said fluorescent dye and said second fluorescent dye are connected by said linker and capable of energy transfer. In some embodiments, said energy transfer is mediated via fluorescence resonance energy transfer (FRET).

In some embodiments, at least a subset of said at least five non-proteinogenic amino acids comprise ring systems. In some embodiments, at least a subset of said at least five non-proteinogenic amino acids comprise water soluble groups. In some embodiments, said water soluble groups are selected from the group consisting of pyridinium groups, imidazolium groups, quaternary ammonium groups, sulfonates, sulfates, phosphates, hydroxyl groups, amines, imines, nitriles, amides, thiols, carboxylic acids, polyethers, aldehydes, boronic acids, and boronic esters. In some embodiments, said water soluble groups are hydroxyl groups.

In some embodiments, at least a subset of said at least five non-proteinogenic amino acids are hydroxyproline moieties. In some embodiments, said linker comprises five or more hydroxyproline moieties. In some embodiments, said linker comprises ten or more hydroxyproline moieties. In some embodiments, said linker comprises twenty or more hydroxyproline moieties. In some embodiments, said linker comprises thirty or more hydroxyproline moieties. In some embodiments, said linker further comprises one or more glycine moieties.

In some embodiments, said linker comprises a repeating unit. In some embodiments, said repeating unit comprises one or more of said at least five non-proteinogenic amino acid moieties. In some embodiments, said repeating unit comprises at least five non-proteinogenic amino acid moieties. In some embodiments, said repeating unit comprises at least ten non-proteinogenic amino acid moieties. In some embodiments, said repeating unit comprises ten hydroxyproline moieties. In some embodiments, said repeating unit comprises a glycine moiety. In some embodiments, said repeating unit is repeated at least three times.

In some embodiments, when said fluorescent labeling reagent is coupled to said substrate, said linker provides an average physical separation between said fluorescent dye moiety and said substrate of at least about 30 Angstroms (Å). In some embodiments, when said fluorescent labeling reagent is coupled to said substrate, said linker provides an average physical separation between said fluorescent dye moiety and said substrate of at least about 60 Angstroms (Å). In some embodiments, when said fluorescent labeling reagent is coupled to said substrate, said linker provides an average physical separation between said fluorescent dye moiety and said substrate of at least about 90 Angstroms (Å).

In some embodiments, said fluorescent labeling reagent further comprises a cleavable group that is configured to be cleaved to separate said fluorescent labeling reagent or portion thereof from said substrate. In some embodiments, said cleavable group is configured to be cleaved to separate a first portion of said fluorescent labeling reagent comprising said fluorescent dye moiety and a first portion of said linker and a second portion of said fluorescent labeling reagent comprising a second portion of said linker. In some embodiments, said cleavable group is selected from the group consisting of an azidomethyl group, a disulfide bond, a hydrocarbyldithiomethyl group, and a 2-nitrobenzyloxy group. In some embodiments, said cleavable group is a disulfide bond. In some embodiments, said cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof.

In some embodiments, said fluorescent labeling reagent comprises a moiety selected from the group consisting of

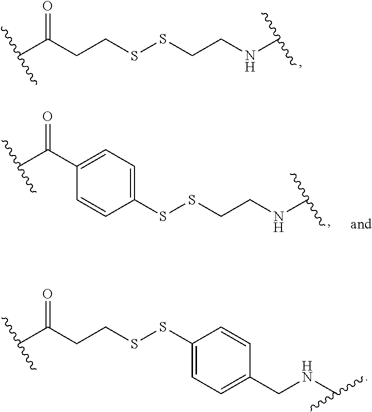 [SMILES: *NCCSSCCC(*)=O ; *NCCSSc1ccc(C(*)=O)cc1 ; *NCc1ccc(SSCCC(*)=O)cc1]

In some embodiments, said substrate is a nucleotide, polynucleotide, protein, lipid, cell, saccharide, polysaccharide, or antibody. In some embodiments, said substrate is a nucleotide and said fluorescent labeling reagent is attached to said nucleotide via the nucleobase of said nucleotide. In some embodiments, said substrate is a protein. In some embodiments, said substrate is a fluorescence quencher, a fluorescence donor, or a fluorescence acceptor.

In an aspect, the present disclosure provides a labeled substrate comprising said substrate and said fluorescent labeling reagent described anywhere herein, or a derivative thereof, wherein said fluorescent labeling reagent is coupled to said substrate.

In some embodiments, said substrate is a nucleotide, polynucleotide, protein, lipid, cell, saccharide, polysaccharide, or antibody. In some embodiments, said substrate is a protein. In some embodiments, said protein is a component of a cell. In some embodiments, said substrate is a nucleotide and said fluorescent labeling reagent is attached to said nucleotide via the nucleobase of said nucleotide.

In some embodiments, said labeled substrate comprises an additional fluorescent labeling reagent coupled thereto, wherein said additional fluorescent labeling reagent comprises an additional fluorescent dye moiety and an additional linker connected to said additional fluorescent dye moiety, wherein said additional linker comprises at least five non-proteinogenic amino acids. In some embodiments, said fluorescent labeling reagent and said additional fluorescent labeling reagent comprise identical chemical structures. In some embodiments, said fluorescent labeling reagent and said additional fluorescent labeling reagent comprise different chemical structures. In some embodiments, said labeled substrate comprises three or more fluorescent labeling reagents coupled thereto.

In some embodiments, said substrate is a fluorescence quencher, a fluorescence donor, or a fluorescence acceptor.

In some embodiments, said labeled substrate reduces quenching relative to another labeled substrate comprising said substrate and another fluorescent labeling reagent comprising said fluorescent dye moiety and another linker that does not comprise said at least twenty non-proteinogenic amino acids.

In some embodiments, said labeled substrate provides a higher signal level upon excitation and optical detection than another labeled substrate comprising said substrate and another fluorescent labeling reagent comprising said fluorescent dye moiety and another linker that does not comprise said at least twenty non-proteinogenic amino acids.

In another aspect, the present disclosure provides a fluorescent labeling reagent comprising: (a) a plurality of fluorescent dye moieties; and (b) a plurality of linkers, wherein a first linker of said plurality of linkers is connected to a first fluorescent dye moiety of said plurality of fluorescent dye moieties, and wherein a second linker of said plurality of linkers is connected to a second fluorescent dye moiety of said plurality of fluorescent dye moieties, wherein said fluorescent labeling reagent is configured to couple to a substrate for fluorescently labelling said substrate, and wherein said first linker comprises a first non-proteinogenic amino acid and said second linker comprises a second non-proteinogenic amino acid.

In some embodiments, said first fluorescent dye moiety and said second fluorescent dye moiety have the same chemical structure. In some embodiments, each fluorescent dye moiety of said plurality of fluorescent dye moieties have the same chemical structure. In some embodiments, each fluorescent dye moiety of said plurality of fluorescent dye moieties fluoresce at or near the same wavelength. In some embodiments, said first fluorescent dye moiety and said second fluorescent dye moiety have different chemical structures.

In some embodiments, said plurality of linkers are connected to one or more lysine moieties. In some embodiments, said fluorescent labeling reagent comprises two or more lysine moieties to which at least a subset of said plurality of linkers are connected. In some embodiments, said fluorescent labeling reagent comprises three or more lysine moieties to which at least a subset of said plurality of linkers are connected. In some embodiments, said first linker is connected to a first lysine moiety of said two or more lysine moieties and said second linker is connected to a second lysine moiety of said two or more lysine moieties. In some embodiments, said first lysine moiety is connected to said second lysine moiety.

In some embodiments, said fluorescent labeling reagent comprises three or more fluorescent dye moieties and three or more linkers.

In some embodiments, said first linker and said second linker have identical chemical structures. In some embodiments, each linker of said plurality of linkers have the same chemical structure. In some embodiments, said first linker and said second linker have different chemical structures.

In some embodiments, said first linker comprises a first plurality of amino acids comprising a first plurality of non-proteinogenic amino acids, wherein said first plurality of non-proteinogenic amino acids comprises said first non-proteinogenic amino acid. In some embodiments, at least a subset of said first plurality of non-proteinogenic amino acids comprises ring systems. In some embodiments, said first plurality of non-proteinogenic amino acids comprises at least five non-proteinogenic amino acids. In some embodiments, said first plurality of non-proteinogenic amino acids comprises at least ten non-proteinogenic amino acids. In some embodiments, said first plurality of non-proteinogenic amino acids comprises at least twenty non-proteinogenic amino acids. In some embodiments, said first plurality of non-proteinogenic amino acids comprises at least one hydroxyproline moiety. In some embodiments, said first plurality of non-proteinogenic amino acids comprises at least five hydroxyproline moieties. In some embodiments, said first plurality of non-proteinogenic amino acids comprises at least ten hydroxyproline moieties. In some embodiments, said first plurality of non-proteinogenic amino acids comprises at least twenty hydroxyproline moieties.

In some embodiments, said second linker comprises a second plurality of amino acids comprising a second plurality of non-proteinogenic amino acids, wherein said second plurality of non-proteinogenic amino acids comprises said second non-proteinogenic amino acid. In some embodiments, at least a subset of said second plurality of non-proteinogenic amino acids comprises ring systems. In some embodiments, said second plurality of non-proteinogenic amino acids comprises at least five non-proteinogenic amino acids. In some embodiments, said second plurality of non-proteinogenic amino acids comprises at least ten non-proteinogenic amino acids. In some embodiments, said second plurality of non-proteinogenic amino acids comprises at least twenty non-proteinogenic amino acids. In some embodiments, said second plurality of non-proteinogenic amino acids comprises at least one hydroxyproline moiety. In some embodiments, said second plurality of non-proteinogenic amino acids comprises at least five hydroxyproline moieties. In some embodiments, said second plurality of non-proteinogenic amino acids comprises at least ten hydroxyproline moieties. In some embodiments, said second plurality of non-proteinogenic amino acids comprises at least twenty hydroxyproline moieties.

In some embodiments, said first non-proteinogenic amino acid or said second non-proteinogenic amino acid comprises a ring system. In some embodiments, said first non-proteinogenic amino acid or said second non-proteinogenic amino acid comprises a water soluble group. In some embodiments, said water soluble group is selected from the group consisting of a pyridinium group, an imidazolium group, a quaternary ammonium group, a sulfonate, a sulfate, a phosphate, a hydroxyl group, an amine, an imine, a nitrile, an amide, a thiol, a carboxylic acid, a polyether, an aldehyde, a boronic acid, and a boronic ester. In some embodiments, said water soluble group is a hydroxyl group.

In some embodiments, said first linker or said second linker comprises three or more hydroxyproline moieties. In some embodiments, said first linker or said second linker comprises ten or more hydroxyproline moieties. In some embodiments, each linker of said plurality of linkers comprises three or more hydroxyproline moieties. In some embodiments, each linker of said plurality of linkers comprises ten or more hydroxyproline moieties. In some embodiments, said first linker or said second linker further comprises a glycine moiety. In some embodiments, said first linker or said second linker further comprises a cysteic acid moiety.

In some embodiments, said first linker or said second linker comprises a repeating unit. In some embodiments, said repeating unit comprises one or more non-proteinogenic amino acid moieties. In some embodiments, said repeating unit comprises five or more non-proteinogenic amino acid moieties. In some embodiments, said repeating unit comprises ten or more non-proteinogenic amino acid moieties. In some embodiments, said repeating unit comprises ten hydroxyproline moieties. In some embodiments, said repeating unit comprises a glycine moiety. In some embodiments, said repeating unit is repeated at least three times.

In some embodiments, when said fluorescent labeling reagent is coupled to said substrate, said first linker provides an average physical separation between said first fluorescent dye moiety and said substrate of at least about 30 Angstroms (Å). In some embodiments, when said fluorescent labeling reagent is coupled to said substrate, said first linker provides an average physical separation between said first fluorescent dye moiety and said substrate of at least about 60 Angstroms (Å). In some embodiments, when said fluorescent labeling reagent is coupled to said substrate, said first linker provides an average physical separation between said first fluorescent dye moiety and said substrate of at least about 90 Angstroms (Å).

In some embodiments, when said fluorescent labeling reagent is coupled to said substrate, said second linker provides an average physical separation between said second fluorescent dye moiety and said substrate of at least about 30 Angstroms (Å). In some embodiments, when said fluorescent labeling reagent is coupled to said substrate, said second linker provides an average physical separation between said second fluorescent dye moiety and said substrate of at least about 60 Angstroms (Å). In some embodiments, when said fluorescent labeling reagent is coupled to said substrate, said second linker provides an average physical separation between said second fluorescent dye moiety and said substrate of at least about 90 Angstroms (Å).

In some embodiments, said fluorescent labeling reagent further comprises a cleavable group that is configured to be cleaved to separate said fluorescent labeling reagent or a portion thereof from said substrate. In some embodiments, said cleavable group is configured to be cleaved to separate a first portion of said fluorescent labeling reagent comprising said plurality of fluorescent dye moieties and said plurality of linkers and a second portion of said fluorescent labeling reagent. In some embodiments, said cleavable group is selected from the group consisting of an azidomethyl group, a disulfide bond, a hydrocarbyldithiomethyl group, and a 2-nitrobenzyloxy group. In some embodiments, said cleavable group is a disulfide bond. In some embodiments, said cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof.

In some embodiments, said fluorescent labeling reagent comprises a moiety selected from the group consisting of

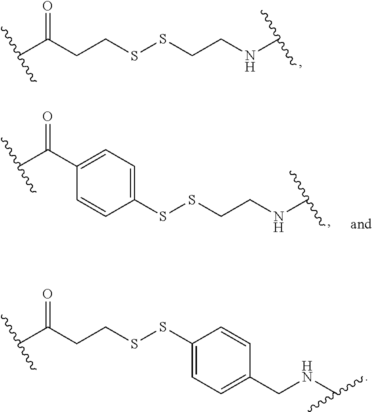 [SMILES: *NCCSSCCC(*)=O ; *NCCSSc1ccc(C(*)=O)cc1 ; *NCc1ccc(SSCCC(*)=O)cc1]

In some embodiments, said substrate is a nucleotide, polynucleotide, protein, lipid, cell, saccharide, polysaccharide, or antibody. In some embodiments, said substrate is a nucleotide and said fluorescent labeling reagent is attached to said nucleotide via the nucleobase of said nucleotide. In some embodiments, said substrate is a protein. In some embodiments, said substrate is a fluorescence quencher, a fluorescence donor, or a fluorescence acceptor.

In another aspect, the present disclosure provides a labeled substrate comprising said substrate and a fluorescent labeling reagent described herein, or a derivative thereof, wherein said fluorescent labeling reagent is coupled to said substrate.

In some embodiments, said substrate is a nucleotide, polynucleotide, protein, lipid, cell, saccharide, polysaccharide, or antibody. In some embodiments, said substrate is a protein. In some embodiments, said protein is a component of a cell. In some embodiments, said substrate is a nucleotide and said fluorescent labeling reagent is attached to said nucleotide via the nucleobase of said nucleotide.

In some embodiments, said labeled substrate comprises an additional fluorescent labeling reagent coupled thereto, wherein said additional fluorescent labeling reagent comprises an additional fluorescent dye moiety and an additional linker connected to said additional fluorescent dye moiety, wherein said additional linker comprises a non-proteinogenic amino acid. In some embodiments, said fluorescent labeling reagent and said additional fluorescent labeling reagent comprise identical chemical structures. In some embodiments, said fluorescent labeling reagent and said additional fluorescent labeling reagent comprise different chemical structures. In some embodiments, said labeled substrate comprises three or more fluorescent labeling reagents coupled thereto.

In some embodiments, said substrate is a fluorescence quencher, a fluorescence donor, or a fluorescence acceptor.

In some embodiments, said labeled substrate reduces quenching relative to another labeled substrate comprising said substrate and another fluorescent labeling reagent that comprises said plurality of fluorescent dye moieties and does not comprise a linker having the same chemical structure as said first linker or said second linker.

In some embodiments, said labeled substrate provides a higher signal level upon excitation and optical detection than another labeled substrate comprising said substrate and another fluorescent labeling reagent that comprises said plurality of fluorescent dye moieties and does not comprise a linker having the same chemical structure as said first linker or said second linker.

In another aspect, the present disclosure provides a composition comprising a solution comprising a fluorescently labeled nucleotide, wherein said fluorescently labeled nucleotide comprises a fluorescent labeling reagent comprising a fluorescent dye moiety that is connected to a nucleotide via a linker, wherein said linker comprises at least five non-proteinogenic amino acids. In some embodiments, at least a subset of said at least five non-proteinogenic amino acids comprises ring systems. In some embodiments, at least a subset of said at least five non-proteinogenic amino acids comprises water soluble groups. In some embodiments, said water soluble groups are selected from the group consisting of pyridinium groups, imidazolium groups, quaternary ammonium groups, sulfonates, sulfates, phosphates, hydroxyl groups, amines, imines, nitriles, amides, thiols, carboxylic acids, polyethers, aldehydes, boronic acids, and boronic esters. In some embodiments, said water soluble groups are hydroxyl groups. In some embodiments, at least a subset of said at five non-proteinogenic amino acids is hydroxyproline moiety.

In some embodiments, said linker comprises at least ten non-proteinogenic amino acids. In some embodiments, said linker comprises at least twenty non-proteinogenic amino acids. In some embodiments, said linker comprises at least thirty non-proteinogenic amino acids. In some embodiments, said linker comprises at least one hydroxyproline moiety. In some embodiments, said linker comprises at least five hydroxyproline moieties. In some embodiments, said linker comprises at least ten hydroxyproline moieties.

said linker comprises at least twenty hydroxyproline moieties. In some embodiments, said linker comprises at least thirty hydroxyproline moieties. In some embodiments, said linker further comprises one or more glycine moieties.

In some embodiments, said linker comprises a repeating unit. In some embodiments, said repeating unit comprises one or more non-proteinogenic amino acid moieties. In some embodiments, said repeating unit comprises at least five non-proteinogenic amino acid moieties. In some embodiments, said repeating unit comprises at least ten non-proteinogenic amino acid moieties. In some embodiments, said repeating unit comprises at least one hydroxyproline moiety. In some embodiments, said repeating unit comprises at least five hydroxyproline moieties. In some embodiments, said repeating unit comprises ten hydroxyproline moieties. In some embodiments, said repeating unit comprises a glycine moiety. In some embodiments, said repeating unit is repeated at least three times.

In some embodiments, said fluorescent labeling reagent further comprises a cleavable group that is configured to be cleaved to separate said fluorescent dye moiety from said nucleotide. In some embodiments, said cleavable group is selected from the group consisting of an azidomethyl group, a disulfide bond, a hydrocarbyldithiomethyl group, and a 2-nitrobenzyloxy group. In some embodiments, said cleavable group is a disulfide bond. In some embodiments, said cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof.

In some embodiments, said linker comprises a moiety selected from the group consisting of

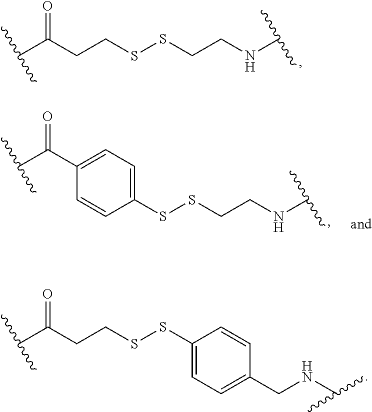 [SMILES: *NCCSSCCC(*)=O ; *NCCSSc1ccc(C(*)=O)cc1 ; *NCc1ccc(SSCCC(*)=O)cc1]

In some embodiments, said linker provides an average physical separation between said fluorescent dye moiety and said nucleotide of at least about 30 Angstroms (Å). In some embodiments, said linker provides an average physical separation between said fluorescent dye moiety and said nucleotide of at least about 60 Angstroms (Å). In some embodiments, said linker provides an average physical separation between said fluorescent dye moiety and said nucleotide of at least about 90 Angstroms (Å).

In some embodiments, said solution comprises a plurality of fluorescently labeled nucleotides, wherein each fluorescently labeled nucleotide of said plurality of said fluorescently labeled nucleotides comprises (i) a fluorescent labeling reagent comprising a fluorescent dye moiety of a same type and a linker of a same type and (ii) a nucleotide of a same type. In some embodiments, each said linker of each fluorescently labeled nucleotide of said plurality of fluorescently labeled nucleotides has the same molecular weight. In some embodiments, said solution further comprises a plurality of unlabeled nucleotides, wherein each nucleotide of said plurality of unlabeled nucleotides is of a same type as each said nucleotide of said plurality of fluorescently labeled nucleotides. In some embodiments, the ratio of said plurality of fluorescently labeled nucleotides to said plurality of unlabeled nucleotides in said solution is at least about 1:4. In some embodiments, said ratio is at least about 1:1.

In another aspect, the present disclosure provides a method comprising providing a composition described herein to a template nucleic acid molecule coupled to a nucleic acid strand.

In some embodiments, the method further comprises subjecting said template nucleic acid molecule and said composition to conditions sufficient to incorporate said fluorescently labeled nucleotide into said nucleic acid strand coupled to said template nucleic acid molecule. In some embodiments, the method further comprises detecting a signal from said fluorescently labeled nucleotide. In some embodiments, the method further comprises contacting said fluorescently labeled nucleotide with a cleavage reagent configured to cleave said plurality of fluorescent dye moieties from said nucleotide. In some embodiments, the method further comprises, subsequent to said contacting said fluorescently labeled nucleotide with said cleavage reagent, subjecting said template nucleic acid molecule and said composition to conditions sufficient to incorporate an additional fluorescently labeled nucleotide into said nucleic acid strand coupled to said template nucleic acid molecule.

In some embodiments, said template nucleic acid molecule is immobilized to a support.

In another aspect, the present disclosure provides a composition comprising a solution comprising a fluorescently labeled nucleotide, wherein said fluorescently labeled nucleotide comprises a fluorescent labeling reagent comprising a plurality of fluorescent dye moieties that are connected to a nucleotide via a plurality of linkers, wherein a first linker of said plurality of linkers is connected to a first fluorescent dye moiety of said plurality of fluorescent dye moieties, and wherein a second linker of said plurality of linkers is connected to a second fluorescent dye moiety of said plurality of fluorescent dye moieties, and wherein said first linker comprises a first non-proteinogenic amino acid and said second linker comprises a second non-proteinogenic amino acid.

In some embodiments, said first fluorescent dye moiety and said second fluorescent dye moiety have the same chemical structure. In some embodiments, each fluorescent dye moiety of said plurality of fluorescent dye moieties have the same chemical structure. In some embodiments, each fluorescent dye moiety of said plurality of fluorescent dye moieties fluoresces at or near the same wavelength. In some embodiments, said first fluorescent dye moiety and said second fluorescent dye moiety have different chemical structures.

In some embodiments, said fluorescent labeling reagent comprises one or more lysine moieties to which said plurality of linkers are connected. In some embodiments, said fluorescent labeling reagent comprises two or more lysine moieties to which at least a subset of said plurality of linkers is connected. In some embodiments, said fluorescent labeling reagent comprises three or more lysine moieties to which at least a subset of said plurality of linkers is connected. In some embodiments, said first linker is connected to a first lysine moiety of said two or more lysine moieties and said second linker is connected to a second lysine moiety of said two or more lysine moieties. In some embodiments, said first lysine moiety is connected to said second lysine moiety.

In some embodiments, said fluorescent labeling reagent comprises three or more fluorescent dye moieties and three or more linkers. In some embodiments, said first linker and said second linker have the same chemical structure. In some embodiments, each linker of said plurality of linkers has the same chemical structure. In some embodiments, said first linker and said second linker have different chemical structures.

In some embodiments, said first linker comprises a first plurality of amino acids comprising a first plurality of non-proteinogenic amino acids, wherein said first plurality of non-proteinogenic amino acids comprises said first non-proteinogenic amino acid. In some embodiments, at least a subset of said first plurality of non-proteinogenic amino acids comprises ring systems. In some embodiments, said first plurality of non-proteinogenic amino acids comprises at least five non-proteinogenic amino acids. In some embodiments, said first plurality of non-proteinogenic amino acids comprises at least ten non-proteinogenic amino acids. In some embodiments, said first plurality of non-proteinogenic amino acids comprises at least twenty non-proteinogenic amino acids.

In some embodiments, said second linker comprises a second plurality of amino acids comprising a second plurality of non-proteinogenic amino acids, wherein said second plurality of non-proteinogenic amino acids comprises said second non-proteinogenic amino acid. In some embodiments, at least a subset of said second plurality of non-proteinogenic amino acids comprises ring systems. In some embodiments, said second plurality of non-proteinogenic amino acids comprises at least five non-proteinogenic amino acids. In some embodiments, said second plurality of non-proteinogenic amino acids comprises at least ten non-proteinogenic amino acids. In some embodiments, said second plurality of non-proteinogenic amino acids comprises at least twenty non-proteinogenic amino acids.

In some embodiments, said first non-proteinogenic amino acid or said second non-proteinogenic amino acid comprises a ring system. In some embodiments, said first non-proteinogenic amino acid or said second non-proteinogenic amino acid comprises a water soluble group. In some embodiments, said water soluble group is selected from the group consisting of a pyridinium group, an imidazolium group, a quaternary ammonium group, a sulfonate, a sulfate, a phosphate, a hydroxyl group, an amine, an imine, a nitrile, an amide, a thiol, a carboxylic acid, a polyether, an aldehyde, a boronic acid, and a boronic ester. In some embodiments, said water soluble group is a hydroxyl group.

In some embodiments, said first linker or said second linker comprises three or more hydroxyproline moieties. In some embodiments, said first linker or said second linker comprises ten or more hydroxyproline moieties. In some embodiments, said first linker or said second linker comprises twenty or more hydroxyproline moieties. In some embodiments, said first linker or said second linker comprises thirty or more hydroxyproline moieties.

In some embodiments, each linker of said plurality of linkers comprises three or more hydroxyproline moieties. In some embodiments, each linker of said plurality of linkers comprises ten or more hydroxyproline moieties. In some embodiments, each linker of said plurality of linkers comprises twenty or more hydroxyproline moieties. In some embodiments, each linker of said plurality of linkers comprises thirty or more hydroxyproline moieties. In some embodiments, said first linker or said second linker further comprises a glycine moiety. In some embodiments, said first linker or said second linker further comprises a cysteic acid moiety.

In some embodiments, said first linker or said second linker comprises a repeating unit. In some embodiments, said repeating unit comprises one or more non-proteinogenic amino acid moieties. In some embodiments, said repeating unit comprises five or more non-proteinogenic amino acid moieties. In some embodiments, said repeating unit comprises ten or more non-proteinogenic amino acid moieties. In some embodiments, said repeating unit comprises at least one hydroxyproline moiety. In some embodiments, said repeating unit comprises at least five hydroxyproline moieties. In some embodiments, said repeating unit comprises ten hydroxyproline moieties. In some embodiments, said repeating unit comprises a glycine moiety. In some embodiments, said repeating unit is repeated at least three times.

In some embodiments, said fluorescently labeled nucleotide further comprises a cleavable group that is configured to be cleaved to separate said first portion of said fluorescently labeled nucleotide comprising said plurality of fluorescent dye moieties from a second portion of said fluorescently labeled nucleotide comprising said nucleotide. In some embodiments, said cleavable group is selected from the group consisting of an azidomethyl group, a disulfide bond, a hydrocarbyldithiomethyl group, and a 2-nitrobenzyloxy group. In some embodiments, said cleavable group is a disulfide bond. In some embodiments, said cleavable group is cleavable by application of one or more members of the group consisting of tris(2-carboxyethyl)phosphine (TCEP), dithiothreitol (DTT), tetrahydropyranyl (THP), ultraviolet (UV) light, and a combination thereof.

In some embodiments, said fluorescently labeled nucleotide comprises a moiety selected from the group consisting of

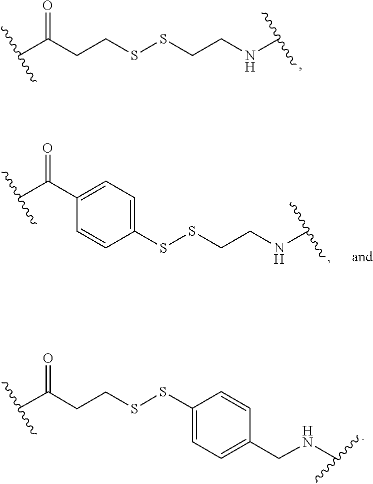 [SMILES: *NCCSSCCC(*)=O ; *NCCSSc1ccc(C(*)=O)cc1 ; *NCc1ccc(SSCCC(*)=O)cc1]

In some embodiments, said first linker provides an average physical separation between said first fluorescent dye moiety and said nucleotide of at least about 30 Angstroms (Å). In some embodiments, said first linker provides an average physical separation between said first fluorescent dye moiety and said nucleotide of at least about 60 Angstroms (Å). In some embodiments, said first linker provides an average physical separation between said first fluorescent dye moiety and said nucleotide of at least about 90 Angstroms (Å).

In some embodiments, said second linker provides an average physical separation between said second fluorescent dye moiety and said nucleotide of at least about 30 Angstroms (Å). In some embodiments, said second linker provides an average physical separation between said second fluorescent dye moiety and said nucleotide of at least about 60 Angstroms (Å). In some embodiments, said second linker provides an average physical separation between said second fluorescent dye moiety and said nucleotide of at least about 90 Angstroms (Å).

In some embodiments, said solution comprises a plurality of fluorescently labeled nucleotides, wherein each fluorescently labeled nucleotide of said plurality of said fluorescently labeled nucleotides comprises (i) a fluorescent labeling reagent comprising a plurality of fluorescent dye moieties of a same type and a plurality of linkers of a same type, and (ii) a nucleotide of a same type. In some embodiments, each said linker of each fluorescently labeled nucleotide of said plurality of fluorescently labeled nucleotides has the same molecular weight. In some embodiments, said solution further comprises a plurality of unlabeled nucleotides, wherein each nucleotide of said plurality of unlabeled nucleotides is of a same type as each said nucleotide of said plurality of fluorescently labeled nucleotides. In some embodiments, the ratio of said plurality of fluorescently labeled nucleotides to said plurality of unlabeled nucleotides in said solution is at least about 1:4. In some embodiments, said ratio is at least about 1:1.

In a further aspect, the present disclosure provides a method comprising providing a composition described herein to a template nucleic acid molecule coupled to a nucleic acid strand.

In some embodiments, the method further comprises subjecting said template nucleic acid molecule and said composition to conditions sufficient to incorporate said fluorescently labeled nucleotide into said nucleic acid strand coupled to said template nucleic acid molecule. In some embodiments, the method further comprises detecting a signal from said fluorescently labeled nucleotide. In some embodiments, the method further comprises contacting said fluorescently labeled nucleotide with a cleavage reagent configured to cleave said plurality of fluorescent dye moieties from said nucleotide. In some embodiments, the method further comprises, subsequent to said contacting said fluorescently labeled nucleotide with said cleavage reagent, subjecting said template nucleic acid molecule and said composition to conditions sufficient to incorporate an additional fluorescently labeled nucleotide into said nucleic acid strand coupled to said template nucleic acid molecule.

In some embodiments, said template nucleic acid molecule is immobilized to a support.

In another aspect, the present disclosure provides a method, comprising: (a) providing a fluorescent labeling reagent described herein; and (b) contacting said fluorescent labeling reagent with a substrate to generate a fluorescently labeled substrate.

In some embodiments, the method further comprises repeating (a) and (b) one or more times with one or more additional fluorescent labeling reagents to provide a fluorescently labeled substrate comprising said fluorescent labeling reagent and said one or more additional fluorescent labeling reagents. In some embodiments, (a) and (b) are repeated at least two times with at least two additional fluorescent labeling reagents. In some embodiments, said at least two additional fluorescent labeling reagents and said fluorescent labeling reagent have identical chemical structures. In some embodiments, at least one of said at least two additional fluorescent labeling reagents and said fluorescent labeling reagent have different chemical structures.

In some embodiments, the method further comprises contacting said fluorescently labeled substrate with a cleavage reagent configured to cleave said fluorescent labeling reagent or a portion thereof from said fluorescently labeled substrate to generate a scarred substrate. In some embodiments, the method further comprises, prior to generating said scarred substrate, subjecting said fluorescently labeled substrate and a nucleic acid molecule to conditions sufficient to incorporate said fluorescently labeled substrate into said nucleic acid molecule. In some embodiments, the method further comprises, prior to generating said scarred substrate, subjecting an additional substrate and said nucleic acid molecule to conditions sufficient to incorporate said additional substrate into said nucleic acid molecule at a position adjacent to said substrate. In some embodiments, the method further comprises, subsequent to generating said scarred substrate, subjecting an additional substrate and said nucleic acid molecule to conditions sufficient to incorporate said additional substrate into said nucleic acid molecule at a position adjacent to said scarred substrate. In some embodiments, said additional substrate does not comprise said fluorescent labeling reagent. In some embodiments, said additional substrate comprises said fluorescent labeling reagent. In some embodiments, the method further comprises, prior to generating said scarred substrate, detecting a signal from said fluorescently labeled substrate.

In some embodiments, said substrate is a nucleotide, polynucleotide, protein, lipid, cell, saccharide, polysaccharide, or antibody. In some embodiments, said substrate is a protein. In some embodiments, said protein is a component of a cell. In some embodiments, said protein is an antibody.

In some embodiments, said fluorescently labeled substrate is immobilized to a support.

In another aspect, the present disclosure provides a kit comprising a plurality of fluorescent labeling reagents described herein.

In some embodiments, said plurality of fluorescent labeling reagents are coupled to one or more substrates. In some embodiments, said plurality of fluorescent labeling reagents are coupled to a single substrate. In some embodiments, a substrate of said one or more substrates comprises at least two fluorescent labeling reagents of said plurality of fluorescent labeling reagents coupled thereto. In some embodiments, said one or more substrates are of different types. In some embodiments, said one or more substrates comprise one or more proteins or antibodies. In some embodiments, said one or more substrates comprise one or more nucleotides. In some embodiments, said one or more substrates comprise a plurality of nucleotides of a first type and a plurality of nucleotides of a second type. In some embodiments, said one or more substrates further comprise a plurality of nucleotides of a third type and a plurality of nucleotides of a fourth type.

In some embodiments, each fluorescent labeling reagent of said plurality of fluorescent labeling reagents comprises an identical chemical structure. In some embodiments, said plurality of fluorescent labeling reagents comprises a first fluorescent labeling reagent having a first chemical structure and a second fluorescent labeling reagent having a second chemical structure, wherein said first chemical structure and said second chemical structure are different. In some embodiments, said first fluorescent labeling reagent includes a first fluorescent dye moiety and said second fluorescent labeling reagent comprises a second fluorescent dye moiety, wherein said first fluorescent dye moiety and said second fluorescent dye moiety have different chemical structures.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a method for constructing a labeled nucleotide comprising a propargyl-derivatized nucleotide, a linker, and a dye.

FIGS. 2A and 2B show an example method for preparing a labeled nucleotide comprising a dGTP analog.

FIGS. 3A-3C show an example method for preparing a labeled nucleotide comprising a guanine analog.

FIG. 4 shows components that may be used to construct dye-labeled nucleotides.

FIG. 5 shows an example fluorescent labeling reagent.

FIG. 6 shows an example method for preparing a labeled nucleotide comprising a guanine analog.

FIG. 7 shows an example sequencing procedure.

FIG. 8 shows a schematic of a bead-based assay for evaluating labeled nucleotides.

FIG. 9 shows results of a bead-based assay for different labeled dUTPs.

FIG. 10 shows results of a bead-based assay for different labeled dATPs.

FIG. 11 shows results of a bead-based assay for different labeled dGTPs.

FIG. 12 shows tolerances of different labeled nucleotides.

FIG. 13 shows a schematic of an assay for evaluating quenching.

FIG. 14 shows quenching results for red dye linkers.

FIG. 15 shows quenching results for green dye linkers.

FIGS. 16A and 16B show examples of incorporation into templates including homopolymeric regions.

FIG. 16C shows signals detected from sequencing a template having a homopolymeric region using labeled nucleotides.

FIG. 17A shows example results of a sequencing analysis utilizing populations of nucleotides comprising 20% fluorophore labeled dNTPs.

FIG. 17B shows fluorescence signal intensity as a function of homopolymer length.

FIG. 18 shows example results of a sequencing analysis utilizing populations of nucleotides comprising 100% fluorophore labeled dNTPs.

FIGS. 19A-19D show signals measured for cystosine, adenine, thymine, and guanine-containing homopolymer sequences using fluorescently labeled nucleotides.

FIG. 20 shows fluorescence of bovine serum albumin labeled with different fluorescent labeling moieties.

FIGS. 21A and 21B show examples of portions of fluorescent labeling reagents including two or more fluorescent dye moieties, while FIG. 21C shows their respective quantum yields. FIG. 21D shows additional examples of portions of fluorescent labeling reagents including two or more fluorescent dye moieties.

FIG. 21E shows a portion of a fluorescent labeling reagent including nine fluorescent dye moieties.

FIGS. 22A and 22B show measured brightness of streptavidin (FIG. 22A) and mouse antibody (FIG. 22B) labeled with different fluorescent labeling moieties.

FIG. 23 shows example dye structures for inclusion in optical labeling reagents.

FIG. 24 shows sequencing data for labeled uracil-containing nucleotides including different cleavable moieties.

FIG. 25 shows brightness (left panel) and homopolymeric incorporation (right panel) for different labeled uracil-containing nucleotides.

FIG. 26 shows relative fluorescence of green and red dyes.

FIGS. 27A-27B show relative fluorescence as a function of homopolymer length.

FIGS. 28A and 28B show sequencing data for sequencing assays performed with varying labeling fractions.

FIG. 29 shows a computer system that is programmed or otherwise configured to implement methods provided herein.

FIG. 30 illustrates an example of the relationship between the excitation spectrum, emission spectrum, and fluorescent intensity of a donor-acceptor fluorophore pair in tandem labeling.

FIG. 31 illustrates an example labeling agent.

FIG. 32 illustrates multiple labeling agents for labeling multiple molecules using one emission laser.

FIG. 33 illustrates two example labeling agents.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “about” and “approximately” shall generally mean an acceptable degree of error or variation for a given value or range of values, such as, for example, a degree of error or variation that is within 20 percent (%), within 15%, within 10%, or within 5% of a given value or range of values.

The term “subject,” as used herein, generally refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. A subject may be a patient. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. Alternatively or additionally, a subject may be known to have previously had a disease or disorder. The subject may have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index, or other physical characteristics. A subject may have a given ethnic or racial heritage, place of birth or residence, nationality, disease or remission state, family medical history, or other characteristics.

As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy, or skin biopsy. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or additionally, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

A sample that is not a cell-free sample (e.g., a sample comprising one or more cells) may be processed to provide a cell-free sample. For example, a sample that includes one or more cells as well as one or more nucleic acid molecules (e.g., DNA and/or RNA molecules) not included within cells (e.g., cell-free nucleic acid molecules) may be obtained from a subject. The sample may be subjected to processing (e.g., as described herein) to separate cells and other materials from the nucleic acid molecules not included within cells, thereby providing a cell-free sample (e.g., comprising nucleic acid molecules not included within cells). The cell-free sample may then be subjected to further analysis and processing (e.g., as provided herein). Nucleic acid molecules not included within cells (e.g., cell-free nucleic acid molecules) may be derived from cells and tissues. For example, cell-free nucleic acid molecules may derive from a tumor tissue or a degraded cell (e.g., of a tissue of a body). Cell-free nucleic acid molecules may comprise any type of nucleic acid molecules (e.g., as described herein). Cell-free nucleic acid molecules may be double-stranded, single-stranded, or a combination thereof. Cell-free nucleic acid molecules may be released into a bodily fluid through secretion or cell death processes, e.g., cellular necrosis, apoptosis, or the like. Cell-free nucleic acid molecules may be released into bodily fluids from cancer cells (e.g., circulating tumor DNA (ctDNA)). Cell free nucleic acid molecules may also be fetal DNA circulating freely in a maternal blood stream (e.g., cell-free fetal nucleic acid molecules such as cffDNA). Alternatively or additionally, cell-free nucleic acid molecules may be released into bodily fluids from healthy cells.

A biological sample may be obtained directly from a subject and analyzed without any intervening processing, such as, for example, sample purification or extraction. For example, a blood sample may be obtained directly from a subject by accessing the subject's circulatory system, removing the blood from the subject (e.g., via a needle), and transferring the removed blood into a receptacle. The receptacle may comprise reagents (e.g., anti-coagulants) such that the blood sample is useful for further analysis. Such reagents may be used to process the sample or analytes derived from the sample in the receptacle or another receptacle prior to analysis. In another example, a swab may be used to access epithelial cells on an oropharyngeal surface of the subject. Following obtaining the biological sample from the subject, the swab containing the biological sample may be contacted with a fluid (e.g., a buffer) to collect the biological fluid from the swab.

Any suitable biological sample that comprises one or more nucleic acid molecules may be obtained from a subject. A sample (e.g., a biological sample or cell-free biological sample) suitable for use according to the methods provided herein may be any material comprising tissues, cells, degraded cells, nucleic acids, genes, gene fragments, expression products, gene expression products, and/or gene expression product fragments of an individual to be tested. A biological sample may be solid matter (e.g., biological tissue) or may be a fluid (e.g., a biological fluid). In general, a biological fluid may include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. Methods for determining sample suitability and/or adequacy are provided. A sample may include, but is not limited to, blood, plasma, tissue, cells, degraded cells, cell-free nucleic acid molecules, and/or biological material from cells or derived from cells of an individual such as cell-free nucleic acid molecules. The sample may be a heterogeneous or homogeneous population of cells, tissues, or cell-free biological material. The biological sample may be obtained using any method that can provide a sample suitable for the analytical methods described herein.

A sample (e.g., a biological sample or cell-free biological sample) may undergo one or more processes in preparation for analysis, including, but not limited to, filtration, centrifugation, selective precipitation, permeabilization, isolation, agitation, heating, purification, and/or other processes. For example, a sample may be filtered to remove contaminants or other materials. In an example, a sample comprising cells may be processed to separate the cells from other material in the sample. Such a process may be used to prepare a sample comprising only cell-free nucleic acid molecules. Such a process may consist of a multi-step centrifugation process. Multiple samples, such as multiple samples from the same subject (e.g., obtained in the same or different manners from the same or different bodily locations, and/or obtained at the same or different times (e.g., seconds, minutes, hours, days, weeks, months, or years apart)) or multiple samples from different subjects may be obtained for analysis as described herein. In an example, the first sample is obtained from a subject before the subject undergoes a treatment regimen or procedure and the second sample is obtained from the subject after the subject undergoes the treatment regimen or procedure. Alternatively or additionally, multiple samples may be obtained from the same subject at the same or approximately the same time. Different samples obtained from the same subject may be obtained in the same or different manner. For example, a first sample may be obtained via a biopsy and a second sample may be obtained via a blood draw. Samples obtained in different manners may be obtained by different medical professionals, using different techniques, at different times, and/or at different locations. Different samples obtained from the same subject may be obtained from different areas of a body. For example, a first sample may be obtained from a first area of a body (e.g., a first tissue) and a second sample may be obtained from a second area of the body (e.g., a second tissue).

A biological sample as used herein (e.g., a biological sample comprising one or more nucleic acid molecules) may not be purified when provided in a reaction vessel. Furthermore, for a biological sample comprising one or more nucleic acid molecules, the one or more nucleic acid molecules may not be extracted when the biological sample is provided to a reaction vessel. For example, ribonucleic acid (RNA) and/or deoxyribonucleic acid (DNA) molecules of a biological sample may not be extracted from the biological sample when providing the biological sample to a reaction vessel. Moreover, a target nucleic acid (e.g., a target RNA or target DNA molecules) present in a biological sample may not be concentrated when providing the biological sample to a reaction vessel. Alternatively, a biological sample may be purified and/or nucleic acid molecules may be isolated from other materials in the biological sample.

A biological sample as described herein may contain a target nucleic acid. As used herein, the terms “template nucleic acid,” “target nucleic acid,” “nucleic acid molecule,” “nucleic acid sequence,” “nucleic acid fragment,” “oligonucleotide,” “polynucleotide,” and “nucleic acid” generally refer to polymeric forms of nucleotides of any length, such as deoxyribonucleotides (dNTPs) or ribonucleotides (rNTPs), or analogs thereof, and may be used interchangeably. Nucleic acids may have any three-dimensional structure, and may perform any function, known or unknown. A nucleic acid molecule may have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

A target nucleic acid or sample nucleic acid as described herein may be amplified to generate an amplified product. A target nucleic acid may be a target RNA or a target DNA. When the target nucleic acid is a target RNA, the target RNA may be any type of RNA, including types of RNA described elsewhere herein. The target RNA may be viral RNA and/or tumor RNA. A viral RNA may be pathogenic to a subject. Non-limiting examples of pathogenic viral RNA include human immunodeficiency virus I (HIV I), human immunodeficiency virus n (HIV 11), orthomyxoviruses, Ebola virus. Dengue virus, influenza viruses (e.g., H1N1, H3N2, H7N9, or H5N1), herpesvirus, hepatitis A virus, hepatitis B virus, hepatitis C (e.g., armored RNA-HCV virus) virus, hepatitis D virus, hepatitis E virus, hepatitis G virus, Epstein-Barr virus, mononucleosis virus, cytomegalovirus, SARS virus, West Nile Fever virus, polio virus, and measles virus.

A biological sample may comprise a plurality of target nucleic acid molecules. For example, a biological sample may comprise a plurality of target nucleic acid molecules from a single subject. In another example, a biological sample may comprise a first target nucleic acid molecule from a first subject and a second target nucleic acid molecule from a second subject.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide).

The term “nucleotide analog,” as used herein, may include, but is not limited to, a nucleotide that may or may not be a naturally occurring nucleotide. For example, a nucleotide analog may be derived from and/or include structural similarities to a canonical nucleotide such as adenine—(A), thymine—(T), cytosine—(C), uracil—(U), or guanine—(G) including nucleotide. A nucleotide analog may comprise one or more differences or modifications relative to a natural nucleotide. Examples of nucleotide analogs include inosine, diaminopurine, 5-fluorouracil, 5-bromouracil, 5-chlorouracil, 5-iodouracil, hypoxanthine, xanthine, deazaxanthine, deazaguanine, isocytosine, isoguanine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids, and modified versions thereof (e.g., by oxidation, reduction, and/or addition of a substituent such as an alkyl, hydroxyalkyl, hydroxyl, or halogen moiety). Nucleic acid molecules (e.g., polynucleotides, double-stranded nucleic acid molecules, single-stranded nucleic acid molecules, primers, adapters, etc.) may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some cases, a nucleotide may include a modification in its phosphate moiety, including a modification to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates), and modifications with selenium moieties (e.g., phosphoroselenoate nucleic acids). A nucleotide or nucleotide analog may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide analog may also comprise a modified linker moiety (e.g., in lieu of a phosphate moiety). Nucleotide analogs may also contain amine-modified groups, such as aminoallyl-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide, for example, higher density in bits per cubic mm, higher safety (resistant to accidental or purposeful synthesis of natural toxins), easier discrimination in photo-programmed polymerases, and/or lower secondary structure. Nucleotide analogs may be capable of reacting or bonding with detectable moieties for nucleotide detection.

The term “homopolymer,” as used herein, generally refers to a polymer or a portion of a polymer comprising identical monomer units. A homopolymer may have a homopolymer sequence. A nucleic acid homopolymer may refer to a polynucleotide or an oligonucleotide comprising consecutive repetitions of a same nucleotide or any nucleotide variants thereof. For example, a homopolymer can be poly(dA), poly(dT), poly(dG), poly(dC), poly(rA), poly(U), poly(rG), or poly(rC). A homopolymer can be of any length. For example, the homopolymer can have a length of at least 2, 3, 4, 5, 10, 20, 30, 40, 50, 100, 200, 300, 400, 500, or more nucleic acid bases. The homopolymer can have from 10 to 500, or 15 to 200, or 20 to 150 nucleic acid bases. The homopolymer can have a length of at most 500, 400, 300, 200, 100, 50, 40, 30, 20, 10, 5, 4, 3, or 2 nucleic acid bases. A molecule, such as a nucleic acid molecule, can include one or more homopolymer portions and one or more non-homopolymer portions. The molecule may be entirely formed of a homopolymer, multiple homopolymers, or a combination of homopolymers and non-homopolymers. In nucleic acid sequencing, multiple nucleotides can be incorporated into a homopolymeric region of a nucleic acid strand. Such nucleotides may be non-terminated to permit incorporation of consecutive nucleotides (e.g., during a single nucleotide flow).

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. An amplicon may be a single-stranded or double-stranded nucleic acid molecule that is generated by an amplification procedure from a starting template nucleic acid molecule. Such an amplification procedure may include one or more cycles of an extension or ligation procedure. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. Amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification, and multiple displacement amplification (MDA). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and touchdown PCR. Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety.

Amplification may be clonal amplification. The term “clonal,” as used herein, generally refers to a population of nucleic acids for which a substantial portion (e.g., greater than about 50%, 60%, 70%, 80%, 90%, 95%, or 99%) of its members have sequences that are at least about 50%, 60%, 70%, 80%, 90%, 95%, or 99% identical to one another. Members of a clonal population of nucleic acid molecules may have sequence homology to one another. Such members may have sequence homology to a template nucleic acid molecule. The members of the clonal population may be double stranded or single stranded. Members of a population may not be 100% identical or complementary, e.g., “errors” may occur during the course of synthesis such that a minority of a given population may not have sequence homology with a majority of the population. For example, at least 50% of the members of a population may be substantially identical to each other or to a reference nucleic acid molecule (i.e., a molecule of defined sequence used as a basis for a sequence comparison). At least 60%, at least 70%, at least 80%, at least 90%, at least 95%, at least 99%, or more of the members of a population may be substantially identical to the reference nucleic acid molecule. Two molecules may be considered substantially identical (or homologous) if the percent identity between the two molecules is at least 60%, 70%, 75%, 80%, 85%, 90%, 95%, 98%, 99%, 99.9% or greater. Two molecules may be considered substantially complementary if the percent complementarity between the two molecules is at least 60%, 70%, 75%, 80%, 85%, 90%, 95%, 98%, 99%, 99.9% or greater. A low or insubstantial level of mixing of non-homologous nucleic acids may occur, and thus a clonal population may contain a minority of diverse nucleic acids (e.g., less than 30%, e.g., less than 10%).

Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). The enhanced signal-to-noise ratio provided by clonal amplification more than outweighs the disadvantages of the cyclic sequencing requirement.

The term “polymerizing enzyme” or “polymerase,” as used herein, generally refers to any enzyme capable of catalyzing a polymerization reaction. A polymerizing enzyme may be used to extend a nucleic acid primer paired with a template strand by incorporation of nucleotides or nucleotide analogs. A polymerizing enzyme may add a new strand of DNA by extending the 3′ end of an existing nucleotide chain, adding new nucleotides matched to the template strand one at a time via the creation of phosphodiester bonds. The polymerase used herein can have strand displacement activity or non-strand displacement activity. Examples of polymerases include, without limitation, a nucleic acid polymerase. An example polymerase is a Φ29 DNA polymerase or a derivative thereof. A polymerase can be a polymerization enzyme. In some cases, a transcriptase or a ligase is used (i.e., enzymes which catalyze the formation of a bond). Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfu-turbo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. In some cases, the polymerase is a single subunit polymerase. The polymerase can have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al, PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such as for example, Sequenase DNA polymerase (ThermoFisher).

A polymerase may be a Family A polymerase or a Family B DNA polymerase. Family A polymerases include, for example, Taq, Klenow, and Bst polymerases. Family B polymerases include, for example, Vent(exo-) and Therminator polymerases. Family B polymerases are known to accept more varied nucleotide substrates than Family A polymerases. Family A polymerases are used widely in sequencing by synthesis methods, likely due to their high processivity and fidelity.

The term “complementary sequence,” as used herein, generally refers to a sequence that hybridizes to another sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds.

The term “denaturation,” as used herein, generally refers to separation of a double-stranded molecule (e.g., DNA) into single-stranded molecules. Denaturation may be complete or partial denaturation. In partial denaturation, a single-stranded region may form in a double-stranded molecule by denaturation of the two deoxyribonucleic acid (DNA) strands flanked by double-stranded regions in DNA.

The term “melting temperature” or “melting point,” as used herein, generally refers to the temperature at which at least a portion of a strand of a nucleic acid molecule in a sample has separated from at least a portion of a complementary strand. The melting temperature may be the temperature at which a double-stranded nucleic acid molecule has partially or completely denatured. The melting temperature may refer to a temperature of a sequence among a plurality of sequences of a given nucleic acid molecule, or a temperature of the plurality of sequences. Different regions of a double-stranded nucleic acid molecule may have different melting temperatures. For example, a double-stranded nucleic acid molecule may include a first region having a first melting point and a second region having a second melting point that is higher than the first melting point. Accordingly, different regions of a double-stranded nucleic acid molecule may melt (e.g., partially denature) at different temperatures. The melting point of a nucleic acid molecule or a region thereof (e.g., a nucleic acid sequence) may be determined experimentally (e.g., via a melt analysis or other procedure) or may be estimated based upon the sequence and length of the nucleic acid molecule. For example, a software program such as MELTING may be used to estimate a melting temperature for a nucleic acid sequence (Dumousseau M, Rodriguez N, Juty N, Le Novère N, MELTING, a flexible platform to predict the melting temperatures of nucleic acids. BMC Bioinformatics. 2012 May 16; 13:101. doi: 10.1186/1471-2105-13-101). Accordingly, a melting point as described herein may be an estimated melting point. A true melting point of a nucleic acid sequence may vary based upon the sequences or lack thereof adjacent to the nucleic acid sequence of interest as well as other factors.

The term “sequencing,” as used herein, generally refers to a process for generating or identifying a sequence of a biological molecule, such as a nucleic acid molecule or a polypeptide. Such sequence may be a nucleic acid sequence, which may include a sequence of nucleic acid bases (e.g., nucleobases). Sequencing may be, for example, single molecule sequencing, sequencing by synthesis, sequencing by hybridization, or sequencing by ligation. Sequencing may be performed using template nucleic acid molecules immobilized on a support, such as a flow cell or one or more beads. A sequencing assay may yield one or more sequencing reads corresponding to one or more template nucleic acid molecules.

The term “read,” as used herein, generally refers to a nucleic acid sequence, such as a sequencing read. A sequencing read may be an inferred sequence of nucleic acid bases (e.g., nucleotides) or base pairs obtained via a nucleic acid sequencing assay. A sequencing read may be generated by a nucleic acid sequencer, such as a massively parallel array sequencer (e.g., Illumina or Pacific Biosciences of California). A sequencing read may correspond to a portion, or in some cases all, of a genome of a subject. A sequencing read may be part of a collection of sequencing reads, which may be combined through, for example, alignment (e.g., to a reference genome), to yield a sequence of a genome of a subject.

The term “detector,” as used herein, generally refers to a device that is capable of detecting or measuring a signal, such as a signal indicative of the presence or absence of an incorporated nucleotide or nucleotide analog. A detector may include optical and/or electronic components that may detect and/or measure signals. Non-limiting examples of detection methods involving a detector include optical detection, spectroscopic detection, electrostatic detection, and electrochemical detection. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

The term “support,” as used herein, generally refers to any solid or semi-solid article on which reagents such as nucleic acid molecules may be immobilized. Nucleic acid molecules may be synthesized, attached, ligated, or otherwise immobilized. Nucleic acid molecules may be immobilized on a support by any method including, but not limited to, physical adsorption, by ionic or covalent bond formation, or combinations thereof. A support may be 2-dimensional (e.g., a planar 2D support) or 3-dimensional. In some cases, a support may be a component of a flow cell and/or may be included within or adapted to be received by a sequencing instrument. A support may include a polymer, a glass, or a metallic material. Examples of supports include a membrane, a planar support, a microtiter plate, a bead (e.g., a magnetic bead), a filter, a test strip, a slide, a cover slip, and a test tube. A support may comprise organic polymers such as polystyrene, polyethylene, polypropylene, polyfluoroethylene, polyethyleneoxy, and polyacrylamide (e.g., polyacrylamide gel), as well as co-polymers and grafts thereof. A support may comprise latex or dextran. A support may also be inorganic, such as glass, silica, gold, controlled-pore-glass (CPG), or reverse-phase silica. The configuration of a support may be, for example, in the form of beads, spheres, particles, granules, a gel, a porous matrix, or a support. In some cases, a support may be a single solid or semi-solid article (e.g., a single particle), while in other cases a support may comprise a plurality of solid or semi-solid articles (e.g., a collection of particles). Supports may be planar, substantially planar, or non-planar. Supports may be porous or non-porous. Supports may have swelling or non-swelling characteristics. A support may be shaped to comprise one or more wells, depressions, or other containers, vessels, features, or locations. A plurality of supports may be configured in an array at various locations. A support may be addressable (e.g., for robotic delivery of reagents), or by detection approaches, such as scanning by laser illumination and confocal or deflective light gathering. For example, a support may be in optical and/or physical communication with a detector. Alternatively, a support may be physically separated from a detector by a distance. An amplification support (e.g., a bead) can be placed within or on another support (e.g., within a well of a second support).

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. A coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction. In another example, a particle may be coupled to a planar support via a magnetic interaction. In another example, a particle may be coupled to a planar support via a covalent interaction. Similarly, a nucleic acid molecule may be coupled to a particle via a covalent interaction. Alternatively or additionally, a nucleic acid molecule may be coupled to a particle via a non-covalent interaction. A coupling between a first object and a second object may comprise a labile moiety, such as a moiety comprising an ester, vicinal diol, phosphodiester, peptidic, glycosidic, sulfone, Diels-Alder, or similar linkage. The strength of a coupling between a first object and a second object may be indicated by a dissociation constant, Kd, that indicates the inclination of a coupled object comprising a first object and a second object to dissociate into the uncoupled first and second objects and may be expressed as a ratio of dissociated (e.g., uncoupled) objects to coupled objects. A smaller dissociation constant is generally indicative of a stronger coupling between coupled objects.

Coupled objects and their corresponding uncoupled components may exist in dynamic equilibrium with one another. For example, a solution comprising a plurality of coupled objects each comprising a first object and a second object may also include a plurality of first objects and a plurality of second objects. At a given point in time, a given first object and a given second object may be coupled to one another or the objects may be uncoupled; the relative concentrations of coupled and uncoupled components throughout the solution will depend upon the strength of the coupling between the first and second objects (reflected in the dissociation constant). For example, a binding moiety may be coupled to a nucleic acid molecule to provide a binding complex. In a solution comprising a plurality of binding complexes each comprising a binding moiety coupled to a nucleic acid molecule, the plurality of binding complexes may exist in equilibrium with their constituent nucleic acid molecules and binding moieties. The association between a given nucleic acid molecule and a given binding moiety may be such that, at a given point in time, at least 50%, such as at least 60%, 65%, 70%, 75%, 80%, 85%, 90%, 95%, 98%, or more, of the nucleic acid molecules may be components of a binding complex of the plurality of binding complexes.

The term “label,” as used herein, generally refers to a moiety that is capable of coupling with a species, such as, for example a nucleotide analog. A label may include an affinity moiety. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), tris(hydroxypropyl)phosphine (THP) or enzymatically cleavable (e.g., via an esterase, lipase, peptidase or protease). In some cases, the label may be luminescent; that is, fluorescent or phosphorescent. For example, the label may be or comprise a fluorescent moiety (e.g., a dye). Dyes and labels may be incorporated into nucleic acid sequences. Dyes and labels may also be incorporated into or attached to linkers, such as linkers for linking one or more beads to one another. For example, labels such as fluorescent moieties may be linked to nucleotides or nucleotide analogs via a linker (e.g., as described herein). Non-limiting examples of dyes include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO labels (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); and SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor labels (e.g., AlexaFluor 350, 405, 430, 488, 532, 546, 555, 568, 594, 610, 633, 635, 647, 660, 680, 700, 750, and 790 dyes), DyLight labels (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, CySQ, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, ATTO 580Q, ATTO 612Q, Atto532 [e.g., Atto 532 succinimidyl ester], and Atto633), and other fluorophores and/or quenchers. Additional examples are included in structures provided herein. Dyes included in structures provided herein are contemplated for use in combination with any linker and substrate described herein. A fluorescent dye may be excited by application of energy corresponding to the visible region of the electromagnetic spectrum (e.g., between about 430-770 nanometers (nm)). Excitation may be done using any useful apparatus, such as a laser and/or light emitting diode. Optical elements including, but not limited to, mirrors, waveplates, filters, monochromators, gratings, beam splitters, and lenses may be used to direct light to or from a fluorescent dye. A fluorescent dye may emit light (e.g., fluoresce) in the visible region of the electromagnetic spectrum ((e.g., between about 430-770 nm). A fluorescent dye may be excited over a single wavelength or a range of wavelengths. A fluorescent dye may be excitable by light in the red region of the visible portion of the electromagnetic spectrum (about 625-740 nm) (e.g., have an excitation maximum in the red region of the visible portion of the electromagnetic spectrum). Alternatively or additionally, fluorescent dye may be excitable by light in the green region of the visible portion of the electromagnetic spectrum (about 500-565 nm) (e.g., have an excitation maximum in the green region of the visible portion of the electromagnetic spectrum). A fluorescent dye may emit signal in the red region of the visible portion of the electromagnetic spectrum (about 625-740 nm) (e.g., have an emission maximum in the red region of the visible portion of the electromagnetic spectrum). Alternatively or additionally, fluorescent dye may emit signal in the green region of the visible portion of the electromagnetic spectrum (about 500-565 nm) (e.g., have an emission maximum in the green region of the visible portion of the electromagnetic spectrum).

Labels may be quencher molecules. The term “quencher,” as used herein, generally refers to molecules that may be energy acceptors. A quencher may be a molecule that can reduce an emitted signal. For example, a template nucleic acid molecule may be designed to emit a detectable signal. Incorporation of a nucleotide or nucleotide analog comprising a quencher can reduce or eliminate the signal, which reduction or elimination is then detected. Luminescence from labels (e.g., fluorescent moieties, such as fluorescent moieties linked to nucleotides or nucleotide analogs) may also be quenched (e.g., by incorporation of other nucleotides that may or may not comprise labels). In some cases, as described elsewhere herein, labelling with a quencher can occur after nucleotide or nucleotide analog incorporation (e.g., after incorporation of a nucleotide or nucleotide analog comprising a fluorescent moiety). In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other. Examples of quenchers include, but are not limited to, Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), and ATTO fluorescent quenchers (ATTO-TEC GmbH) (e.g., ATTO 540Q, ATTO 580Q, and ATTO 612Q). Fluorophore donor molecules may be used in conjunction with a quencher. Examples of fluorophore donor molecules that can be used in conjunction with quenchers include, but are not limited to, fluorophores such as Cy3B, Cy3, or Cy5; Dy-Quenchers (Dyomics) (e.g., DYQ-660 and DYQ-661); and ATTO fluorescent quenchers (ATTO-TEC GmbH) (e.g., ATTO 540Q, 580Q, and 612Q).

The term “labeling fraction,” as used herein, generally refers to the ratio of dye-labeled nucleotide or nucleotide analog to natural/unlabeled nucleotide or nucleotide analog of a single canonical type in a flow solution. The labeling fraction can be expressed as the concentration of the labeled nucleotide or nucleotide analog divided by the sum of the concentrations of labeled and unlabeled nucleotide or nucleotide analog. The labeling fraction may be expressed as a % of labeled nucleotides included in a solution (e.g., a nucleotide flow). The labeling fraction may be at least about 0.5%, 1%, 2%, 3%, 4%, 5%, 10%, 15%, 20%, 30%, 40%, 50%, 60%, 70%, 80%, 90%, or higher. For example, the labeling fraction may be at least about 20%. The labeling fraction may be about 100%. The labeling fraction may also be expressed as a ratio of labeled nucleotides to unlabeled nucleotides included in a solution. For example, the ratio of labeled nucleotides to unlabeled nucleotides may be at least about 1:5, 1:4, 1:3, 1:2, 1:1, 2:1, 3:1, 4:1, 5:1, or higher. For example, the ratio of labeled nucleotides to unlabeled nucleotides may be at least about 1:4. For example, the ratio of labeled nucleotides to unlabeled nucleotides may be at least about 1:1. For example, the ratio of labeled nucleotides to unlabeled nucleotides may be at least about 5:1.

The term “labeled fraction,” as used herein, generally refers to the actual fraction of labeled nucleic acid (e.g., DNA) resulting after treatment of a primer-template with a mixture of the dye-labeled and natural nucleotide or nucleotide analog. The labeled fraction may be about the same as the labeling fraction. For example, if 20% of nucleotides in a nucleotide flow are labeled, about 20% of nucleotides incorporated into a growing nucleic acid strand (e.g., during nucleic acid sequencing) may be labeled. Alternatively, the labeled fraction may be greater than the labeled fraction. For example, if 20% of nucleotides in a nucleotide flow are labeled, greater than 20% of nucleotides incorporated into a growing nucleic acid strand (e.g., during nucleic acid sequencing) may be labeled. Alternatively, the labeled fraction may be less than the labeled fraction. For example, if 20% of nucleotides in a nucleotide flow are labeled, less than 20% of nucleotides incorporated into a growing nucleic acid strand (e.g., during nucleic acid sequencing) may be labeled.

When a solution including less than 100% labeled nucleotides or nucleotide analogs is used in an incorporation process such as a sequencing process (e.g., as described herein), both labeled (“bright”) and unlabeled (“dark”) nucleotides or nucleotide analogs may be incorporated into a growing nucleic acid strand. The term “tolerance,” as used herein, generally refers to the ratio of the labeled fraction (e.g., “bright” incorporated fraction) to the labeling fraction (e.g., “bright” fraction in solution). For example, if a labeling fraction of 0.2 is used resulting in a labeled fraction of 0.4 the tolerance is 2. Similarly, if an incorporation process such as a sequencing process is performed using 2.5% labeled fraction in solution (b_{f}, bright solution fraction) and 5% is labeled (bᵢ, bright incorporated fraction), the tolerance may be 2 (e.g., tolerance). This model may be linear for low labeling fractions (e.g., 10% or lower labeling fraction). For higher labeling fractions, tolerance may take into account competing dark incorporation. Tolerance may refer to a comparison of the ratio of bright incorporated fraction to dark incorporated fraction (bᵢ/dᵢ) to the ratio of bright solution fraction to dark solution fraction (b_{f}/d_{f}):

$$Tolerance = \frac{{b}_{i} / {d}_{i}}{{b}_{f} / {d}_{f}}$$

where dᵢ=1−bᵢ (e.g., dark incorporated fraction and bright incorporated fraction sum to 1 assuming 100% bright fraction is normalized to 1)

Though dᵢ cannot easily be measured, bᵢ, the bright incorporated fraction, can be measured (e.g., as described herein) and used to determine tolerance by fitting a curve of bright solution fraction (b_{f}) vs. bright incorporated fraction (bᵢ):

$${b}_{i} = \frac{tol ( {b}_{f} / {d}_{f} )}{1 + tol ( {b}_{f} / {d}_{f} )}$$

A “positive” tolerance number (>1) indicates that at 50% labeling fraction, more than 50% is labeled. A “negative” tolerance number (<1) indicates that at 50% labeling fraction, less than 50% is labeled.

The term “context,” as used herein, generally refers to the sequence of the neighboring nucleotides, or context, has been observed to affect the tolerance in an incorporation reaction. The nature of the enzyme, the pH, and other factors may also affect the tolerance. Reducing context effects to a minimum greatly simplifies base determination.

The term “scar,” as used herein, generally refers to a residue left on a previously labeled nucleotide or nucleotide analog after cleavage of an optical (e.g., fluorescent) dye and, optionally, all or a portion of a linker attaching the optical dye to the nucleotide or nucleotide analog. Examples of scars include, but are not limited to, hydroxyl moieties (e.g., resulting from cleavage of an azidomethyl group, hydrocarbyldithiomethyl linkage, or 2-nitrobenzyloxy linkage), thiol moieties (e.g., resulting from cleavage of a disulfide linkage), and benzyl moieties. For example, a scar may comprise an aromatic group such as a phenyl or benzyl group. The size and nature of a scar may affect subsequent incorporations.

The term “misincorporation,” as used herein, generally refers to occurrences when the DNA polymerase incorporates a nucleotide, either labeled or unlabeled, that is not the correct Watson-Crick partner for the template base. Misincorporation can occur more frequently in methods that lack competition of all four bases in an incorporation event, and leads to strand loss, and thus limits the read length of a sequencing method.

The term “mispair extension,” as used herein, generally refers to occurrences when the DNA polymerase incorporates a nucleotide, either labeled or unlabeled, that is not the correct Watson-Crick partner for the template base, then subsequently incorporates the correct Watson-Crick partner for the following base. Mispair extension generally results in lead phasing and limits the read length of a sequencing method.

Regarding quenching, dye-dye quenching between two dye moieties linked to different nucleotides (e.g., adjacent nucleotides in a growing nucleic acid strand, or nucleotides in a nucleic acid strand that are separated by one or more other nucleotides) may be strongly dependent on the distance between the two dye moieties. The distance between two dye moieties may be at least partially dependent on the properties of linkers connecting the two dye moieties to respective nucleotides or nucleotide analogs, including the linker compositions and functional lengths. Features of the linkers, including composition and functional length, may be affected by temperature, solvent, pH, and salt concentration (e.g., within a solution). Quenching may also vary based on the nature of the dyes used. Quenching may also take place between dye moieties and nucleobase moieties (e.g., between a fluorescent dye and a nucleobase of a nucleotide with which it is associated). Controlling quenching phenomena may be a key feature of the methods described herein.

Regarding flows, a nucleotide flow can consist of a mixture of labeled and unlabeled nucleotides or nucleotide analogs (e.g., nucleotides or nucleotide analogs of a single canonical type). For example, a solution comprising a plurality of optically (e.g., fluorescently) labeled nucleotides and a plurality of unlabeled nucleotides may be contacted with, e.g., a sequencing template (as described herein). The plurality of optically labeled nucleotides and a plurality of unlabeled nucleotides may each comprise the same canonical nucleotide or nucleotide analog. A flow may include only labeled nucleotides or nucleotide analogs. Alternatively, a flow may include only unlabeled nucleotides or nucleotide analogs. A flow may include a mixture of nucleotide or nucleotide analogs of different types (e.g., A and G).

A wash flow (e.g., a solution comprising a buffer) may be used to remove any nucleotides that are not incorporated into a nucleic acid complex (e.g., a sequencing template, as described herein). A cleavage flow (e.g., a solution comprising a cleavage reagent) may be used to remove dye moieties (e.g., fluorescent dye moieties) from optically (e.g., fluorescently) labeled nucleotides or nucleotide analogs. In some cases, different dyes (e.g., fluorescent dyes) may be removable using different cleavage reagents. In other cases, different dyes (e.g., fluorescent dyes) may be removable using the same cleavage reagents. Cleavage of dye moieties from optically labeled nucleotides or nucleotide analogs may comprise cleavage of all or a portion of a linker connecting a nucleotide or nucleotide analog to a dye moiety.

The term “cycle,” as used herein, generally refers to a process in which a nucleotide flow, a wash flow, and a cleavage flow corresponding to each canonical nucleotide (e.g., dATP, dCTP, dGTP, and dTTP or dUTP, or modified versions thereof) are used (e.g., provided to a sequencing template, as described herein). Multiple cycles may be used to sequence and/or amplify a nucleic acid molecule. The order of nucleotide flows can be varied.

Phasing can be lead or lag phasing. Lead phasing generally refers to the phenomenon in which a population of strands show incorporation of a nucleotide a flow ahead of the expected cycle (e.g., due to contamination in the system). Lag phasing refers to the phenomenon in which a population of strands shows incorporation of a nucleotide a flow behind the expected cycle (e.g., due to incompletion of extension in an earlier cycle).

Compounds and chemical moieties described herein, including linkers, may contain one or more asymmetric centers and thus give rise to enantiomers, diastereomers, and other stereoisomeric forms that are defined, in terms of absolute stereochemistry, as (R)- or (S)-, and, in terms of relative stereochemistry, as (D)- or (L)-. The D/L system relates molecules to the chiral molecule glyceraldehyde and is commonly used to describe biological molecules including amino acids. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a phenyl ring. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

Compounds and chemical moieties described herein, including linkers, may exist as tautomers. A “tautomer” refers to a molecule wherein a proton shift from one atom of a molecule to another atom of the same molecule is possible. In circumstances where tautomerization is possible, a chemical equilibrium of the tautomers will exist. Unless otherwise stated, chemical structures depicted herein are intended to include structures which are different tautomers of the structures depicted. For example, the chemical structure depicted with an enol moiety also includes the keto tautomer form of the enol moiety. The exact ratio of the tautomers depends on several factors, including physical state, temperature, solvent, and pH. Some examples of tautomeric equilibrium include:

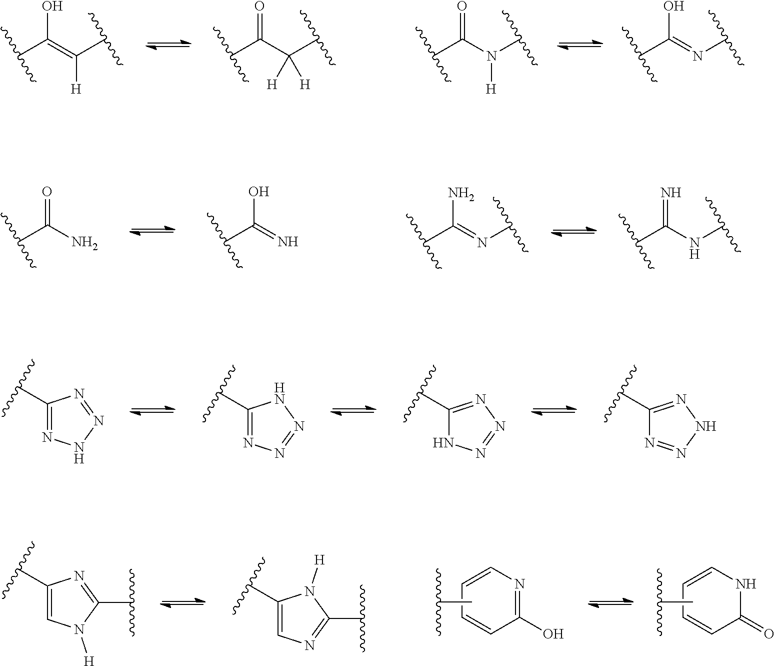

Compounds and chemical moieties described herein, including linkers and dyes, may be provided in different enriched isotopic forms. For example, compounds may be enriched in the content of ²H, ³H, ¹³C and/or ¹⁴C. For example, a linker, substrate (e.g., nucleotide or nucleotide analog), or dye may be deuterated in at least one position. In some examples, a linker, substrate (e.g., nucleotide or nucleotide analog), or dye may be fully deuterated. Such deuterated forms can be made by the procedure described in U.S. Pat. Nos. 5,846,514 and 6,334,997, each of which are herein incorporated by reference in their entireties. As described in U.S. Pat. Nos. 5,846,514 and 6,334,997, deuteration can improve the metabolic stability and or efficacy, thus increasing the duration of action of drugs.

Unless otherwise stated, structures depicted and described herein are intended to include compounds which differ only in the presence of one or more isotopically enriched atoms. For example, compounds and chemical moieties having the present structures except for the replacement of a hydrogen by a deuterium or tritium, or the replacement of a carbon by ¹³C- or ¹⁴C-enriched carbon are within the scope of the present disclosure.

The compounds and chemical moieties of the present disclosure may contain unnatural proportions of atomic isotopes at one or more atoms that constitute such compounds. For example, a compound or chemical moiety such as a linker, substrate (e.g., nucleotide or nucleotide analog), or dye, or a combination thereof, may be labeled with one or more isotopes, such as deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds and chemical moieties described herein, whether radioactive or not, are encompassed within the scope of the present disclosure.

### Linkers for Optical Detection

The present disclosure provides an optical (e.g., fluorescent) labeling reagent comprising a dye (e.g., fluorescent dye) and a linker that is connected to the dye and configured to couple to a substrate for optically (e.g., fluorescently) labeling the substrate. The substrate can be any suitable molecule, analyte, cell, tissue, or surface that is to be optically labeled. Examples include cells, including eukaryotic cells, prokaryotic cells, healthy cells, and diseased cells; cellular receptors; antibodies; proteins; lipids; metabolites; saccharides; polysaccharides; probes; reagents; nucleotides and nucleotide analogs (e.g., as described herein); polynucleotides; and nucleic acid molecules. For example, the substrate may be a nucleotide or nucleotide analog. In another example, the substrate may be a protein such as an antibody, such as a protein (e.g., antibody) that is a component of a cell. An association between a linker and a substrate can be any suitable association including a covalent or non-covalent bond. For example, a linker of an optical labeling reagent may be coupled to a substrate (e.g., nucleotide or nucleotide analog) via a nucleobase of a nucleotide, such as a nucleotide in a nucleic acid molecule, via, e.g., a propargyl or propargylamino moiety. In another example, a linker of an optical labeling reagent may be coupled to a substrate (e.g., protein, such as an antibody) via an amino acid of a polypeptide or protein. In some cases, an association between a linker and a substrate may be a biotin-avidin interaction. In other cases, an association between a linker and a substrate may be via a propargylamino moiety. In some cases, an association between a linker and a substrate may be via an amide bond (e.g., a peptide bond). A labeling reagent may comprise a cleavable moiety configured to be cleaved to separate the labeling reagent or a portion thereof from a substrate to which it is attached.

In an aspect, the present disclosure provides a labeling reagent (e.g., a fluorescent labeling reagent) comprising an optically detectable moiety such as a fluorescent dye moiety. A labeling reagent may comprise multiple optically detectable moieties, such as multiple fluorescent dye moieties, that may have the same or different chemical structures and may generate signal (e.g., fluoresce) at the same or different wavelengths. A labeling reagent may also comprise a linker that is connected to an optically detectable moiety (e.g., a fluorescent dye moiety). The linker may comprise one or more components, including one or more semi-rigid portions, spacer portions, cleavable portions, etc. The linker may comprise at least one non-proteinogenic amino acid, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more non-proteinogenic amino acids. For example, the linker may comprise at least 10 non-proteinogenic amino acids, such as at least 10 hydroxyprolines. In another example, the linker may comprise at least 20 non-proteinogenic amino acids. Non-proteinogenic amino acids of a linker may be included in any useful portion of the linker and may be included in sequence or separated by one or more other chemical moieties (e.g., as described herein). The linker may be configured to couple to a substrate for optically (e.g., fluorescently) labeling the substrate. The substrate may be, for example, a nucleotide or nucleotide analog, nucleic acid molecule, polynucleotide, protein, antibody, cell, saccharide, polysaccharide, lipid, or any other substrate described herein. The labeling reagent may comprise a cleavable moiety configured to be cleaved to separate the labeling reagent or a portion thereof from the substrate.

In another aspect, the present disclosure provides a labeling reagent (e.g., a fluorescent labeling reagent) comprising an optically detectable moiety such as a fluorescent dye moiety. A labeling reagent may comprise multiple optically detectable moieties, such as multiple fluorescent dye moieties, that may have the same or different chemical structures and may generate signal (e.g., fluoresce) at the same or different wavelengths. A labeling reagent may also comprise a linker that is connected to an optically detectable moiety (e.g., a fluorescent dye moiety). The linker may comprise one or more components, including one or more semi-rigid portions, spacer portions, cleavable portions, etc. For example, the linker may comprise a semi-rigid portion. The semi-rigid portion of the linker may provide physical separation between a substrate to which the labeling reagent couples and an optically detectable moiety, which physical separation may facilitate, e.g., effective labeling of the substrate with the labeling reagent, effective detection of the labeling reagent coupled to the substrate, effective labeling of the substrate with additional labeling reagents (e.g., in the case of incorporation into homopolymeric regions of a nucleic acid template, as described herein), etc. The semi-rigid portion may provide physical separation of, on average, at least 9 Angstrom (A) between a substrate to which a labeling reagent is coupled and an optically detectable moiety of the labeling reagent. For example, the semi-rigid portion may provide physical separation of, on average, at least 9 Å, 12 Å, 15 Å, 18 Å, 21 Å, 24 Å, 27 Å, 30 Å, 33 Å, 36 Å, 39 Å, 42 Å, 45 Å, 48 Å, 51 Å, 54 Å, 57 Å, 60 Å, 63 Å, 66 Å, 69 Å, 72 Å, 75 Å, 78 Å, 81 Å, 84 Å, 87 Å, 90 Å, or more between a substrate to which a labeling reagent is coupled and an optically detectable moiety of the labeling reagent. This average separation may vary with environmental conditions including, for example, solvents (or lack thereof), temperature, pH, pressure, etc. In an example, a semi-rigid portion of a linker may comprise a secondary structure such as a helical structure that establishes and maintains a degree of physical separation between a substrate and an optically detectable moiety. For example, a semi-rigid portion of a linker may comprise a second structure such as a helical structure comprising 3 or more prolines and/or hydroxyprolines. The linker may comprise at least one non-proteinogenic amino acid, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more non-proteinogenic amino acids. For example, the linker may comprise at least 10 non-proteinogenic amino acids, such as at least 10 hydroxyprolines. In another example, the linker may comprise at least 20 non-proteinogenic amino acids. Non-proteinogenic amino acids of a linker may be included in any useful portion of the linker and may be included in sequence or separated by one or more other chemical moieties (e.g., as described herein). For example, a linker may comprise a first semi-rigid portion and a second semi-rigid portion separated by another moiety, where the first and second semi-rigid portions comprise secondary structures such as helical structures. The linker may be configured to couple to a substrate for optically (e.g., fluorescently) labeling the substrate. The substrate may be, for example, a nucleotide or nucleotide analog, polynucleotide, nucleic acid molecule, protein, antibody, cell, saccharide, polysaccharide, lipid, or any other substrate described herein. The labeling reagent may comprise a cleavable moiety configured to be cleaved to separate the labeling reagent or a portion thereof from the substrate.

In another aspect, the present disclosure provides a labeling reagent (e.g., a fluorescent labeling reagent) comprising an optically detectable moiety such as a fluorescent dye moiety. A labeling reagent may comprise multiple optically detectable moieties, such as multiple fluorescent dye moieties, that may have the same or different chemical structures and may generate signal (e.g., fluoresce) at the same or different wavelengths. A labeling reagent may comprise the general structure: (cleavable linker moiety)-(semi-rigid linker moiety)-(optically detectable moiety). Each component of this general structure may be separated by one or more additional moieties, including one or more spacer moieties. In some cases, a labeling reagent may comprise a scaffold that permits the inclusion of multiple semi-rigid linker moieties and/or optically detectable moieties (e.g., fluorescent dye moieties). For example, a labeling reagent may comprise a branching or dendritic structure. A labeling reagent may also comprise one or more additional features including one or more spacer portions. The labeling reagent may comprise at least one non-proteinogenic amino acid, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more non-proteinogenic amino acids. For example, the linker may comprise at least 10 non-proteinogenic amino acids, such as at least 10 hydroxyprolines. In another example, the linker may comprise at least 20 non-proteinogenic amino acids. Non-proteinogenic amino acids of a linker may be included in any useful portion of the linker and may be included in sequence or separated by one or more other chemical moieties (e.g., as described herein). One or more non-proteinogenic amino acids may be included in a semi-rigid linker portion. For example, a semi-rigid linker portion may comprise a secondary structure such as a helical portion comprising one or more prolines and/or hydroxyprolines. The labeling reagent may be configured to couple to a substrate for optically (e.g., fluorescently) labeling the substrate. The substrate may be, for example, a nucleotide or nucleotide analog, polynucleotide, nucleic acid molecule, protein, antibody, cell, saccharide, polysaccharide, lipid, or any other substrate described herein. The labeling reagent may comprise a cleavable moiety configured to be cleaved to separate the labeling reagent or a portion thereof from the substrate.

A linker may comprise one or more regions having a semi-rigid structure. For example, a linker may comprise at least one region having a semi-rigid structure, such as 1, 2, 3, 4, 5, 6, 7, 8, 9, or more regions having a semi-rigid structure. A region of a linker having a semi-rigid structure may be adjacent to another region of a linker having a semi-rigid structure. Alternatively or in addition, a region of a linker having a semi-rigid linker may be adjacent to another region of a linker that does not have a semi-rigid structure. Similarly, an optical (e.g., fluorescent) labeling reagent may comprise one or more regions having a semi-rigid structure. For example, an optical (e.g., fluorescent) labeling reagent may comprise at least one region having a semi-rigid structure, such as 1, 2, 3, 4, 5, 6, 7, 8, 9, or more regions having a semi-rigid structure. Semi-rigid structures of an optical (e.g., fluorescent) labeling reagent may be included in the same or different linkers. For example, an optical (e.g., fluorescent) labeling reagent may comprise a first linker having a first semi-rigid structure and a second linker having a second semi-rigid structure, where the first and second semi-rigid structures may have the same or different chemical structures. Two or more semi-rigid structures with the same or different chemical structures may be coupled to separate portions of a structure of a labeling reagent. For example, a labeling reagent may comprise a scaffold, such as a scaffold comprising one or more lysine moieties, to which multiple different semi-rigid structures may couple at different locations to provide a branched or dendritic labeling reagent structure. Alternatively or additionally, a given linker of an optical (e.g., fluorescent) labeling reagent may comprise multiple semi-rigid structures (e.g., adjacent to one another or separated by one or more other moieties, such as by one or more amino acids that do not contribute to a semi-rigid structure. For example, a first semi-rigid structure may be separated from a second semi-rigid structure by at least a glycine moiety.

The semi-rigid nature of a linker, or portion thereof, may be attributable, at least in part, to a structure that comprises a series of ring systems (e.g., aliphatic and aromatic rings). As used herein, a ring (e.g., ring structure) is a cyclic moiety comprising any number of atoms connected in a closed, essentially circular fashion, as used in the field of organic chemistry. A ring may be defined by any number of atoms. For example, a ring may include between 3-12 atoms, such as between 3-12 carbon atoms. In certain examples, a ring may be a five-membered ring (i.e., a pentagon) or a six-membered ring (i.e., a hexagon). A ring can be aromatic or non-aromatic. A ring may be aliphatic. A ring may comprise one or more double bonds.

A ring (e.g., ring structure) may be a component of a ring system that may comprise one or more ring structures (e.g., a multi-cycle system). For example, a ring system may comprise a monocycle. In another example, a ring system may be a bicycle or bridged system. A ring structure may be a carbocycle or component thereof formed of carbon atoms. A carbocycle may be a saturated, unsaturated, or aromatic ring in which each atom of the ring is carbon. A carbocycle includes 3- to 10-membered monocyclic rings, 4- to 12-membered bicyclic rings (e.g., 6- to 12-membered bicyclic rings), and 5- to 12-membered bridged rings. Each ring of a bicyclic carbocycle may be selected from saturated, unsaturated, and aromatic rings. For example, a bicyclic carbocycle may include an aromatic ring (e.g., phenyl) fused to a saturated or unsaturated ring (e.g., cyclohexane, cyclopentane, or cyclohexene). A bicyclic carbocycle may include any combination of saturated, unsaturated, and aromatic bicyclic rings, as valence permits. A bicyclic carbocycle may include any combination of ring sizes such as 4-5 fused ring systems, 5-5 fused ring systems, 5-6 fused ring systems, and 6-6 fused ring systems. A carbocycle may be, for example, cyclopropyl, cyclobutyl, cyclopentyl, cyclohexyl, cyclohexenyl, adamantyl, phenyl, indanyl, or naphthyl. A saturated carbocycle includes no multiple bonds (e.g., double or triple bonds). A saturated carbocycle may be, for example, cyclopropane, cyclobutane, cyclopentane, or cyclohexane. An unsaturated carbocycle includes at least one multiple bond (e.g., double or triple bond) but is not an aromatic carbocycle. An unsaturated carbocycle may be, for example, cyclohexadiene, cyclohexene, or cyclopentene. Other examples of carbocycles include, but are not limited to, cyclopropane, cyclobutane, cyclopentane, cyclopentadiene, cyclohexane, cycloheptane, cycloheptene, naphthalene, and adamantine. An aromatic carbocycle (e.g., aryl moiety) may be, for example, phenyl, naphthyl, or dihydronaphthyl.

In some cases, a ring may include one or more heteroatoms, such as one or more oxygen, nitrogen, silicon, phosphorous, boron, or sulfur atoms. A ring may be a heterocycle or component thereof including one or more heteroatoms. A heterocycle may be a saturated, unsaturated, or aromatic ring in which at least one atom is a heteroatom. A heteroatom includes 3- to 10-membered monocyclic rings, 6- to 12-membered bicyclic rings, and 6- to 12-membered bridged rings. A bicyclic heterocycle may include any combination of saturated, unsaturated and aromatic bicyclic rings, as valence permits. For example, a heteroaromatic ring (e.g., pyridyl) may be fused to a saturated or unsaturated ring (e.g., cyclohexane, cyclopentane, morpholine, piperidine or cyclohexene). A bicyclic heterocycle may include any combination of ring sizes such as 4-5 fused ring systems, 5-5 fused ring systems, 5-6 fused ring systems, and 6-6 fused ring systems. An unsaturated heterocycle includes at least one multiple bond (e.g., double or triple bond) but is not an aromatic heterocycle. An unsaturated heterocycle may be, for example, dihydropyrrole, dihydrofuran, oxazoline, pyrazoline, or dihydropyridine. Additional examples of heterocycles include, but are not limited to, indole, benzothiophene, benzthiazole, benzoxazole, benzimidazole, oxazolopyridine, imidazopyridine, thiazolopyridine, furan, oxazole, pyrrole, pyrazole, imidazole, thiophene, thiazole, isothiazole, and isoxazole. A heteroaryl moiety may be an aromatic single ring structure, such as a 5- to 7-membered ring, including at least one heteroatom, such as one to four heteroatoms. Alternatively, a heteroaryl moiety may be a polycyclic ring system having two or more cyclic rings in which two or more atoms are common to two adjoining rings wherein at least one of the rings is heteroaromatic. Heteroaryl groups include, for example, pyrrole, furan, thiophene, imidazole, oxazole, thiazole, pyrazole, pyridine, pyrazine, pyridazine, and pyrimidine, and the like.

A ring can be substituted or un-substituted. A substituent replaces a hydrogen atom on one or more atoms of a ring or a substitutable heteroatom of a ring (e.g., NH or NH₂). Substitution is in accordance with permitted valence of the various components of the ring system and provides a stable compound (e.g., a compound that does not undergo spontaneous transformation by, for example, rearrangement, elimination, or cyclization). A substituent may replace a single hydrogen atom or multiple hydrogen atoms (e.g., on the same ring atom or different ring atoms). A substituent on a ring may be, for example, halogen, hydroxy, oxo, thioxo, thiol, amido, amino, carboxy, nitrilo, cyano, nitro, imino, oximo, hydrazino, alkoxy, alkenyl, alkynyl, aryl, aralkyl, aralkenyl, aralkynyl, cycloalkyl, cycloalkylalkyl, alkylcycloalkyl, heterocycloalkyl, heterocycyl, alkylheterocycyl, or any other useful substituent. A substituent may be water-soluble. Examples of water-soluble substituents include, but are not limited to, a pyridinium, an imidazolium, a quaternary ammonium group, a sulfonate, a sulfate, a phosphate, an alcohol, an amine, an imine, a nitrile, an amide, a thiol, a carboxylic acid, a polyether, an aldehyde, a boronic acid, and a boronic ester.

A linker, or a semi-rigid portion thereof, can have any number of rings, including at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more rings. The rings can share an edge in some cases (e.g., be components of a bicyclic ring system). In general, the ring portion of the linker can provide a degree of physical rigidity to the linker and/or can serve to physically separate the dye (e.g., fluorescent dye) on one end of the linker from the substrate to be labeled and/or from a second dye (e.g., fluorescent dye) associated with the substrate and/or associated with the linker. A ring can be a component of an amino acid (e.g., a non-proteinogenic amino acid, as described herein). For example, a linker may comprise a proline moiety. In another example, a linker may comprise a hydroxyproline moiety. For example, a linker, or a semi-rigid portion thereof, may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more proline or hydroxyproline moieties.

In some cases, a linker may comprise a “fully rigid” (e.g., substantially inflexible) portion. For example, a linker may comprise a region including ring systems that may not be separated by any sp² or sp³ carbon atoms. In general, sp² and sp³ carbon atoms (e.g., between ring systems) provide a linker or portion thereof with a degree of physical flexibility. sp³ carbon atoms in particular can confer significant flexibility. Without limitation, flexibility can allow a polymerase to accept a substrate (e.g., a nucleotide or nucleotide analog) modified with the linker and the dye (e.g., fluorescent dye), or otherwise improve the performance of a labeled system. However, in a multiple dye system (e.g., a system comprising multiple fluorescent labeling reagents, such as a polynucleotide including two or more nucleotides coupled to two or more fluorescent labeling reagents), an overly flexible linker may defeat the feature of rigidity and allow two dyes (e.g., fluorescent dyes) to come into close association and be quenched. Accordingly, ring systems of a linker or portion thereof may be connected to each other by a limited number of sp³ bonds, such as by no more than two sp³ bonds (e.g., 0, 1, or 2 sp³ bonds), to, e.g., confer a degree of rigidity to the linker or portion thereof. For example, at least two ring systems of a linker or portion thereof may be connected to each other by no more than two sp³ bonds (e.g., by 0, 1, or 2 sp³ bonds). For example, at least two ring systems of a linker or portion thereof may be connected to each other by a no more than two sp² bonds, such as by no more than 1 sp² bond. Ring systems of a linker or portion thereof may be connected to each other by a limited number of atoms, such as by no more than 2 atoms. For example, at least two ring systems of a linker or portion thereof may be connected to each other by no more than 2 atoms, such as by only 1 atom or by no atoms (e.g., directly connected).

A series of ring systems of a linker or portion thereof may comprise aromatic and/or aliphatic rings. At least two ring systems of a linker or portion thereof may be connected to each other directly without an intervening carbon atom. A linker may comprise at least one amino acid that may comprise a ring system, such as a proline or hydroxyproline moiety. For example, a linker may comprise a hydroxyproline. A linker may comprise at least one non-proteinogenic amino acid (e.g., as described herein), such as a hydroxyproline. A linker may comprise a plurality of amino acids including ring systems in sequence. For example, a linker may comprise at least two amino acids in sequence, where each of the at least two amino acids includes a ring system (e.g., ring systems having the same or different structures). The at least two amino acids may comprise at least two non-proteinogenic amino acids, such as hydroxyprolines. In another example, a linker may comprise at least three amino acids in sequence, where each of the at least three amino acids includes a ring system (e.g., ring systems having the same or different structures). The at least three amino acids may comprise at least three non-proteinogenic amino acids. For example, the linker may comprise at least three hydroxyprolines, such as at least 3, 4, 5, 6, 7, 8, 9, 10, or more hydroxyprolines. Two or more non-proteinogenic amino acids may be included in sequence. For example, two or more non-proteinogenic amino acids may be adjacent to one another without an intervening feature or other chemical structure. For example, 3, 4, 5, 6, 7, 8, 9, 10, 11, 12, 13, 14, 15, 16, 17, 18, 19, 20, 21, 22, 23, 24, 25, 26, 27, 28, 29, 30, or more non-proteinogenic amino acids may be included in sequence. A linker may comprise a first sequence of amino acids including ring systems and a second sequence of amino acids including ring systems, where the first sequence and the second sequence may be separated by one or more moieties that do not include ring systems, such as one or more glycines. For example, a linker may comprise a first sequence of hydroxyprolines and a second sequence of hydroxyprolines, where the first sequence and the second sequence may be separated by at least a glycine. In another example, a linker may comprise a first sequence of amino acids including ring systems, a second sequence of amino acids including ring systems, and a third sequence of amino acids including ring systems, where the first, second, and third sequences may be separated by one or more moieties that do not include ring systems, such as one or more glycines. An optical (e.g., fluorescent) labeling reagent may comprise one or more linkers, such as one or more linkers each comprising two or more amino acids (e.g., non-proteinogenic amino acids). For example, an optical labeling reagent may comprise a first linker comprising a first sequence of amino acids and a second linker comprising a second sequence of amino acids, where the first sequence comprises two or more amino acids (e.g., non-proteinogenic amino acids) comprising ring systems and the second sequence comprises two or more amino acids (e.g., non-proteinogenic amino acids) comprising ring systems. In an example, an optical labeling reagent may comprise a first linker comprising a first sequence of hydroxyprolines and a second linker comprising a second sequence of hydroxyprolines. The first and second linkers may be connected to different portions of a scaffold. The first linker may be coupled, directly or indirectly, to a first optically detectable moiety and the second linker may be coupled, directly or indirectly, to a second optically detectable moiety, where the first and second optically detectable moieties may be of the same or different types.

A linker or portion thereof of a labeling reagent provided herein may comprise a secondary structure, such as a helical structure. For example, a labeling reagent may comprise a polyproline or polyhydroxyproline helix. A helical structure comprising prolines and/or hydroxyprolines may comprise three or more prolines and/or hydroxyprolines in sequence. For example, an optical labeling reagent may comprise a first linker comprising a first secondary structure (e.g., helical structure) comprising a first sequence of hydroxyprolines and a second linker comprising a second secondary structure (e.g., helical structure) comprising a second sequence of hydroxyprolines. The first and second linkers may be connected to different portions of a scaffold. The first linker may be coupled, directly or indirectly, to a first optically detectable moiety and the second linker may be coupled, directly or indirectly, to a second optically detectable moiety, where the first and second optically detectable moieties may be of the same or different types. In a helical structure comprising prolines and/or hydroxyprolines, or derivatives thereof, a given proline, hydroxyproline, or derivative thereof may provide a physical separation of approximately 3 Å between moieties to which it is connected. For example, a helical or semi-helical structure comprising three prolines, hydroxyprolines, or similar structures may provide physical separation of approximately 9 Å between moieties to which they are connected. In some cases, a secondary structure such as a helical structure may provide a physical separation between moieties to which they are connected of at least about 9 Å, such as at least about 9 Å, 12 Å, 15 Å, 18 Å, 21 Å, 24 Å, 27 Å, 30 Å, or more. In some cases, several such secondary structures will be included in a single linker moiety, optionally separated by one or more features such as another chemical moiety. For example, two helical structures comprising prolines, hydroxyprolines, or derivatives thereof may be separated by a glycine. In some cases, multiple secondary structures will be included in an optical labeling reagent but may not necessarily be included in sequence. For example, an optical labeling reagent may comprise a first linker comprising a first helical structure and a second linker comprising a second helical structure. The first linker or the second linker may additionally comprise a third helical structure and, in some cases, a fourth helical structure.

The structural features of a linker, including the number of rings, the rigidity of the linker or a portion thereof, and the like, can combine to establish a functional distance between an optically detectable moiety (e.g., fluorescent dye moiety) and a substrate (e.g., protein, nucleotide or nucleotide analog, cell, etc.) labeled by a labeling reagent. In some cases, the distance corresponds to the length (and/or the functional length) of the linker. A functional length of a labeling reagent or portion thereof may be an average value representing an average over various molecular and solvent motions. In some cases, the functional length varies based on one or more of the temperature, solvent, pH, and/or salt concentration of the solution in which the length is measured or estimated. The functional length can be measured in a solution in which an optical (e.g., fluorescent) signal from the substrate is measured. The functional length may an average or ensemble value of a distribution of functional lengths (e.g., over rotational, vibrational, and translational motions) and may differ based on, e.g., temperature, solvent, pH, and/or salt concentrations. The functional length may be estimated (e.g., based on bond lengths and steric considerations, such as by use of a chemical drawing or modeling program) and/or measured (e.g., using molecular imaging and/or crystallographic techniques). For an optical (e.g., fluorescent) labeling reagent comprising one or more linkers, such as one or more linkers connecting one or more dye moieties to a substrate, one or more different functional distances may be established between dye moieties and a substrate.

A labeling reagent can establish any suitable functional length between an optically detectable moiety (e.g., fluorescent dye) and a substrate (e.g., protein, nucleotide or nucleotide analog, cell, etc.) labeled by the labeling reagent. In some cases, the functional length is at most about 500 nanometers (nm), about 200 nm, about 100 nm, about 75 nm, about 50 nm, about 40 nm, about 30 nm, about 20 nm, about 10 nm, about 5 nm, about 2 nm, about 1.0 nm, about 0.5 nm, about 0.3 nm, about 0.2 nm, or less. In some instances, the functional length is at least about 0.2 nanometers (nm), at least about 0.3 nm, at least about 0.5 nm, at least about 1.0 nm, at least about 2 nm, at least about 5 nm, at least about 10 nm, at least about 20 nm, at least about 30 nm, at least about 40 nm, at least about 50 nm, at least about 75 nm, at least about 100 nm, at least about 200 nm, at least about 500 nm, or more. In some instances, the functional length is between about 0.5 nm and about 50 nm. In some cases, the functional length may be at least about 9 Å, 12 Å, 15 Å, 18 Å, 21 Å, 24 Å, 27 Å, 30 Å, 33 Å, 36 Å, 39 Å, 42 Å, 45 Å, 48 Å, 51 Å, 54 Å, 57 Å, 60 Å, 63 Å, 66 Å, 69 Å, 72 Å, 75 Å, 78 Å, 81 Å, 84 Å, 87 Å, 90 Å, or more.

Many applications of optical (e.g., fluorescent) labeling reagents (e.g., nucleic acid sequencing reactions and protein/cell labeling) can be performed in aqueous solutions. In some cases, a linker that has too high of a proportion of carbon and hydrogen atoms and/or a lack of charged chemical groups can be insufficiently water-soluble to be useful in an aqueous solution. Accordingly, a labeling reagent may comprise one or more water-soluble groups. A water-soluble group may be incorporated into a labeling reagent at any useful position. For example, a linker of a labeling reagent, or a semi-rigid portion thereof, may include one or more water-soluble groups. A labeling reagent may also or alternatively include one or more water-soluble groups at or near a point of attachment to an optically detectable moiety (e.g., a fluorescent dye moiety, as described herein). Alternatively or additionally, a labeling reagent may comprise a water-soluble group at or near a point of attachment to a substrate (e.g., a protein, nucleotide or nucleotide analog, cell, etc.). Alternatively or additionally, a labeling reagent may comprise a water-soluble group between points of attachment to an optically detectable moiety (e.g., fluorescent dye moiety, as described herein) and a substrate (e.g., a protein, nucleotide or nucleotide analog, cell, etc.). One or more rings of a labeling reagent or linker thereof may comprise a water-soluble group incorporated therein or appended thereto. For example, a given ring of a labeling reagent, such as a ring included in a linker portion of a labeling reagent, may comprise one or more water-soluble moieties. For example, a ring of a linker may comprise two water-soluble moieties. A water-soluble group may be a constituent part of the backbone of a ring structure. Alternatively or additionally, a water-soluble group may be appended to a ring structure (e.g., as a substituent). For example, a labeling reagent may comprise at least one hydroxyproline, which hydroxyproline comprises a five-membered ring having a hydroxyl group appended thereto. Water-soluble moieties of a labeling reagent may be of the same or different types. For example, a labeling reagent may comprise at least one water-soluble moiety of a first type and at least one water-soluble moiety of a second type that is different from the first type. In an example, a labeling reagent may comprise multiple water-soluble moieties of a given type, such as multiple hydroxyl moieties. In some cases, a water-soluble group may be positively charged. Examples of suitable water-soluble groups include, but are not limited to, a pyridinium, an imidazolium, a quaternary ammonium group, a sulfonate, a sulfate, a phosphate, an alcohol, an amine, an imine, a nitrile, an amide, a thiol, a carboxylic acid, a polyether, an aldehyde, and a boronic acid or boronic ester.

A water-soluble group can be any functional group that decreases (including making more negative) the log P of the optical (e.g., fluorescent) labeling reagent. Log P is the partition coefficient for a molecule between water and π-octanol. A greasy molecule is more likely to partition into octanol, giving a positive and large log P value. A formula for Log P can be represented as log P_{octanol/water}−log (|solute|_{octanol}/[solute]_{water}), where [solute]_{octanol} is the concentration of the solute (i.e., the labeling reagent) in octanol and [solute]_{water} is the concentration of the solute in water. Therefore, the more a compound partitions into water compared to octanol, the more negative the log P. Log P can be measured experimentally or predicted using software algorithms. The water-soluble group can have any suitable Log P value. In some cases, the Log P is less than about 2, less than about 1.5, less than about 1, less than about 0.5, less than about 0, less than about −0.5, less than about −1, less than about −1.5, less than about −2, or lower. In some cases, the Log P is between about 2.0 and about −2.0.

A linker may include one or more asymmetric (e.g., chiral) centers (e.g., as described herein). All stereochemical isomers of linkers are contemplated, including racemates and enantiomerically pure linkers.

A labeling reagent or component thereof, and/or a substrate (e.g., protein, nucleotide or nucleotide analog, cell, etc.) to which it may be coupled, may include one or more isotopic (e.g., radio) labels (e.g., as described herein). All isotopic variations of linkers are contemplated.

A labeling reagent may comprise a polymer having a regularly repeating unit. Alternatively, a labeling reagent may comprise a co-polymer without a regularly repeating unit. A repeating unit may comprise a sequence of amino acids (e.g., non-proteinogenic amino acids). For example, a repeating unit may comprise at least 3 prolines, hydroxyprolines, or derivatives thereof, such as at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or more prolines, hydroxyprolines, or derivatives thereof. A repeating unit may comprise two or more different amino acids. For example, a repeating unit may comprise a first amino acid (X) and a second amino acid (Y). One or more of the first or second amino acids may be included. For example, a labeling reagent may comprise a moiety having the formula (XₙYₘ)ᵢ, where n is at least 1, m is at least 1, and i is at least 2 and X and Y are different amino acids. In an example, X may be glycine, n is 1, and Y is hydroxyproline. In such an instance, m may be at least 3 (e.g., 3, 4, 5, 6, 7, 8, 9, 10, 11, 12, 13, 14, 15, 16, 17, 18, 19, 20, or more) and i may be, for example, at least 2 (e.g., 2, 3, 4, 5, 6, 7, 8, or more). An example of such a linker component is shown below:

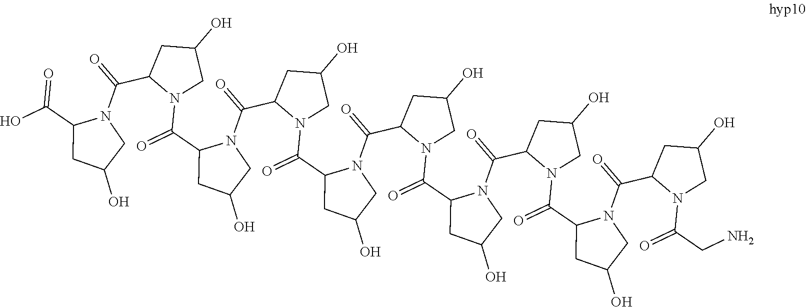 [SMILES: NCC(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)O]

The structure shown above includes 10 hydroxyproline moieties and a glycine moiety and is referred to herein as “H” or “hyp10.” Alternative representations may include Hyp10, hyp₁₀, and Hyp₁₀. Note that the representation hyp10 may also include sequences of 10 hydroxyprolines without a glycine moiety. In some cases, a hyp10 sequence including a glycine may alternatively be represented as hyp10-Gly or similar. One or more such structures may be included in a labeling reagent or linker portion thereof. For example, a hyp10 structure may be a repeating unit in a linker. Two hyp10 structures in sequence may be referred to herein as hyp20. Such a structure may include 20 hydroxyproline moieties and, in some cases, one or more glycines. Similarly, three hyp10 structures in sequence may be referred to herein as hyp30. Such a structure may include 30 hydroxyproline moieties and, in some cases, one or more glycines. For example, a hyp30 sequence may have include three sets of ten hydroxyprolines separated by glycines. Alternatively, a hyp30 structure may include thirty hydroxyprolines with no intervening structures. Related structures including different numbers of hydroxyprolines (e.g., hypn or hypₙ) may also be included in a labeling reagent. Additional details of such structures are provided elsewhere herein. As described herein, all stereoisomers of hyp10, hyp20, and hyp30, as well as combinations thereof, are contemplated.

A polymer or co-polymer structure may be included in a linker portion of a labeling reagent. A polymer or co-polymer structure may be prepared according to any useful method and may not be the result of a polymerization process. In general, a polymerization process can generate products having a variety of degrees of polymerization and molecular weights. In contrast, the labeling reagents provided herein may have a defined (i.e., known) molecular weight.

A labeling reagent may comprise a straight and/or contiguous chain. For example, a labeling reagent may have the general structure: (optional cleavable linker portion)—(semi-rigid linker portion)—(optically detectable moiety). Each moiety may be separated by one or more additional features including, e.g., a spacer portion. A labeling reagent may comprise multiple straight and/or contiguous chains linked to a central structure (e.g., scaffold, as described herein). A linker portion of a labeling reagent may comprise a branchpoint that facilitates connection of multiple optically detectable moieties to a given linker portion. Alternatively, a linker portion of a labeling reagent may be configured to connect to a single optically detectable moiety.

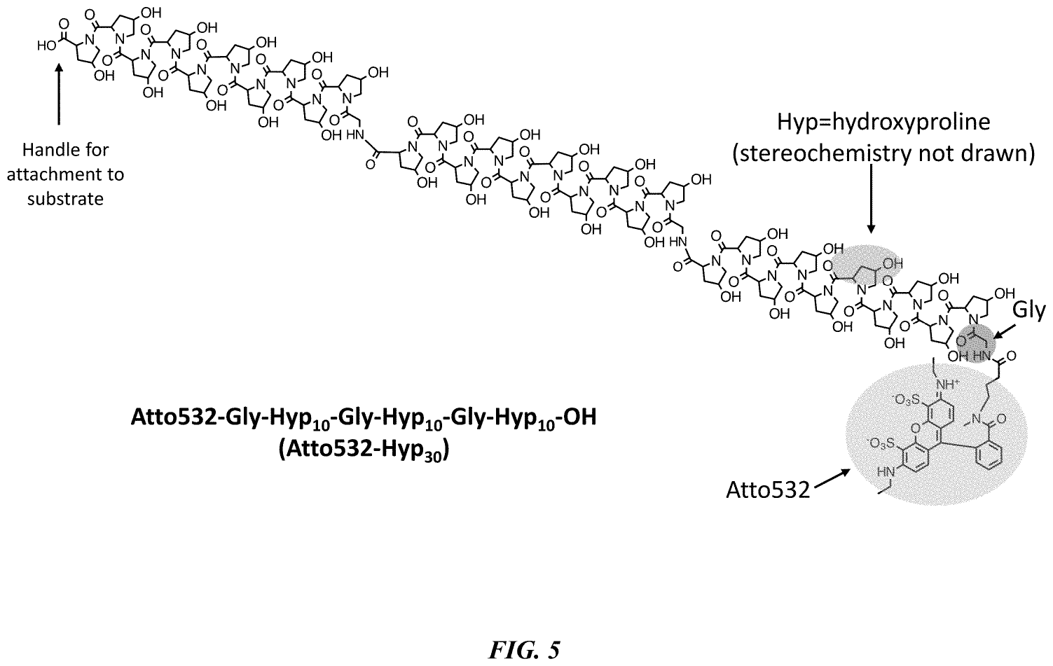

FIG. 5 shows an example structure for inclusion in a labeling reagent. The example structure includes a linker comprising three sequences of ten hydroxyprolines separated by glycines. The ten hydroxyproline portion may be represented herein as Hyp10, hyp10, Hyp₁₀, or hyp₁₀. The linker including the three sequences of ten hydroxyprolines separated by glycines may be represented as, for example, Hyp10-Gly-Hyp10-Gly-Hyp10-Gly or, in the alternative, Gly-Hyp10-Gly-Hyp10-Gly-Hyp10. The linker including the three sequences of ten hydroxyprolines separated by glycines may also be represented as, for example, Hyp30, hyp30, Hyp₃₀, or hyp₃₀. The structure also includes an optical dye moiety coupled to the linker via a glycine. The optical dye moiety included in FIG. 5 fluoresces at approximately 532 nanometers (nm). However, any other useful dye moiety may be used (e.g., as described herein). The structure shown in FIG. 5 also includes a handle for attachment to one or more additional moieties, including a cleavable linker moiety and/or spacer moiety via which the structure may be linked to a substrate (e.g., as described herein). In some cases, a linker may not include a cleavable linker moiety and the handle may provide a connection to a substrate. In some cases, the illustrated structure or a similar structure may be connected to a scaffold, optionally with an intervening cleavable moiety, which scaffold may facilitate the inclusion of multiple optically detectable moieties in a single labeling reagent.

### Branched and Dendritic Labeling Reagents

In some instances, a labeling reagent may comprise a branched structure. A labeling reagent may be capable of labeling a substrate (e.g., as described herein) with a plurality of optically detectable moieties (e.g., fluorescent dyes). For example, a labeling reagent may comprise a scaffold configured to link multiple optically detectable moieties to a single substrate via multiple separate linker portions. Such a scaffold may comprise multiple points of connection (e.g., “handles”) for attachment of linker moieties, which linker moieties may each be coupled to one or more optically detectable moieties. For example, a scaffold may comprise two or more amino moieties that may be functionalized with linkers. In an example, a scaffold may comprise a lysine. A scaffold may comprise a repeating moiety, such as two or more of a same moiety. For example, a scaffold may comprise two or more lysines. A labeling reagent may comprise multiple branch points. For example, a labeling reagent may comprise a dendron or dendrimer structure.

Accordingly, in an aspect, the present disclosure provides a labeling reagent (e.g., fluorescent labeling reagent) comprising a plurality of optically detectable moieties (e.g., fluorescent dye moieties) and a plurality of linkers. The plurality of optically detectable moieties may comprise the same number of optically detectable moieties as the plurality of linkers comprises linkers. Alternatively, the labeling reagent may comprise more linkers than optically detectable moieties, or more optically detectable moieties than linkers. In an example, the labeling reagent comprises two optically detectable moieties and two linkers. In another example, the labeling reagent comprises three optically detectable moieties and three linkers. The labeling reagent may comprise at least two linkers, such as at least 2, 3, 4, 5, 6, 7, 8, 9, or more linkers. Similarly, the labeling reagent may comprise at least two optically detectable moieties, such as at least 2, 3, 4, 5, 6, 7, 8, 9, or more optically detectable moieties. The plurality of linkers may comprise a first linker that is coupled (e.g., connected) to a first optically detectable moiety (e.g., first fluorescent dye moiety) of the plurality of optically detectable moieties and a second linker that is coupled (e.g., connected) to a second optically detectable moiety (e.g., second fluorescent dye moiety) of the plurality of optically detectable moieties. The first optically detectable moiety and the second optically detectable moiety may have the same chemical structure (e.g., may fluoresce at or near the same wavelengths). Where a labeling reagent comprises three or more optically detectable moieties, each optically detectable moiety may have the same chemical structure (e.g., may fluoresce at or near the same wavelengths). Alternatively, the first optically detectable moiety and the second optically detectable moiety may have different chemical structures (e.g., may fluoresce at different wavelengths). The labeling reagent may be configured to couple to a substrate (e.g., as described herein) for labeling (e.g., fluorescently labeling) the substrate. The substrate may be, for example, a protein, antibody, saccharide, polysaccharide, nucleotide, nucleotide analog, polynucleotide, nucleic acid molecule, cell, cell surface marker, or any other useful moiety (e.g., as described herein). The plurality of linkers may be connected to a scaffold such a scaffold comprising one or more lysines (e.g., 1, 2, 3, or more lysines). The first linker may be connected to a first lysine moiety and the second linker may be connected to a second lysine moiety, which second lysine moiety may be connected to the first lysine moiety. The labeling reagent may also comprise a cleavable group (e.g., as described herein), which cleavable group may connect the substrate to a scaffold of a labeling reagent. The first linker and the second linker may have the same or different structures. For example, the first linker may comprise a first semi-rigid portion and the second linker may comprise a second semi-rigid portion having the same structure as the first semi-rigid portion. The first linker and/or the second linker may have any useful features described herein, including amino acids (e.g., non-proteinogenic amino acids), ring structures, water-soluble groups, cleavable linker portions, semi-rigid portions, secondary structures (e.g., helical structures), etc. The first linker and/or the second linker may comprise one or more amino acids, such as one or more non-proteinogenic amino acids. For example, the first linker may comprise a first amino acid (e.g., a first non-proteinogenic amino acid) and the second linker may comprise a second amino acid (e.g., a second non-proteinogenic amino acid). The first amino acid and the second amino acid may be of the same or different types. In an example, the first linker may comprise at least one hydroxyproline or derivative thereof and the second linker may comprise at least one hydroxyproline or derivative thereof. For example, the first linker and/or the second linker may comprise at least 2 hydroxyprolines, such as at least 3, 4, 5, 6, 7, 8, 9, 10, 11, 12, 13, 14, 15, 16, 17, 18, 19, 20, 21, 22, 23, 24, 25, 26, 27, 28, 29, 30, or more hydroxyprolines. The first linker and/or the second linker may comprise the same or different numbers of any given species. For example, the first linker may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids (e.g., non-proteinogenic amino acids). Similarly, the second linker may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids (e.g., non-proteinogenic amino acids). For example, the first linker and/or the second linker may comprise a hyp10 moiety (e.g., 10 hydroxyprolines, as described herein), a hyp20 moiety (e.g., 20 hydroxyprolines, as described herein), or a hyp30 moiety (e.g., 30 hydroxyprolines, a described herein). The first linker and/or the second linker may comprise at least one glycine. Similarly, the first linker and/or the second linker may comprise at least one cysteic acid moiety. The first linker and/or the second linker may comprise a repeating unit (e.g., as described herein), which repeating unit may comprise one or more non-proteinogenic amino acids (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or more non-proteinogenic amino acids), such as one or more hydroxyprolines.

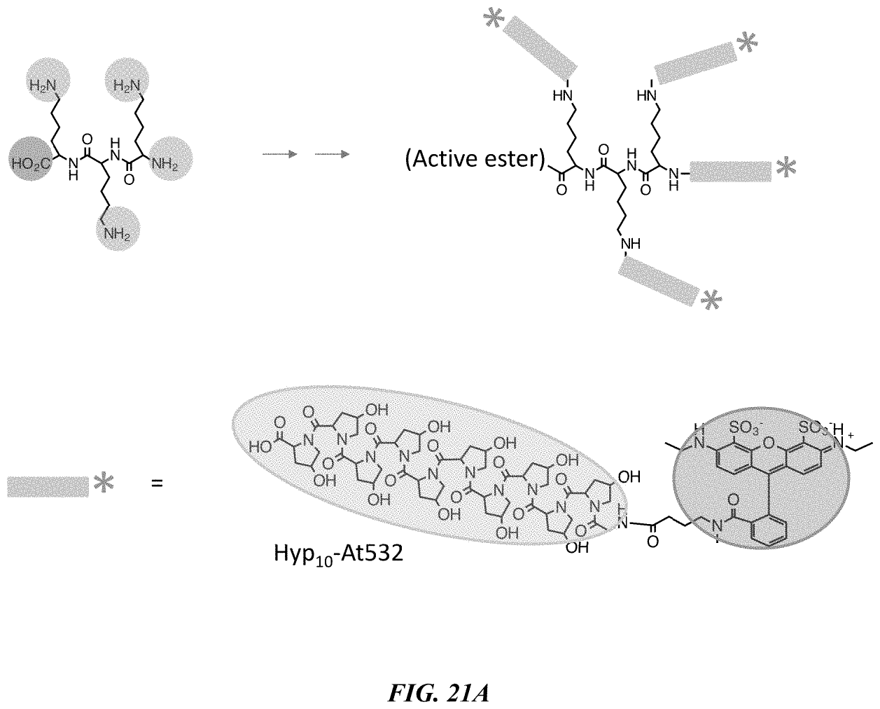

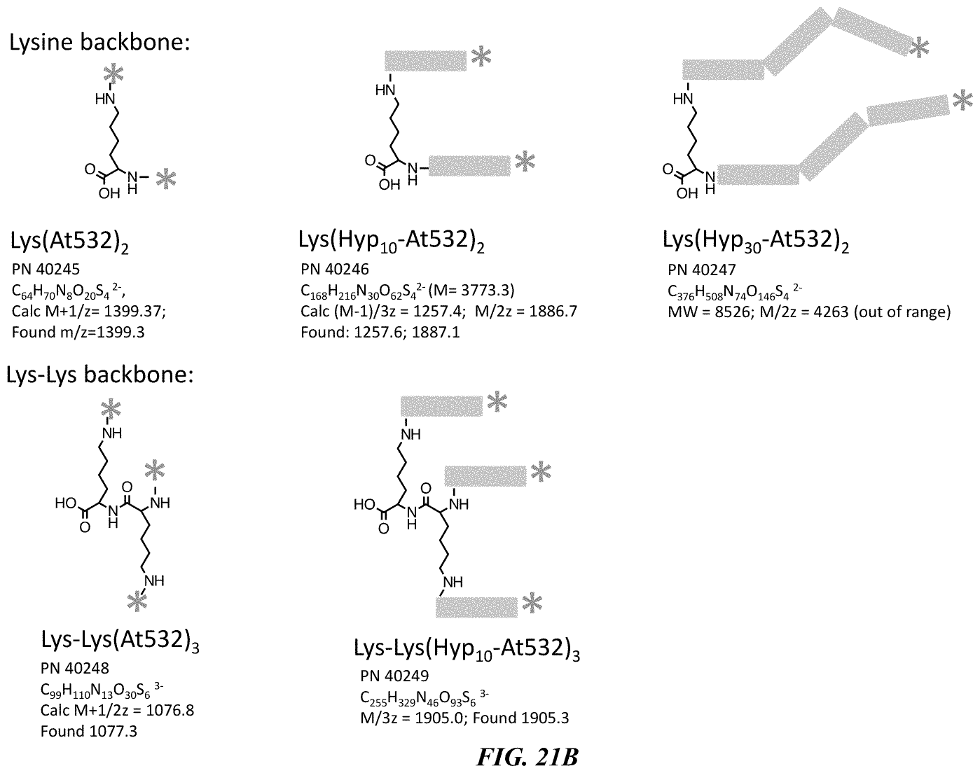

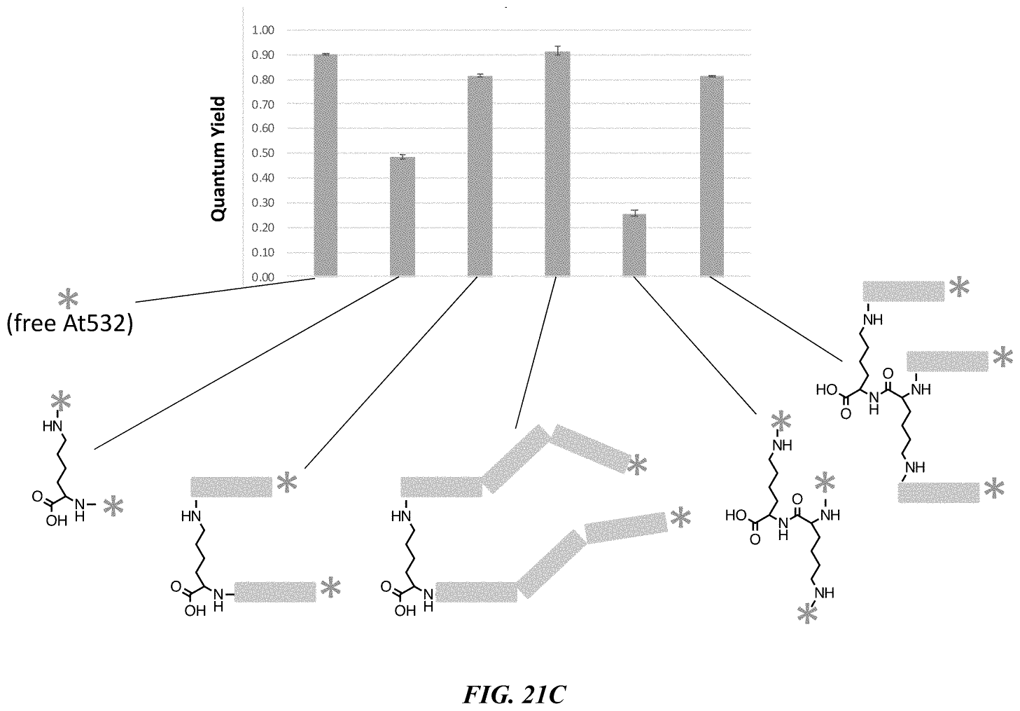

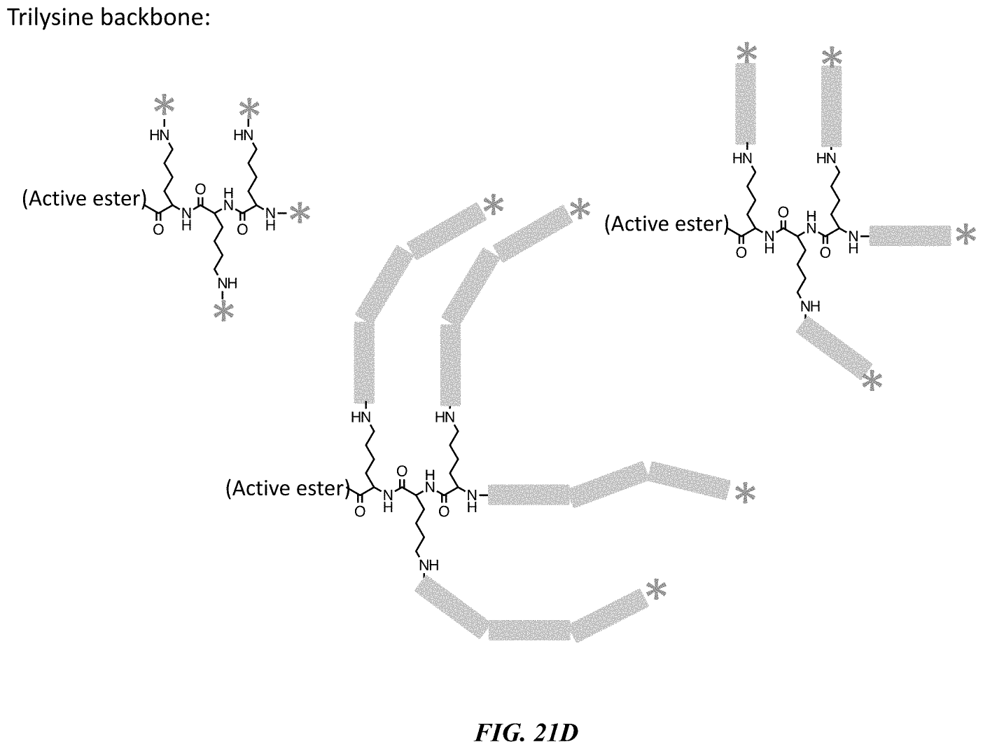

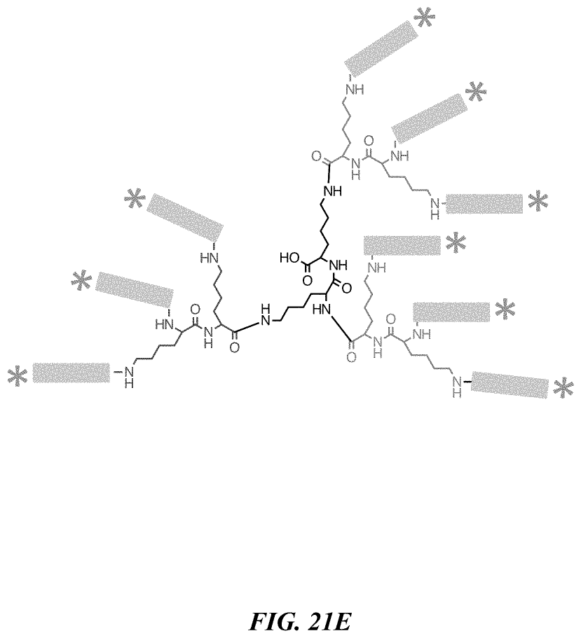

FIG. 21A shows an example scaffold structure for inclusion in a labeling reagent. The scaffold structure includes multiple lysine moieties, each of which comprises an amino group via which a linker moiety may be connected. The illustrated scaffold includes three lysine moieties, which enables incorporation of four linker moieties. In the figure, each linker moiety has the same chemical structure and is connected to an optically detectable moiety having a same structure; however, any useful combination of linkers and optically detectable moieties may be used (e.g., as described herein). The trilysine scaffold also includes an activatable ester moiety that may be connected to additional components of a labeling reagent, such as a cleavable moiety and optional spacer moiety. Alternatively, the activatable ester may facilitate direct connection to a substrate (e.g., as described herein).

FIG. 21B shows additional examples of structures for inclusion in labeling reagents. The upper panel shows structures including a scaffold including a single lysine, which enables incorporation of two linkers and two optically detectable moieties. In the figure, each linker moiety has the same chemical structure and is connected to an optically detectable moiety having a same structure; however, any useful combination of linkers and optically detectable moieties may be used (e.g., as described herein). Three examples of single-lysine-based structures are shown: a structure in which optically detectable moieties (e.g., dyes) are directly linked to amino groups of the lysine, a structure in which optically detectable moieties are linked to amino groups of the lysine via hyp10 moieties (e.g., a linker comprising at least 10 hydroxyprolines), and a structure in which optically detectable moieties are linked to amino groups of the lysine via hyp30 moieties (e.g., a linker comprising at least 30 hydroxyprolines). The lower panel shows structures including a scaffold including two lysines, which enables incorporation of three linkers and three optically detectable moieties. As in the upper panel, though each linker moiety illustrated has the same chemical structure and is connected to an optically detectable moiety having a same structure, any useful combination of linkers and optically detectable moieties may be used (e.g., as described herein). Two examples of dilysine-based structures are shown: a structure in which optically detectable moieties (e.g., dyes) are directly linked to amino groups of the lysine, and a structure in which optically detectable moieties are linked to amino groups of the lysine via hyp10 moieties (e.g., a linker comprising at least 10 hydroxyprolines). FIG. 21C shows relative quantum yields corresponding to each structure illustrated in FIG. 21B as well as for the free optically detectable moiety (here, Atto532). FIG. 21D shows additional structures including a trilysine (e.g., Lys-Lys-Lys) backbone. The structures include a linker having optically detectable moieties coupled directly to the trilysine backbone and linkers having optically detectable moieties coupled to the trilysine backbone via hyp10 or hyp30 moieties. As in the preceding examples, though each linker moiety illustrated has the same chemical structure and is connected to an optically detectable moiety having a same structure, any useful combination of linkers and optically detectable moieties may be used (e.g., as described herein).

FIG. 21E shows a structure for inclusion in a labeling reagent that comprises three dilysines coupled to a fourth dilysine. The structure includes nine linkers and nine optically detectable moieties. Though the linkers and optically detectable moieties are shown as having the same chemical structures, any combination of linkers and optically detectable moieties may be used (e.g., as described herein). The dye moieties in FIG. 21E are shown using the “*” symbol; however, any useful optically detectable moiety may be used. Though optically detectable moieties are represented as single fluorescent dye moieties, dye pairs may also be used. For example, a dye pair may comprise a fluorescence donor and a fluorescence acceptor. A dye pair may comprise a first dye and a second dye. An example dye pair may include AF488 and Atto532 dyes. To evaluate energy transfer between dye pairs, antibodies may be labeled with biotin and then mixed with streptavidin-phycoerythrin (SAPE). This mixture may be used to label cells that may be analyzed using flow cytometry. Alternatively, streptavidin-labeled magnetic beads may be used in place of cells. Biotinylated BSA may be added in excess to beads and then washed. Brightness may then be measured using flow cytometry.

### Amino Acids

A labeling reagent may include a plurality of amino acids in one or more portions of the labeling reagent. For example, an amino acid or plurality of amino acids, such as one or more lysines, may serve as a scaffold to which one or more linkers may attach (e.g., as described herein). Alternatively, or additionally, a linker of a labeling reagent may include one or more amino acids (e.g., as described herein). A labeling reagent may include any useful number of amino acids, such as at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more amino acids. At least a subset of the amino acids of a labeling reagent may be included in sequence (e.g., adjacent to one another). A labeling reagent may comprise multiple different subsets of amino acids, such as multiple different sequences of amino acids. As described herein, amino acids may be arranged in a secondary structure such as a helical structure. For example, a labeling reagent (e.g., a linker of a labeling reagent) may comprise a portion comprising a secondary structure such as a helical structure, such as a helical structure comprising a plurality of prolines, hydroxyprolines, or derivatives thereof. A labeling reagent comprising multiple linkers may comprise multiple sets of amino acids, and each linker of a labeling reagent may comprise a shared or different chemical structure (e.g., an identical sequence of amino acids).

An amino acid may be a natural amino acid or a non-natural amino acid. An amino acid may be a proteinogenic amino acid or a non-proteinogenic amino acid. A “proteinogenic amino acid,” as used herein, generally refers to a genetically encoded amino acid that may be incorporated into a protein during translation. Proteinogenic amino acids include arginine, histidine, lysine, aspartic acid, glutamic acid, serine, threonine, asparagine, glutamine, cysteine, selenocysteine, glycine, proline, alanine, isoleucine, leucine, methionine, phenylalanine, tryptophan, tyrosine, valine, selenocysteine, and pyrrolysine. A “non-proteinogenic amino acid,” as used herein, is an amino acid that is not a proteinogenic amino acid. A non-proteinogenic amino acid may be a naturally occurring amino acid or a non-naturally occurring amino acid. Non-proteinogenic amino acids include amino acids that are not found in proteins and/or are not naturally encoded or found in the genetic code of an organism. Examples of non-proteinogenic amino acids include, but are not limited to, hydroxyproline, selenomethionine, hypusine, 2-aminoisobutyric acid, αγ-aminobutyric acid, ornithine, citrulline, β-alanine (3-aminopropanoic acid), δ-aminolevulinic acid, 4-aminobenzoic acid, dehydroalanine, carboxyglutamic acid, pyroglutamic acid, norvaline, norleucine, alloisoleucine, t-leucine, pipecolic acid, allothreonine, homocysteine, homoserine, α-amino-n-heptanoic acid, α,β-diaminopropionic acid, α, γ-diaminobutyric acid, β-amino-n-butyric acid, β-aminoisobutyric acid, isovaline, sarcosine, N-ethyl glycine, N-propyl glycine, N-isopropyl glycine, N-methyl alanine, N-ethyl alanine, N-methyl β-alanine, N-ethyl β-alanine, isoserine, and α-hydroxy-γ-aminobutyric acid. Additional examples of non-proteinogenic amino acids include the non-natural amino acids described herein. A non-proteinogenic amino acid may comprise a ring structure. For example, a non-proteinogenic amino acid may be trans-4-aminomethylcyclohexane carboxylic acid or 4-hydrazinobenzoic acid. Such compounds may be FMOC-protected with FMOC (fluorenylmethyloxycarbohyl chloride) and utilized in solid-phase peptide synthesis. The structures of these compounds are shown below:

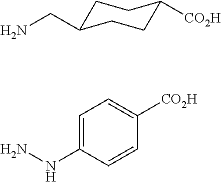 [SMILES: NCC1CCC(C(=O)O)CC1 ; NNc1ccc(C(=O)O)cc1]

Where a labeling reagent or a linker thereof comprises multiple amino acids, such as multiple non-proteinogenic amino acids, an amine moiety adjacent to a ring moiety (e.g., the amine moiety in the hydrazine moiety) can function as a water-solubilizing group. To synthesize a water-soluble peptide, a hybrid linker can be made that comprises alternating non-water-soluble amino acids and water-soluble amino acids (e.g., hydroxyproline). Other moieties can be used to increase water-solubility. For example, linking amino acids with oxamate moieties can provide water-solubility through the additional hydrogen bonding without adding any sp³ linkages. The structure of the oxamate precursor 2-amino-2-oxoacetic acid is shown below:

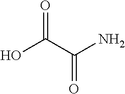

In some cases, a component (e.g., a monomer unit) of a linker may have an amino group, a carboxy group, and a water-solubilizing moiety. In some cases, a monomer may be deconstructed as two “half-monomers.” That is, by using two different units, one that contains two amino groups and another that contains two carboxy groups, an amino acid moiety can be constructed, which amino acid moiety may be a unit (e.g., a repeated unit) of a linker. One or both units may include one or more water solubilizing moieties. For example, at least one unit may include a water-soluble group (e.g., as described herein). For example, 2,5-diaminohydroquinone can be one half-monomer (A), and 2,5-dihydroxyterephthalic acid may be the other half-monomer (B). Such a scheme is shown below:

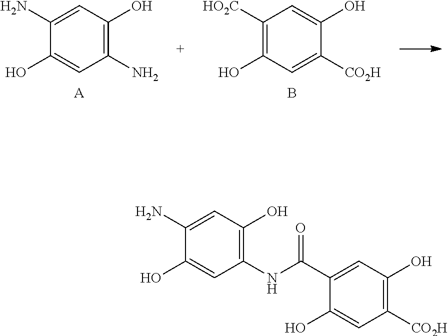

As shown above, A is a diamine and B is a diacid. Accordingly, non-proteinogenic (e.g., non-natural) amino acids may be constructed from diamines and diacids. An additional example of such a construction is shown below:

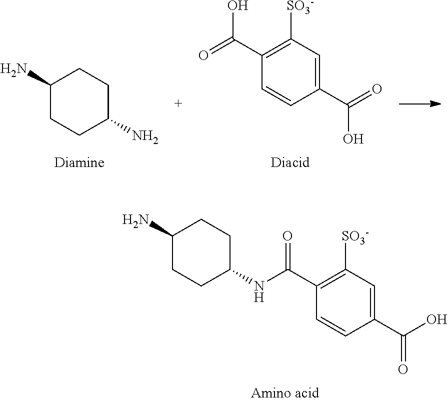

A polymer based on two half-monomers (e.g., as shown above) can be constructed via solid phase synthesis. Because the half-monomers can be homobifunctional in the linking moiety, in some cases no FMOC protection is required. For example, the dicarboxylic acid can be appended to the solid support, then an excess of the diamine added with appropriate coupling reagent (HBTU/HOBT/collidine). After washing away excess reagent, an excess of the dicarboxylic acid can be added with the coupling reagent. Side-products consisting of one molecule of the fluid phase reagent reacting with two solid-phase attached reagent can result in truncation of the synthesis. These side products can be separated from a product after cleavage from the support and purification by HPLC.

An advantage of the half-monomers approach can be increased flexibility in creating polymers. The diamine (A) can be replaced in a subsequent step by a different diamine (A′) to change the properties of the polymer, in a repeating or non-repeating manner. Such a scheme may facilitate construction of a polymer such as ABA′BABA′B.

Additional examples of half-monomers for use according to the schemes described above include 2,5-diaminopyridine and 2,5-dicarboxypyridine, both of which are shown below, as well as the other moieties shown below:

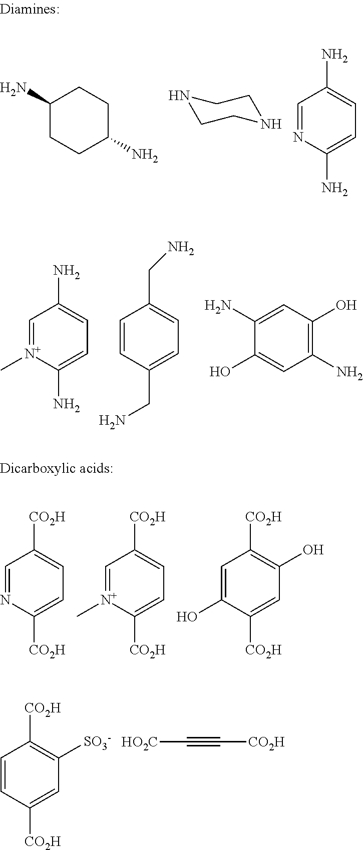

As described above, an amino acid (e.g., a non-proteinogenic amino acid that may be a non-natural amino acid) may be constructed from a diamine and a dicarboxylic acid. An amino acid (e.g., a non-proteinogenic amino acid that may be a non-natural amino acid) may also be constructed from an amino thiol and a thiol carboxylic acid. Examples of amino thiols and thiol carboxylic acids are shown below:

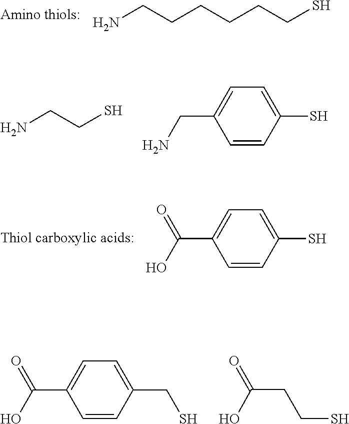

Examples of amino acids (e.g., non-natural amino acids) constructed from an amino thiol and a thiol carboxylic acid are shown below:

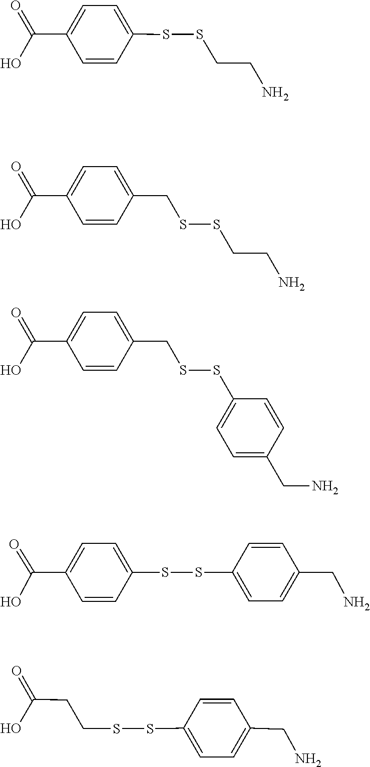 [SMILES: NCCSSc1ccc(C(=O)O)cc1 ; NCCSSCc1ccc(C(=O)O)cc1 ; NCc1ccc(SSCc2ccc(C(=O)O)cc2)cc1 ; NCc1ccc(SSc2ccc(C(=O)O)cc2)cc1 ; NCc1ccc(SSCCC(=O)O)cc1]

As shown above, amino acids constructed using an amino thiol and a thiol carboxylic acid may include a disulfide bond. As described elsewhere herein, a disulfide bond may be cleavable using a cleavage reagent (e.g., as described herein). Accordingly, an amino acid constructed from an amino thiol and a thiol carboxylic acid may serve as a cleavable portion of a linker. An amino acid constructed from an amino thiol and a carboxylic acid may be a component of a linker (e.g., as described herein) that may couple labeling moiety (e.g., a fluorescent dye) to a substrate (e.g., a nucleotide or nucleotide analog). The various structures allow different hydrophobicities for incorporation and may provide different “scar” moieties subsequent to interaction with a cleavage reagent (e.g., as described herein). Two or more amino acids, such as two or more amino acids constructed from an amino thiol and a thiol carboxylic acid, may be included in a linker. For example, two or more amino acids may be included in a linker and separated by no more than 2 sp³ carbon atoms, such as by no more than 2 sp² carbon atoms or by no more than 2 atoms. Where two or more amino acids formed of amino thiols and thiol carboxylic acids are connected to one another within a linker, cleavage may be more rapid as there will be multiple possible sites for cleavage. An example of a portion of a linker including such a component is shown below:

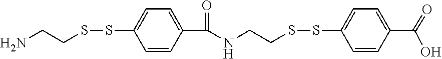 [SMILES: NCCSSc1ccc(C(=O)NCCSSc2ccc(C(=O)O)cc2)cc1]

As described above, two half-monomers may combine to provide an amino acid (e.g., a non-proteinogenic amino acid, such as a non-natural amino acid). Accordingly, a non-natural amino acid may include any known non-natural amino acid, as well as any non-natural amino acid that may be constructed as described herein.

Half-monomers such as those described herein can be constructed into polypeptide polymers. An example of a nucleotide constructed with two repeating units of an amino acid is shown below.

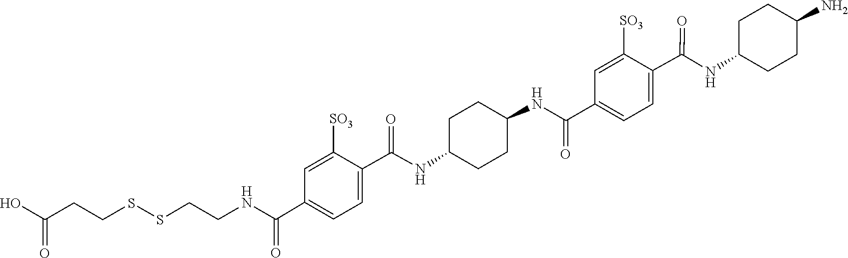

In some cases, before or after peptide coupling, the nitrogen in a nitrogen-containing ring can be quaternized to provide pyridinium moieties, thereby improving water-solubility of the final product. An example linker sequence generated in this manner is shown below:

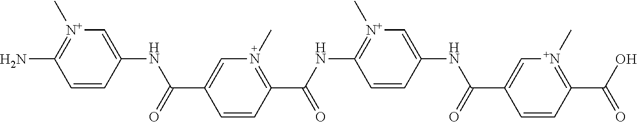

Water-solubilizing linkages that can work with the half-monomer method include, for example, those that have symmetrical functional groups, such as secondary amides, bishydrazides, and ureas. Examples of such moieties are shown below:

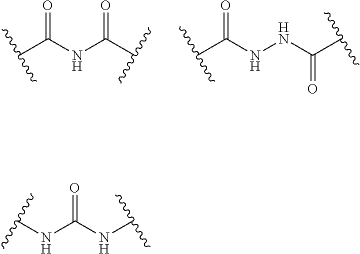 [SMILES: *C(=O)NC(*)=O ; *C(=O)NNC(*)=O ; *NC(=O)N*]

Amino acid linker subunits may be assembled into polymers by peptide synthesis methods. For example, a solid support method known as SPPS (Solid Phase Peptide Synthesis) or by liquid-phase synthesis may be used to assemble amino acids into a linker. SPPS methods can use a solid phase bead where the initial step is attachment of the C-terminal amino acid via its carboxylic acid moiety, leaving its free amine ready for coupling. Peptide synthesis can be initiated by flowing FMOC amine-protected monomers with peptide coupling reagents such as HBTU and an organic base. Excess reagent can be washed away and the next monomer is introduced. After one or more amino acids have been appended the final peptide can be cleaved from the beads and purified by HPLC. Liquid phase synthesis can use the same reagents (except the beads) but purification occurs after each step. The advantage of either stepwise polymerization process is that the resultant linkers can have a defined molecular weight that may be confirmed by mass spectrometry.

A labeling reagent may include any useful combination of amino acids, including any combination of natural and non-natural amino acids and/or proteinogenic and non-proteinogenic amino acids. As described herein, a labeling reagent may comprise a sequence of hydroxyprolines such as a hyp10, hyp20, hyp30, or similar moiety (e.g., hypn).

### Cleavable Moieties

A labeling reagent may include one or more cleavable moieties (e.g., as described herein). A cleavable moiety may comprise a cleavable group such as a disulfide moiety. A cleavable moiety may comprise a chemical handle for attachment to a substrate (e.g., as described herein). Accordingly, a cleavable moiety may be included in a labeling reagent at a position adjacent to a substrate to which the labeling reagent is attached. A cleavable moiety may be coupled to a linker component of a labeling reagent via, for example, reaction between a free carboxyl moiety of the linker component and an amino moiety of a cleavable moiety (e.g., cleavable linker portion).

Examples of cleavable linker portions include, but are not limited to, the structures E, B, and Y shown below:

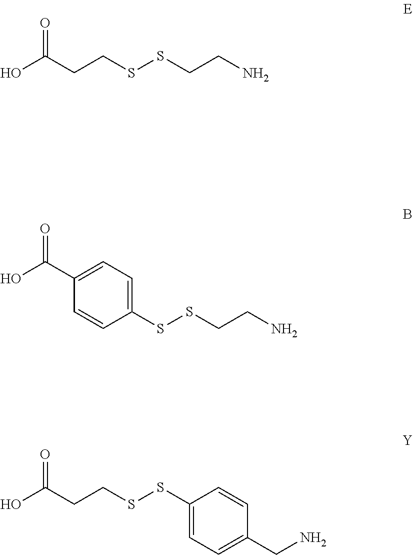 [SMILES: NCCSSCCC(=O)O ; NCCSSc1ccc(C(=O)O)cc1 ; NCc1ccc(SSCCC(=O)O)cc1]

In the structures shown above, the disulfide moieties may be cleaved (e.g., as described herein) to provide thiol scars. Variations of the structures shown above are also contemplated. For example, one or more substituents such as one or more alkyl, hydroxyl, alkoxy, or halo moieties may be attached to a ring structure or an available carbon atom in any of the above structures. Similarly, though para attachment of carboxyl and disulfide moieties is illustrated, meta and ortho attachments may also be used. Moreover, an optionally substituted alkyl group may be incorporated between a ring structure and a disulfide moiety. A cleavable linker portion may be attached to a substrate upon reaction between a carboxyl moiety of the cleavable linker moiety and an amine moiety attached to a substrate (e.g., protein, nucleotide or nucleotide analog, cell, etc., as described herein) to provide the substrate attached to the cleavable linker portion via an amide moiety. For example, the substrate may be a nucleotide or nucleotide analog including a propargylamino moiety, and a fluorescent labeling reagent comprising a dye and a linker described herein may be configured to associate with the substrate via the propargylamino moiety. Examples of such substrates are shown below:

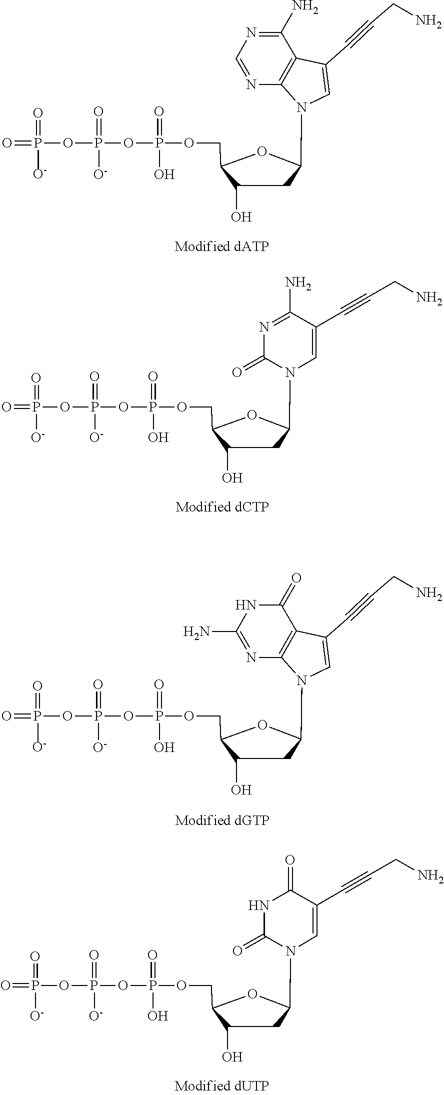

### Optically Detectable Moieties

As described herein, a labeling reagent may comprise one or more optically detectable moieties. Multiple optically detectable moieties (e.g., fluorescent dye moieties) included in a given labeling reagent may have the same or different chemical structures. Similarly, multiple optically detectable moieties (e.g., fluorescent dye moieties) included in a given labeling reagent may fluoresce at or near the same wavelengths or may fluoresce at or near different wavelengths. A given linker component (e.g., semi-rigid linker component) may be configured to couple to a single optically detectable moiety. Alternatively, a given linker component (e.g., semi-rigid linker component) may be configured to couple to two or more optically detectable moieties that may have the same or different chemical structures. A labeling reagent may include multiple linkers coupled to multiple optically detectable moieties via, e.g., a scaffold such as a lysine or polylysine scaffold (e.g., as described herein). Optically detectable moieties coupled to a labeling reagent may facilitate optical (e.g., fluorescent) labeling of a substrate to which the labeling reagent may attach. For example, the labeling reagent may be used to optically label a protein, nucleotide, nucleotide analog, polynucleotide, antibody, cell, saccharide, polysaccharide, lipid, cell surface marker, or any other useful substrate (e.g., as described herein) with one or more optically detectable moieties. When coupled to a substrate, a labeling reagent comprising multiple optically detectable moieties configured to provide a similar optical signal (e.g., configured to fluoresce at or near the same wavelengths) may provide an enhanced signal relative to a labeling reagent comprising a single optically detectable moiety.

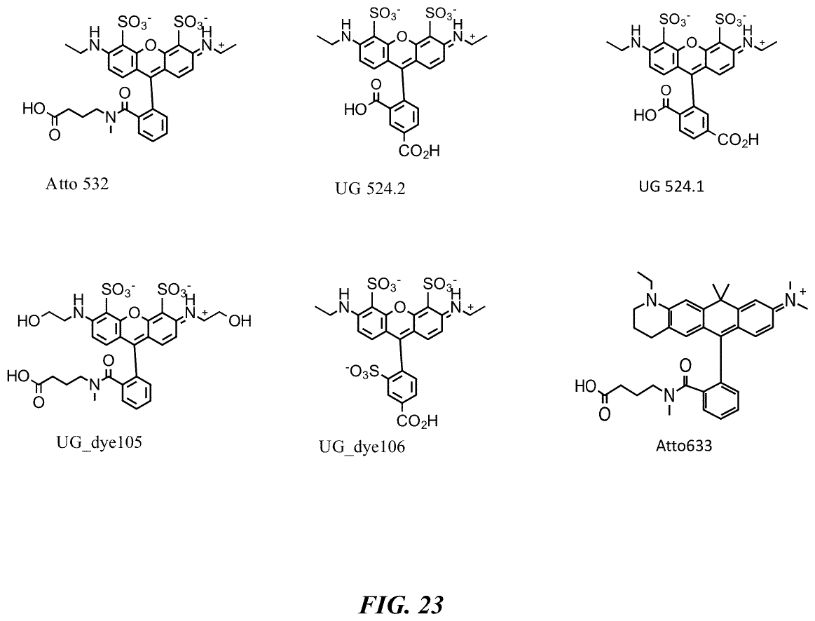

An optically detectable moiety may comprise a dye (e.g., a fluorescent dye). Non-limiting examples of dyes (e.g., fluorescent dyes) include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO dyes (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor dyes (e.g., AlexaFluor 350, 405, 430, 488, 532, 546, 555, 568, 594, 610, 633, 635, 647, 660, 680, 700, 750, and 790 dyes), DyLight dyes (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (from Molecular Probes/Invitrogen)(e.g., QSY7, QSY9, QSY21, and QSY35), Dabcyl, Dabsyl. CySQ, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, 580Q, 612Q, 532, and 633), and other fluorophores and quenchers (e.g., as described herein). Additional examples of dyes are shown in FIG. 23.

A fluorescent dye may be excited over a single wavelength or a range of wavelengths. In some cases, an optical labeling reagent may comprise an optically detectable moiety configured to fluoresce in the red region of the electromagnetic spectrum (e.g., (about 625-740 nm). For example, a labeling reagent may include a fluorescent dye that may emit signal in the red region of the visible portion of the electromagnetic spectrum (about 625-740 nm) (e.g., have an emission maximum in the red region of the visible portion of the electromagnetic spectrum). Alternatively or additionally, an optical labeling reagent may comprise an optically detectable moiety configured to fluoresce in the green region of the electromagnetic spectrum (e.g., about 500-565 nm). For example, a labeling reagent may include a fluorescent dye that may emit signal in the green region of the visible portion of the electromagnetic spectrum (about 500-565 nm) (e.g., have an emission maximum in the green region of the visible portion of the electromagnetic spectrum). Similarly, a fluorescent dye may be excitable by light in the red region of the visible portion of the electromagnetic spectrum (about 625-740 nm) (e.g., have an excitation maximum in the red region of the visible portion of the electromagnetic spectrum). Alternatively or additionally, fluorescent dye may be excitable by light in the green region of the visible portion of the electromagnetic spectrum (about 500-565 nm) (e.g., have an excitation maximum in the green region of the visible portion of the electromagnetic spectrum). In an example, an optical labeling reagent may include a plurality of optically detectable moieties configured to fluoresce in the red region of the visible portion of the electromagnetic spectrum, which plurality of optically detectable moieties may have the same or different structures. In another example, an optical labeling reagent may include a plurality of optically detectable moieties configured to fluoresce in the green region of the visible portion of the electromagnetic spectrum, which plurality of optically detectable moieties may have the same or different structures.

In some cases, the label may be a type that does not self-quench or exhibit proximity quenching. Non-limiting examples of a label type that does not self-quench or exhibit proximity quenching include Bimane derivatives such as Monobromobimane. Additional dyes included in structures provided herein may also be utilized in combination with any of the linkers provided herein, and with any substrate described herein, regardless of the context of their disclosure. In some cases, an optically detectable moiety may comprise a dye pair (e.g., two or more dye structures). A labeling reagent including any useful optically detectable moiety, or any combination of optically detectable moieties, may be useful in, for example, labeling a nucleotide or nucleotide analog for use in a sequencing assay. For example, a sequencing assay performed with a nucleotide labeled with a red-fluorescing dye and a sequencing assay performed with a nucleotide labeled a green-fluorescing dye may have sequencing quality and signal-to-noise ratios, as well as other performance metrics.

### Labeled Substrates

An optical (e.g., fluorescent) labeling reagent may be configured to associate with a substrate such as a nucleotide or nucleotide analog (e.g., as described herein). Alternatively or additionally, an optical (e.g., fluorescent) labeling reagent may be configured to associate with a substrate such as a protein, cell, lipid, or antibody. For example, the optical labeling reagent may be configured to associate with a protein. A protein substrate may be any protein, and may include any useful modification, mutation, or label, including any isotopic label. For example, a protein may be an antibody such as a monoclonal antibody. A protein associated with one or more optical (e.g., fluorescent) labeling reagents (e.g., as described herein) may be, for example, an antibody (e.g., a monoclonal antibody) useful for labeling a cell, which labeled cell may be analyzed and sorted using flow cytometry.

An optical (e.g., fluorescent) labeling reagent (e.g., as described herein) can decrease quenching (e.g., between dyes coupled to nucleotides or nucleotide analogs incorporated into a growing nucleic acid strand, such as during nucleic acid sequencing). For example, an optical (e.g., fluorescent) signal emitted by a substrate (e.g., a nucleotide or nucleotide analog that may be incorporated into a growing nucleic acid strand) can be proportional to the number of optical (e.g., fluorescent) labels associated with the substrate (e.g., to the number of optical labels incorporated adjacent or in proximity to the substrate). For example, multiple optical labeling reagents including substrates of the same or different types (e.g., nucleotides or nucleotide analogs of a same or different type) may be incorporated in proximity to one another in a growing nucleic acid strand (e.g., during nucleic acid sequencing). In such a system, signal emitted by the collective substrates may be approximately proportional (e.g., linearly proportional) to the number of dye-labeled substrates incorporated. In other words, quenching may not significantly impact the signal emitted. This may be observable in a system in which 100% labeling fractions are used. Where less than 100% of substrates are labeled (e.g., less than 100% of nucleotides in a nucleotide flow are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a homopolymer region of the growing nucleic acid strands. Similarly, where less than 100% of substrates are labeled (e.g., less than 100% of nucleotides in each of successive nucleotide flows are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a heteropolymeric and/or homopolymer region of the growing nucleic acid strands. In some such cases, the intensity of a measured optical (e.g., fluorescent) signal may be linearly proportional to the length of a heteropolymeric and/or homopolymeric region into which substrates have incorporated. For example, a measured optical (e.g., fluorescent) signal may be linearly proportional with a slope of approximately 1.0 when optical (e.g., fluorescent) signal is plotted against the length in substrates of a heteropolymeric and/or homopolymeric region into which substrates have incorporated.

An optical (e.g., fluorescent) labeling reagent (e.g., as described herein) can decrease quenching in a protein system. When labeling proteins, quenching may start to happen at a fluorophore to protein ratio (F/P) of around 3. Using optical labeling reagents provided herein, higher F/P ratios, and thus brighter reagents, may be obtained. This may be useful for analyzing proteins (e.g., using imaging) and/or for analyzing cells labeled with proteins (e.g., antibodies) associated with one or more optical (e.g., fluorescent) labeling reagents.

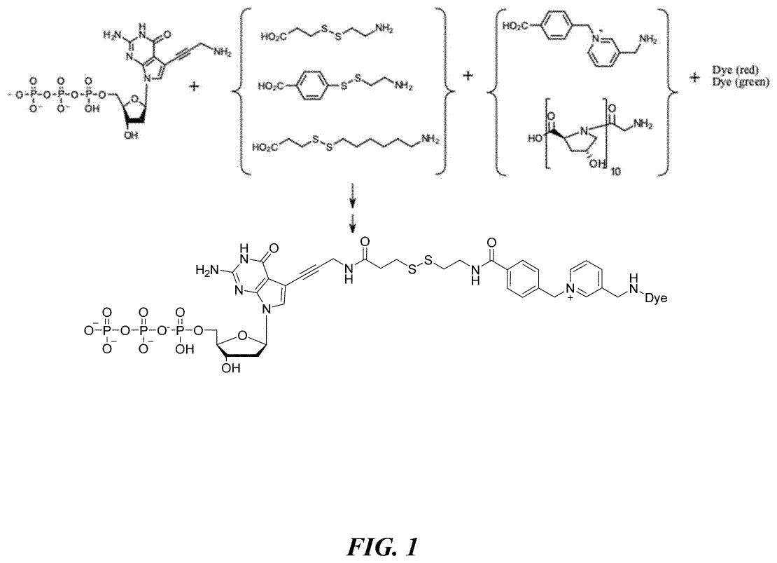

Examples of labeling reagents provided herein, or components thereof, are included in, e.g., FIGS. 1, 2A, 2B, 6, 7, 8, 3A-3C, 14A, 14B, 16, and 17. Additional examples are included elsewhere herein, including in the Examples below. Any useful labeling reagent may be used to label any substrate of interest.

In an aspect, the present disclosure provides a labeled substrate comprising a substrate (e.g., as described herein) and an optical labeling reagent (e.g., as described herein), or a derivative thereof, where the optical labeling reagent is coupled to the substrate. The substrate may be, for example, a nucleotide, polynucleotide, protein, lipid, cell, saccharide, polysaccharide, or antibody. For example, the substrate may be a protein. Alternatively or additionally, the substrate may be a component of a cell. In another example, the substrate may be a nucleotide or nucleotide analog and the optical labeling reagent may be coupled to the nucleotide via the nucleobase of the nucleotide. The substrate may be a fluorescence quencher, a fluorescence donor, or a fluorescence acceptor. The labeled substrate may reduce quenching relative to another labeled substrate comprising the substrate and another fluorescent labeling reagent that comprises one or more optically detectable moieties but does not include a linker provided herein. Similarly, the labeled substrate may provide a higher signal level upon excitation and optical detection relative to another labeled substrate comprising the substrate and another fluorescent labeling reagent that comprises one or more optically detectable moieties but does not include a linker provided herein.

The substrate may comprise an additional optical labeling reagent (e.g., fluorescent labeling reagent) coupled thereto. The additional optical labeling reagent may comprise an optically detectable moiety (e.g., fluorescent dye moiety) and a linker connected to the optically detectable moiety. The linker and optically detectable moiety of the additional optical labeling reagent may be coupled to the substrate via a cleavable linker portion (e.g., as described herein). The additional optical labeling reagent may include a scaffold to which multiple linkers and optically detectable moieties may be coupled (e.g., as described herein). An optically detectable moiety of a first optical labeling reagent coupled to a substrate and an optically detectable moiety of a second optical labeling reagent coupled to the same substrate may have identical chemical structures. Alternatively or additionally, an optically detectable moiety of a first optical labeling reagent coupled to a substrate and an optically detectable moiety of a second optical labeling reagent coupled to the same substrate may have different chemical structures.

In an aspect, the present disclosure provides an oligonucleotide molecule comprising a fluorescent labeling reagent or derivative thereof (e.g., as described herein). The oligonucleotide molecule may comprise one or more additional fluorescent labeling reagents of a same type (e.g., comprising linkers having the same chemical structure, dyes comprising the same chemical structure, and/or associated with substrates (e.g., nucleotides) of a same type). The fluorescent labeling reagent and one or more additional fluorescent labeling reagents of the oligonucleotide molecule may be associated with nucleotides. For example, the fluorescent labeling reagents may be connected to nucleobases of nucleotides of the oligonucleotide molecule. A fluorescent labeling reagent and one or more additional fluorescent labeling reagent may be connected to adjacent nucleotides of the oligonucleotide molecule. Alternatively or additionally, the fluorescent labeling reagent and the one or more additional fluorescent labeling reagents may be connected to nucleotides of the oligonucleotide molecule that are separated by one or more nucleotides that are not connected to fluorescent labeling reagents. The oligonucleotide molecule may be a single-stranded molecule. Alternatively, the oligonucleotide molecule may be a double-stranded or partially double-stranded molecule. A double-stranded or partially double-stranded molecule may comprise fluorescent labeling reagents associated with a single strand or both strands. The oligonucleotide molecule may be a deoxyribonucleic acid molecule. The oligonucleotide molecule may a ribonucleic acid molecule. The oligonucleotide molecule may be generated and/or modified via a nucleic acid sequencing process (e.g., as described herein).

The fluorescent labeling reagent may comprise a cleavable group (e.g., as described herein) that is configured to be cleaved to separate the fluorescent dye of the fluorescent labeling reagent from a substrate (e.g., nucleotide) with which it is associated. For example, the labeling reagent may comprise a cleavable group comprising an azidomethyl group, a disulfide bond, a hydrocarbyldithiomethyl group, or a 2-nitrobenzyloxy group. The cleavable group may be configured to be cleaved by application of one or more members of the group consisting of tris(2-carboxyethyl)phosphine (TCEP), dithiothreitol (DTT), tetrahydropyranyl (THP), ultraviolet (UV) light, and a combination thereof. The oligonucleotide molecule comprising a fluorescent labeling reagent may be configured to emit a fluorescent signal (e.g., upon excitation at an appropriate range of energy, as described herein).

In another aspect, the present disclosure provides a kit comprising a plurality of linkers (e.g., as described herein). A linker may be a component of an optical labeling reagent provided herein. A linker may be linked to a scaffold such as a lysine or polylysine scaffold. A linker may comprise a cleavable group (e.g., as described herein) configured to be cleaved to separate a linker from a substrate to which it may be attached. A linker may comprise one or more amino acids, such as one or more non-proteinogenic amino acids. For example, a linker may comprise at least one hydroxyproline. A linker may comprise a hyp10, hyp20, hyp30, or other hypn moieties. Alternatively or additionally, a linker may comprise a non-natural amino acid (e.g., as described herein). A linker may be configured to provide a functional separation between an optically detectable moiety and a substrate of at least, e.g., about 9 Å, such as at least 12 Å, 15 Å, 20 Å, 25 Å, 30 Å, 36 Å, or more (e.g., as described herein). A linker may be connected to an optically detectable moiety (e.g., fluorescent dye; as described herein) and/or associated with a substrate (e.g., as described herein). For example, the linker may be connected to a fluorescent dye and coupled to a substrate selected from a nucleotide, a protein, a lipid, a cell, and an antibody. For example, the linker may be connected to an optically detectable moiety (e.g., fluorescent dye) and a substrate such as a nucleotide.

A linker may comprise a plurality of amino acids, such as a plurality of non-proteinogenic (e.g., non-natural) amino acids. For example, the linker may comprise a plurality of hydroxyprolines (e.g., a hyp10 moiety or other hypn moieties). A linker may comprise a cleavable group that is configured to be cleaved to separate a first portion of the linker from a second portion of the linker. The cleavable group may be selected from the group consisting of an azidomethyl group, a disulfide bond, a hydrocarbyldithiomethyl group, and a 2-nitrobenzyloxy group. The cleavable group may be cleavable by application of one or more members of the group consisting of tris(2-carboxyethyl)phosphine (TCEP), dithiothreitol (DTT), tetrahydropyranyl (THP), ultraviolet (UV) light, and a combination thereof. The linker may comprise a cleavable linker portion comprising a moiety selected from the group consisting of

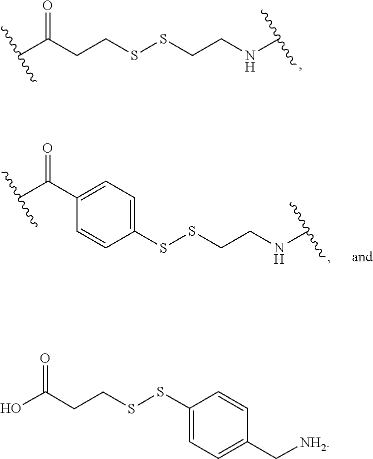 [SMILES: *NCCSSCCC(*)=O ; *NCCSSc1ccc(C(*)=O)cc1 ; NCc1ccc(SSCCC(=O)O)cc1]

The plurality of linkers of the kit may comprise a first linker associated with a first substrate (e.g., a first nucleotide) and a second linker associated with a second substrate (e.g., a second nucleotide). The first substrate and the second substrate may be of different types (e.g., different canonical nucleotides). The first substrate and the second substrate may be nucleotides comprising nucleobases of different types (e.g., A, C, G, U, and T). The first linker and the second linker may comprise the same chemical structure. Similarly, the first linker may be connected to a first fluorescent dye and the second linker may be connected to a second fluorescent dye. The first fluorescent dye and the second fluorescent dye may be of different types. For example, the first and second fluorescent dyes may fluoresce at different wavelengths and/or have different maximum excitation wavelengths. The first and second fluorescent dyes may fluoresce at similar wavelengths and/or have similar maximum excitation wavelengths regardless of whether they share the same chemical structure.

The plurality of linkers of the kit may further comprise a third linker associated with a third substrate and a fourth linker associated with a fourth substrate. The first substrate, the second substrate, the third substrate, and the fourth substrate may be of different types. For example, the first substrate, the second substrate, the third substrate, and the fourth substrate may be nucleotides comprising nucleobases of different types (e.g., A, C, G, and U/T). The first linker and the third linker may comprise different chemical structures. The first and third linker may comprise a same chemical group, such as a same cleavable group (e.g., as described herein). For example, the first linker and the third linker may each comprise a moiety comprising a disulfide bond. Similarly, the first linker and the fourth linker may comprise different chemical structures. The first and fourth linker may comprise a same chemical group, such as a same cleavable group (e.g., as described herein). For example, the first linker and the fourth linker may each comprise a moiety comprising a disulfide bond.

In an example, the first linker comprises a hyp10 moiety and a first cleavable moiety, the second linker comprises a hyp10 moiety and a second cleavable moiety, the third linker comprises a third cleavable moiety and does not comprise a hyp10 moiety, and the fourth linker comprises a fourth cleavable moiety and does not comprise a hyp10 moiety. The second cleavable moiety may have a chemical structure that is different from the first cleavable moiety. Alternatively, the second cleavable moiety and the first cleavable moiety may have the same chemical structures. The third cleavable moiety and the fourth cleavable moiety may have the same chemical structure. Alternatively, the third cleavable moiety and the fourth cleavable moiety may have different chemical structures. In an example, the first linker and the second linker each have a first chemical structure and the third linker and the fourth linker each have a second chemical structure, which second structure is different from the first chemical structure. In another example, the first linker, the second linker, the third linker, and the fourth linker all have the same chemical structure. In another example, the first linker, the second linker, the third linker, and the fourth linker all have different chemical structures.

One or more linkers in a kit may be components of a labeling reagent. Accordingly, in an aspect, the present disclosure provides a kit comprising a plurality of labeling reagents (e.g., as described herein). The plurality of labeling reagents may have identical chemical structures. Alternatively, the plurality of labeling reagents may comprise at least a first plurality of labeling reagents having a first chemical structure and a second plurality of labeling reagents having a second chemical structure different from the first chemical structure. A labeling reagent of a kit may have any useful features, as described herein. For example, a labeling reagent of a kit may comprise a cleavable portion configured to be cleaved to separate a substrate from a portion of the labeling reagent (e.g., as described herein); a semi-rigid linker portion comprising, for example, one or more sequences of hydroxyprolines (e.g., a hyp10, hyp20, or hyp30 moiety, as described herein); an optically detectable moiety (e.g., a fluorescent dye moiety, as described herein); and a scaffold to which a linker may be coupled (e.g., a lysine, dilysine, or other polylysine structure, as described herein).

### Methods for Using the Optical Labeling Reagents

There are several different types of quenching that can be reduced and different types of applications that can be performed using the optical (e.g., fluorescent) labeling reagents described herein.

The methods described herein can be used to reduce quenching, including G-quenching. Attachment of dyes (e.g., fluorescent dyes) to nucleotides (e.g., via a linker provided herein) can result in dye-quenching for many dyes, particularly when the dye is attached to a guanosine nucleotide. Dye quenching may take place between a dye and a nucleotide with which it is associated, as well as between dye moieties, such as between dye moieties coupled to different nucleotides (e.g., adjacent nucleotides or nucleotides separated by one or more other nucleotides). Use of the linkers provided herein can alleviate the quenching allowing more sensitive detection of sequences containing G. In addition, a dye-labeled nucleotide in proximity to a G-homopolymer region may show reduced fluorescence. Any nucleic acid sequencing method that requires attachment of a dye to dGTP may benefit from these linkers, including single molecule detection, sequencing using 3′-blocked nucleotides, and sequencing by hybridization.

The methods described herein can be used to reduce dye-dye quenching on adjacent or neighboring nucleotides (e.g., nucleotides separated by one, two, or more other nucleotides) on the same DNA strand. Methods that require dyes on adjacent or neighboring nucleotides can result in proximity quenching; that is, two dyes next to each other are less bright than twice the brightness of one dye, or often, less bright than even a single dye. Use of the linkers provided herein may alleviate the quenching, allowing quantitative detection of multiple dyes. For example, in sequencing methods such as mostly natural nucleotide flow sequencing, the fraction of labeled dye is typically less than 5%, since homopolymers are not linear in signal to homopolymer length at higher fractions due to the quenching problem. The reagents described herein can allow more (e.g., more than 5%, in some cases up to 100%) of the nucleotides to be labeled while facilitating sensitive and accurate detection of incorporated nucleotides.

The use of a labeled nucleotide (e.g., dye-linker-nucleotide) provided herein may result in more efficient incorporation into a growing nucleic acid strand (e.g., increased tolerance) by a polymerase (e.g., as described herein), compared to a dye-nucleotide lacking the linker (e.g., during nucleic acid sequencing). The result may be that a lower amount of the dye-labeled nucleotide is used to achieve the same signal.

The use of a labeled nucleotide (e.g., dye-linker-nucleotide) provided herein may result in less misincorporation by a polymerase (e.g., as described herein) (e.g., during nucleic acid sequencing). The result may be less loss of template strands, and thus longer sequencing reads.

The use of a labeled nucleotide (e.g., dye-linker-nucleotide) provided herein may result in less mispair extension (e.g., during nucleic acid sequencing), and thus reduced lead phasing.

The methods described herein can be used to reduce dye-dye quenching in multi-dye applications. Hybridization assays can also benefit from linkers that prevent quenching. Quenching effects may result in non-linearity of target to signal.

The methods described herein can be used in combination with oligomers and dendrimers for signal amplification. Non-quenching linkers may allow the synthesis of very bright polymers for antibody labeling. These bright antibodies may be used for cell-surface labeling in flow cytometry or for antigen detection methods such as lateral flow tests and fluorescent immunoassays.

The optical (e.g., fluorescent) labeling reagent of the present disclosure may be used as a molecular ruler. The substrate can be a fluorescence quencher, a fluorescence donor, or a fluorescence acceptor. In some cases, the substrate is a nucleotide. The linker can be attached to the nucleotide on the nucleobase as shown below, where the dye is Atto633:

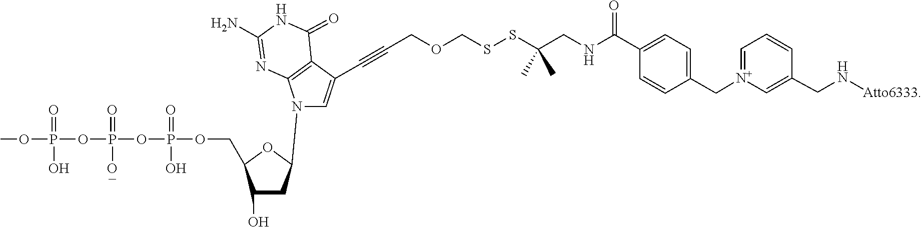

The structure shown above is an optical (e.g., fluorescent) labeling reagent comprising a cleavable (via the disulfide bond) moiety and a fluorescent dye attached via a pyridinium linker to a dGTP analog (dGTP-SS-py-Atto633). Additional examples of optical labeling reagents are provided elsewhere herein.

The labeled nucleotides (e.g., dye-linker-nucleotides) described herein can be used in a sequencing by synthesis method using a mixture of dye-labeled and natural nucleotides in a flow-based scheme. Such methods often use a low percentage of labeled nucleotides compared to natural nucleotides. However, using a low percentage of labeled nucleotides compared to natural nucleotides in flow mixtures (e.g., less than 20%) can have multiple drawbacks: (a) since a small fraction of the template provides sequence information, the method requires a high template copy number; (b) variability in DNA polymerase extension rates between labeled and unlabeled nucleotides can result in context-dependent labeling fractions, thus increasing the difficulty of distinguishing a single base incorporation from multiple base incorporations; and (c) the low fraction of labeling moieties can result in high binomial noise in the populations of labeled product. Methods for flow-based sequencing using mostly natural nucleotides are further described in U.S. Pat. No. 8,772,473, which is incorporated herein by reference in its entirety for all purposes.

In general, the use of labeling reagents including multiple optically detectable moieties, and/or high labeling fractions of dye-labeled nucleotides, may improve signal contrast. For example, signal-to-noise effects may decrease significantly as labeling fraction increases. The labeling reagents comprising semi-rigid linkers provided herein may allow a labeled fraction of dye-labeled nucleotide to natural nucleotide in each flow to be sufficiently high (e.g., 20-100% labeling) to avoid or reduce the effect of the aforementioned disadvantages of, e.g., various sequencing schemes. This higher percentage labeling can result in greater optical (e.g., fluorescent) signal and thus a lower template requirement. If 100% labeling is used, the binomial noise and context variation may be essentially eliminated. The key technical barrier overcome by the solution described herein is that the dye-labeled nucleotides on adjacent or nearby nucleotides must show minimal quenching. The overall result of the combined advantages may be more accurate DNA sequencing. The use of high labeling fractions (e.g., 20-100% labeling) may be facilitated by the use of non- or minimally quenching labeled nucleotides (e.g., as described herein). Quenching between dye molecules may be reduced using labeled nucleotides labeled with labeling reagents provided herein.

Scars generated upon cleavage of a labeling reagent or portion thereof used to label an incorporated nucleotide may pose challenges to subsequent incorporation events. For example, introduction of a scar to a growing nucleic acid strand via cleavage of a labeling reagent may prevent incorporation of a subsequent labeled or non-labeled nucleotide at an adjacent or nearby position. Different polymerases useful in sequencing reactions may have different tolerances for incorporation after a scar. Examples of polymerases that may be useful in a sequencing reaction using labeled nucleotides include, for example, Bst3.0, Pol19, Pol 22, Pol47, Pol49, and Pol50.

The present disclosure provides a method for sequencing a nucleic acid molecule. The method can comprise contacting the nucleic acid molecule with a primer under conditions sufficient to hybridize the primer to the nucleic acid molecule, thereby generating a sequencing template. The sequencing template may then be contacted with a polymerase (e.g., as described herein) and a solution (e.g., a nucleotide flow) comprising a plurality of optically (e.g., fluorescently) labeled nucleotides (e.g., as described herein). Each optically (e.g., fluorescently) labeled nucleotide of the plurality of optically (e.g., fluorescently) labeled nucleotides may comprise the same chemical structure (e.g., each labeled nucleotide may comprise a dye of a same type, a linker of a same type, and a nucleotide or nucleotide analog of a same type). An optically labeled nucleotide of the plurality of optically labeled nucleotides may be complementary to the nucleic acid molecule at a plurality of positions adjacent to the primer hybridized to the nucleic acid molecule. Accordingly, one or more optically labeled nucleotides of the plurality of optically labeled nucleotides may be incorporated into the sequencing template. Where the nucleic acid molecule includes a homopolymeric region, multiple nucleotides (e.g., labeled and unlabeled nucleotides) may be incorporated. Incorporation of multiple nucleotides adjacent to one another may be facilitated by the use of non-terminated nucleotides. The solution comprising the plurality of optically labeled nucleotides may then be washed away from the sequencing template (e.g., using a wash flow, as described herein). An optical (e.g., fluorescent) signal from the sequencing template may be measured. Where two or more labeled nucleotides are incorporated into a homopolymeric region, the intensity of the measured optical (e.g., fluorescent) signal may be greater than an optical (e.g., fluorescent) signal that may be measured if a single optically (e.g., fluorescently) labeled nucleotide of the plurality of optically (e.g., fluorescently) labeled nucleotides had been incorporated into the sequencing template. Such a method may be particularly useful for sequencing of homopolymers or portions of nucleic acids that are homopolymeric (i.e., have a plurality of the same base in a row). An optically labeled nucleotide of the plurality of optically labeled nucleotides may comprise a dye (e.g., fluorescent dye) and a linker connected to the dye and a nucleotide (e.g., as described herein). The linker may comprise (i) one or more water soluble groups and (ii) two or more ring systems, wherein at least two of the two or more ring systems are connected to each other by no more than two sp³ carbon atoms, such as by no more than two atoms. The linker may comprise a non-proteinogenic amino acid comprising a ring system of the two or more ring systems. For example, the linker may comprise a hydroxyproline or an amino acid constructed from, e.g., a diamine and a dicarboxylic acid or an amino thiol and a thiol carboxylic acid. The linker may be configured to establish a functional length between the dye and the nucleotide of at least about 0.5 nanometers.

The intensity of the measured optical (e.g., fluorescent) signal may be proportional to the number of optically (e.g., fluorescently) labeled nucleotides incorporated into the sequencing template (e.g., where 100% labeling fraction is used). In other words, quenching may not significantly impact the signal emitted. For example, the intensity may be linearly proportional to the number of optically (e.g., fluorescently) labeled nucleotides incorporated into the sequencing template. The intensity of the measured optical (e.g., fluorescent) signal may be linearly proportional with a slope of approximately 1.0 when plotted against the number of optically (e.g., fluorescently) labeled nucleotides incorporated into the sequencing template. Where less than 100% of substrates are labeled (e.g., less than 100% of nucleotides in a nucleotide flow are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a homopolymer region of the growing nucleic acid strands. Similarly, where less than 100% of substrates are labeled (e.g., less than 100% of nucleotides in each of successive nucleotide flows are labeled), an optical (e.g., fluorescent) signal emitted by substrates (e.g., nucleotides or nucleotide analogs) incorporated into a plurality of growing nucleic acid strands (e.g., a plurality of growing nucleic acid strands coupled to sequencing templates coupled to a support, as described herein) may be proportional to the length of a heteropolymeric and/or homopolymer region of the growing nucleic acid strands. In such cases, the intensity of a measured optical (e.g., fluorescent) signal may be linearly proportional to the length of a heteropolymeric and/or homopolymeric region into which substrates have incorporated. For example, a measured optical (e.g., fluorescent) signal may be linearly proportional with a slope of approximately 1.0 when optical (e.g., fluorescent) signal is plotted against the length in substrates of a heteropolymeric and/or homopolymeric region into which substrates have incorporated

The solution comprising the plurality of optically (e.g., fluorescently) labeled nucleotides may also contain unlabeled nucleotides (e.g., the labeling fraction may be less than 100%). For example, at least about 20% of nucleotides in the solution may be optically labeled, and at least about 80% of nucleotides in the solution may not be optically labeled. In some cases, the majority of the nucleotides in the solution may be optically labeled (e.g., between about 50-100%). Alternatively, only labeled nucleotides may be used (e.g., 100% labeling fraction).

In some cases, two or more optically (e.g., fluorescently) labeled nucleotides of the plurality of optically (e.g., fluorescently) labeled nucleotides are incorporated into the sequencing template (e.g., into a homopolymeric region). In some cases, three or more optically (e.g., fluorescently) labeled nucleotides of the plurality of optically (e.g., fluorescently) labeled nucleotides are incorporated into the sequencing template. The number of optically labeled nucleotides incorporated into the sequencing template during a given nucleotide flow may depend on the homopolymeric nature of the nucleic acid molecule. In some cases, a first optically (e.g., fluorescently) labeled nucleotide of the plurality of optically (e.g., fluorescently) labeled nucleotides is incorporated within four positions of a second optically (e.g., fluorescently) labeled nucleotide of the plurality of optically (e.g., fluorescently) labeled nucleotides.

An optically (e.g., fluorescently) labeled nucleotide may comprise a cleavable group to facilitate cleavage of the optical (e.g., fluorescent) label (e.g., as described herein). In some cases, a method may further comprise, subsequent to incorporation of the one or more optically (e.g., fluorescently) labeled nucleotides and washing away of residual solution, cleaving optical (e.g., fluorescent) labels of the one or more optically (e.g., fluorescently) labeled nucleotides incorporated into the sequencing template (e.g., as described herein). The cleavage flow may be followed by an additional wash flow.

In some cases, a nucleotide flow and wash flow may be followed by a “chase” flow comprising unlabeled nucleotides and no labeled nucleotides. The chase flow may be used to complete the sequencing reaction for a given nucleotide position or positions of the sequencing template (e.g., across a plurality of such templates immobilized to a support). The chase flow may precede detection of an optical signal from a template. Alternatively, the chase flow may follow detection of an optical signal from a template. The chase flow may precede a cleavage flow. Alternatively, the chase flow may follow a cleavage flow. The chase flow may be followed by a wash flow.

The methods provided herein can also be used to sequence heteropolymers and/or heteropolymeric regions of a nucleic acid molecule (i.e., portions that are not homopolymeric). Accordingly, the methods described herein can be used to sequence a nucleic acid molecule having any degree of heteropolymeric or homopolymeric nature.

Regarding homopolymers, a nucleotide flow at a homopolymer region may incorporate several nucleotides in a row. Contacting a sequencing template comprising a nucleic acid molecule (e.g., a nucleic acid molecule hybridized to an unextended primer) comprising a homopolymer region with a solution comprising a plurality of nucleotides (e.g., labeled and unlabeled nucleotides), where each nucleotide of the plurality of nucleotides is of a same type, may result in multiple nucleotides of the plurality of nucleotides being incorporated into the sequencing template. In some cases, at least 2, at least 3, at least 4, at least 5, at least 6, at least 7, at least 8, at least 9, or at least 10 nucleotides are incorporated (i.e., in a homopolymeric region of a nucleic acid molecule). The plurality of nucleotides incorporated into the sequencing template may comprise a plurality of labeled nucleotides (e.g., optically labeled, such as fluorescently labeled), as described herein. In such an instance, one or more of said nucleotides incorporated into a homopolymer region may be labeled, and may either occupy adjacent or non-adjacent positions to other labeled nucleotides incorporated into the homopolymeric region. The intensity of a signal obtained from a nucleic acid molecule may be proportional to the number of incorporated labeled nucleotides (e.g., where a labeling fraction of 100% is used). For example, the intensity of an optical signal (e.g., fluorescent signal) obtained from a nucleic acid molecule containing two labeled nucleotides may be of greater intensity than the optical signal obtained from a nucleic acid molecule containing one labeled nucleotide. Furthermore, the intensity of a signal obtained from a nucleic acid molecule may depend on the relative positioning of labeled nucleotides within a nucleic acid molecule. For example, a nucleic acid molecule containing two labeled nucleotides in non-adjacent positions may provide a different signal intensity than a nucleic acid molecule containing two labeled nucleotides in adjacent positions. Quenching in such systems may be optimized by careful selection of linkers and dyes (e.g., fluorescent dyes). In some cases, a plot of optical signal (e.g., fluorescence) vs. homopolymer length can be linear. For example, measured optical signal for an ensemble of growing nucleic acid strands including homopolymeric regions into which labeled nucleotides are incorporated may be approximately linearly proportional to the nucleotide length of the homopolymeric region.

In another aspect, the present disclosure provides a method for sequencing a nucleic acid molecule. The method can comprise contacting the nucleic acid molecule with a primer under conditions sufficient to hybridize the primer to the nucleic acid molecule, thereby generating a sequencing template. The sequencing template may then be contacted with a polymerase and a first solution comprising a plurality of first optically (e.g., fluorescently) labeled nucleotides (and, optionally, a plurality of first unlabeled nucleotides). Each first optically (e.g., fluorescently) labeled nucleotide of the plurality of first optically (e.g., fluorescently) labeled nucleotides is of a same type. A first optically (e.g., fluorescently) labeled nucleotide of the plurality of first optically (e.g., fluorescently) labeled nucleotides may be complementary to the nucleic acid molecule to be sequenced at a position adjacent to the primer. A first optically (e.g., fluorescently) labeled nucleotide of the plurality of first optically (e.g., fluorescently) labeled nucleotides may thus be incorporated into the sequencing template to generate an extended primer. The first solution comprising the plurality of first optically (e.g., fluorescently) labeled nucleotides may then be washed away from the sequencing template (e.g., using a wash solution). A first optical (e.g., fluorescent) signal emitted by the sequencing template may then be measured (e.g., as described herein). The sequencing template may then be contacted with a polymerase and a second solution comprising a plurality of second optically (e.g., fluorescently) labeled nucleotides (and, optionally, a plurality of second unlabeled nucleotides). Each second optically (e.g., fluorescently) labeled nucleotide of the plurality of second optically (e.g., fluorescently) labeled nucleotides may be of a same type. A second optically (e.g., fluorescently) labeled nucleotide of the plurality of second optically (e.g., fluorescently) labeled nucleotides may be complementary to the nucleic acid molecule to be sequenced at a position adjacent to the extended primer. A second optically (e.g., fluorescently) labeled nucleotide of the plurality of second optically (e.g., fluorescently) labeled nucleotides may thus be incorporated into the sequencing template. The second solution comprising the plurality of second optically (e.g., fluorescently) labeled nucleotides may then be washed away from the sequencing template. A second optical (e.g., fluorescent) signal emitted by the sequencing template may then be measured. In some cases, the intensity of the second optical (e.g., fluorescent) signal may be greater than the intensity of the first optical (e.g., fluorescent) signal.

A first optically labeled nucleotide of the plurality of first optically labeled nucleotides may comprise a first dye (e.g., fluorescent dye) and a first linker connected to the first dye and a first nucleotide (e.g., as described herein). Similarly, a second optically labeled nucleotide of the plurality of second optically labeled nucleotides may comprise a second dye (e.g., fluorescent dye) and a second linker connected to the second dye and a second nucleotide (e.g., as described herein). The first linker may comprise a first semi-rigid portion, which semi-rigid portion may comprise one or more amino acids (e.g., non-proteinogenic amino acids). Similarly, the second linker may comprise a second semi-rigid portion, which semi-rigid portion may comprise one or more amino acids (e.g., non-proteinogenic amino acids). For example, the first linker may comprise at least one hydroxyproline (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more hydroxyprolines, as described herein). The first and second semi-rigid portions may have the same or different structures. The first linker and/or second linker may be connected to a cleavable group configured to be cleaved with a cleavage reagent (e.g., as described herein) to separate a nucleotide from all or a portion of a labeling reagent comprising the linker to which it is coupled. The first linker and the second linker may have the same structure. Alternatively, the first linker and the second linker may have different structures. The first linker and the second linker may comprise a shared structural motif, such as a shared cleavable component (e.g., as described herein).

The first solution comprising the plurality of first optically (e.g., fluorescently) labeled nucleotides may not contain any unlabeled nucleotides (e.g., 100% labeling fraction may be used). Alternatively, the first solution comprising the plurality of first optically (e.g., fluorescently) labeled nucleotides may also contain first unlabeled nucleotides. For example, about 20% of the nucleotides of the first solution may be unlabeled. In some cases, at least 20% of the nucleotides of the first solution may be optically labeled, such as at least 50% or at least 80%. The unlabeled nucleotides may comprise the same nucleotide moiety (e.g., canonical nucleotide moiety) as the optically labeled nucleotides. Similarly, the second solution comprising the plurality of second optically (e.g., fluorescently) labeled nucleotides may not contain any unlabeled nucleotides (e.g., 100% labeling fraction may be used). Alternatively, the second solution comprising the plurality of first optically labeled nucleotides may also contain second unlabeled nucleotides. For example, about 20% of the nucleotides of the second solution may be unlabeled. In some cases, at least 20% of the nucleotides of the second solution may be optically labeled, such as at least 50% or at least 80%. The unlabeled nucleotides may comprise the same nucleotide moiety (e.g., canonical nucleotide moiety) as the optically labeled nucleotides.

The plurality of first optically (e.g., fluorescently) labeled nucleotides may be different from the plurality of second optically (e.g., fluorescently) labeled nucleotides. For example, the plurality of first optically (e.g., fluorescently) labeled and the plurality of second optically (e.g., fluorescently) labeled nucleotides may comprise the same optical (e.g., fluorescent) label (e.g., the same dye) and different nucleotides. Alternatively, the plurality of first optically (e.g., fluorescently) labeled and the plurality of second optically (e.g., fluorescently) labeled nucleotides may comprise different optical (e.g., fluorescent) labels (e.g., different dyes) and the same nucleotides. In some cases, the plurality of first optically (e.g., fluorescently) labeled and the plurality of second optically (e.g., fluorescently) labeled nucleotides may comprise different optical (e.g., fluorescent) labels (e.g., different dyes) and different nucleotides. The first dye of the first plurality of optically labeled nucleotides and the second dye of the second plurality of optically labeled nucleotides may emit signal at approximately the same wavelength or range of wavelengths (e.g., whether the first and second dyes have the same or different chemical structures). For example, the first dye and the second dye may both emit signal in the green region of the visible portion of the electromagnetic spectrum.

In some cases, two or more first optically (e.g., fluorescently) labeled nucleotides may be incorporated into the sequencing template (e.g., in a homopolymeric region of the nucleic acid molecule). In some cases, two or more second optically (e.g., fluorescently) labeled nucleotides may be incorporated into the sequencing template.

Additional optically (e.g., fluorescently) labeled nucleotides may also be provided and incorporated into the sequencing template (e.g., in successive nucleotide flows, as described herein). For example, the method may further comprise contacting the sequencing template with a polymerase and a third solution comprising a plurality of third optically (e.g., fluorescently) labeled nucleotides, wherein each third optically (e.g., fluorescently) labeled nucleotide of the plurality of third optically (e.g., fluorescently) labeled nucleotides is of a same type, and wherein a third optically (e.g., fluorescently) labeled nucleotide of the plurality of third optically (e.g., fluorescently) labeled nucleotides is complementary to the nucleic acid molecule at a position adjacent to the further extended primer hybridized to the nucleic acid molecule, thereby incorporating a third optically (e.g., fluorescently) labeled nucleotide of the plurality of third optically (e.g., fluorescently) labeled nucleotides into the sequencing template; washing the third solution comprising the plurality of third optically (e.g., fluorescently) labeled nucleotides away from the sequencing template; and measuring a third optical (e.g., fluorescent) signal emitted by the sequencing template. In some cases, the intensity of the third optical signal may be greater than the intensity of the first optical (e.g., fluorescent) signal and the intensity of the second optical (e.g., fluorescent) signal. This process may be repeated with a fourth solution, etc. The third and fourth solutions may comprise optically (e.g., fluorescently) labeled nucleotides having different nucleotides than the first and second solutions, such that each canonical nucleotide (A, C, G, and U/T) may be provided in sequence to the sequencing template. A cycle in which each canonical nucleotide is provided to the sequencing template may be repeated one or more times to sequence and/or amplify the nucleic acid molecule.

A third optically labeled nucleotide of the plurality of third optically labeled nucleotides may comprise a third dye (e.g., fluorescent dye) and a third linker connected to the third dye and a third nucleotide (e.g., as described herein). The third linker may comprise a third semi-rigid portion, which semi-rigid portion may comprise one or more amino acids (e.g., non-proteinogenic amino acids). For example, the third linker may comprise at least one hydroxyproline (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more hydroxyprolines, as described herein). The third linker may be connected to a cleavable group configured to be cleaved with a cleavage reagent (e.g., as described herein) to separate a nucleotide from all or a portion of a labeling reagent comprising the linker to which it is coupled. The first linker and the third linker may have the same structure. Alternatively, the first linker and the third linker may have different structures. The first linker and the third linker may comprise a shared structural motif, such as a shared cleavable component (e.g., as described herein). Similarly, the second linker and the third linker may have the same structure. Alternatively, the second linker and the third linker may have different structures. The second linker and the third linker may comprise a shared structural motif, such as a shared cleavable component (e.g., as described herein). The third dye may have the same or a different structure as the first dye. Similarly, the third dye may have the same or a different structure as the second dye. The third dye and the first and/or second dye may emit at approximately the same wavelength or range of wavelengths (e.g., whether these dyes have the same or different chemical structures). Further, the third nucleotide may be of a same or different type as the first nucleotide, or the third nucleotide may be of a same or different type as the second nucleotide.

The method may further comprise, subsequent to washing a given solution (e.g., nucleotide flow) away (e.g., using a wash solution), cleaving the optical (e.g., fluorescent) label of its respective nucleotides. For example, after the first solution is washed away, the optical (e.g., fluorescent) label of the first optically (e.g., fluorescently) labeled nucleotide incorporated into the sequencing template may be cleaved (e.g., using a cleavage reagent to cleave a cleavable group of a linker of the first optically labeled nucleotide, as described herein). For example, the fluorescent dye(s) of the first optically labeled nucleotide(s) incorporated into the sequencing template may be cleaved prior to contacting the sequencing template with second optically labeled nucleotides (e.g., in a second nucleotide flow, as described herein). Accordingly, signal may be detected from one or more first optically labeled nucleotides prior to incorporation of one or more second optically labeled nucleotides into the sequencing template. Separation of the fluorescent dye (s) of the first optically labeled nucleotide(s) incorporated into the sequencing template may provide a scarred nucleotide(s) comprising a portion of the linker of the first optically labeled nucleotide, or a derivative thereof. Similarly, after the second solution (e.g., second nucleotide flow) is washed away, the optical (e.g., fluorescent) label of the second optically (e.g., fluorescently) labeled nucleotide incorporated into the sequencing template may be cleaved. All of a portion of the first and second linkers may be cleaved during the respective cleaving processes.

In another aspect, provided herein is a method for sequencing a nucleic acid molecule. The method can comprise providing a solution comprising a plurality of optically (e.g., fluorescently) labeled nucleotides, wherein each optically (e.g., fluorescently) labeled nucleotide of the plurality of optically (e.g., fluorescently) labeled nucleotides is of a same type. A given optically (e.g., fluorescently) labeled nucleotide of the plurality of fluorescently labeled nucleotides may comprise an optical (e.g., fluorescent) dye that is connected to a nucleotide via a semi-rigid water-soluble linker having a defined molecular weight. The linker connecting the dye and nucleotide may provide a functional length of at least about 0.5 nanometers (nm) between the dye and nucleotide. The nucleic acid molecule may then be contacted with a primer under conditions sufficient to hybridize the primer to a nucleic acid molecule to be sequenced to generate a sequencing template. The sequencing template may then be contacted with a polymerase and the solution containing the plurality of optically (e.g., fluorescently) labeled nucleotides, wherein an optically (e.g., fluorescently) labeled nucleotide of the plurality of optically (e.g., fluorescently) labeled nucleotides is complementary to the nucleic acid molecule to be sequenced at a position adjacent to the primer. One or more optically (e.g., fluorescently) labeled nucleotides of the plurality of optically (e.g., fluorescently) labeled nucleotides may thus be incorporated into the sequencing template. The solution comprising the plurality of optically (e.g., fluorescently) labeled nucleotides may be washed away from the sequencing template (e.g., using a wash solution). An optical (e.g., fluorescent) signal emitted by the sequencing template may then be measured.

The linker may have any useful features provided herein. For example, the linker may comprise a semi-rigid portion. The linker may comprise or be coupled to a cleavable group (e.g., as described herein). The linker may comprise an amino acid (e.g., a non-proteinogenic amino acid, as described herein). For example, the linker may comprise one or more hydroxyproline moieties (e.g., as described herein), such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more hydroxyprolines. The linker may establish a functional length between the fluorescent dye and the nucleotide of at least about 9 Å, such as at least about 30 Å, 60 Å, 90 Å, or more (e.g., as described herein).

The measured optical (e.g., fluorescent) signal may be proportional to the number of optically (e.g., fluorescently) labeled nucleotides that were incorporated into the sequencing template. For example, where 100% labeling fraction is used (e.g., all nucleotides in the solution are labeled), quenching may not diminish the emitted signal. In such a system, the measured optical (e.g., fluorescent) signal can be linearly proportional to the number of optically (e.g., fluorescently) labeled nucleotides that were incorporated into the sequencing template. The measured optical (e.g., fluorescent) signal may be linearly proportional with a slope of approximately 1.0 when plotted against the number of optically (e.g., fluorescently) labeled nucleotides that were incorporated into the sequencing template. Where less than 100% of nucleotides are labeled (e.g., less than 100% of nucleotides in the solution are labeled), an optical (e.g., fluorescent) signal emitted by nucleotides incorporated into a plurality of growing nucleic acid strands (e.g., a plurality of growing nucleic acid strands coupled to sequencing templates coupled to a support, as described herein) may be proportional to the length of a homopolymer region of the growing nucleic acid strands. Similarly, where less than 100% of nucleotides are labeled, an optical (e.g., fluorescent) signal emitted by nucleotides incorporated into a plurality of growing nucleic acid strands (e.g., a plurality of growing nucleic acid strands coupled to sequencing templates coupled to a support, as described herein) may be proportional to the length of a heteropolymeric and/or homopolymer region of the growing nucleic acid strands. In some such cases, the intensity of a measured optical (e.g., fluorescent) signal may be linearly proportional to the length of a heteropolymeric and/or homopolymeric region into which nucleotides have incorporated. For example, a measured optical (e.g., fluorescent) signal may be linearly proportional with a slope of approximately 1.0 when optical (e.g., fluorescent) signal is plotted against the length in nucleotides of a heteropolymeric and/or homopolymeric region into which nucleotides have incorporated

In some cases, the solution containing an optically (e.g., fluorescently) labeled nucleotide also contains unlabeled nucleotides. The unlabeled nucleotides may comprise the same nucleotide moiety (e.g., the same canonical nucleotide). In some embodiments, about 5%, about 10%, about 20%, about 30%, about 40%, about 50%, about 60%, about 70%, about 80%, about 90%, or about 100% of nucleotides in the solution are fluorescently labeled. In some cases, at least about 5%, at least about 10%, at least about 20%, at least about 30%, at least about 40%, at least about 50%, at least about 60%, at least about 70%, at least about 80%, at least about 90%, or more of nucleotides in the solution are fluorescently labeled. In some cases, at least about 5%, at least about 10%, at least about 20%, at least about 30%, at least about 40%, at least about 50%, at least about 60%, at least about 70%, at least about 80%, at least about 90%, or more of nucleotides in the solution are not fluorescently labeled.

A plurality of labeled nucleotides can be incorporated at locations along a nucleic acid molecule in proximity to each other. In some cases, a first optically (e.g., fluorescently) labeled nucleotide is incorporated within 4 positions, within 3 positions, within 2 positions, or next to a second optically (e.g., fluorescently) labeled nucleotide (e.g., a second optically labeled nucleotide of a same or different nucleotide type). In some cases, the method further comprises cleaving the optical (e.g., fluorescent) labels from the nucleotides after measuring the optical (e.g., fluorescent) signal (e.g., as described herein). Cleaving an optical (e.g., fluorescent) label may leave behind a scar (e.g., as described herein). A nucleic acid sequencing assay may be used to evaluate dye-labeled nucleotides. The assay may use a nucleic acid template having a known sequence, which sequence may include one or more homopolymeric regions. The template may be immobilized to a support (e.g., as described herein) via an adapter. A primer having a sequence at least partly complementary to the adapter or a portion thereof may hybridize to the adapter or portion thereof and provide a starting point for generation of a nucleic acid strand having a sequence complementary to that of the template via incorporation of labeled and unlabeled nucleotides (e.g., as described herein). The sequencing assay may use four distinct four nucleotide flows including different canonical nucleobases that may be repeated in cyclical fashion (e.g., cycle 1: A, G, C, U; cycle 2 Å, G, C, U; etc.). Each nucleotide flow may include nucleotides including nucleobases of a single canonical type (or analogs thereof), some of which may be include optical labeling reagents provided herein. The labeling fraction (e.g., % of nucleotides included in the flow that are attached to an optical labeling reagent) may be varied between, e.g., 0.5% to 100%. Labeling fractions may be different for different nucleotide flows. Nucleotides may not be terminated to facilitate incorporation into homopolymeric regions. The template may be contacted with a nucleotide flow, followed by one or more wash flows (e.g., as described herein). The template may also be contacted with a cleavage flow (e.g., as described herein) including a cleavage reagent configured to cleave a portion of the optical labeling reagents attached to labeled nucleotides incorporated into the growing nucleic acid strand. A wash flow may be used to remove cleavage reagent and prepare the template for contact with a subsequent nucleotide flow. Emission may be detected from labeled nucleotides incorporated into the growing nucleic acid strand after each nucleotide flow.

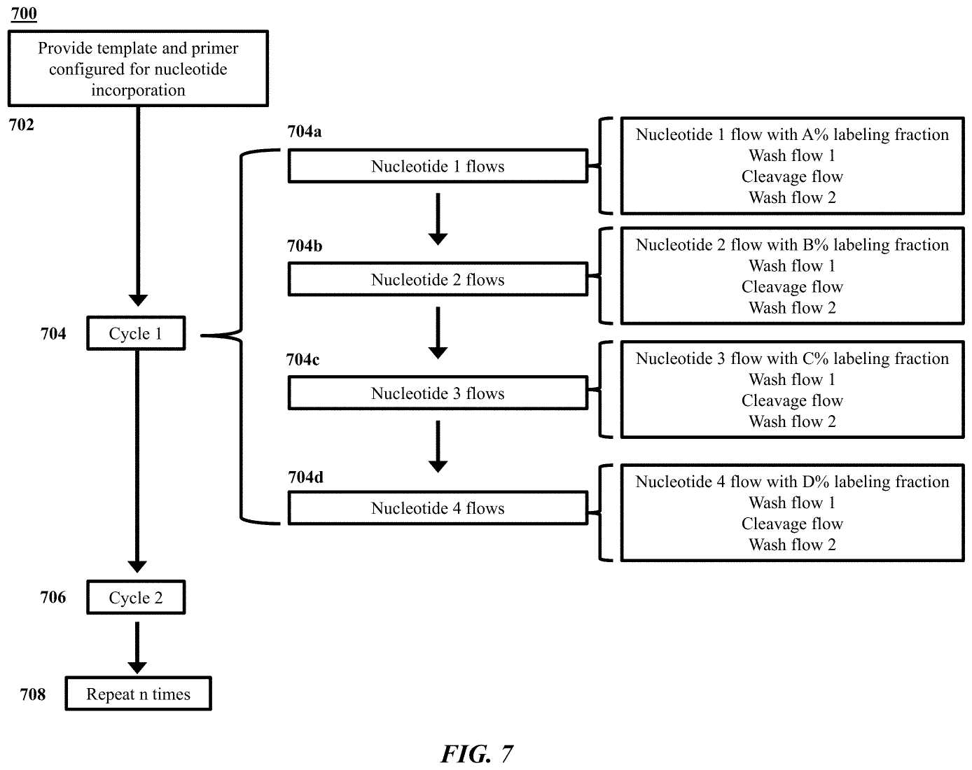

An example sequencing procedure 700 is provided in FIG. 7. In process 702, a template and primer configured for nucleotide incorporation are provided. A first sequencing cycle 704 is subsequently performed. First sequencing cycle 704 includes four flow processes 704a, 704b, 704c, and 704d, each of which multiple flows. Nucleotides 1, 2, 3, and 4 may each include nucleobases of different canonical types (e.g., A, G, C, and U). A given nucleotide flow may include both labeled nucleotides (e.g., nucleotides labeled with an optical labeling reagent provided herein) and unlabeled nucleotides. The labeling fraction of each nucleotide flow may be different. That is, A, B, C, and D in FIG. 7 may be the same or different and may range from 0% to 100% (e.g., as described herein). Labels and linkers used to label nucleotides 1, 2, 3, and 4 may be of the same or different types. For example, nucleotide 1 may have a linker including a cleavable linker and a hyp10 linker and a first green dye, and nucleotide 2 may have a linker including a cleavable linker but not a hyp10 linker and a second green dye. The first green dye may be the same as or different from the first green dye. The cleavable linkers associated with the different nucleotides may be the same or different. Flow process 704a may include a nucleotide flow (e.g., a flow including a plurality of nucleotides of type Nucleotide 1, A % of which may be labeled). During this flow, labeled and unlabeled nucleotides may be incorporated into the growing strand (e.g., using a polymerase enzyme). A first wash flow (“wash flow 1”) may be used to remove unincorporated nucleotides and associated reagents. A cleavage flow including a cleavage reagent may be provided to all or portions of the optical labeling reagents attached to incorporated nucleotides. For example, labeled nucleotides may include a cleavable linker portion that may by cleaved upon contact with the cleavage reagent to provide a scarred nucleotide. A second wash flow (“wash flow 2”) may be used to remove the cleavage reagent and cleaved materials. Nucleotide flow process 704a may also include a “chase” process in which a nucleotide flow including only unlabeled nucleotides of type Nucleotide 1 may be flowed. Such a chase process may be followed by a wash flow. The chase process and its accompanying wash flow may take place after the initial nucleotide flow and wash flow 1, or after the cleavage flow and wash flow 2. The next nucleotide flow process 704b may then begin and proceed in similar fashion. Following completion of processes 704b, 704c, and 704d, the first flow cycle 704 may be complete. A second flow cycle 706 may begin. Cycle 706 may include the same flow processes in the same or different order. Additional cycles may be performed until all or a portion of the template has been sequenced. Detection of incorporated nucleotides via emission detection may be performed after nucleotide flows and initial wash flows and before cleavage flows for each nucleotide flow process (e.g., flow process 704a may include a detection process between wash flow 1 and cleavage flow, etc.). A template interrogated by such a sequencing process may be immobilized to a support (e.g., as described herein). A plurality of such templates (e.g., at least about 100, 200, 500, 1000, 10000, 100,000, 500,000, 1,000,000, or more templates) may be interrogated contemporaneously in this fashion (e.g., in clonal fashion). In such a system, incorporation of nucleotides may be detected as an average over the plurality of templates, which may permit the use of labeling fractions of less than 100%.

In some cases, for any of the preceding methods, the nucleotide is guanine (G) and the linker decreases quenching between the nucleotide and the dye (e.g., fluorescent) dye.

In some cases, for any of the preceding methods, an optically (e.g., fluorescently) labeled nucleotide comprising a linker provided herein is more efficiently incorporated into a sequencing template than another optically (e.g., fluorescently) labeled nucleotide that comprises the same nucleotide and optical (e.g., fluorescent) dye but does not include the linker. In some cases, for any of the preceding methods, an optically (e.g., fluorescently) labeled nucleotide comprising a linker provided herein is incorporated into a sequencing template with higher fidelity than another optically (e.g., fluorescently) labeled nucleotide that comprises the same nucleotide and optical (e.g., fluorescent) dye but does not include the linker.

For any of the sequencing methods provided herein, the polymerase used may be a Family A polymerase such as Taq, Klenow, or Bst polymerase. Alternatively, for any of the sequencing methods provided herein, the polymerase may be a Family B polymerase such as Vent(exo-) or Therminator™ polymerase. The polymerase may be, for example, Bst3.0, Pol19, Pol22, Pol47, Pol49, Pol50, or any other useful polymerase.

In an aspect, the present disclosure provides methods for sequencing a nucleic acid molecule using the optically (e.g., fluorescently) labeled nucleotides described herein. A method may comprise providing a plurality of nucleic acid molecules, which plurality of nucleic acid molecules may comprise or be part of a colony or a plurality of colonies. The plurality of nucleic acid molecules may have sequence homology to a template sequence. The method may comprise contacting the plurality of nucleic acid molecules with a solution comprising a plurality of nucleotides (e.g., a solution comprising a plurality of optically labeled nucleotides) under conditions sufficient to incorporate a subset of the plurality of nucleotides into a plurality of growing nucleic acid strands that is complementary to the plurality of nucleic acid molecules. In some instances, at least about 20% of the subset of the plurality of nucleotides are optically (e.g., fluorescently) labeled nucleotides (e.g., as described herein). For example, at least about 20%, 25%, 50%, 75%, 90%, or great of the subset of the plurality of nucleotides may be labeled nucleotides. In some cases, 100% of the nucleotides may be labeled nucleotides. The method may comprise detecting one or more signals or signal changes from the labeled nucleotides incorporated into the plurality of growing nucleic acid strands, wherein the one or more signals or signal changes are indicative of the labeled nucleotides having incorporated into the plurality of growing nucleic acid strands.

The optically (e.g., fluorescently) labeled nucleotides of the plurality of nucleotides may be non-terminated. In such cases, the growing strands may incorporate one or more consecutive nucleotides during (e.g., a complimentary base to the plurality of nucleotides in solution is not present at a plurality of positions adjacent to the primer hybridized to the nucleic acid molecule). The one or more signals or signal changes detected from the optically (e.g., fluorescently) labeled nucleotides may be indicative of consecutive nucleotides having incorporated into the plurality of growing nucleic acid strands. Methods for determining a number of fluorophores from the detected signals or signal changes are described elsewhere herein.

Alternatively, the optically (e.g., fluorescently) labeled nucleotides may be terminated. In such cases, each growing strand may incorporate no more than one nucleotide per flow cycle until synthesis is terminated. The one or more signals or signal changes detected from the optically (e.g., fluorescently) labeled nucleotides may be indicative of nucleotides having incorporated into the plurality of growing nucleic acid strands. Prior to, during, or subsequent to detection, a terminating group of the labeled nucleotides may be cleaved (e.g., to facilitate sequencing of homopolymers, and/or to reduce potential context and/or quenching issues).

Alternatively or additionally, the optically (e.g., fluorescently) labeled nucleotides may include a mixture of terminated and non-terminated nucleotides. In such cases, the growing strands may incorporate one or more consecutive nucleotides generating an extended primer. The solution comprising the plurality of terminated and non-terminated nucleotides may then be washed away from the sequencing template. Unlabeled nucleotides of the plurality of nucleotides may comprise nucleotide moieties of the same type as labeled nucleotides of the plurality of nucleotides (e.g., the same canonical nucleotide).

In an aspect, the present disclosure provides compositions comprising one or more fluorescently labeled nucleotides and methods of using the same. A composition may comprise a solution comprising a fluorescently labeled nucleotide (e.g., as described herein). The fluorescently labeled nucleotide may comprise a fluorescent labeling reagent (e.g., as described herein) comprising a fluorescent dye that is connected to a nucleotide or nucleotide analog (e.g., as described herein) via a linker (e.g., as described herein). The linker may comprise any useful feature described herein. For example, the linker may comprise a semi-rigid portion. The linker may comprise a plurality of amino acids (e.g., non-proteinogenic amino acids). For example, the linker may comprise a plurality of hydroxyprolines. For example, the linker may comprise at least one hydroxyproline, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more hydroxyprolines. The fluorescently labeled nucleotide may be configured to emit a fluorescent signal. The labeling reagent may comprise a cleavable group (e.g., an azidomethyl group, a disulfide bond, a hydrocarbyldithiomethyl group, and a 2-nitrobenzyloxy group) that is configured to be cleaved to separate the fluorescent dye from the nucleotide.

The solution (e.g., nucleotide flow) may comprise a plurality of fluorescently labeled nucleotides, each or which may comprise a fluorescent dye of a same type, a linker of a same type, and a nucleotide of a same type. Each linker of each fluorescently labeled nucleotide of the plurality of fluorescently labeled nucleotides may have the same molecular weight (e.g., they might not comprise polymers with a range of molecular weights). The solution may also comprise a plurality of unlabeled nucleotides, in which each nucleotide of the plurality of unlabeled nucleotides is of a same type as each nucleotide of the plurality of fluorescently labeled nucleotides. The ratio of the plurality of fluorescently labeled nucleotides to the plurality of unlabeled nucleotides in the solution may be at least about 1:4 (e.g., the labeling fraction may be at least 20%). For example, the ratio may be at least 1:1 (e.g., the labeling fraction may be at least 50%). Alternatively, the solution may not comprise any unlabeled nucleotides and the labeling fraction may be 100%.

The solution (e.g., nucleotide flow) may be provided to a template nucleic acid molecule coupled to a nucleic acid strand. The template nucleic acid molecule may be immobilized to a support (e.g., as described herein). For example, the template nucleic acid molecule may be immobilized to a support via an adapter. For example, the template nucleic acid molecule may be immobilized to a support via a primer to which it is hybridized. The nucleic acid strand may be at least partially complementary to a portion of the template nucleic acid molecule. The template nucleic acid molecule and nucleic acid strand coupled thereto may be subjected to conditions sufficient to incorporate a fluorescently labeled nucleotide of the solution into the nucleic acid strand coupled to the template nucleic acid molecule. Incorporation of the fluorescently labeled nucleotide may be accomplished using a polymerase enzyme (e.g., as described herein). More than one fluorescently labeled nucleotide of the solution may be incorporated, such as into a homopolymeric region of the template nucleic acid molecule. Alternatively or additionally, an unlabeled nucleotide may be incorporated (e.g., adjacent to the fluorescently labeled nucleotide), such as into a homopolymeric region of the template nucleic acid molecule. A signal (e.g., a fluorescent signal) may be detected from the fluorescently labeled nucleotide incorporated into the nucleic acid strand. Prior to detection of the signal, a wash solution may be used to remove fluorescently labeled nucleotides that are not incorporated into the nucleic acid strand. After detection of the signal, the fluorescently labeled nucleotide incorporated into the nucleic acid strand may be contacted with a cleavage reagent configured to cleave the fluorescent dye from the nucleotide. The cleavage reagent may be configured to cleave the linker to provide the nucleotide attached to a portion of the linker, which portion may comprise a thiol moiety, an aromatic moiety, or a combination thereof. The nucleic acid strand, such as a nucleic acid strand of a plurality of nucleic acid strands coupled to a plurality of template nucleic acid molecules, may be contacted with a chase flow comprising only unlabeled nucleotides of a same nucleotide type (e.g., before or after detection of a signal). The nucleic acid strand coupled to the template nucleic acid molecule may also be contacted with one or more additional wash flows. The nucleic acid strand coupled to the template nucleic acid molecule may be contacted with an additional solution comprising an additional fluorescently labeled nucleotide, such as an additional fluorescently labeled nucleotide including a nucleotide of a different type. The dye of the additional fluorescently labeled nucleotide may be of a same type as the dye of the fluorescently labeled nucleotide. Similarly, the linker of the additional fluorescently labeled nucleotide may be of a same type as the linker of the fluorescently labeled nucleotide.

In another aspect, the present disclosure provides a method comprising providing a fluorescent labeling reagent (e.g., as described herein). The fluorescent labeling reagent may comprise a fluorescent dye and a linker that is connected to the fluorescent dye. The fluorescent labeling reagent may have any useful features provided herein. For example, the labeling reagent may comprise a scaffold to which a plurality of linkers may be connected. The linker of the labeling reagent may comprise a semi-rigid portion. The linker may comprise a plurality of amino acids (e.g., non-proteinogenic amino acids). For example, the linker may comprise a plurality of hydroxyprolines. For example, the linker may comprise at least one hydroxyproline, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more hydroxyprolines. The fluorescent labeling reagent may be configured to emit a fluorescent signal.

A substrate may be contacted with the fluorescent labeling reagent to generate a fluorescently labeled substrate, in which the linker connected to the fluorescent dye is associated with the substrate. The substrate may be a nucleotide or nucleotide analog (e.g., as described herein). Alternatively, the substrate may be a protein, lipid, cell, or antibody, or any other substrate described herein. The fluorescently labeled substrate may be configured to emit a fluorescent signal (e.g., upon excitation at an appropriate energy range), which signal may be detected (e.g., using imaging-based detection). The labeling reagent may comprise a cleavable group (e.g., an azidomethyl group, a disulfide bond, a hydrocarbyldithiomethyl group, and a 2-nitrobenzyloxy group) that is configured to be cleaved to separate the fluorescent dye from the substrate. The fluorescently labeled substrate may be contacted with a cleavage reagent configured to cleave the fluorescent labeling reagent or a portion thereof from the fluorescently labeled substrate to generate a scarred substrate. The scarred substrate may comprise a thiol moiety, an aromatic moiety, or a combination thereof. Prior to generating the scarred substrate, the fluorescently labeled substrate and a nucleic acid molecule may be subjected to conditions sufficient to incorporate the fluorescently labeled substrate into the nucleic acid molecule. Incorporation may be accomplished using a polymerase enzyme (e.g., as described herein). More than one fluorescently labeled substrate may be incorporated, such as into a homopolymeric region of the nucleic acid molecule. For example, an additional fluorescently labeled substrate may be incorporated into a position adjacent to the position into which the fluorescently labeled substrate is incorporated. Alternatively or additionally, an unlabeled substrate (e.g., a nucleotide of a same type as the nucleotide of a fluorescently labeled nucleotide) may also be incorporated into the nucleic acid molecule, such as into adjacent positions of the nucleic acid molecule. Incorporation of an additional fluorescently labeled substrate may be done before or after generation of the scarred substrate. Similarly, incorporation of an unlabeled substrate may be done before or after generation of the scarred substrate.

The nucleic acid molecule, such as a nucleic acid molecule of a plurality of nucleic acid molecules, may be contacted with a chase flow comprising only unlabeled substrates of a same type (e.g., before or after detection of a signal from the nucleic acid molecule). The nucleic acid molecule may also be contacted with one or more additional wash flows. The nucleic acid molecule may be contacted with an additional solution comprising an additional fluorescently labeled substrate, such as an additional fluorescently labeled substrate including a nucleotide of a different type. The dye of the additional fluorescently labeled substrate may be of a same type as the dye of the fluorescently labeled substrate. Similarly, the linker of the additional fluorescently labeled substrate may be of a same type as the linker of the fluorescently labeled substrate.

The nucleic acid molecule may be immobilized to a support (e.g., as described herein). For example, the nucleic acid molecule may be immobilized to a support via an adapter. For example, the nucleic acid molecule may be immobilized to a support via a primer to which it is hybridized. The nucleic acid molecule may comprise a first nucleic acid strand that is at least partially complementary to a portion of a second nucleic acid strand. The second nucleic acid strand may comprise a template nucleic acid sequence, or a complement thereof.

The labeled nucleotides of the present disclosure may be used during sequencing operations that involve a high fraction of labeled nucleotides. For example, the present disclosure provides a method comprising contacting a nucleic acid molecule (e.g., a template nucleic acid molecule) with a solution comprising a plurality of nucleotides under conditions sufficient to incorporate a first labeled nucleotide and a second labeled nucleotide of the plurality of nucleotides into a growing strand that is at least partially complementary to the nucleic acid molecule. The first labeled nucleotide and the second labeled nucleotide may be of a same canonical base type. The first nucleotide may comprise a fluorescent dye (e.g., as described herein), which fluorescent dye may be associated with the first nucleotide via a linker (e.g., as described herein). The second nucleotide may comprise the same fluorescent dye (e.g., associated with the second nucleotide via a linker having the same chemical structure of the linker associating the first nucleotide and the fluorescent dye). A fluorescent dye coupled to a nucleotide (e.g., the first and/or second nucleotide) may be cleavable (e.g., upon application of a cleavage reagent). At least about 20% of the plurality of nucleotides may be labeled nucleotides. For example, at least 20% of the plurality of nucleotides may be associated with a fluorescent labeling reagent (e.g., as described herein). For example, at least about 50%, 70%, 80%, 90%, 95%, or 99% of the plurality of nucleotides may be labeled nucleotides. For example, all of the nucleotides of the plurality of nucleotides may be labeled nucleotides (e.g., the labeling fraction may be 100%). One or more signals or signal changes may be detected from the first labeled nucleotide and the second labeled nucleotide (e.g., as described herein). The one or more signals or signal changes may comprise fluorescent signals or signal changes. The one or more signals or signal changes may be indicative of incorporation of the first labeled nucleotide and the second labeled nucleotide. The one or more signals or signal changes may be resolved to determine a sequence of the nucleic acid molecule, or a portion thereof. Resolving the one or more signals or signal changes may comprise determining a number of consecutive nucleotides from the solution that incorporated into the growing strand. The number of consecutive nucleotides may be selected from the group consisting of 2, 3, 4, 5, 6, 7, or 8 nucleotides. Resolving the one or more signals or signal changes may comprise processing a tolerance of the solution. A third nucleotide may also be incorporated into the growing strand (e.g., before or after detection of the one or more signals or signal changes). The third nucleotide may be a nucleotide of the plurality of nucleotides of the solution. Alternatively, the third nucleotide may be provided in a separate solution, such as in a “chase” flow (e.g., as described herein). The third nucleotide may be unlabeled. Alternatively, the third nucleotide may be labeled. The first labeled nucleotide and the third nucleotide may be of a same canonical base type. Alternatively, the first labeled nucleotide and the third nucleotide may be of different canonical base types.

The method may further comprise cleaving the fluorescent dye coupled to the first labeled nucleotide. The fluorescent dye may be cleaved by application of a cleavage reagent configured to cleave a linker associating the first labeled nucleotide and the fluorescent dye. The nucleic acid molecule may be contacted with a second solution comprising a second plurality of nucleotides under conditions sufficient to incorporate a third labeled nucleotide of the second plurality of nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure.

Alternatively, the method may comprise contacting the nucleic acid molecule with a second solution comprising a second plurality of nucleotides under conditions sufficient to incorporate a third labeled nucleotide of the second plurality of nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure. Contacting the nucleic acid molecule with the second solution may be performed in absence of cleaving a fluorescent dye from the first labeled nucleotide or the second labeled nucleotide. This process may be repeated one or more times, such as 1, 2, 3, 4, 5, or more times, each with a different solution of nucleotides, in absence of cleaving a fluorescent dye from the first labeled nucleotide or the second labeled nucleotide. One or more of these different solutions of nucleotides may comprise at least 20% labeled nucleotides.

The present disclosure also provides a method comprising contacting a nucleic acid molecule with a solution comprising a plurality of non-terminated nucleotides under conditions sufficient to incorporate a labeled nucleotide and a second nucleotide of the plurality of non-terminated nucleotides into a growing strand that is at least partly complementary to the nucleic acid molecule, or a portion thereof. The labeled nucleotide and the second nucleotide may be of a same canonical base type. Alternatively, the labeled nucleotide and the second nucleotide may be of different canonical base types. The labeled nucleotide may comprise a fluorescent dye (e.g., as described herein), which fluorescent dye may be associated with the labeled nucleotide via a linker (e.g., as described herein). The second nucleotide may be a labeled nucleotide. For example, the second nucleotide may comprise the same fluorescent dye (e.g., associated with the second nucleotide via a linker having the same chemical structure of the linker associating the first nucleotide and the fluorescent dye). Alternatively, the second nucleotide may not be coupled to a fluorescent dye (e.g., the second nucleotide may be unlabeled). A fluorescent dye coupled to a nucleotide (e.g., the first and/or second nucleotide) may be cleavable (e.g., upon application of a cleavage reagent). The plurality of non-terminated nucleotides may comprise nucleotides of a same canonical base type. At least about 20% of said plurality of nucleotides may be labeled nucleotides. For example, at least 20% of the plurality of nucleotides may be associated with a fluorescent labeling reagent (e.g., as described herein). For example, at least about 50%, 70%, 80%, 90%, 95%, or 99% of the plurality of non-terminated nucleotides may be labeled nucleotides. For example, substantially all of the plurality of non-terminated nucleotides may be labeled nucleotides. For example, all of the nucleotides of the plurality of non-terminated nucleotides may be labeled nucleotides (e.g., the labeling fraction may be 100%). One or more signals or signal changes may be detected from the labeled nucleotide (e.g., as described herein). The one or more signals or signal changes may comprise fluorescent signals or signal changes. The one or more signals or signal changes may be indicative of incorporation of the labeled nucleotide. The one or more signals or signal changes may be resolved to determine a sequence of the nucleic acid molecule, or a portion thereof. Resolving the one or more signals or signal changes may comprise determining a number of consecutive nucleotides from the solution that incorporated into the growing strand. The number of consecutive nucleotides may be selected from the group consisting of 2, 3, 4, 5, 6, 7, or 8 nucleotides. Resolving the one or more signals or signal changes may comprise processing a tolerance of the solution. A third nucleotide may also be incorporated into the growing strand (e.g., before or after detection of the one or more signals or signal changes). The third nucleotide may be a nucleotide of the plurality of non-terminated nucleotides of the solution. Alternatively, the third nucleotide may be provided in a separate solution, such as in a “chase” flow (e.g., as described herein). The third nucleotide may be unlabeled. Alternatively, the third nucleotide may be labeled. The labeled nucleotide and the third nucleotide may be of a same canonical base type. Alternatively, the labeled nucleotide and the third nucleotide may be of different canonical base types.

The method may further comprise cleaving the fluorescent dye coupled to the labeled nucleotide. The fluorescent dye may be cleaved by application of a cleavage reagent configured to cleave a linker associating the labeled nucleotide and the fluorescent dye. The nucleic acid molecule may be contacted with a second solution comprising a second plurality of non-terminated nucleotides under conditions sufficient to incorporate a third labeled nucleotide of the second plurality of non-terminated nucleotides into the growing strand. At least about 20% of the second plurality of non-terminated nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure.

Alternatively, the method may comprise contacting the nucleic acid molecule with a second solution comprising a second plurality of non-terminated nucleotides under conditions sufficient to incorporate a third labeled nucleotide of the second plurality of non-terminated nucleotides into the growing strand. At least about 20% of the second plurality of nucleotides may be labeled nucleotides (e.g., as described herein). One or more second signals or signal changes may be detected from the third labeled nucleotide (e.g., as described herein). The one or more second signals or signal changes may be resolved to determine a second sequence of the nucleic acid molecule, or a portion thereof. The first labeled nucleotide and the third labeled nucleotide may be different canonical base types (e.g., A, C, U/T, or G). The third labeled nucleotide may comprise the fluorescent dye. The fluorescent dye may be coupled to the third labeled nucleotide via a linker (e.g., as described herein), which linker may have the same chemical structure as the linker connecting the fluorescent dye to the first labeled nucleotide or a different chemical structure. Contacting the nucleic acid molecule with the second solution may be performed in absence of cleaving a fluorescent dye from the first labeled nucleotide or the second labeled nucleotide. This process may be repeated one or more times, such as 1, 2, 3, 4, 5, or more times, each with a different solution of nucleotides, in absence of cleaving a fluorescent dye from the first labeled nucleotide or the second labeled nucleotide. One or more of these different solutions of nucleotides may comprise at least 20% labeled nucleotides.

### Methods for Synthesis of Optical Labeling Reagents

In some cases, the linkers provided herein may be prepared using peptide synthesis chemistry.

For example, a linker comprising a pyridinium moiety may be prepared using peptide synthesis chemistry. Such a method may use four bifunctional reagents to make the linker, namely: (a) R¹A, (b) BB, (c) AA, and (d) AR². Reagent A reacts with B to form a pyridinium group; R¹ and R² are hetero-bifunctional attachment groups. The synthesis begins with the group R¹A (or R²A). Excess BB is added to R¹A to form R¹A-BB. The product is precipitated and washed in a less polar solvent (such as ethyl acetate or tetrahydrofuran) to remove excess BB. Excess AA is added with heat in N-methylpyrrolidone (NMP) to produce R¹A-BB-AA. The product is precipitated and washed in a less polar solvent. The synthesis proceeds until a linker of a particular length is formed. The group AR² is appended in the final step.
1) R¹A+10BB→R¹A-BB (wash away excess BB)
2) R¹A-BB+10 AA→R¹A-BB-AA (wash away excess AA)
3) R¹A-BB-AA+10 BB→R¹A-BB-AA-BB (wash away excess BB)
4) R¹A-BB-AA-BB+AR²→R¹A-BB-AA-BB-AR² (use terminating reagent)

Synthetic methods for preparing optical labeling reagents (e.g., as described herein) are described elsewhere and in the Examples below.

### Methods for Constructing Labeled Nucleotides

In an aspect, the present disclosure provides methods for constructing labeled nucleotides (e.g., optically labeled nucleotides).

Labeled nucleotides can be constructed using modular chemical building blocks. A nucleotide or nucleotide analog can be derivatized with, e.g., a propargylamino moiety to provide a handle for attachment to a linker or detectable label (e.g., dye). One or more detectable labels, such as one or more dyes, can be attached to a nucleotide or nucleotide analog via a covalent bond. Alternatively or additionally, one or more detectable labels can be attached to a nucleotide or nucleotide analog via a non-covalent bond. A detectable label may be attached to a nucleotide or nucleotide analog via a linker (e.g., as described herein). A linker may include one or more moieties. For example, a linker may include a first moiety including a disulfide bond within it to facilitate cleaving the linker and releasing the detectable label (e.g., during a sequencing process). Additional linker moieties can be added using sequential peptide bonds. Linker moieties can have various lengths and charges. A linker moiety may include one or more different components, such as one or more different ring systems, and/or a repeating unit (e.g., as described herein). Examples of linkers include, but are not limited to, aminoethyl-SS-propionic acid (epSS), aminoethyl-SS-benzoic acid, aminohexyl-SS-propionic acid, hyp10, and hyp20.

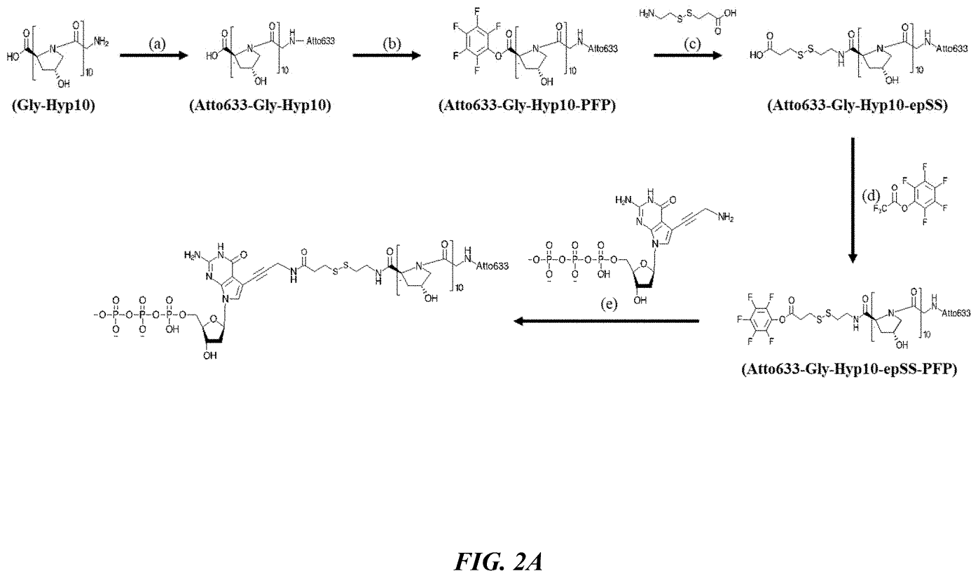

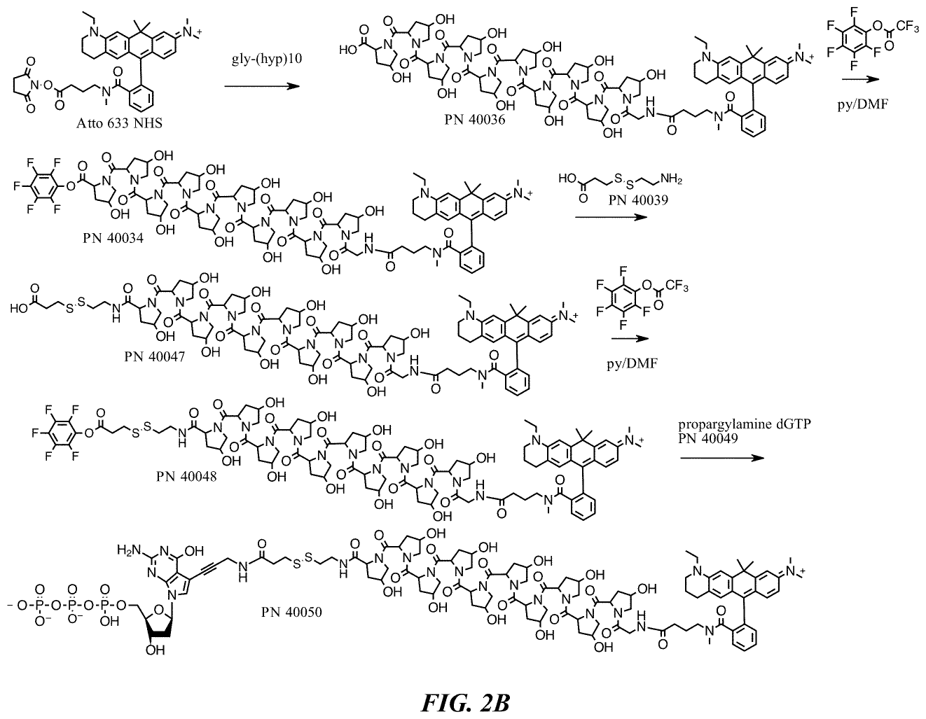

Examples of methods for constructing labeled nucleotides are shown in FIGS. 1, 2A, and 2B. As shown in FIG. 1, a labeled nucleotide may be constructed from a nucleotide, a dye, and one or more linker moieties. The one or more linker moieties together comprise a linker as described herein. A nucleotide functionalized with a propargylamino moiety can be attached to a first linker moiety via a peptide bond. This first linker moiety may comprise a cleavable moiety, such as a disulfide moiety. The first linker moiety can also be attached to one or more additional linker moieties in linear or branching fashions. For example, a second linker moiety may include two or more ring systems, wherein at least two of the two or more ring systems are separated by no more than two sp³ carbon atoms, such as by no more than two atoms. For example, at least two of the two or more ring systems may be connected to each other by a sp² carbon atom. The linker may comprise a non-proteinogenic amino acid comprising a ring system of the two or more ring systems. For example, the second linker moiety may comprise a two or more hydroxyproline moieties. An amine handle on a linker moiety may be used to attach the linker and a dye, such as a dye that fluoresces in the red or green portions of the visible electromagnetic spectrum. The labeled nucleotide generated in FIG. 1 comprises a modified deoxyadeninosine triphosphate moiety, a linker comprising a first linker moiety including a disulfide moiety and a second linker moiety including at least two ring systems, and a dye.

Construction of a labeled nucleotide can begin from either the nucleotide terminus or the dye terminus. Construction from the dye terminus permits the use of unlabeled, unactivated amino acid moieties, while construction from the nucleotide terminus may require amine-protected, carboxy-activated amino acid moieties.

FIGS. 2A and 2B show an example synthesis of a labeled nucleotide including a propargylamino functionalized dGTP moiety, a first linker moiety including a disulfide group, a second linker moiety that is hyp10, and the dye moiety Atto633. Details of this synthesis are provided in Example 2 below.

A nucleotide or nucleotide analog of a labeled nucleotide may include one or more modifications, such as one or more modifications on the nucleobase. Alternatively, a nucleotide or nucleotide analog of a labeled nucleotide may include one or more modifications not on the nucleobase. Modifications can include, but are not limited to, covalent attachment of one or more linker or label moieties, alkylation, amination, amidation, esterification, hydroxylation, halogenation, sulfurylation, and/or phosphorylation.

A nucleotide or nucleotide analog of a labeled nucleotide may include one or more modifications that are configured prevent subsequent nucleotide additions to a position adjacent to the labeled nucleotide upon its incorporation into a growing nucleic acid strand. For example, the labeled nucleotide may include a terminating or blocking group (e.g., dimethoxytrityl, phosphoramidite, or nitrobenzyl molecules). In some instances, the terminating or blocking group may be cleavable.

### Tandem Labeling

The present disclosure provides reagents and methods for tandem labeling. Tandem labeling may comprise an additional fluorescent labeling agent to a fluorescent labeling agent. Fluorescent labeling agents involved in tandem labeling or otherwise an energy transfer may be referred to herein as “tandem labeling agents.” In some cases, tandem labeling may comprise two or more tandem labeling agents. Tandem labeling may comprise an energy transfer between two tandem labeling agents. In some cases, an energy transfer between two tandem labeling agents may comprise Forster resonance energy transfer or fluorescence resonance energy transfer (FRET), resonance energy transfer (RET), or electronic energy transfer (EET). In some cases, an energy transfer between two tandem labeling agents may comprise radiationless or non-radiative energy transfer between two labeling agents. In other cases, an energy transfer between two tandem labeling agents may also comprise radiative energy transfer between two labeling agents.

In some instances, a tandem labeling agent may comprise a fluorophore. A fluorophore, in some cases, may absorb light in the ultraviolet (UV) (wavelengths approximately: 200-400 nm) or visible range (wavelengths approximately: 400-800 nm) and re-emit part of the absorbed light as radiation. In some instances, a tandem labeling agent may comprise a chromophore. A chromophore, in some cases, may absorb light in the UV and visible range and re-emit absorbed light in the visible range. In other cases, a tandem labeling agent may also comprise a phosphorescent or a chemiluminescent agent.

In some instances, a fluorophore may comprise a dye, such as a fluorescent dye. In some cases, a fluorescent dye may comprise a chemical compound. A chemical compound may be organic or inorganic. A dye or a fluorescent dye may comprise any dyes or fluorescent dyes described herein and thereof.

In some instances, a fluorophore may comprise an organic fluorescent dye. An organic fluorescent dye, in some cases, may comprise a π-conjugated polymer. A π-conjugated polymer, in some cases, may comprise a network of π-orbital that allows for electron delocalization. The electron delocalization may allow the π-conjugated polymer to absorb light from the UV to near infrared (IR) range and re-emits the absorbed light as fluorescence. In some cases, the π-orbital network may not be confined to a discrete set of atoms, the electron delocalization may spread across different polymer subunits. Such property may allow the subunits to act cooperatively in energy transfer. In some cases, a π-conjugated polymer may have a molecular extinction coefficient of about 1×10\hat{ }6 M⁻¹ cm⁻¹. In some cases, a π-conjugated polymer may comprise Brilliant Violet as described in Chattopadhyay et al., Cytometry A. 2012 June; 81(6):456-66. & U.S. Pat. Nos. 10,641,777, each of which is herein incorporated by reference in their entireties. Alternatively or in addition, a fluorescent dye may comprise an inorganic compound.

In some instances, a fluorophore may comprise a peptide, polypeptide, protein, or derivative thereof. Such a peptide, polypeptide, protein, or derivative may comprise a fluorescent protein. In some cases, a fluorescent protein may comprise phycoerythrin (PE) or APC. PE, in some cases, may have an extinction coefficient of about 1.96×10\hat{ }6 cm⁻¹M⁻¹ and quantum efficiency of about 0.82. APC, in some cases, may have an extinction coefficient of about 7×10\hat{ }5 cm⁻¹M⁻¹ and quantum efficiency of about 0.68. A fluorophore may comprise a nanoparticle. Such a nanoparticle may comprise a Quantum Dot. A Quantum Dot may be excited by EV-violet light and re-emit light at wavelengths between 525 nm to 800 nm.

In some instances, a tandem labeling mechanism may comprise a donor tandem labeling agent and an acceptor tandem labeling agent. In some instances, a donor tandem labeling agent and an acceptor tandem labeling agent may belong to the same class of molecule (e.g., a donor fluorescent dye and an acceptor fluorescent dye). In other cases, a donor tandem labeling agent and an acceptor tandem labeling agent may belong to two different classes of molecules (e.g., a donor fluorescent protein and an acceptor fluorescent dye). A donor tandem labeling agent may comprise any tandem labeling agent described herein. An acceptor tandem labeling agent may comprise any tandem labeling agent described herein. In some cases, a donor labeling agent may comprise a donor fluorophore, and an acceptor labeling agent may comprise an acceptor fluorophore.

In some instances, energy transfer may occur between a donor tandem labeling agent and an acceptor tandem labeling agent via FRET. In FRET, a donor fluorophore in an electron excited state may transfer its excitation energy to an acceptor fluorophore. The acceptor fluorophore may re-emit the transferred energy into light as radiation or fluorescence. This transfer of energy may be dependent on the proximity and orientation of the donor and acceptor fluorophores. Because the donor and acceptor fluorophores may have different excitation and emission spectrums, the donor-acceptor fluorophore pair using FRET may provide a different combination of excitation and emission spectrum than those of the donor or acceptor fluorophore alone.

In FRET, the energy transfer may occur via radiationless or non-radiative energy transfer. One such radiationless or non-radiative energy transfer may comprise dipole-dipole intermolecular coupling. In some cases, the efficiency of this energy transfer is inversely proportional to the sixth power of the distance between donor and acceptor fluorophores, as described thereof. In other cases, a donor fluorophore may also transfer its excitation energy to an acceptor fluorophore via radiative energy transfer.

According to Forster's theory, the rate of energy transfer KT is given by the below equation:

KT=(1/τD)·[R₀/r]⁶

where τD is the donor fluorophore's fluorescence lifetime in the absence of the acceptor fluorophore, R₀ is the Forster critical distance between the pair of donor and acceptor fluorophores, and r is the distance separating the donor and acceptor fluorophores. The energy transfer efficiency in FRET is inversely proportional to the sixth power of the distance between the donor and the acceptor fluorophores.

The efficiency of energy transfer, ET, is a measure of the fraction of photons absorbed by the donor fluorophore that are transferred to the acceptor fluorophore. It may be related to the distance separating the donor and acceptor fluorophores r, by the below equation:

ET=(R₀/r)⁶·1/τD, or

ET=1−(τDA/τD)

where τDA is the donor's fluorescence lifetime in the presence of the acceptor fluorophore.

In some instances, a donor fluorophore and an acceptor fluorophore are separated by a distance to allow for FRET to occur. Such a distance may be about 1 nanometer (nm), about 1.1 nm, about 1.2 nm, about 1.3 nm, about 1.4 nm, about 1.5 nm, about 1.6 nm, about 1.7 nm, about 1.8 nm, about 1.9 nm, about 2 nm, about 2.1 nm, about 2.2 nm, about 2.3 nm, about 2.4 nm, about 2.5 nm, about 2.6 nm, about 2.7 nm, about 2.8 nm, about 2.9 nm, about 3 nm, about 3.1 nm, about 3.2 nm, about 3.3 nm, about 3.4 nm, about 3.5 nm, about 3.6 nm, about 3.7 nm, about 3.8 nm, about 3.9 nm, about 4 nm, about 4.1 nm, about 4.2 nm, about 4.3 nm, about 4.4 nm, about 4.5 nm, about 4.6 nm, about 4.7 nm, about 4.8 nm, about 4.9 nm, about 5 nm, about 5.1 nm, about 5.2 nm, about 5.3 nm, about 5.4 nm, about 5.5 nm, about 5.6 nm, about 5.7 nm, about 5.8 nm, about 5.9 nm, about 6 nm, about 6.1 nm, about 6.2 nm, about 6.3 nm, about 6.4 nm, about 6.5 nm, about 6.6 nm, about 6.7 nm, about 6.8 nm, about 6.9 nm, about 7 nm, about 7.1 nm, about 7.2 nm, about 7.3 nm, about 7.4 nm, about 7.5 nm, about 7.6 nm, about 7.7 nm, about 7.8 nm, about 7.9 nm, about 8 nm, about 8.1 nm, about 8.2 nm, about 8.3 nm, about 8.4 nm, about 8.5 nm, about 8.6 nm, about 8.7 nm, about 8.8 nm, about 8.9 nm, about 9 nm, about 9.1 nm, about 9.2 nm, about 9.3 nm, about 9.4 nm, about 9.5 nm, about 9.6 nm, about 9.7 nm, about 9.8 nm, about 9.9 nm, about 10 nm, or more than about 10 nm. Such a distance may also be from about 1 to 1.1 nm, from about 1.05 to 1.15 nm, from about 1.2 to 1.3 nm, from about 1.15 to 1.25 nm, from about 1.3 to 1.4 nm, from about 1.25 to 1.35 nm, from about 1.4 to 1.5 nm, from about 1.35 to 1.45 nm, from about 1.5 to 1.6 nm, from about 1.45 to 1.55 nm, from about 1.6 to 1.7 nm, from about 1.55 to 1.65 nm, from about 1.7 to 1.8 nm, from about 1.65 to 1.75 nm, from about 1.8 to 1.9 nm, from about 1.75 to 1.85 nm, from about 1.9 to 2 nm, from about 1.85 to 1.95 nm, from about 2 to 2.1 nm, from about 1.95 to 2.05 nm, from about 2.1 to 2.2 nm, from about 2.05 to 2.15 nm, from about 2.2 to 2.3 nm, from about 2.15 to 2.25 nm, from about 2.3 to 2.4 nm, from about 2.25 to 2.35 nm, from about 2.4 to 2.5 nm, from about 2.35 to 2.45 nm, from about 2.5 to 2.6 nm, from about 2.45 to 2.55 nm, from about 2.6 to 2.7 nm, from about 2.55 to 2.65 nm, from about 2.7 to 2.8 nm, from about 2.65 to 2.75 nm, from about 2.8 to 2.9 nm, from about 2.75 to 2.85 nm, from about 2.9 to 3 nm, from about 2.85 to 2.95 nm, from about 3 to 3.1 nm, from about 2.95 to 3.05 nm, from about 3.1 to 3.2 nm, from about 3.05 to 3.15 nm, from about 3.2 to 3.3 nm, from about 3.15 to 3.25 nm, from about 3.3 to 3.4 nm, from about 3.25 to 3.35 nm, from about 3.4 to 3.5 nm, from about 3.35 to 3.45 nm, from about 3.5 to 3.6 nm, from about 3.45 to 3.55 nm, from about 3.6 to 3.7 nm, from about 3.55 to 3.65 nm, from about 3.7 to 3.8 nm, from about 3.65 to 3.75 nm, from about 3.8 to 3.9 nm, from about 3.75 to 3.85 nm, from about 3.9 to 4 nm, from about 3.85 to 3.95 nm, from about 4 to 4.1 nm, from about 3.95 to 4.05 nm, from about 4.1 to 4.2 nm, from about 4.05 to 4.15 nm, from about 4.2 to 4.3 nm, from about 4.15 to 4.25 nm, from about 4.3 to 4.4 nm, from about 4.25 to 4.35 nm, from about 4.4 to 4.5 nm, from about 4.35 to 4.45 nm, from about 4.5 to 4.6 nm, from about 4.45 to 4.55 nm, from about 4.6 to 4.7 nm, from about 4.55 to 4.65 nm, from about 4.7 to 4.8 nm, from about 4.65 to 4.75 nm, from about 4.8 to 4.9 nm, from about 4.75 to 4.85 nm, from about 4.9 to 5 nm, from about 4.85 to 4.95 nm, from about 5 to 5.1 nm, from about 4.95 to 5.05 nm, from about 5.1 to 5.2 nm, from about 5.05 to 5.15 nm, from about 5.2 to 5.3 nm, from about 5.15 to 5.25 nm, from about 5.3 to 5.4 nm, from about 5.25 to 5.35 nm, from about 5.4 to 5.5 nm, from about 5.35 to 5.45 nm, from about 5.5 to 5.6 nm, from about 5.45 to 5.55 nm, from about 5.6 to 5.7 nm, from about 5.55 to 5.65 nm, from about 5.7 to 5.8 nm, from about 5.65 to 5.75 nm, from about 5.8 to 5.9 nm, from about 5.75 to 5.85 nm, from about 5.9 to 6 nm, from about 5.85 to 5.95 nm, from about 6 to 6.1 nm, from about 5.95 to 6.05 nm, from about 6.1 to 6.2 nm, from about 6.05 to 6.15 nm, from about 6.2 to 6.3 nm, from about 6.15 to 6.25 nm, from about 6.3 to 6.4 nm, from about 6.25 to 6.35 nm, from about 6.4 to 6.5 nm, from about 6.35 to 6.45 nm, from about 6.5 to 6.6 nm, from about 6.45 to 6.55 nm, from about 6.6 to 6.7 nm, from about 6.55 to 6.65 nm, from about 6.7 to 6.8 nm, from about 6.65 to 6.75 nm, from about 6.8 to 6.9 nm, from about 6.75 to 6.85 nm, from about 6.9 to 7 nm, from about 6.85 to 6.95 nm, from about 7 to 7.1 nm, from about 6.95 to 7.05 nm, from about 7.1 to 7.2 nm, from about 7.05 to 7.15 nm, from about 7.2 to 7.3 nm, from about 7.15 to 7.25 nm, from about 7.3 to 7.4 nm, from about 7.25 to 7.35 nm, from about 7.4 to 7.5 nm, from about 7.35 to 7.45 nm, from about 7.5 to 7.6 nm, from about 7.45 to 7.55 nm, from about 7.6 to 7.7 nm, from about 7.55 to 7.65 nm, from about 7.7 to 7.8 nm, from about 7.65 to 7.75 nm, from about 7.8 to 7.9 nm, from about 7.75 to 7.85 nm, from about 7.9 to 8 nm, from about 7.85 to 7.95 nm, from about 8 to 8.1 nm, from about 7.95 to 8.05 nm, from about 8.1 to 8.2 nm, from about 8.05 to 8.15 nm, from about 8.2 to 8.3 nm, from about 8.15 to 8.25 nm, from about 8.3 to 8.4 nm, from about 8.25 to 8.35 nm, from about 8.4 to 8.5 nm, from about 8.35 to 8.45 nm, from about 8.5 to 8.6 nm, from about 8.45 to 8.55 nm, from about 8.6 to 8.7 nm, from about 8.55 to 8.65 nm, from about 8.7 to 8.8 nm, from about 8.65 to 8.75 nm, from about 8.8 to 8.9 nm, from about 8.75 to 8.85 nm, from about 8.9 to 9 nm, from about 8.85 to 8.95 nm, from about 9 to 9.1 nm, from about 8.95 to 9.05 nm, from about 9.1 to 9.2 nm, from about 9.05 to 9.15 nm, from about 9.2 to 9.3 nm, from about 9.15 to 9.25 nm, from about 9.3 to 9.4 nm, from about 9.25 to 9.35 nm, from about 9.4 to 9.5 nm, from about 9.35 to 9.45 nm, from about 9.5 to 9.6 nm, from about 9.45 to 9.55 nm, from about 9.6 to 9.7 nm, from about 9.55 to 9.65 nm, from about 9.7 to 9.8 nm, from about 9.65 to 9.75 nm, from about 9.8 to 9.9 nm, from about 9.75 to 9.85 nm, or from about 9.9 to 10 nm. In some instances, linking a donor fluorophore and an acceptor fluorophore using a linker described herein (e.g., a hyp10 or hyp20 linker) may facilitate the energy transfer by positioning the fluorophores physically close enough to allow FRET to occur. Such distance may be any distance described herein.

In some instances, the emission spectrum of a donor fluorophore and the excitation spectrum of an acceptor fluorophore may overlap to allow for FRET to occur. Such an overlap may be 5%, 10%, 15%, 20%, 25%, 30%, 35%, 40%, 45%, 50%, 55%, 60%, 65%, 70%, 75%, 80%, 85%, 90%, 95%, or 100%. In some cases, the emission spectrum of a donor fluorophore and the excitation spectrum of an acceptor fluorophore may overlap by about 1 to 10%, 5 to 15%, 10 to 20%, 15 to 25%, 20 to 30%, 25 to 35%, 30 to 40%, 35 to 45%, 40 to 50%, 45 to 55%, 50 to 60%, 55 to 65%, 60 to 70%, 65 to 75%, 70 to 80%, 75 to 85%, 80 to 90%, 85 to 95%, or 90 to 100%. In other cases, the emission spectrum of a donor fluorophore and the excitation spectrum of an acceptor fluorophore may not overlap to allow for FRET to occur. In some cases, the choice of a donor fluorophore may determine whether the emission spectrum of the donor fluorophore and the excitation spectrum of an acceptor fluorophore need to overlap. For example, when using a π-conjugated polymer (e.g., Brilliant Violet) as a donor fluorophore, the mission spectrum of the donor fluorophore and the excitation spectrum of the acceptor fluorophore may not overlap to allow for FRET to occur. In other cases, for example, when using PE or APC as a donor fluorophore, the emission spectrum of the donor fluorophore and the excitation spectrum of the acceptor fluorophore may overlap by the extent described herein and thereof to allow for FRET to occur. In some instances, the energy transferred between a donor fluorophore and an acceptor fluorophore may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In other cases, the energy transferred between a donor fluorophore and an acceptor fluorophore may be about 1 to 10%, 5 to 15%, 10 to 20%, 15 to 25%, 20 to 30%, 25 to 35%, 30 to 40%, 35 to 45%, 40 to 50%, 45 to 55%, 50 to 60%, 55 to 65%, 60 to 70%, 65 to 75%, 70 to 80%, 75 to 85%, 80 to 90%, 85 to 95%, or 90 to 100%. Donor fluorophores may comprise any dyes or fluorescent dyes described herein, any derivatives thereof, and any combination herein and thereof.

In some instances, the maximum excitation wavelength (Exₘₐₓ) of a donor fluorophore may be about 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm, 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 551 nm, 552 nm, 553 nm, 554 nm, 555 nm, 556 nm, 557 nm, 558 nm, 559 nm, 560 nm, 561 nm, 562 nm, 563 nm, 564 nm, 565 nm, 566 nm, 567 nm, 568 nm, 569 nm, 570 nm, 571 nm, 572 nm, 573 nm, 574 nm, 575 nm, 576 nm, 577 nm, 578 nm, 579 nm, 580 nm, 581 nm, 582 nm, 583 nm, 584 nm, 585 nm, 586 nm, 587 nm, 588 nm, 589 nm, 590 nm, 591 nm, 592 nm, 593 nm, 594 nm, 595 nm, 596 nm, 597 nm, 598 nm, 599 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 651 nm, 652 nm, 653 nm, 654 nm, 655 nm, 656 nm, 657 nm, 658 nm, 659 nm, 660 nm, 661 nm, 662 nm, 663 nm, 664 nm, 665 nm, 666 nm, 667 nm, 668 nm, 669 nm, 670 nm, 671 nm, 672 nm, 673 nm, 674 nm, 675 nm, 676 nm, 677 nm, 678 nm, 679 nm, 680 nm, 681 nm, 682 nm, 683 nm, 684 nm, 685 nm, 686 nm, 687 nm, 688 nm, 689 nm, 690 nm, 691 nm, 692 nm, 693 nm, 694 nm, 695 nm, 696 nm, 697 nm, 698 nm, 699 nm, 700 nm, 701 nm, 702 nm, 703 nm, 704 nm, 705 nm, 706 nm, 707 nm, 708 nm, 709 nm, 710 nm, 711 nm, 712 nm, 713 nm, 714 nm, 715 nm, 716 nm, 717 nm, 718 nm, 719 nm, 720 nm, 721 nm, 722 nm, 723 nm, 724 nm, 725 nm, 726 nm, 727 nm, 728 nm, 729 nm, 730 nm, 731 nm, 732 nm, 733 nm, 734 nm, 735 nm, 736 nm, 737 nm, 738 nm, 739 nm, 740 nm, 741 nm, 742 nm, 743 nm, 744 nm, 745 nm, 746 nm, 747 nm, 748 nm, 749 nm, or 750 nm. In some cases, the Exₘₐₓ of a donor fluorophore may comprise 335 nm, 404 nm, 405 nm, 407 nm, 415 nm, 482 nm, 488 nm, 494 nm, 495 nm, 496 nm, 532 nm, 561 nm, 633 nm, 635 nm, 640 nm, 650 nm, or 696 nm.

In some instances, the maximum emission wavelength (Emₘₐₓ) of a donor fluorophore may be about 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm, 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 551 nm, 552 nm, 553 nm, 554 nm, 555 nm, 556 nm, 557 nm, 558 nm, 559 nm, 560 nm, 561 nm, 562 nm, 563 nm, 564 nm, 565 nm, 566 nm, 567 nm, 568 nm, 569 nm, 570 nm, 571 nm, 572 nm, 573 nm, 574 nm, 575 nm, 576 nm, 577 nm, 578 nm, 579 nm, 580 nm, 581 nm, 582 nm, 583 nm, 584 nm, 585 nm, 586 nm, 587 nm, 588 nm, 589 nm, 590 nm, 591 nm, 592 nm, 593 nm, 594 nm, 595 nm, 596 nm, 597 nm, 598 nm, 599 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 651 nm, 652 nm, 653 nm, 654 nm, 655 nm, 656 nm, 657 nm, 658 nm, 659 nm, 660 nm, 661 nm, 662 nm, 663 nm, 664 nm, 665 nm, 666 nm, 667 nm, 668 nm, 669 nm, 670 nm, 671 nm, 672 nm, 673 nm, 674 nm, 675 nm, 676 nm, 677 nm, 678 nm, 679 nm, 680 nm, 681 nm, 682 nm, 683 nm, 684 nm, 685 nm, 686 nm, 687 nm, 688 nm, 689 nm, 690 nm, 691 nm, 692 nm, 693 nm, 694 nm, 695 nm, 696 nm, 697 nm, 698 nm, 699 nm, 700 nm, 701 nm, 702 nm, 703 nm, 704 nm, 705 nm, 706 nm, 707 nm, 708 nm, 709 nm, 710 nm, 711 nm, 712 nm, 713 nm, 714 nm, 715 nm, 716 nm, 717 nm, 718 nm, 719 nm, 720 nm, 721 nm, 722 nm, 723 nm, 724 nm, 725 nm, 726 nm, 727 nm, 728 nm, 729 nm, 730 nm, 731 nm, 732 nm, 733 nm, 734 nm, 735 nm, 736 nm, 737 nm, 738 nm, 739 nm, 740 nm, 741 nm, 742 nm, 743 nm, 744 nm, 745 nm, 746 nm, 747 nm, 748 nm, 749 nm, 750 nm, 751 nm, 752 nm, 753 nm, 754 nm, 755 nm, 756 nm, 757 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 788 nm, 789 nm, 790 nm, 791 nm, 792 nm, 793 nm, 794 nm, 795 nm, 796 nm, 797 nm, 798 nm, 799 nm, or 800 nm.

In some instances, an acceptor fluorophore for FRET may comprise FITC, PE, APC, a n-conjugated polymer (e.g., Brilliant Violet), Cy-5, Cy-5.5, Cy-7, AlexaFluor dyes (e.g., Alexa Fluor 488, 594, 647, or 700 dyes), Atto-633 dyes, Peridinin-Chlorophyll-Protein, any derivative thereof, or any combination herein and thereof. Acceptor fluorophores may comprise any dyes or fluorescent dyes described herein, any derivatives thereof, and any combination herein and thereof.

In some instances, the Emₘₐₓ of an acceptor fluorophore may be about 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm, 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 551 nm, 552 nm, 553 nm, 554 nm, 555 nm, 556 nm, 557 nm, 558 nm, 559 nm, 560 nm, 561 nm, 562 nm, 563 nm, 564 nm, 565 nm, 566 nm, 567 nm, 568 nm, 569 nm, 570 nm, 571 nm, 572 nm, 573 nm, 574 nm, 575 nm, 576 nm, 577 nm, 578 nm, 579 nm, 580 nm, 581 nm, 582 nm, 583 nm, 584 nm, 585 nm, 586 nm, 587 nm, 588 nm, 589 nm, 590 nm, 591 nm, 592 nm, 593 nm, 594 nm, 595 nm, 596 nm, 597 nm, 598 nm, 599 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 651 nm, 652 nm, 653 nm, 654 nm, 655 nm, 656 nm, 657 nm, 658 nm, 659 nm, 660 nm, 661 nm, 662 nm, 663 nm, 664 nm, 665 nm, 666 nm, 667 nm, 668 nm, 669 nm, 670 nm, 671 nm, 672 nm, 673 nm, 674 nm, 675 nm, 676 nm, 677 nm, 678 nm, 679 nm, 680 nm, 681 nm, 682 nm, 683 nm, 684 nm, 685 nm, 686 nm, 687 nm, 688 nm, 689 nm, 690 nm, 691 nm, 692 nm, 693 nm, 694 nm, 695 nm, 696 nm, 697 nm, 698 nm, 699 nm, 700 nm, 701 nm, 702 nm, 703 nm, 704 nm, 705 nm, 706 nm, 707 nm, 708 nm, 709 nm, 710 nm, 711 nm, 712 nm, 713 nm, 714 nm, 715 nm, 716 nm, 717 nm, 718 nm, 719 nm, 720 nm, 721 nm, 722 nm, 723 nm, 724 nm, 725 nm, 726 nm, 727 nm, 728 nm, 729 nm, 730 nm, 731 nm, 732 nm, 733 nm, 734 nm, 735 nm, 736 nm, 737 nm, 738 nm, 739 nm, 740 nm, 741 nm, 742 nm, 743 nm, 744 nm, 745 nm, 746 nm, 747 nm, 748 nm, 749 nm, 750 nm, 751 nm, 752 nm, 753 nm, 754 nm, 755 nm, 756 nm, 757 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 788 nm, 789 nm, 790 nm, 791 nm, 792 nm, 793 nm, 794 nm, 795 nm, 796 nm, 797 nm, 798 nm, 799 nm, or 800 nm. In some cases, the Emₘₐₓ of an acceptor fluorophore may be 421 nm, 448 nm, 510 nm, 519 nm, 520 nm, 578 nm, 602 nm, 612 nm, 650 nm, 660 nm, 667 nm, 668 nm, 678 nm, 695 nm, 711 nm, 719 nm, 785 nm, or 786 nm.

In some instances, the Exₘₐₓ of an acceptor fluorophore may be about 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm, 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 551 nm, 552 nm, 553 nm, 554 nm, 555 nm, 556 nm, 557 nm, 558 nm, 559 nm, 560 nm, 561 nm, 562 nm, 563 nm, 564 nm, 565 nm, 566 nm, 567 nm, 568 nm, 569 nm, 570 nm, 571 nm, 572 nm, 573 nm, 574 nm, 575 nm, 576 nm, 577 nm, 578 nm, 579 nm, 580 nm, 581 nm, 582 nm, 583 nm, 584 nm, 585 nm, 586 nm, 587 nm, 588 nm, 589 nm, 590 nm, 591 nm, 592 nm, 593 nm, 594 nm, 595 nm, 596 nm, 597 nm, 598 nm, 599 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 651 nm, 652 nm, 653 nm, 654 nm, 655 nm, 656 nm, 657 nm, 658 nm, 659 nm, 660 nm, 661 nm, 662 nm, 663 nm, 664 nm, 665 nm, 666 nm, 667 nm, 668 nm, 669 nm, 670 nm, 671 nm, 672 nm, 673 nm, 674 nm, 675 nm, 676 nm, 677 nm, 678 nm, 679 nm, 680 nm, 681 nm, 682 nm, 683 nm, 684 nm, 685 nm, 686 nm, 687 nm, 688 nm, 689 nm, 690 nm, 691 nm, 692 nm, 693 nm, 694 nm, 695 nm, 696 nm, 697 nm, 698 nm, 699 nm, 700 nm, 701 nm, 702 nm, 703 nm, 704 nm, 705 nm, 706 nm, 707 nm, 708 nm, 709 nm, 710 nm, 711 nm, 712 nm, 713 nm, 714 nm, 715 nm, 716 nm, 717 nm, 718 nm, 719 nm, 720 nm, 721 nm, 722 nm, 723 nm, 724 nm, 725 nm, 726 nm, 727 nm, 728 nm, 729 nm, 730 nm, 731 nm, 732 nm, 733 nm, 734 nm, 735 nm, 736 nm, 737 nm, 738 nm, 739 nm, 740 nm, 741 nm, 742 nm, 743 nm, 744 nm, 745 nm, 746 nm, 747 nm, 748 nm, 749 nm, 750 nm, 751 nm, 752 nm, 753 nm, 754 nm, 755 nm, 756 nm, 757 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 788 nm, 789 nm, 790 nm, 791 nm, 792 nm, 793 nm, 794 nm, 795 nm, 796 nm, 797 nm, 798 nm, 799 nm, or 800 nm.

Additional example of tandem labeling agents include, e.g., those described in U.S. Pat. Nos. 8,927,212, 9,616,141, and 10,641,777, each of which is entirely incorporated herein by reference for all purposes.

In some instances, a donor tandem labeling agent and an acceptor tandem labeling agent may be conjugated or linked in tandem labeling. In other cases, a donor fluorophore and an acceptor fluorophore may be conjugated or linked in tandem labeling. A conjugation or link, in some cases, may comprise a covalent interaction. Such a conjugation or link may also comprise a linker. A linker may comprise any linkers or derivatives described herein and thereof. In some cases, a linker may comprise a hyp10 or hyp20 linker. Linkage and conjugation may occur via one of the hydroxyproline moieties in a hyp10 or hyp20 linker. A linker, such as a hyp10 or hyp20 linker, in some cases, may allow FRET to occur between a donor fluorophore and an acceptor fluorophore in tandem labeling. In other cases, a linker (e.g., a hyp10 or hyp20 linker) may facilitate FRET to occur between a donor fluorophore and an acceptor fluorophore in tandem labeling.

In some instances, a substrate described herein may be linked or conjugated to a donor tandem labeling agent by a linker. In some cases, a substrate described herein may be linked or conjugated to an acceptor tandem labeling agent by a linker. A linker may comprise any linkers or derivatives described herein and thereof. In some cases, a linker may comprise a hyp10 or hyp20 linker. In some cases, a substrate described herein may be linked or conjugated to a donor tandem labeling agent and an acceptor labeling agent by a linker (e.g., a hyp10 or hyp20 linker). In other cases, a substrate described herein may be linked or conjugated to a donor tandem labeling agent without a linker. In some cases, a substrate described herein may be linked or conjugated to an acceptor tandem labeling agent without a linker. For example, the donor tandem labeling agent or the acceptor tandem labeling agent may comprise the substrate as a single chemical entity.

In some instances, a donor-acceptor fluorophore pair may comprise donor fluorophore described herein, conjugated or linked to any acceptor fluorophore described herein. In some cases, a donor-acceptor fluorophore pair may comprise a π-conjugated polymer (e.g., Brilliant Violet) as a donor fluorophore. In some cases, a donor-acceptor fluorophore pair comprising a π-conjugated polymer as a donor fluorophore may have an Exₘₐₓ at 404 nm, 405 nm, 407 nm, or 415 nm. In some cases, a donor-acceptor fluorophore pair comprising a π-conjugated polymer as a donor fluorophore may be excited by a violet laser. In some cases, a donor-acceptor fluorophore pair comprising π-conjugated polymer as a donor fluorophore may have an Emₘₐₓ at 421 nm, 448 nm, 510 nm, 570 nm, 602 nm, 603 nm, 646 nm, 650 nm, 711 nm, 750 nm, 785 nm, or 786 nm. In other cases, the Emₘₐₓ of a donor-acceptor fluorophore pair comprising a π-conjugated polymer as a donor fluorophore may be modified to any wavelength described herein using method described (Chattopadhyay et al., Cytometry A. 2012 June; 81(6):456-66. which is herein incorporated by reference in its entirety). In some instances, a donor-acceptor fluorophore pair may comprise APC as a donor fluorophore. In some cases, a donor-acceptor fluorophore pair comprising APC as a donor fluorophore may have an Exₘₐₓ at 650 nm or 696 nm. In some cases, a donor-acceptor fluorophore pair comprising APC as a donor fluorophore may be excited by a red laser. In some cases, a donor-acceptor fluorophore pair comprising APC as a donor may comprise Cy-7, Atto-633 dye, Alexa Fluor 647 dye, Alexa Fluor 700 dye, or derivatives herein as an acceptor fluorophore. In some cases, a donor-acceptor fluorophore pair comprising APC as a donor fluorophore may have an Emₘₐₓ at 660 nm, 668 nm, 669 nm, 719 nm, 785 nm, 787 nm, or 807 nm. In some instances, a donor-acceptor fluorophore pair may comprise PE as a donor fluorophore. In some cases, a donor-acceptor fluorophore pair comprising PE as a donor fluorophore may have an Exₘₐₓ at 496 nm or 565 nm. In some cases, a donor-acceptor fluorophore pair comprising PE as a donor fluorophore may be excited by a blue laser or a yellow-green laser. In some cases, a donor-acceptor fluorophore pair comprising PE as a donor may comprise R-PE, CF-594 dye, Cy-5, Cy-5.5, Cy-7, Atto-633 dye, Alexa Fluor 647 dye, or derivatives herein as an acceptor fluorophore. In some cases, a donor-acceptor fluorophore pair comprising PE as a donor fluorophore may have an Emₘₐₓ at 610 nm, 660 nm, 668 nm, 669 nm, 719 nm, 785 nm, 787 nm, or 807 nm. In some cases, a donor-acceptor fluorophore pair may comprise any maximum excitation wavelength of a donor as described herein. In other cases, a donor-acceptor fluorophore pair may comprise any maximum emission wavelength of an acceptor fluorophore as described herein. In some cases, different maximum emission wavelength of an acceptor fluorophore can be created or modified using the dyes or fluorescent dyes as described herein.

In some instances, the emission spectrum of a donor fluorophore and the emission spectrum of an acceptor fluorophore may overlap or may not overlap. In some cases, the overlap of the emission spectrums of a donor and acceptor fluorophores may be 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%. In some cases, the emission spectrums of a donor fluorophore and an acceptor fluorophore may overlap by about 1 to 10%, 5 to 15%, 10 to 20%, 15 to 25%, 20 to 30%, 25 to 35%, 30 to 40%, 35 to 45%, 40 to 50%, 45 to 55%, 50 to 60%, 55 to 65%, 60 to 70%, 65 to 75%, 70 to 80%, 75 to 85%, 80 to 90%, 85 to 95%, or 90 to 100%. In some cases, the overlap of the emission spectrums of a donor and acceptor fluorophores may not overlap. In some instances, an acceptor fluorophore, when paired with a donor fluorophore in tandem labeling, may change the relationship of the light excitation and emission. For example, when not paired, a donor fluorophore may be excited with light at wavelength Dₑₓ and emit light at wavelength Dₑₘ, and an acceptor fluorophore may be excited with light at wavelength Aₑₓ and emit light at wavelength Aₑₘ. When paired in tandem labeling, the pair of donor-acceptor fluorophore may be excited with light at wavelength Dₑₓ and emit light at wavelength Aₑₘ. The overlap between Dₑₘ and Aₑₘ may comprise any percentage described herein.

In some instances, FRET between a donor fluorophore and an acceptor fluorophore in a donor-acceptor fluorophore pair may alter the Stokes shift of the donor fluorophore and the acceptor fluorophore. In some cases, FRET between a donor fluorophore and an acceptor fluorophore in a donor-acceptor fluorophore pair may increase the Stokes shift of the donor fluorophore and the acceptor fluorophore. Stokes shift is the difference in the wavelength in which of a molecule is excited with light and emits light. In some cases, Stokes shift is the difference of the Exₘₐₓ and Emₘₐₓ of a fluorophore. For example, a donor fluorophore may have an Exₘₐₓ and Emₘₐₓ at Dₑₓ and Dₑₘ, respectively, wherein Dₑₘ>Dₑₓ. An acceptor fluorophore may have an Exₘₐₓ and Emₘₐₓ at Aₑₓ and Aₑₘ, respectively, wherein Aₑₘ>Aₑₓ. The Stokes shift of the donor fluorophore and the acceptor fluorophore may be Dₑₓ-Dₑₘ and Aₑₓ−Aₑₘ, respectively, wherein Aₑₓ>Dₑₓ and Aₑₘ>Dₑₘ. When the donor and the acceptor fluorophores combine to form a donor-acceptor fluorophore using FRET to transfer energy, the Stokes shift of a donor-acceptor fluorophore may become Aₑₓ−Dₑₘ. In some cases, the Stokes shift of a donor-acceptor fluorophore may be about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, or 750 nm larger than that of a donor fluorophore or an acceptor fluorophore alone. In some cases, the Stokes shift of a donor-acceptor fluorophore may be from 1 to 20 nm, from 10 to 30 nm, from 20 to 40 nm, from 30 to 50 nm, from 40 to 60 nm, from 50 to 70 nm, from 60 to 80 nm, from 70 to 90 nm, from 80 to 100 nm, from 90 to 110 nm, from 100 to 120 nm, from 110 to 130 nm, from 120 to 140 nm, from 130 to 150 nm, from 140 to 160 nm, from 150 to 170 nm, from 160 to 180 nm, from 170 to 190 nm, from 180 to 200 nm, from 190 to 210 nm, from 200 to 220 nm, from 210 to 230 nm, from 220 to 240 nm, from 230 to 250 nm, from 240 to 260 nm, from 250 to 270 nm, from 260 to 280 nm, from 270 to 290 nm, from 280 to 300 nm, from 290 to 310 nm, from 300 to 320 nm, from 310 to 330 nm, from 320 to 340 nm, from 330 to 350 nm, from 340 to 360 nm, from 350 to 370 nm, from 360 to 380 nm, from 370 to 390 nm, from 380 to 400 nm, from 390 to 410 nm, from 400 to 420 nm, from 410 to 430 nm, from 420 to 440 nm, from 430 to 450 nm, from 440 to 460 nm, from 450 to 470 nm, from 460 to 480 nm, from 470 to 490 nm, from 480 to 500 nm, from 490 to 510 nm, from 500 to 520 nm, from 510 to 530 nm, from 520 to 540 nm, from 530 to 550 nm, from 540 to 560 nm, from 550 to 570 nm, from 560 to 580 nm, from 570 to 590 nm, from 580 to 600 nm, from 590 to 610 nm, from 600 to 620 nm, from 610 to 630 nm, from 620 to 640 nm, from 630 to 650 nm, from 640 to 660 nm, from 650 to 670 nm, from 660 to 680 nm, from 670 to 690 nm, from 680 to 700 nm, from 690 to 710 nm, from 700 to 720 nm, from 710 to 730 nm, from 720 to 740 nm, or from 730 to 750 nm larger than that of a donor fluorophore or an acceptor fluorophore alone.

In some instances, the emission spectrum of a donor fluorophore and the emission spectrum of an acceptor fluorophore may be essentially the same (e.g., the emission spectrums of a donor and acceptor fluorophores may overlap by at least 80%, 81%, 82%, 83%, 84%, 85%, 86%, 87%, 88%, 89%, 90%, 91%, 92%, 93%, 94%, 95%, 96%, 97%, 98%, 99%, or 100%). In some cases, an acceptor fluorophore, when paired with a donor fluorophore in tandem labeling, may maintain the relationship of the light excitation and emission as when the donor and acceptor fluorophores are not paired. For example, when not paired, a donor fluorophore may be excited with light at wavelength Dₑₓ and emit light at wavelength Dₑₘ, and an acceptor fluorophore may be excited with light at wavelength Dₑₓ and emit light at wavelength Dₑₘ. When paired in tandem labeling, the pair of donor-acceptor fluorophore may be excited with light at wavelength Dₑₓ and emit light at wavelength Dₑₘ. In some cases, a donor-acceptor fluorophore pair may have increased fluorescent intensity than a donor fluorophore or an acceptor fluorophore not paired.

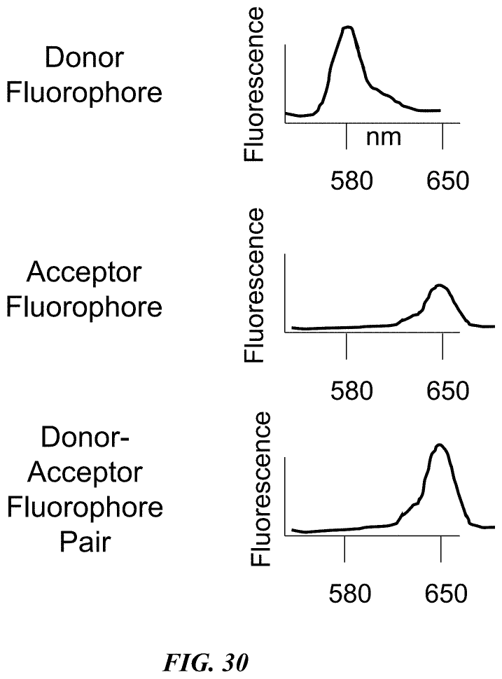

FIG. 30 illustrates an example of the relationship between the excitation spectrum, emission spectrum, and fluorescent intensity of a donor-acceptor fluorophore pair in tandem labeling. A donor fluorophore and an acceptor fluorophore may have an Exₘₐₓ at 405 nm. Once excited, the donor fluorophore may emit light with Emₘₐₓ at 580 nm at a high fluorescent intensity, and the acceptor fluorophore may emit light with Emₘₐₓ at 650 nm at a low fluorescent intensity. In a donor-acceptor fluorophore pair using the same donor and acceptor fluorophores, the pair may still have an Exₘₐₓ at 405 nm. Once excited, the pair may emit light with Emₘₐₓ at 650 nm with a high fluorescent intensity.

In some instances, a donor or an acceptor fluorophore alone may emit light at the same wavelength as that of a donor-acceptor fluorophore pair in tandem labeling. In some cases, a donor-acceptor fluorophore pair in a tandem pairing may emit more photons per emission than that of a donor or an acceptor not paired. In some cases, a donor-acceptor fluorophore pair in a tandem pairing may emit about 10%, 20%, 30%, 40%, 50%, 60%, 70%, 80%, 90%, 100%, 200%, 300%, 400%, 500%, 600%, 700%, 800% 900%, 1000%, 2000%, 3000%, 4000%, 5000%, 6000%, 7000%, 8000%, 9000%, or 10000% more photons than a donor or an acceptor not paired does. In some cases, a donor-acceptor fluorophore pair in a tandem pairing may emit about from about 10 to 100%, from 50 to 200%, from 100 to 300%, from 150 to 400%, from 200 to 500%, from 250 to 600%, from 300 to 700%, from 350 to 800%, from 400 to 900%, from 450 to 1000%, from 500 to 2000%, from 1500 to 3000%, from 2500 to 4000%, from 3500 to 5000%, from 4500 to 6000%, from 5500 to 7000%, from 6500 to 8000%, from 7500 to 9000%, or from 8500 to 10000% more photons than a donor or an acceptor not paired does.

In some instances, a donor-acceptor fluorophore pair in a tandem labeling may provide 10%, 20%, 30%, 40%, 50%, 60%, 70%, 80%, 90%, 100%, 200%, 300%, 400%, 500%, 600%, 700%, 800% 900%, 1000%, 2000%, 3000%, 4000%, 5000%, 6000%, 7000%, 8000%, 9000%, or 10000% higher sensitivity for the detection of the fluorescent intensity than a donor or an acceptor not paired does. In some cases, a donor-acceptor fluorophore pair in a tandem pairing may provide from about 10 to 100%, from 50 to 200%, from 100 to 300%, from 150 to 400%, from 200 to 500%, from 250 to 600%, from 300 to 700%, from 350 to 800%, from 400 to 900%, from 450 to 1000%, from 500 to 2000%, from 1500 to 3000%, from 2500 to 4000%, from 3500 to 5000%, from 4500 to 6000%, from 5500 to 7000%, from 6500 to 8000%, from 7500 to 9000%, or from 8500 to 10000% higher sensitivity for the detection of the fluorescent intensity than a donor or an acceptor not paired does.

In some instances, tandem labeling may comprise exciting a donor fluorophore in a donor-acceptor fluorophore pair with a laser. In some cases, a laser may comprise a ultraviolet laser (355 nm), a violet laser (405 nm), a blue laser (488 nm), a green laser (532 nm), a yellow-green laser (561 nm), or a red laser (633 nm). In some cases, tandem labeling may allow emission of 1 donor-acceptor fluorophore pair using one laser. In other cases, tandem labeling may allow emission of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different donor-acceptor fluorophore pairs using one laser. In some cases, a first donor-acceptor fluorophore pair may be distinguished from a second donor-acceptor fluorophore pair. To be distinguishable, the first donor-acceptor fluorophore pair and the second donor-acceptor fluorophore pair may comprise two different Emₘₐₓ. In some cases, the first donor-acceptor fluorophore pair and the second donor-acceptor fluorophore pair may be distinguishable by the two non-overlapping emission spectrums of the pairs. In other cases, the first donor-acceptor fluorophore pair and the second donor-acceptor fluorophore pair may comprise overlapping emission spectrum but are distinguishable by the nonoverlapping region of their respective emission spectrums. Each donor-acceptor fluorophore pair, in some cases, may comprise a unique Emₘₐₓ. In some cases, tandem labeling may allow emission of from 1 to 10, from 5 to 15, from 10 to 20, from 15 to 25, from 20 to 30, from 25 to 35, from 30 to 40, from 35 to 45, from 40 to 50 different donor-acceptor fluorophore pairs using one laser. In some cases, the emission of different donor-acceptor fluorophore pairs excited by one laser may be record individually. In some cases, the emission of different donor-acceptor fluorophore pairs excited by one laser may be record simultaneously.

In some instances, each donor-acceptor fluorophore pair may be conjugated to one molecule. In some cases, such a molecule conjugated to a donor-acceptor fluorophore pair with a unique Emₘₐₓ or emission spectrum may bind to a unique cellular molecule or a unique set of cellular molecules. In some cases, a molecule conjugated to a donor-acceptor fluorophore pair with a unique Emₘₐₓ or emission spectrum may comprise a peptide, a nucleic acid or a chemical compound. In some cases, a peptide, a nucleic acid or a chemical compound may bind to a nucleotide, a nucleotide sequence, an amino acid, a peptide, a carbohydrate, or a lipid. In some instances, a conjugation or link may comprise a covalent or non-covalent interaction. Such a conjugation or link may also comprise a linker. A linker may comprise any linkers described herein or derivatives thereof. In some cases, a linker may comprise a hyp10 or hyp20 linker. Linkage and conjugation may occur via one of the hydroxyproline moieties in a hyp10 or hyp10 linker. In some cases, a molecule being conjugated or linked with a labeling agent may comprise a nucleotide, an amino acid, a lipid, a carbohydrate, any derivative herein and thereof, and any combination herein and thereof. For example, a fluorescent dye may be conjugated to a deoxyribose nucleotide or a ribose nucleotide as described herein. In other case, a tandem labeling agent other than a donor-acceptor fluorophore pair, such as a chromophore, may be conjugated or linked to another molecule.

In some instances, a peptide conjugated to a donor-acceptor fluorophore pair may comprise an antibody. In some cases, an antibody conjugated to a donor-acceptor fluorophore pair may comprise IgA, IgD, IgE, IgG, IgM, any derivatives herein and thereof, or any combinations herein and thereof. In some cases, an antibody conjugated to a donor-acceptor fluorophore pair may comprise a murine, human, chimeric, or humanized antibody. In other cases, an antibody conjugated to a donor-acceptor fluorophore pair may comprise a polyclonal or monoclonal antibody. In some instances, an antibody an antibody conjugated to a donor-acceptor fluorophore pair may comprise an antibody from chickens, goats, guinea pigs, hamsters, horses, mice, rats, sheep, monkeys, chimpanzees, humans, camels, sharks, rabbits, alpaca, llama, or any combinations thereof. In some cases, an antibody conjugated to a donor-acceptor fluorophore pair may comprise an intact antibody or an antibody fragment. In some cases, an antibody conjugated to a donor-acceptor fluorophore pair may comprise IgA1, IgA2, IgG1, IgG2, IgG3, IgG4, any derivatives herein and thereof, or any combinations herein and thereof. In some cases, an antibody conjugated to a donor-acceptor fluorophore pair may comprise a bispecific antibody, monoclonal antibody, single-chain variable fragment (scFv), single-chain antigen-binding fragment (scFab), Dual-variable domains Ig (DVD-Ig), scFv-IgG fusion, scFv-Fc (constant region), heavy chain antibody (HcAb), new antigen receptor antibody (IgNAR), domain antibody (dAb), single-dAb (sdAb), diabody, intrabody, trioMab, F(ab)2 bispecific antibody, F(ab)3 trispecific antibody, BiTE antibody, DART antibody, t and antibody, minibody, Bis-scFv, triabody, tetrabody, camel Ig, shark Ig, fragments herein and thereof, derivatives herein and thereof, or any combinations herein and thereof. In some instances, a chemical compound conjugated to a donor-acceptor fluorophore pair may also comprise any dyes or fluorescent dyes described herein.

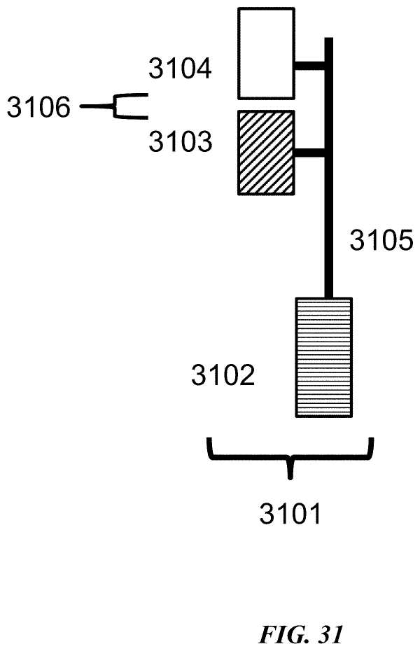

FIG. 31 illustrates an example labeling agent 3101 for tandem labeling. Labeling agent 3101 comprises a substrate 3102 (e.g., an antibody) conjugated to a donor fluorescent dye 3103 and acceptor fluorescent dye 3104 using linker 3105. For example, the substrate 3102 may bind an antigen. Linker 3105 may be a hyp10 or hyp20 linker. 3103 and 3104 are separated by distance 3106. 3106 may be from 1 to 10 nm so that FRET may occur between 3103 and 3104.

In some instances, multiple molecules conjugated to multiple donor-acceptor fluorophore pairs, each pair with a unique Emₘₐₓ or emission spectrum and binding to a unique cellular molecule or a unique set of cellular molecules, may facilitate assaying of the unique cellular molecule or the unique set of cellular molecules using one laser, one Exₘₐₓ, or one excitation spectrum. In other cases, such assaying may comprise simultaneously or sequentially recording the fluorescent intensity of multiple donor-acceptor fluorophore pairs using one laser, one Exₘₐₓ, or one excitation spectrum. For example, multiple antibodies, each conjugated to a donor-acceptor fluorophore pair with a unique Emₘₐₓ or emission spectrum and able to bind a specific antigen, may be incubated with a cell. The incubation may allow bindings of the antibody-donor-acceptor fluorophore pairs with their respective antigens. The cell may be washed thoroughly after the incubation. Such washing step may remove any unbound antibody-donor-acceptor fluorophore pairs. The cell may then be imaged using a laser that can elicit the fluorescent intensity of the antibody-donor-acceptor fluorophore pairs. The presence of the fluorescent intensity may indicate the presence of a specific antigen in the cell. In other cases, other fluorescent intensity methods described herein and thereof may also be used.

In some instances, a donor-acceptor fluorophore pair with a linker described herein (e.g., a hyp10 or hyp20 linker) in a tandem labeling may be used in cytometry. In some cases, tandem labeling may allow a cell to be labeled with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different markers using at least an equivalent number of donor-acceptor fluorophore pairs. In some cases, more than one or all markers may be made visible or measurable using one emission laser. Such visibility or measurability may derive from the emission of the donor-acceptor fluorophore pair. In other cases, the number of emission lasers may be fewer than the number of donor-acceptor fluorophore pairs. In some cases, the number of emission lasers may be fewer than the number of distinctive emission spectrums of the donor-acceptor fluorophore pairs. In some cases, one emission laser may excite the emission of every donor-acceptor fluorophore pair in tandem labeling. In some cases, two emission lasers may excite the emission of every donor-acceptor fluorophore pair in tandem labeling. In some cases, three emission lasers may excite the emission of every donor-acceptor fluorophore pair in tandem labeling. In some cases, four emission lasers may excite the emission of every donor-acceptor fluorophore pair in tandem labeling. In some cases, five emission lasers may excite the emission of every donor-acceptor fluorophore pair in tandem labeling. In some cases, six emission lasers may excite the emission of every donor-acceptor fluorophore pair in tandem labeling.

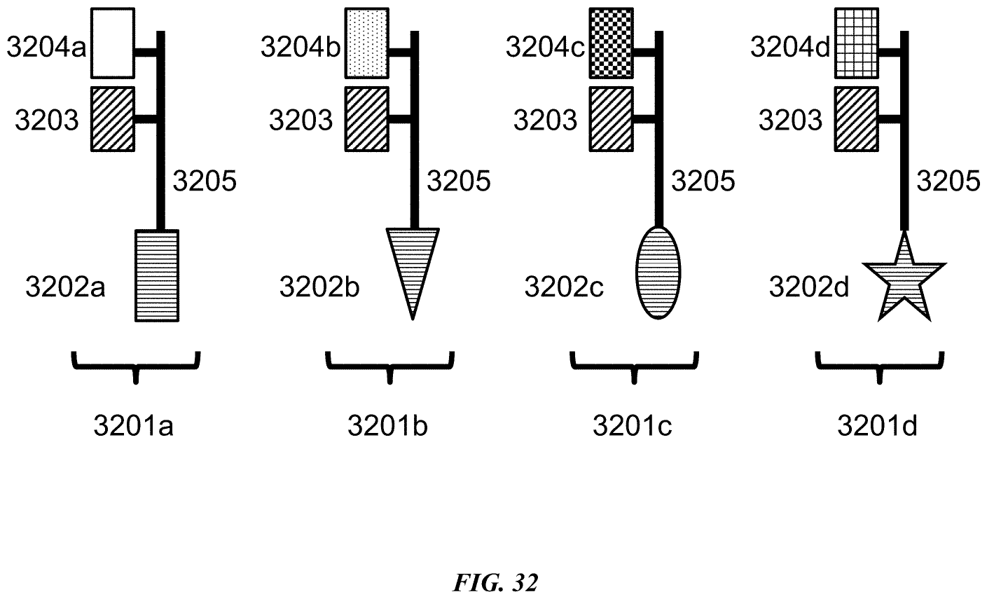

FIG. 32 illustrates multiple labeling agents for labeling multiple molecules using one emission laser. Labeling agents 3201a, 3201b, 3201c, and 3201d are used to label different target molecules. 3201a contains substrate 3202a, 3201b contains substrate 3202b, 3201c contains substrate 3202c, and 3201d contains substrate 3202d. Each of 3202a, 3202b, 3202c, and 3202d binds to a different target molecule. Each of 3202a, 3202b, 3202c, and 3202d is connected to a donor fluorophore 3203 and an acceptor fluorophore 3204a, 3204b, 3204c, and 3204d, respectively, by linker 3205. 3205 may be a hyp10 or hyp20 linker or other linkers described herein. Each of 3204a, 3204b, 3204c, and 3204d has a distinct Emₘₐₓ. 3203 may transfer its excitation energy to 3204a, 3204b, 3204c, or 3204c via FRET. Once 3201a, 3201b, 3201c, or 3201d binds to their respective target molecule by 3202a, 3202b, 3202c, or 3202c, respectively, one laser may be used to excite 3203 so that the excitation energy is transferred to 3204a, 32024, 3204c, and 3204d via FRET. The energy transferred then allows of 3204a, 3204b, 3204c, and 3204d to emit light at their respective Emₘₐₓ. Therefore, one laser is sufficient to label multiple target molecules.

In some instances, a donor-acceptor fluorophore pair with a linker described herein may be more resistant to degradation or more stable than a donor-acceptor fluorophore pair without the linker. In some cases, a donor-acceptor fluorophore pair with a hyp10 or hyp20 linker described herein may be more resistant to degradation or more stable than a donor-acceptor fluorophore pair without the linker. In some cases, a donor-acceptor fluorophore pair with a hyp10 or hyp20 linker may be about 10%, 20%, 30%, 40%, 50%, 60%, 70%, 80%, 90%, 100%, 200%, 300%, 400%, 500%, 600%, 700%, 800% 900%, 1000%, 2000%, 3000%, 4000%, 5000%, 6000%, 7000%, 8000%, 9000%, or 10000% more resistant to degradation or more stable than a donor-acceptor fluorophore pair without the linker. In some cases, a donor-acceptor fluorophore pair with a hyp10 or hyp20 linker may be from 10 to 100%, from 50 to 200%, from 100 to 300%, from 150 to 400%, from 200 to 500%, from 250 to 600%, from 300 to 700%, from 350 to 800%, from 400 to 900%, from 450 to 1000%, from 500 to 2000%, from 1500 to 3000%, from 2500 to 4000%, from 3500 to 5000%, from 4500 to 6000%, from 5500 to 7000%, from 6500 to 8000%, from 7500 to 9000%, or from 8500 to 10000% more resistant to degradation or more stable than a donor-acceptor fluorophore pair without the linker.

In some instances, a donor-acceptor fluorophore pair with a linker described herein may have a higher brightness than a donor-acceptor fluorophore pair without the linker. In some cases, a donor-acceptor fluorophore pair with a hyp10 or hyp20 linker described herein may have a higher brightness than a donor-acceptor fluorophore pair without the linker. In some cases, a donor-acceptor fluorophore pair with a hyp10 or hyp20 linker may have a brightness about 10%, 20%, 30%, 40%, 50%, 60%, 70%, 80%, 90%, 100%, 200%, 300%, 400%, 500%, 600%, 700%, 800% 900%, 1000%, 2000%, 3000%, 4000%, 5000%, 6000%, 7000%, 8000%, 9000%, or 10000% higher than a donor-acceptor fluorophore pair without the linker does. In some cases, a donor-acceptor fluorophore pair with a hyp10 or hyp20 linker may have a brightness from 10 to 100%, from 50 to 200%, from 100 to 300%, from 150 to 400%, from 200 to 500%, from 250 to 600%, from 300 to 700%, from 350 to 800%, from 400 to 900%, from 450 to 1000%, from 500 to 2000%, from 1500 to 3000%, from 2500 to 4000%, from 3500 to 5000%, from 4500 to 6000%, from 5500 to 7000%, from 6500 to 8000%, from 7500 to 9000%, or from 8500 to 10000% higher than a donor-acceptor fluorophore pair without the linker does.

In some instances, the brightness of a fluorophore may be measured by power or radiant flux. In some cases, the brightness of a fluorophore may also be measured by the molar extinction coefficient and quantum yield of the fluorophore. In some cases, the molar extinction coefficient (ε) is defined as the number of photons that can be absorbed by a fluorophore at a given wavelength and is measured in M⁻¹ cm⁻¹. The quantum yield (Φ) is calculated as the number of photons that are emitted by the fluorophore divided by the number of photons that are absorbed to arrive to the efficiency of a fluorophore. The brightness of a fluorophore is the product of ε and Φ.

In some instances, the molar extinction coefficient (ε) of a donor-acceptor fluorophore pair with a linker described herein (e.g., a hyp10 or hyp20 linker) may be about 1×10\hat{ }4 M⁻¹ cm⁻¹, 2×10\hat{ }4 M⁻¹ cm⁻¹, 3×10\hat{ }4 M⁻¹ cm⁻¹, 4×10\hat{ }4 M⁻¹ cm⁻¹, 5×10\hat{ }4 M⁻¹ cm⁻¹, 6×10\hat{ }4 M⁻¹ cm⁻¹, 7×10\hat{ }4 M⁻¹ cm⁻¹, 8×10\hat{ }4 M⁻¹ cm⁻¹, 9×10\hat{ }4 M⁻¹ cm⁻¹, 1×10\hat{ }5 M⁻¹ cm⁻¹, 2×10\hat{ }5 M⁻¹ cm⁻¹, 3×10\hat{ }5 M⁻¹ cm⁻¹, 4×10\hat{ }5 M⁻¹ cm⁻¹, 5×10\hat{ }5 M⁻¹ cm⁻¹, 6×10\hat{ }5 M⁻¹ cm⁻¹, 7×10\hat{ }5 M⁻¹ cm⁻¹, 8×10\hat{ }5 M⁻¹ cm⁻¹, 9×10\hat{ }5 M⁻¹ cm⁻¹, 1×10\hat{ }6 M⁻¹ cm⁻¹, 2×10\hat{ }6 M⁻¹ cm⁻¹, 3×10\hat{ }6 M⁻¹ cm⁻¹, 4×10\hat{ }6 M⁻¹ cm⁻¹, 5×10\hat{ }6 M⁻¹ cm⁻¹, 6×10\hat{ }6 M⁻¹ cm⁻¹, 7×10\hat{ }6 M⁻¹ cm⁻¹, 8×10\hat{ }6 M⁻¹ cm⁻¹, or 9×10\hat{ }6 M⁻¹ cm⁻¹. In some cases, the molar extinction coefficient (c) of a donor-acceptor fluorophore pair with a linker described herein (e.g., a hyp10 or hyp20 linker) may be from 1×10\hat{ }4 to 2×10\hat{ }4 M⁻¹ cm⁻¹, from 1.5×10\hat{ }4 to 2.5×10\hat{ }4 M⁻¹ cm⁻¹, from 2×10\hat{ }4 to 3×10\hat{ }4 M⁻¹ cm⁻¹, from 2.5×10\hat{ }4 to 3.5×10\hat{ }4 M⁻¹ cm⁻¹, from 3×10\hat{ }4 to 4×10\hat{ }4 M⁻¹ cm⁻¹, from 3.5×10\hat{ }4 to 4.5×10\hat{ }4 M⁻¹ cm⁻¹, from 4×10\hat{ }4 to 5×10\hat{ }4 M⁻¹ cm⁻¹, from 4.5×10\hat{ }4 to 5.5×10\hat{ }4 M⁻¹ cm⁻¹, from 5×10\hat{ }4 to 6×10\hat{ }4 M⁻¹ cm⁻¹, from 5.5×10\hat{ }4 to 6.5×10\hat{ }4 M⁻¹ cm⁻¹, from 6×10\hat{ }4 to 7×10\hat{ }4 M⁻¹ cm⁻¹, from 6.5×10\hat{ }4 to 7.5×10\hat{ }4 M⁻¹ cm⁻¹, from 7×10\hat{ }4 to 8×10\hat{ }4 M⁻¹ cm⁻¹, from 7.5×10\hat{ }4 to 8.5×10\hat{ }4 M⁻¹ cm⁻¹, from 8×10\hat{ }4 to 9×10\hat{ }4 M⁻¹ cm⁻¹, from 8.5×10\hat{ }4 to 9.5×10\hat{ }4 M⁻¹ cm⁻¹, from 9×10\hat{ }4 to 1×10\hat{ }5 M⁻¹ cm⁻¹, from 9.5×10\hat{ }4 to 1.5×10\hat{ }5 M⁻¹ cm⁻¹, from 1×10\hat{ }5 to 2×10\hat{ }5 M⁻¹ cm⁻¹, from 1.5×10\hat{ }5 to 2.5×10\hat{ }5 M⁻¹ cm⁻¹, from 2×10\hat{ }5 to 3×10\hat{ }5 M⁻¹ cm⁻¹, from 2.5×10\hat{ }5 to 3.5×10\hat{ }5 M⁻¹ cm⁻¹, from 3×10\hat{ }5 to 4×10\hat{ }5 M⁻¹ cm⁻¹, from 3.5×10\hat{ }5 to 4.5×10\hat{ }5 M⁻¹ cm⁻¹, from 4×10\hat{ }5 to 5×10\hat{ }5 M⁻¹ cm⁻¹, from 4.5×10\hat{ }5 to 5.5×10\hat{ }5 M⁻¹ cm⁻¹, from 5×10\hat{ }5 to 6×10\hat{ }5 M⁻¹ cm⁻¹, from 5.5×10\hat{ }5 to 6.5×10\hat{ }5 M⁻¹ cm⁻¹, from 6×10\hat{ }5 to 7×10\hat{ }5 M⁻¹ cm⁻¹, from 6.5×10\hat{ }5 to 7.5×10\hat{ }5 M⁻¹ cm⁻¹, from 7×10\hat{ }5 to 8×10\hat{ }5 M⁻¹ cm⁻¹, from 7.5×10\hat{ }5 to 8.5×10\hat{ }5 M⁻¹ cm⁻¹, from 8×10\hat{ }5 to 9×10\hat{ }5 M⁻¹ cm⁻¹, from 8.5×10\hat{ }5 to 9.5×10\hat{ }5 M⁻¹ cm⁻¹, from 9×10\hat{ }5 to 1×10\hat{ }6 M⁻¹ cm⁻¹, from 9.5×10\hat{ }5 to 1.5×10\hat{ }6 M⁻¹ cm⁻¹, from 1×10\hat{ }6 to 2×10\hat{ }6 M⁻¹ cm⁻¹, from 1.5×10\hat{ }6 to 2.5×10\hat{ }6 M⁻¹ cm⁻¹, from 2×10\hat{ }6 to 3×10\hat{ }6 M⁻¹ cm⁻¹, from 2.5×10\hat{ }6 to 3.5×10\hat{ }6 M⁻¹ cm⁻¹, from 3×10\hat{ }6 to 4×10\hat{ }6 M⁻¹ cm⁻¹, from 3.5×10\hat{ }6 to 4.5×10\hat{ }6 M⁻¹ cm⁻¹, from 4×10\hat{ }6 to 5×10\hat{ }6 M⁻¹ cm⁻¹, from 4.5×10\hat{ }6 to 5.5×10\hat{ }6 M⁻¹ cm⁻¹, from 5×10\hat{ }6 to 6×10\hat{ }6 M⁻¹ cm⁻¹, from 5.5×10\hat{ }6 to 6.5×10\hat{ }6 M⁻¹ cm⁻¹, from 6×10\hat{ }6 to 7×10\hat{ }6 M⁻¹ cm⁻¹, from 6.5×10\hat{ }6 to 7.5×10\hat{ }6 M⁻¹ cm⁻¹, from 7×10\hat{ }6 to 8×10\hat{ }6 M⁻¹ cm⁻¹, from 7.5×10\hat{ }6 to 8.5×10\hat{ }6 M⁻¹ cm⁻¹, or from 8×10\hat{ }6 to 9×10\hat{ }6 M⁻¹ cm⁻¹.

In some instances, the quantum yield (D) of a donor-acceptor fluorophore pair with a linker described herein (e.g., a hyp10 or hyp20 linker) may be at least about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, or more. In some cases, the quantum yield (D) of a donor-acceptor fluorophore pair with a linker described herein (e.g., a hyp10 or hyp20 linker) may be from 0.01 to 0.02, from 0.015 to 0.025, from 0.02 to 0.03, from 0.025 to 0.035, from 0.03 to 0.04, from 0.035 to 0.045, from 0.04 to 0.05, from 0.045 to 0.055, from 0.05 to 0.06, from 0.055 to 0.065, from 0.06 to 0.07, from 0.065 to 0.075, from 0.07 to 0.08, from 0.075 to 0.085, from 0.08 to 0.09, from 0.085 to 0.095, from 0.09 to 0.1, from 0.095 to 0.105, from 0.1 to 0.11, from 0.105 to 0.115, from 0.11 to 0.12, from 0.115 to 0.125, from 0.12 to 0.13, from 0.125 to 0.135, from 0.13 to 0.14, from 0.135 to 0.145, from 0.14 to 0.15, from 0.145 to 0.155, from 0.15 to 0.16, from 0.155 to 0.165, from 0.16 to 0.17, from 0.165 to 0.175, from 0.17 to 0.18, from 0.175 to 0.185, from 0.18 to 0.19, from 0.185 to 0.195, from 0.19 to 0.2, from 0.195 to 0.205, from 0.2 to 0.21, from 0.205 to 0.215, from 0.21 to 0.22, from 0.215 to 0.225, from 0.22 to 0.23, from 0.225 to 0.235, from 0.23 to 0.24, from 0.235 to 0.245, from 0.24 to 0.25, from 0.245 to 0.255, from 0.25 to 0.26, from 0.255 to 0.265, from 0.26 to 0.27, from 0.265 to 0.275, from 0.27 to 0.28, from 0.275 to 0.285, from 0.28 to 0.29, from 0.285 to 0.295, from 0.29 to 0.3, from 0.295 to 0.305, from 0.3 to 0.31, from 0.305 to 0.315, from 0.31 to 0.32, from 0.315 to 0.325, from 0.32 to 0.33, from 0.325 to 0.335, from 0.33 to 0.34, from 0.335 to 0.345, from 0.34 to 0.35, from 0.345 to 0.355, from 0.35 to 0.36, from 0.355 to 0.365, from 0.36 to 0.37, from 0.365 to 0.375, from 0.37 to 0.38, from 0.375 to 0.385, from 0.38 to 0.39, from 0.385 to 0.395, from 0.39 to 0.4, from 0.395 to 0.405, from 0.4 to 0.41, from 0.405 to 0.415, from 0.41 to 0.42, from 0.415 to 0.425, from 0.42 to 0.43, from 0.425 to 0.435, from 0.43 to 0.44, from 0.435 to 0.445, from 0.44 to 0.45, from 0.445 to 0.455, from 0.45 to 0.46, from 0.455 to 0.465, from 0.46 to 0.47, from 0.465 to 0.475, from 0.47 to 0.48, from 0.475 to 0.485, from 0.48 to 0.49, from 0.485 to 0.495, from 0.49 to 0.5, from 0.495 to 0.505, from 0.5 to 0.51, from 0.505 to 0.515, from 0.51 to 0.52, from 0.515 to 0.525, from 0.52 to 0.53, from 0.525 to 0.535, from 0.53 to 0.54, from 0.535 to 0.545, from 0.54 to 0.55, from 0.545 to 0.555, from 0.55 to 0.56, from 0.555 to 0.565, from 0.56 to 0.57, from 0.565 to 0.575, from 0.57 to 0.58, from 0.575 to 0.585, from 0.58 to 0.59, from 0.585 to 0.595, from 0.59 to 0.6, from 0.595 to 0.605, from 0.6 to 0.61, from 0.605 to 0.615, from 0.61 to 0.62, from 0.615 to 0.625, from 0.62 to 0.63, from 0.625 to 0.635, from 0.63 to 0.64, from 0.635 to 0.645, from 0.64 to 0.65, from 0.645 to 0.655, from 0.65 to 0.66, from 0.655 to 0.665, from 0.66 to 0.67, from 0.665 to 0.675, from 0.67 to 0.68, from 0.675 to 0.685, from 0.68 to 0.69, from 0.685 to 0.695, from 0.69 to 0.7, from 0.695 to 0.705, from 0.7 to 0.71, from 0.705 to 0.715, from 0.71 to 0.72, from 0.715 to 0.725, from 0.72 to 0.73, from 0.725 to 0.735, from 0.73 to 0.74, from 0.735 to 0.745, from 0.74 to 0.75, from 0.745 to 0.755, from 0.75 to 0.76, from 0.755 to 0.765, from 0.76 to 0.77, from 0.765 to 0.775, from 0.77 to 0.78, from 0.775 to 0.785, from 0.78 to 0.79, from 0.785 to 0.795, from 0.79 to 0.8, from 0.795 to 0.805, from 0.8 to 0.81, from 0.805 to 0.815, from 0.81 to 0.82, from 0.815 to 0.825, from 0.82 to 0.83, from 0.825 to 0.835, from 0.83 to 0.84, from 0.835 to 0.845, from 0.84 to 0.85, from 0.845 to 0.855, from 0.85 to 0.86, from 0.855 to 0.865, from 0.86 to 0.87, from 0.865 to 0.875, from 0.87 to 0.88, from 0.875 to 0.885, from 0.88 to 0.89, from 0.885 to 0.895, from 0.89 to 0.9, from 0.895 to 0.905, from 0.9 to 0.91, from 0.905 to 0.915, from 0.91 to 0.92, from 0.915 to 0.925, from 0.92 to 0.93, from 0.925 to 0.935, from 0.93 to 0.94, from 0.935 to 0.945, from 0.94 to 0.95, from 0.945 to 0.955, from 0.95 to 0.96, from 0.955 to 0.965, from 0.96 to 0.97, from 0.965 to 0.975, from 0.97 to 0.98, from 0.975 to 0.985, from 0.98 to 0.99, from 0.985 to 0.995, from 0.99 to 1.

In some instances, a donor-acceptor fluorophore pair with a linker described herein may be more resistant to photodegradation or photobleaching than a donor-acceptor fluorophore pair without the linker. In some cases, a donor-acceptor fluorophore pair with a hyp10 or hyp20 linker described herein may be more resistant to more resistant to photodegradation or photobleaching than a donor-acceptor fluorophore pair without the linker. In some cases, a donor-acceptor fluorophore pair with a hyp10 or hyp20 linker may be about 10%, 20%, 30%, 40%, 50%, 60%, 70%, 80%, 90%, 100%, 200%, 300%, 400%, 500%, 600%, 700%, 800% 900%, 1000%, 2000%, 3000%, 4000%, 5000%, 6000%, 7000%, 8000%, 9000%, or 10000% more resistant to photodegradation or photobleaching than a donor-acceptor fluorophore pair without the linker. In some cases, a donor-acceptor fluorophore pair with a hyp10 or hyp20 linker may be from 10 to 100%, from 50 to 200%, from 100 to 300%, from 150 to 400%, from 200 to 500%, from 250 to 600%, from 300 to 700%, from 350 to 800%, from 400 to 900%, from 450 to 1000%, from 500 to 2000%, from 1500 to 3000%, from 2500 to 4000%, from 3500 to 5000%, from 4500 to 6000%, from 5500 to 7000%, from 6500 to 8000%, from 7500 to 9000%, or from 8500 to 10000% more resistant to photodegradation or photobleaching than a donor-acceptor fluorophore pair without the linker.

In some cases, the increase in stability or brightness or decrease in degradation, photodegradation, or photobleaching of a donor-acceptor fluorophore pair with a linker described herein (e.g., a hyp10 or hyp20 linker) over a donor-acceptor fluorophore pair without a linker may be maintained even if the fluorophores are under fixation or permeabilization. In some cases, the increase in stability or brightness or decrease in degradation, photodegradation, or photobleaching of a donor-acceptor fluorophore pair with a linker described herein (e.g., a hyp10 or hyp20 linker) over a donor-acceptor fluorophore pair without a linker may be maintained even if the fluorophores are maintained at about −80° C., −79° C., −78° C., −77° C., −76° C., −75° C., −74° C., −73° C., −72° C., −71° C., −70° C., −69° C., −68° C., −67° C., −66° C., −65° C., −64° C., −63° C., −62° C., −61° C., −60° C., −59° C., −58° C., −57° C., −56° C., −55° C., −54° C., −53° C., −52° C., −51° C., −50° C., −49° C., −48° C., −47° C., −46° C., −45° C., −44° C., −43° C., −42° C., −41° C., −40° C., −39° C., −38° C., −37° C., −36° C., −35° C., −34° C., −33° C., −32° C., −31° C., −30° C., −29° C., −28° C., −27° C., −26° C., −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C. In some cases, the increase in stability or brightness or decrease in degradation, photodegradation, or photobleaching of a donor-acceptor fluorophore pair with a linker described herein (e.g., a hyp10 or hyp20 linker) over a donor-acceptor fluorophore pair without a linker may be maintained even if the fluorophores are maintained at from −80 to −70° C., from −75 to −65° C., from −70 to −60° C., from −65 to −55° C., from −60 to −50° C., from −55 to −45° C., from −50 to −40° C., from −45 to −35° C., from −40 to −30° C., from −35 to -25° C., from −30 to −20° C., from −25 to −15° C., from −20 to −10° C., from −15 to −5° C., from −10 to 0° C., from −5 to 5° C., from 0 to 10° C., from 5 to 15° C., from 10 to 20° C., from 15 to 25° C., from 20 to 30° C., from 25 to 35° C., from 30 to 40° C., from 35 to 45° C., or from 40 to 50° C. In some cases, the increase in stability or brightness or decrease in degradation, photodegradation, or photobleaching of a donor-acceptor fluorophore pair with a linker described herein (e.g., a hyp10 or hyp20 linker) over a donor-acceptor fluorophore pair without a linker may be maintained even if the fluorophores are maintained at pH 0, 1, 2, 3, 4, 5, 6, 7, 8, 9, 10, 11, 12, 13, 14. In some cases, the increase in stability or brightness or decrease in degradation, photodegradation, or photobleaching of a donor-acceptor fluorophore pair with a linker described herein (e.g., a hyp10 or hyp20 linker) over a donor-acceptor fluorophore pair without a linker may be maintained even if the fluorophores are maintained at pH from 1-5, 2-6, 3-7, 4-8, 5-9, 6-10, 7-11, 8-12, 9-13, or 10-14.

In some instances, the energy transferred from a donor fluorophore to acceptor fluorescence via FRET may be measured by an increase in fluorescence intensity of the acceptor fluorescence. The increase or decrease in fluorescent intensity may be measured by computing the difference between the baseline fluorescent intensity level (e.g., the fluorescent intensity level before the energy transfer) and the fluorescent intensity level after the energy transfer. In some cases, the energy transferred from a donor fluorophore to an acceptor fluorescence via FRET may be measured by a decrease in fluorescence intensity of the donor fluorescence (i.e., quenching of the donor fluorophore). In other cases, the energy transferred from a donor fluorophore to an acceptor fluorescence via FRET may also be measured by an increase in fluorescence intensity of the acceptor fluorescence and a decrease in fluorescence intensity of the donor fluorescence. In some cases, when measuring a decrease in fluorescence intensity level in a donor fluorophore, a nonfluorescent acceptor molecule may replace an acceptor fluorophore. Using a nonfluorescent acceptor molecule may facilitate the measurement of the donor fluorophore. Such a facilitation may comprise a lack of fluorescence interference by the acceptor molecule.

In some instances, a donor fluorophore may be paired with or conjugated to more than one acceptor fluorophore using a linker described herein (e.g., a hyp10 or hyp20 linker). In some cases, a donor fluorophore may be paired with or conjugated to 2, 3, 4, 5 or more acceptor fluorophores. For example, a donor fluorophore may be paired with or conjugated to a second acceptor fluorophore and a third fluorophore. The conjugation using a linker described herein (e.g., a hyp10 or hyp20 linker) may allow FRET to transfer energy from the donor fluorophore to each of the conjugated acceptor fluorophores. In some cases, the energy transferred from a donor fluorophore to each of the conjugated acceptor fluorophores may be sufficient to cause emission of each of the conjugated acceptor fluorophore. In some instances, each of the acceptor fluorophores may distinguishable by fluorescent emission. For example, the emission spectrum or Exₘₐₓ. In some cases, each of the acceptor fluorophore may have not have an overlap of the emission spectrum. In some cases, each of the acceptor fluorophore may have an overlap of the emission spectrum. In other cases, the overlap may not be more than 1%, 2%, 3%, 4%, 5%, 6%, 7%, 8%, 9%, 10%, 15%, 20%, 25%, 30%, 35%, 40%, 45%, 50%, 55%, 60%, 65%, or 70%. The overlap may also be about 1 to 10%, 5 to 15%, 10 to 20%, 15 to 25%, 20 to 30%, 25 to 35%, 30 to 40%, 35 to 45%, 40 to 50%, 45 to 55%, 50 to 60%, 55 to 65%, or 60 to 70%.

In some instances, a tandem labeling agent may comprise more than one donor-acceptor fluorophore pair. In some cases, a tandem labeling agent may comprise 2, 3, 4, 5, 6, 7, 8, 9, or 10 donor-acceptor fluorophore pairs. Each of the donor-acceptor pairs may be linked or conjugated together with a linker described herein (e.g., a hyp10 or hyp20 linker). In some cases, the emission spectrum or Emₘₐₓ of each donor-acceptor fluorophore pair may be different from those of other donor-acceptor fluorophore pairs in a tandem labeling agent comprising more than one donor-acceptor fluorophore pair. In some cases, the emission spectrum or Emₘₐₓ of one donor-acceptor fluorophore pair may be different from that of another donor-acceptor fluorophore pair in a tandem labeling agent comprising more than one donor-acceptor fluorophore pair. In some instances, a first tandem labeling agent may be distinguishable from a second tandem labeling agent. For example, a first tandem labeling agent and a second tandem labeling agent may have different emission spectrums or Emₘₐₓ. In other case, a first tandem labeling agent may be distinguishable from a second tandem labeling agent even though they may have the same Emₘₐₓ. In some cases, the fluorescent intensity of a tandem labeling agent might be quantitative in respect to the number of donor-acceptor fluorophore pair. For example, a first tandem labeling agent may have one first donor-acceptor fluorophore pair, and a second tandem labeling agent may have two first donor-acceptor fluorophore pairs. The first and second tandem labeling agent may have the same Exₘₐₓ, but the fluorescent intensity of the second tandem labeling agent may be two time higher than that of the first tandem labeling agent. In some instances, a tandem labeling agent may comprise any combinations, numbers, or configuration of donor and acceptor fluorophores as described herein and thereof.

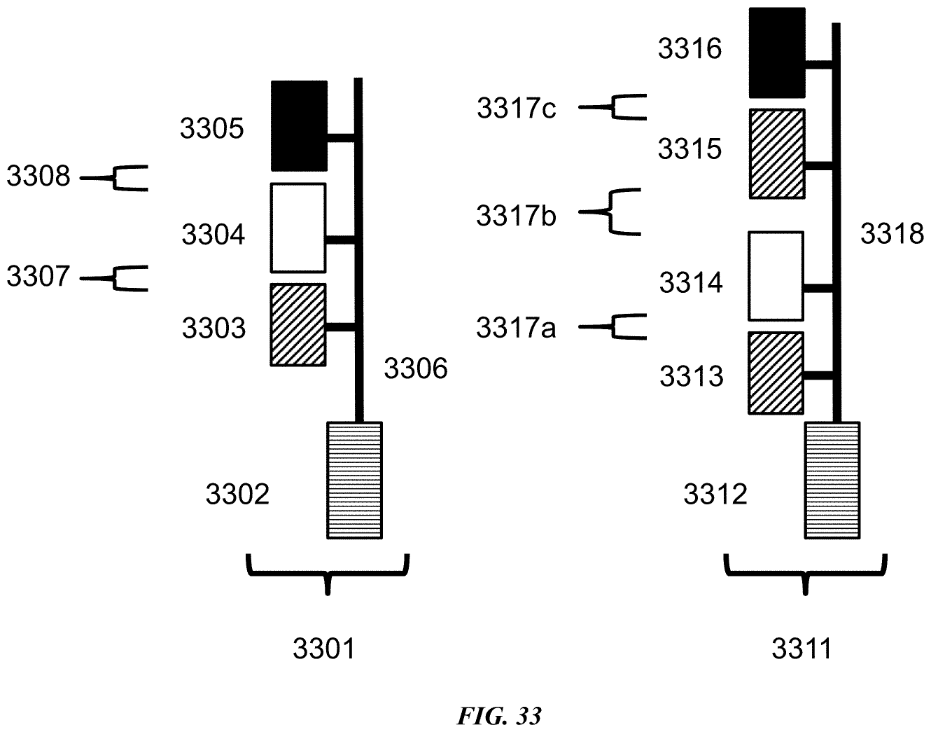

FIG. 33 illustrates two example labeling agents. Label agent 3301 contains substrate 3302 (e.g., an antibody). 3302 is conjugated to donor fluorophore 3304 and acceptor fluorophores 3303 and 3305 with linker 3306. 3303 and 3304 are separated by distance 3307. 3305, and 3304 are separated by distance 3308. 3307 allows FRET to occur between 3303 and 3304. 3308 allows FRET to occur between 3304 and 3305. Labeling agent 3311 contains substrate 3312 (e.g., an antibody). 3312 is conjugated to donor fluorophore 3313/3315 and acceptor fluorophores 3314/3316 with linker 3318. 3313 and 3314 are separated by distance 3317a. 3315 and 3316 are separated by distance 3317c. 3314 and 3315 are separated by distance 3317b. 3317a allows FRET to occur between 3313 and 3314. 3317c allows FRET to occur between 3315 and 3316. 3317b does not allow FRET to occur between 3315 and 3314. 3313 and 3315 may have the same Exₘₐₓ and Emₘₐₓ.

### Computer Systems

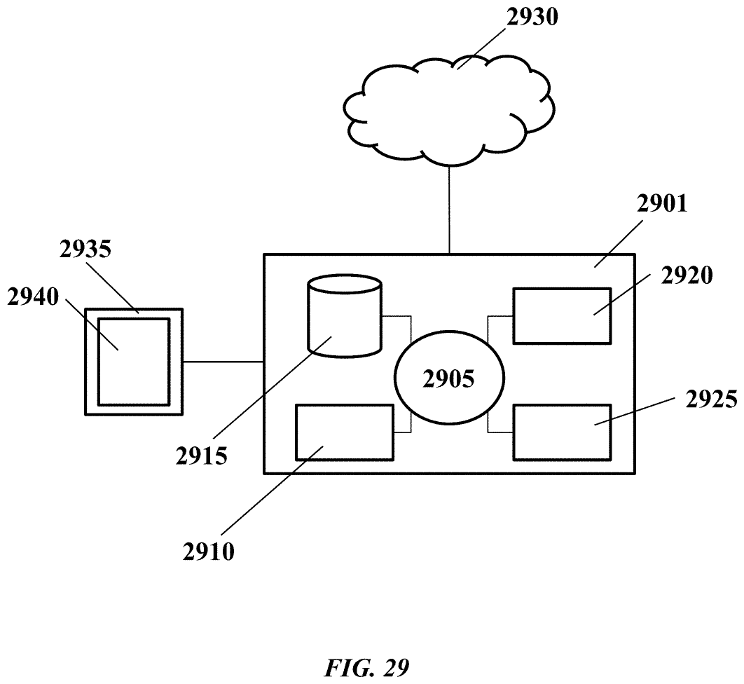

The present disclosure provides computer systems that are programmed to implement methods of the disclosure. FIG. 29 shows a computer system 2901 that is programmed or otherwise configured to perform nucleic acid sequencing. The computer system 2901 can determine sequence reads based at least in part on intensities of detected optical signals. The computer system 2901 can regulate various aspects of the present disclosure, such as, for example, performing nucleic acid sequencing, sequence analysis, and regulating conditions of transient binding and non-transient binding (e.g., incorporation) of nucleotides. The computer system 2901 can be an electronic device of a user or a computer system that is remotely located with respect to the electronic device. The electronic device can be a mobile electronic device.

The computer system 2901 includes a central processing unit (CPU, also “processor” and “computer processor” herein) 2905, which can be a single core or multi core processor, or a plurality of processors for parallel processing. The computer system 2901 also includes memory or memory location 2910 (e.g., random-access memory, read-only memory, flash memory), electronic storage unit 2915 (e.g., hard disk), communication interface 2920 (e.g., network adapter) for communicating with one or more other systems, and peripheral devices 2925, such as cache, other memory, data storage and/or electronic display adapters. The memory 2910, storage unit 2915, interface 2920 and peripheral devices 2925 are in communication with the CPU 29 29605 through a communication bus (solid lines), such as a motherboard. The storage unit 2915 can be a data storage unit (or data repository) for storing data. The computer system 2901 can be operatively coupled to a computer network (“network”) 2930 with the aid of the communication interface 2920. The network 2930 can be the Internet, an internet and/or extranet, or an intranet and/or extranet that is in communication with the Internet. The network 2930 in some cases is a telecommunication and/or data network. The network 2930 can include one or more computer servers, which can enable distributed computing, such as cloud computing. The network 2930, in some cases with the aid of the computer system 2901, can implement a peer-to-peer network, which may enable devices coupled to the computer system 2901 to behave as a client or a server.

The CPU 2905 can execute a sequence of machine-readable instructions, which can be embodied in a program or software. The instructions may be stored in a memory location, such as the memory 2910. The instructions can be directed to the CPU 2905, which can subsequently program or otherwise configure the CPU 2905 to implement methods of the present disclosure. Examples of operations performed by the CPU 2905 can include fetch, decode, execute, and writeback.

The CPU 2905 can be part of a circuit, such as an integrated circuit. One or more other components of the system 2901 can be included in the circuit. In some cases, the circuit is an application specific integrated circuit (ASIC).

The storage unit 2915 can store files, such as drivers, libraries and saved programs. The storage unit 2915 can store user data, e.g., user preferences and user programs. The computer system 2901 in some cases can include one or more additional data storage units that are external to the computer system 2901, such as located on a remote server that is in communication with the computer system 2901 through an intranet or the Internet.

The computer system 2901 can communicate with one or more remote computer systems through the network 2930. For instance, the computer system 2901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 29 29601 via the network 2930.

Methods as described herein can be implemented by way of machine (e.g., computer processor) executable code stored on an electronic storage location of the computer system 2901, such as, for example, on the memory 2910 or electronic storage unit 2915. The machine executable or machine readable code can be provided in the form of software. During use, the code can be executed by the processor 2905. In some cases, the code can be retrieved from the storage unit 2915 and stored on the memory 2910 for ready access by the processor 2905. In some situations, the electronic storage unit 2915 can be precluded, and machine-executable instructions are stored on memory 2910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 2901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2901 can include or be in communication with an electronic display 2935 that comprises a user interface (UI) 2940 for providing, for example, results of nucleic acid sequence and optical signal detection (e.g., sequence reads, intensity maps, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2905. The algorithm can, for example, implement methods and systems of the present disclosure, such as determine sequence reads based at least in part on intensities of detected optical signals.

### EXAMPLES

### Example 1: General Synthetic Principles

Certain examples of the following examples illustrate various methods of making linkers and labeled substrates described herein. It is understood that one skilled in the art may be able to make these compounds by similar methods or by combining other methods known to one skilled in the art. It is also understood that one skilled in the art would be able to make other compounds in a similar manner as described below by using the appropriate starting materials and modifying synthetic routes as needed. In general, starting materials and reagents can be obtained from commercial vendors or synthesized according to sources known to those skilled in the art or prepared as described herein.

Unless otherwise noted, reagents and solvents used in synthetic methods described herein are obtained from commercial suppliers. Anhydrous solvents and oven-dried glassware may be used for synthetic transformations sensitive to moisture and/or oxygen. Yields may not be optimized. Reaction times may be approximate and may not be optimized. Materials and instrumentation used in synthetic procedures may be substituted with appropriate alternatives. Column chromatography and thin layer chromatography (TLC) may be performed on reverse-phase silica gel unless otherwise noted. Nuclear magnetic resonance (NMR) and mass spectra may be obtained to characterize reaction products and/or monitor reaction progress.

### Example 2: Synthesis of dGTP-AP-SS-hyp10-Atto633

Described herein is a method for constructing the labeled nucleotide dGTP-AP-SS-hyp10-Atto633. FIG. 2A illustrates an example method for the synthesis of a fluorescently labeled dGTP reagent. FIG. 2B illustrates the same synthesis with the full structures of the dye and linker. The method involves formation of a covalent linkage between Gly-Hyp10 and the fluorophore Atto633 (process (a)), esterification to couple Atto633-Gly-Hyp10 with pentafluorophenol (process (b)), substitution with the linker molecule epSS (process(c)), esterification to form Atto633-Gly-Hyp10-epSS-PFP (process (d)), and substitution with dGTP to provide the fluorescently labeled nucleotide (process (e)). Details of the synthesis are provided below.

Preparation of Atto633-Gly-Hyp10. (FIG. 2A process (a)) A stock solution of Gly-Hyp10 (also referred to herein as “hyp10”) in bicarbonate is prepared by dissolving 25 milligrams (mg) of the 11 amino acid peptide in 500 microliters (4) of 0.2 molar (M) sodium bicarbonate in a 1.5 milliliter (mL) Eppendorf tube. 7 mg of Atto633-NHS is weighed into another Eppendorf tube and dissolved in 200 μL of dimethylformamide (DMF). A volume of 300 μL of the peptide solution is added to the solution containing Atto633-NHS. The resulting solution is mixed and heated to 50° C. for 20 minutes (min). The extent of the reaction is followed with reverse-phase thin layer chromatography (TLC). A 1 μL aliquot of the reaction solution is removed and dissolved in 40 μL water and spotted on reverse phase TLC. A co-spot with Atto633 acid is included, and Atto633 is also run alone. The plate is eluted with a 2:1 solution of acetonitrile 0.1 M triethylammonium acetate (TEAA). Atto633 acid and Atto633-NHS both have an R_{f} of zero, while Gly-Hyp10 has an R_{f} of 0.4. The product is purified by injecting the solution onto a C18 reverse phase column using the gradient 20%→50% acetonitrile vs. 0.1M TEAA over 16 minutes at 2.5 mL/min. The desired product is the major product, Atto633-Gly-Hyp10, eluting at 15.2 minutes. The fractions containing the desired material are collected in Eppendorf tubes and dried, yielding a blue solid. A major peak was observed on ESI mass spec: m/z calculated for C₈₇H₁₁₅N₁₄O₂₄⁺, [M]⁺=1739.8; found: 1740.6.

Preparation of Atto633-Gly-Hyp10-PFP. (FIG. 2A process (b)) Atto633-Gly-Hyp10 is suspended in 100 μl DMF in a 1.5 mL Eppendorf tube. Pyridine (20 μL) and pentafluorophenyl trifluoroacetate (PFP-TFA, 20 μL) are added to the tube. The reaction mixture is warmed to 50° C. in a heat block for 20 min. The reaction is monitored by removing 1 μL aliquots and adding to 1 mL of dilute HCl (0.4%). When the reaction is complete the aqueous solution is colorless. After 10 min the dilute HCl solution is light blue. Additional PFP-TFA (30 μL) is added. After another 100 min at 50° C. a retest of precipitation gives a colorless solution. The remaining reaction mixture is precipitated into 1 mL dilute HCl in 20 μl portions. 20 μl is added to 1 mL dilute HCl, the tube spun down, and aqueous solution discarded. The process is repeated until all of the product is precipitated. The residue is thoroughly dried. After drying, the solid is washed twice with 1 mL methyl tert-butyl ether (MTBE). The product is a dark blue powder. The product gives a major peak on electrospray ionization (ESI)-mass spectrometry (MS): m/z calculated for C₉₃H₁₁₅F₅N₁₄O₂₄²⁺, [M+H]²⁺=1906.8/2=953.4; found: 953.4.

Preparation of Atto633-Gly-Hyp10-epSS. (FIG. 2A process (c)) Atto633-Gly-Hyp10-PFP (1.6 micromoles (μmol)) is dissolved in 100 μl DMF in an Eppendorf tube. A solution of aminoethyl-SS-propionic acid (Broadpharm; 6 mg in 200 μl 0.1 M bicarbonate) is mixed with the Atto633-gly-hyp10-PFP and heated to 50° C. in a heat block for 20 min. Atto633-Gly-Hyp10-epSS is purified from the resulting reaction mixture by reverse phase HPLC using a gradient of 20%→50% acetonitrile over 16 min. Atto633-Gly-Hyp10 elutes at 15 min and Atto633-Gly-Hyp10-epSS elutes at 15.6 min. The fractions containing the product, Atto633-Gly-Hyp10-epSS, are combined and dried. The product has a major peak on ESI-MS: m/z calculated for C₉₂H₁₂₄N₁₅O₂₅S₂⁺, [M]⁺=1902.8; Found: 1902.6.

Preparation of Atto633-Gly-Hyp10-epSS-PFP. (FIG. 2A process (d)) Atto633-Gly-Hyp10-epSS is dissolved in 100 μl DMF in an Eppendorf tube. Pyridine (20 μl) and PFP-TFA (20 μL) are added and the mixture is heated to 50° C. in a heat block for 20 min. A test aliquot (1 μL) in dilute HCl gives a colorless solution and a blue precipitate. The reaction is precipitated in 20 μl aliquots in 1 mL dilute HCl, the tube spun down, and the aqueous solution discarded. The process is repeated until all the PFP ester is precipitated. The residue is thoroughly dried under vacuum and washed with MTBE.

Preparation of dGTP-AP-SS-Atto633. (FIG. 2A process (e)) A solution of aminopropargyl dGTP (Trilink; 1 μmol in 100 μl of 0.2 M bicarbonate) is added to 50 μL of a DMF solution comprising Atto633-gly-hyp10-epSS-PFP. The mixture is heated to 50° C. for 10 min. The product, dGTP-AP-epSS-Atto633, is purified by reverse-phase HPLC using a gradient of 20%→50% acetonitrile 16 min. The product elutes at 15.3 min. Preparative HPLC provides 0.65 μmol. The product gives a major peak on ESI-MS: m/z calculated for C₁₀₆H₁₃₉N₂₀O₃₇P₃S₂²⁻, [M−H]²⁻, 1220.4; found: 1220.6.

While synthesis of dGTP-Atto633-Gly-Hyp10-epSS-PFP is described, a skilled practitioner will recognize that other fluorescently labeled nucleotides can be produced in a similar manner using appropriate starting materials.

### Example 3: Preparation of Dye-Labeled Nucleotides

A set of dye-labeled nucleotides designed for excitation at about 530 nm is prepared. Excitation at 530 nm may be achieved using a green laser, which may be readily available, high-powered, and stable. There are many commercially available fluorescent dyes with excitation at or near 530 nm that are inexpensive and have a variety of properties (hydrophobic, hydrophilic, positively charged, negatively charged). Synthetic routes to such dyes may be shorter and cheaper than those for longer wavelength dyes. Moreover, certain green dyes may have significantly less self-quenching than red dyes, potentially allowing for the use of higher labeling fractions (e.g., as described herein).

A viable reagent set for use in, e.g., a sequencing application consists of each of four canonical nucleotides or analogs thereof with cleavable green dyes that perform well in sequencing. An optimal set may be prepared by varying each component of a labeled nucleotide structure to obtain an array of candidate labeled nucleotides with varying properties. The resultant nucleotides are evaluated (e.g., as described below), and certain labeled nucleotides are optimized for concentration and labeling fraction (the ratio of labeled to unlabeled nucleotide in a flow).

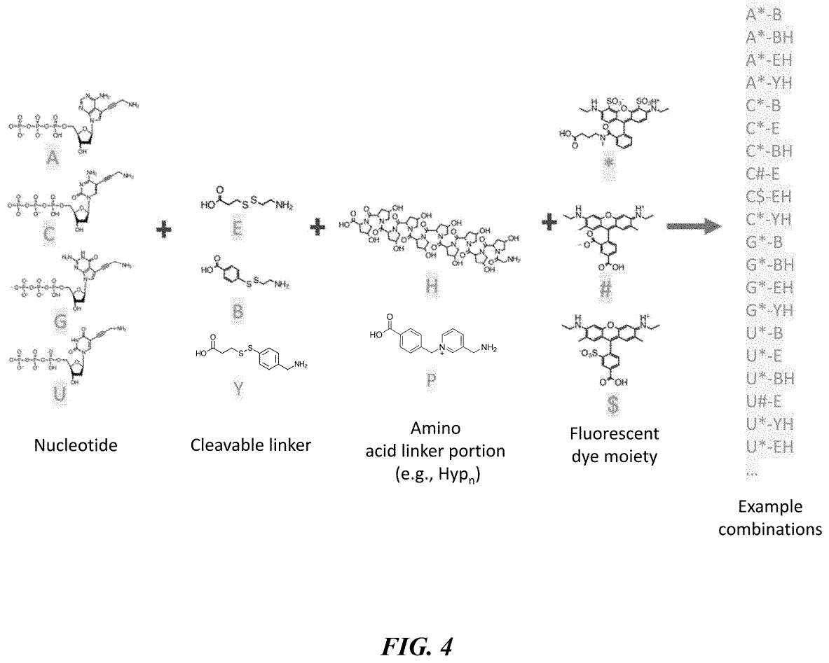

FIG. 4 shows a variety of components that may be used in the construction of labeled nucleotides. A nucleotide can be modified with a cleavable linker moiety, a semi-rigid linker moiety such a linker moiety comprising one or more amino acids, and a fluorescent dye moiety. The nucleotides shown in FIG. 4 are propargylamino functionalized nucleotides (A, C, G, and U), but any other useful nucleotide or nucleotide analog with any other useful chemical handle can be used. Cleavable linker moieties include, for example, the structures shown as “E,” “B,” and “Y”. Each cleavable linker moiety includes a cleavable group (e.g., as described herein). For example, cleavable linker moieties E, B, and Y include disulfide bonds. A linker moiety (e.g., a semi-rigid linker moiety) may comprise one or more amino acid moieties, including, for example, one or more hydroxyproline moieties (e.g., as described herein). For example, a linker moiety may comprise a hydroxyproline linker (hype). The “H” linker moiety illustrated in FIG. 4 is hyp10 moiety. In some cases, a fluorescently labeled nucleotide may comprise multiple hyp10 moieties in the same or different regions of the chemical structure. For example, a linker moiety may comprise 2 or more hyp10 moieties (e.g., a hyp20 or hyp30 moiety, each of which may include 10 hydroxyproline moieties and, in some cases, another moiety such as a glycine moiety, as described herein) in sequence, which moieties may be separated by one or more other moieties or features. In some cases, a linker moiety may comprise the “P” moiety shown in FIG. 4. A linker may include multiple different portions including multiple different amino acid sequences including 2 or more amino acids (e.g., as described herein). In some cases, a fluorescently labeled nucleotide may comprise a branched or dendritic structure (e.g., as described herein) comprising multiple linker moieties (e.g., multiple sets of hydroxyproline moieties connected at different branch points to a central structure), which linker moieties may be the same or different. A fluorescently labeled nucleotide may also include one or more fluorescent dye moieties. A fluorescent dye moiety may be a structure shown in FIG. 4 as “*,” “#,” “$,” or any other useful structure. Throughout the application, these labels are used to refer to specific dye structures. However, wherever such labels are used, any other dye moiety may be substituted, including any other fluorescent dye moiety described herein. In some cases, a dye may be represented as “‡” which symbol is intended to represent any useful dye moiety or combination of dye moieties (e.g., dye pairs). Such dyes may fluoresce at or near 530 nm, or in any other useful range of the electromagnetic spectrum (e.g., as described herein). For example, red-fluorescing dyes may also be utilized. Additional examples of dye moieties are included throughout the application. There are numerous possible variations of fluorescently labeled nucleotides. Some example combinations are included in FIG. 4. For example, a fluorescently labeled nucleotide may be U*-YH (e.g., a fluorescently labeled uracil-containing nucleotide comprising a Y cleavable linker and a hyp10 moiety and a * fluorescent dye moiety), U*-YHH (e.g., a fluorescently labeled uracil-containing nucleotide comprising a Y cleavable linker and two hyp10 moieties and a * fluorescent dye moiety), U #-E (e.g., a fluorescently labeled uracil-containing nucleotide comprising an E cleavable linker and a #fluorescent dye moiety and lacking a hyp10 or similar moiety), a G*-B (e.g., a fluorescently labeled guanine-containing nucleotide comprising a B cleavable linker and a * fluorescent dye moiety and lacking a hyp10 or similar moiety), etc. Labeled nucleotides may be prepared according to synthetic routes and principles described herein. An example synthesis of the G*-B-H labeled nucleotide is described in Example 4.

### Example 4: Synthesis of G*-B-H Labeled Nucleotide

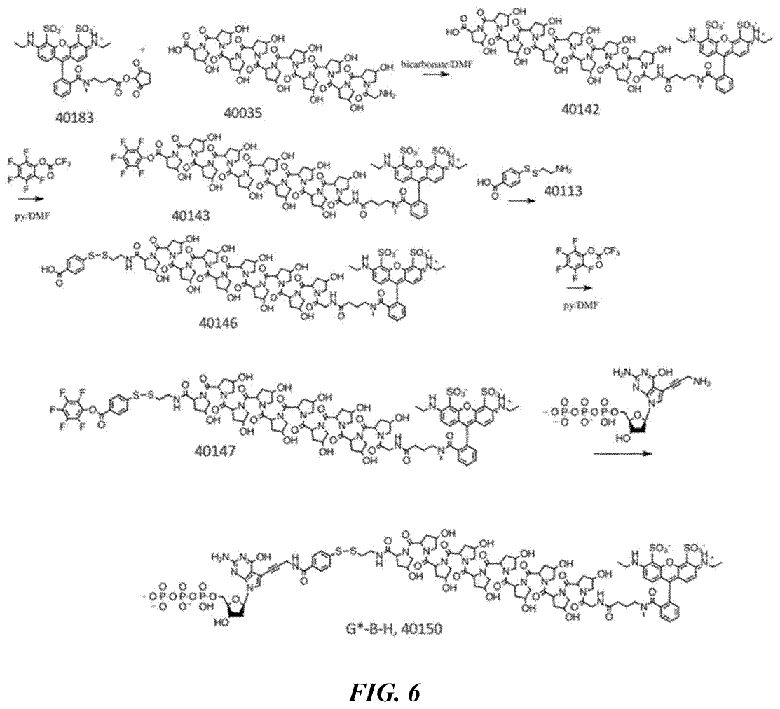

A synthetic method for preparing G*-B-H (see Example 3) is shown in FIG. 6. Similar methods may be used to prepare other labeled nucleotides described in Example 5 and elsewhere herein. As the components used include amino acids, there are multiple routes to the final product. Synthetic considerations include the tendency for hydrolysis of the triphosphate (to the diphosphate and monophosphates) under heat or acidic conditions, the tendency for disulfide to decompose in the presence of triethylamine and ammonia, preventing the use of acid-labile protecting groups, and preventing the use of trifluoroacetamide or FMOC protecting groups.

Preparation of PN 40142. A solution of Atto 532 succinimidyl ester (Atto-tec, PN 40183; 5 mg=4.6 μmol) in 100 μL of DMF is mixed with gly-hyp-hyp-hyp-hyp-hyp-hyp-hyp-hyp-hyp-hyp (custom synthesis from Genscript, PN 40035; 8.5 mg=7 μmol) in 170 μL 0.1 M bicarbonate in a 1.5 mL Eppendorf tube. The reaction is purified on a Phenomenex reverse phase C18 semi-prep column (Gemini 5 μM C18, 250×10 mm) using a gradient of 10%→40% acetonitrile vs. 0.1 M triethylammonium acetate over 16 minutes. The fractions containing product 40142 are combined and concentrated to dryness. The yield is determined by diluting a fraction and measuring the optical density (OD) at 633 nm and using an extinction coefficient for the dye of 130,000 cm⁻¹M⁻¹. The yield is 50%. The structure is confirmed by mass spectrometry in negative ion mode: m/z calculated for C₈₁H₁₀₃N₁₄O₃₁S₂⁻, 1831.6; found: 1831.8.

Preparation of PN 40143. PN 40142 (4 μmol) is suspended in 100 μL DMF in a 1.5 mL eppendorf tube. Pyridine (20 μL) and pentafluorophenyl trifluoroacetate (20 μL) are added to the DMF solution and heated to 50° C. for five minutes. A portion (1 μL) of the reaction mixture is precipitated into 0.4% HCl; the aqueous solution remains colorless, indicating complete conversion to the active pentafluorophenyl ester. The remainder of the reaction is precipitated into the dilute acidic solution and the aqueous solution pipetted off. The residue is washed with hexane and dried to a highly colored solid (PN 40143)

Preparation of PN 40146. PN 40143 is dissolved in 100 μL DMF and mixed with disulfide PN 40113 (5 mg, 20 μmop in DMF. Diisopropylethylamine (5 μL) is added to the mixture. The mixture is purified on reverse phase HPLC using a gradient of 20%→50% acetonitrile vs. 0.1 M TEAA over 16 minutes. Two dye-colored fractions are obtained at 8.8 min and 9.5 min. The fraction at 9.5 min is identified by mass spectrometry to be the desired product: m/z calculated for C₉₀H₁₁₁N₁₅O₃₂S₄²⁻, [M−H]²⁻, 1020.84; found: 1021.1.

Preparation of PN 40147. PN 40146 is suspended in 100 μL DMF in a 1.5 mL eppendorf tube. Pyridine (20 μL) and pentafluorophenyl trifluoroacetate (20 μL) are added to the DMF solution and heated to 50° C. for five minutes. A portion (1 μL) of the reaction mixture is precipitated into 0.4% HCl; the aqueous solution remains colorless, indicating complete conversion to the active, pentafluorophenyl ester. The remainder of the reaction is precipitated into the dilute acidic solution and the aqueous solution pipetted off. The residue is washed with hexane and dried to a highly colored solid (PN 40147)

Preparation of PN 40150. PN 40147 is dissolved in 50 μL DMF in a 1.5 mL eppendorf tube. A solution of 0.5 μmol 7-deaza-7-propargylamino-2′-deoxyguanosine-5′-triphosphate in 50 μL 1 M bicarbonate is prepared and added to the tube. After remaining overnight at 4° C. the product is purified on HPLC; the fraction at 12 min using a 20%→50% acetonitrile vs. 0.1 M TEAA gradient over 16 minutes contains the desired product: m/z calculated for C₁₀₄H₁₂₉N₂₀O₄₄P₃S₄²⁻, [M−H]²⁻, 1291.33; found: 1292.4.

### Example 5: Dye-Labeled Nucleotides Including Guanine or Analogs Thereof

Nucleotides including guanine or analogs thereof may perform more poorly in sequencing applications (e.g., as described herein) in base-calling accuracy. This may be related to photoinduced electron transfer from the nucleobase to a dye linked to the nucleobase, which may quench signal emitted by the dye and thus less dynamic range of signal. Accordingly, various dye-labeled nucleotides including guanine or analogs thereof are prepared and evaluated as provided herein. Examples of such dye-labeled nucleotides include:

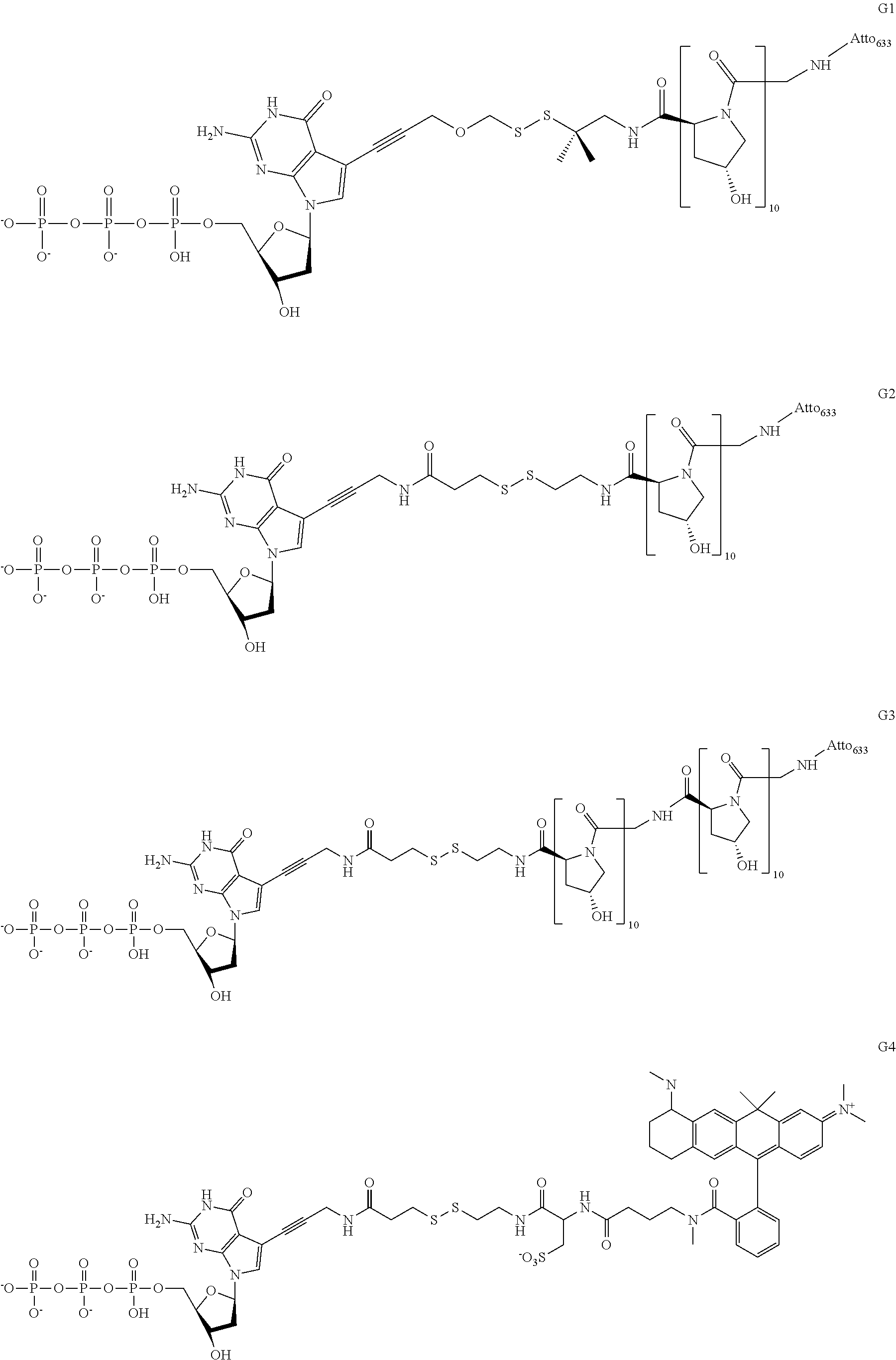

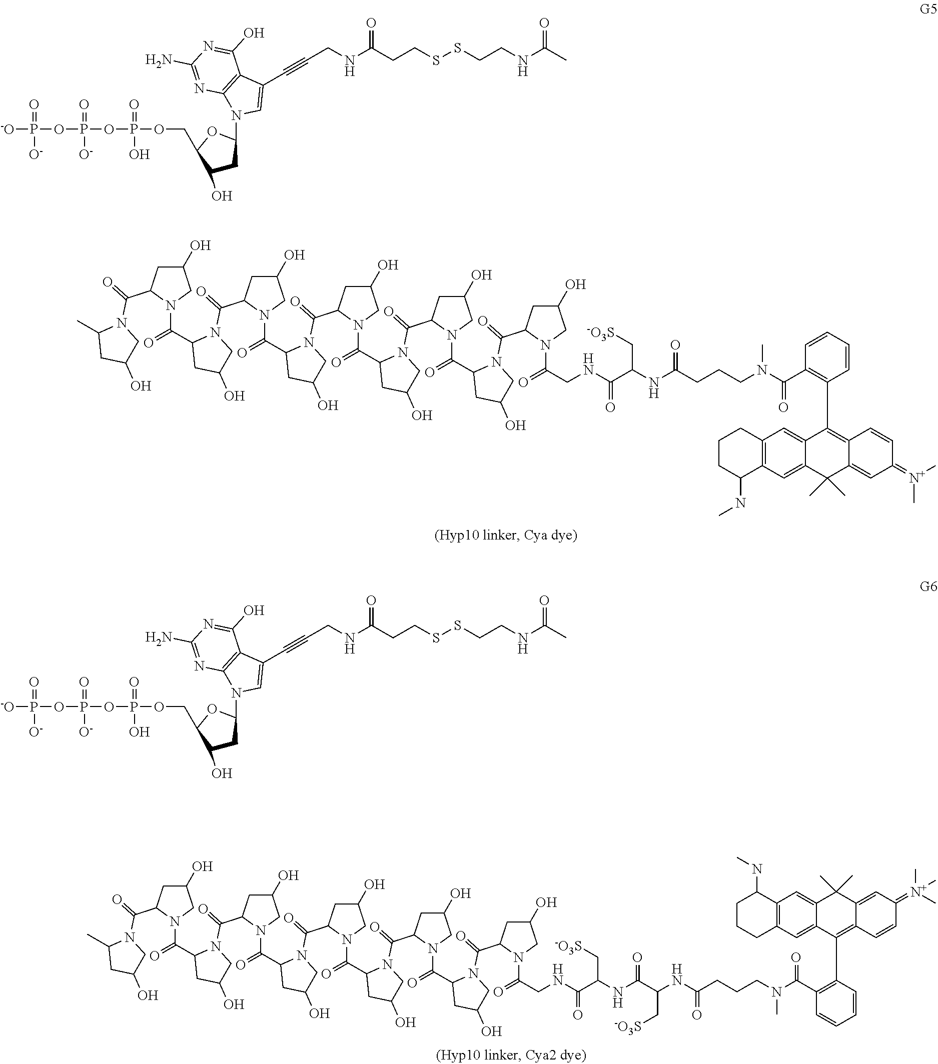

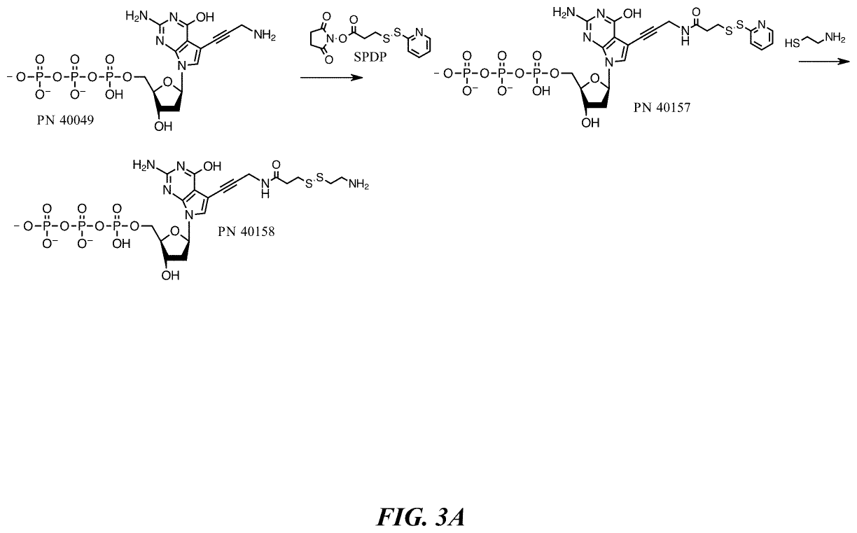

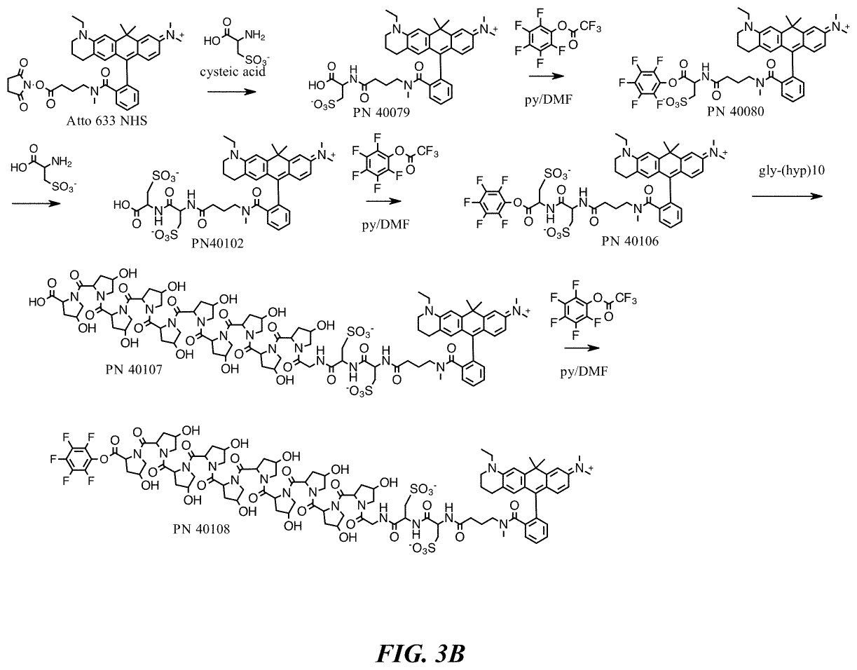

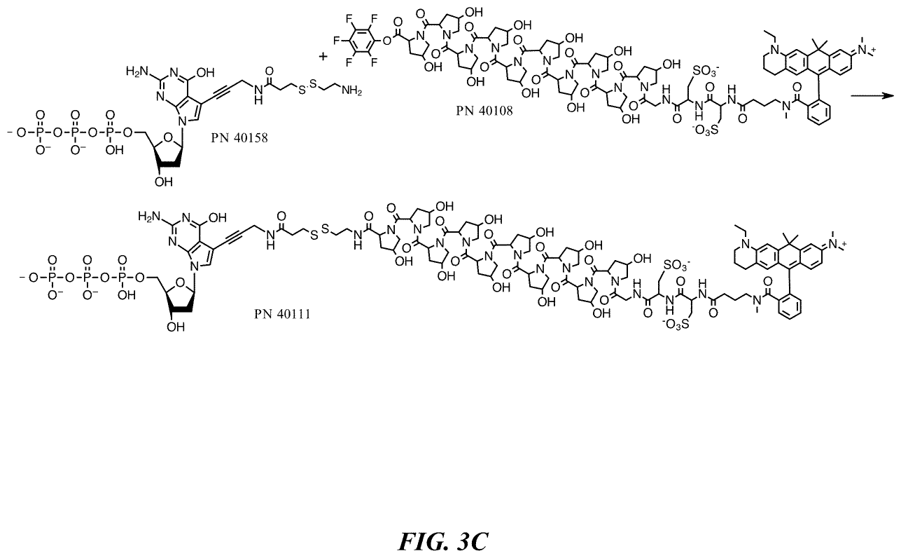

Several of the structures shown above include the hyp10 linker which includes the sequence Gly-Hyp-Hyp-Hyp-Hyp-Hyp-Hyp-Hyp-Hyp-Hyp-Hyp from the N-terminal end. G4, which lacked the hyp10 linker, is highly quenched. The remaining dye-labeled nucleotides are evaluated in a sequencing assay, as described herein. Of the structures shown, G6 provides the highest accuracy. A synthetic route for preparation of G6 is shown in FIGS. 3A-3C. Additional structures including different numbers of hydroxyprolines, including hyp20 and hyp30 moieties, may also be incorporated into fluorescent labeling reagents.

### Example 6: Evaluation of Dye-Labeled Nucleotides

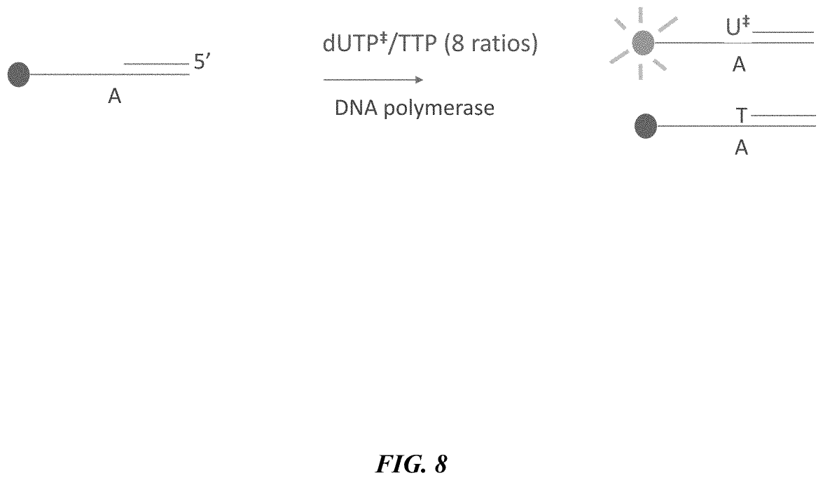

A bead-based assay is used to evaluate dye-labeled nucleotides of Example 5. A streptavidin bead is prepared with a 5′-biotinylated template strand annealed to a primer strand. The primer strand is designed so that the next cognate base incorporated by a DNA polymerase is a thymidine. A DNA polymerase is bound to the bead complex. Various mixtures containing different ratios of the dye-labeled nucleotide (dUTP*) and the natural base (TTP) is then presented to the beads. After washing away excess reagent, the fluorescence of the beads is read on a flow cytometer using the PE channel (excitation=488 nm, emission=580 nm). A schematic of this assay is shown in FIG. 8.

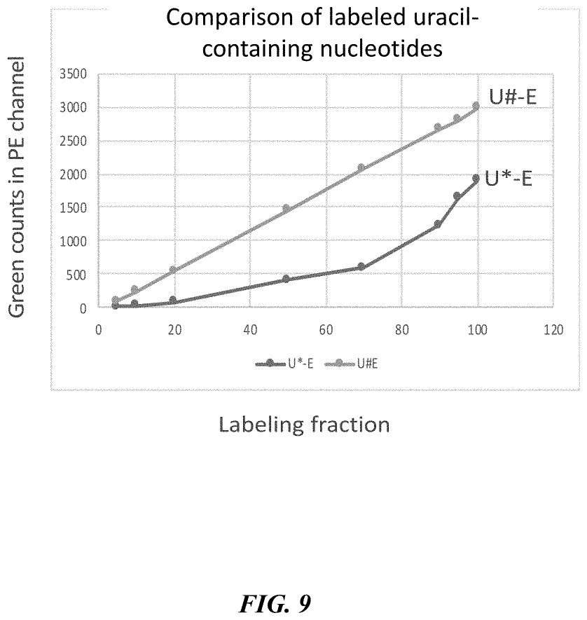

The results of the bead assay for different labeled dUTPs are shown in FIG. 9. The total concentration of the sum of the nucleotides is maintained at 2 μM; a labeling fraction of 10% means 0.2 μM of dUTP* and 1.8 μM of TTP. The behavior for the two nucleotides is noticeably different: U #-E has a “tolerance” of about one, meaning that there is no difference in incorporation of the dye-labeled vs the natural nucleotide over all the ratios tested; i.e., a 50% labeling fraction results in 50% of the beads getting labeled. U*-E, on the other hand, has a negative tolerance, meaning that at every ratio it falls below the line drawn between zero and the signal at 100% labeled. A negative tolerance suggests that the dye-label makes the nucleotide a worse substrate than the natural substrate. This result is consistent with the observation that negatively charged dyes such as Atto532 (the dye denoted by U*-E) inhibit incorporation by many polymerases while dyes such as 5-carboxyrhodamine-6G (the dye denoted by U #-E) are zwitterionic and are known to be good substrates.

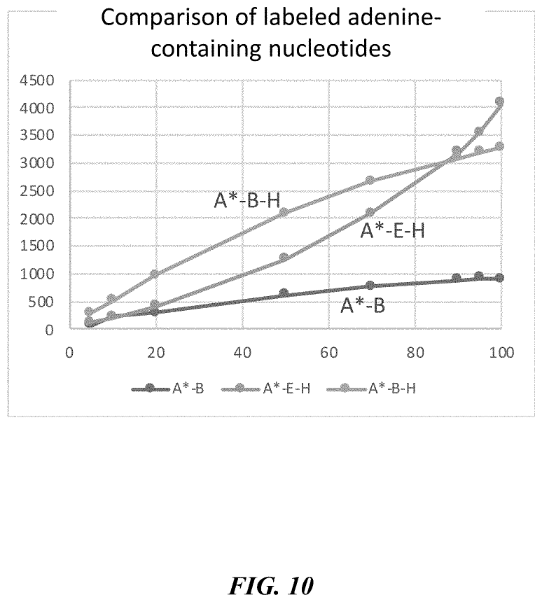

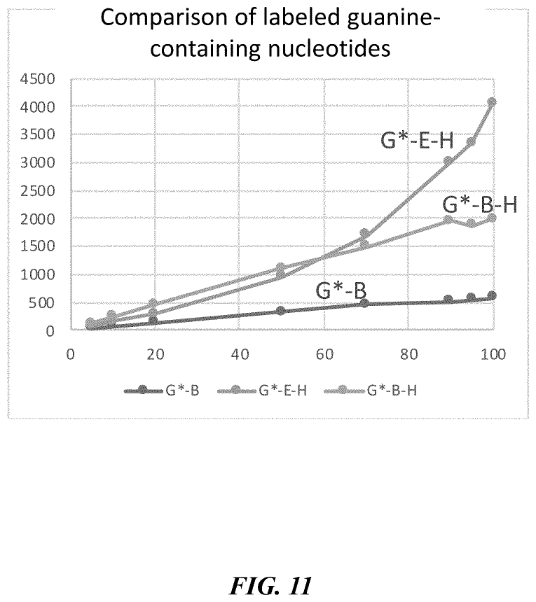

Additional labeled nucleotides were evaluated using a similar assay. FIG. 10 shows the result of the bead assay for labeled dATPs. FIG. 11 shows the result of the bead assay for labeled dGTPs. For labeled dATPs, very low fluorescence is observed at 100% labeling for A*-B compared to A*-B-H and A*-E-H. This indicates that the hydroxyproline linker (H) relieves quenching of the dye by the nucleotide. A similar result is observed for labeled dGTPs. This result is expected for labeled dGTP, as G quenching via photoinduced electron transfer is well known. A quenching effect from the disulfide linker, B, may also contribute to the lower fluorescence observed for labeled dATPs and dGTPs.

### Example 7: Sequencing Using Dye-Labeled Nucleotides

A nucleic acid sequencing assay may be used to evaluate dye-labeled nucleotides (e.g., as described herein). An example procedure is shown in FIG. 7.

Sequencing may be performed using an instrument outfitted with a light emitting device (LED) and/or a laser. Each nucleotide evaluated may include a dye that is configured for excitement and emission over similar wavelengths (e.g., all red or all green emission). One or more different nucleotide types may be coupled to different dyes. Sequencing performance may be evaluated based on base calling quality, phase lag, phase lead, and homopolymer completion.

Beads with amplified templates are primed, immobilized on a support, and incubated with a tight-binding DNA polymerase. Beads are then subjected to multiple cycles of sequencing. Each sequencing cycle may comprise incubation with U*/T (a fixed ratio of dye-labeled and natural TTP), a “chase” process (TTP alone), imaging, and a cleavage process (10 mM tris(hydroxypropyl)phosphine (THP)) to release the dye. Each process may have a wash process in between. This process may be repeated for A, C, and G-including nucleotides or nucleotide analogs. This sequencing procedure may effectively identify homopolymeric regions of at least 2, 3, 4, 5, 6, 7, 8, or more nucleotides.

Sequencing is also evaluated for an all hyp-linker set in which dye-labeled nucleotides including each canonical nucleotide include the hyp10 or hyp20 linker. This evaluation is performed to identify a set where higher fractions may be used with minimal quenching. Higher quenching may lead to higher scarring (e.g., as described herein), which may reduce incorporation efficiency by a polymerase enzyme. However, family B enzymes such as PolD may perform well with scars. Sequencing may be evaluated with 2.5% and 20% labeling fractions with a dye such as Atto633.

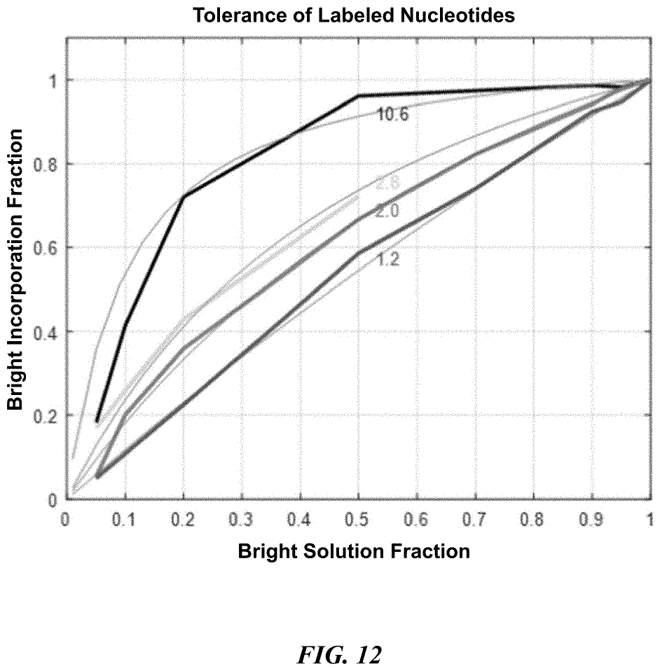

Sequencing may be used to evaluate the tolerance for various labeled nucleotides. FIG. 12 shows normalized bead data for nucleotides labeled with a red-emitting dye. Bright solution fraction (b_{f}) is plotted against bright incorporation fraction (bᵢ). The curves are fitted to the following equation:

$${b}_{i} = \frac{t o l ( {b}_{f} / {d}_{f} )}{1 + t o l ( {b}_{f} / {d}_{f} )}$$

in which d_{f} is the dark solution fraction. In FIG. 12, the calculated tolerances are 10.6 for G*, 2.8 for A*, 2.0 for U*, and 1.2 for C*. The positive tolerance numbers indicate that at 50% labeling fraction, more than 50% is labeled. Reagents with a tolerance of 1 may have the least “context” in sequencing. Reagents with a very negative tolerance (e.g., tolerance<<1) may have issues with uniform incorporation across a plurality of templates coupled to a support because they must be used at such low concentrations that they may fall below saturation and be consumed at an uneven rate.

### Example 8: Evaluation of Quenching

The dye-labeled nucleotides provided herein may improve quenching between nucleobases and the dyes to which they are attached and/or between dyes in a nucleic acid molecule (e.g., a growing nucleic acid strand), such as in a homopolymeric region of a nucleic acid molecule. Quenching may be evaluated in an enzyme-independent manner.

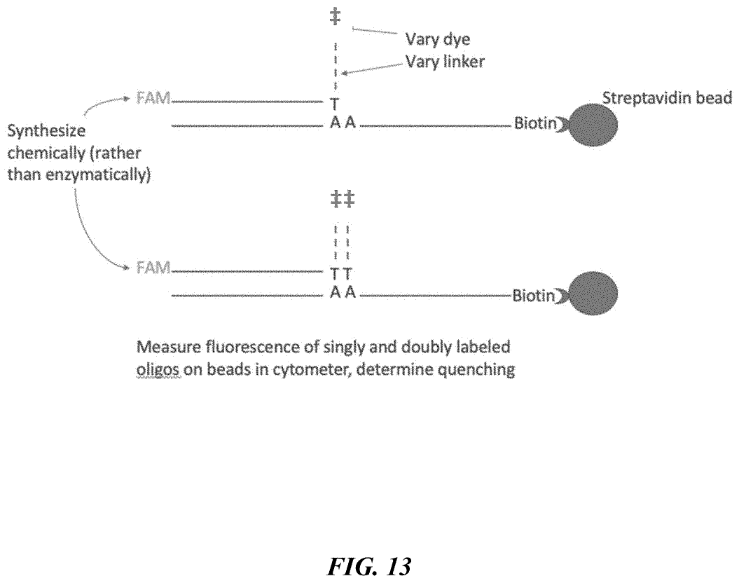

FIG. 13 shows a schematic for evaluating quenching. Synthetic oligos are constructed with one or two “linker arm nucleotides”. Linker arm nucleotides are thymidine analogs with a linker arm containing a primary amine. The oligonucleotide containing the linker arm nucleotide can be labeled with linkers and dyes and HPLC purified. The advantage of using the bead-labeled assay is that exact quantitation of the reagents is not necessary; a large excess can be used in each step and the beads washed, ensuring that only stoichiometric amounts of oligonucleotides are bound to the template. Each dye-linker is put on both oligonucleotides. The beads are measured on the flow cytometer in the APC (red) channel. The percent quenching is determined by the formula: % quenching=100×(1−Fl_{bis}/(2*Flₘₒₙₒ)).

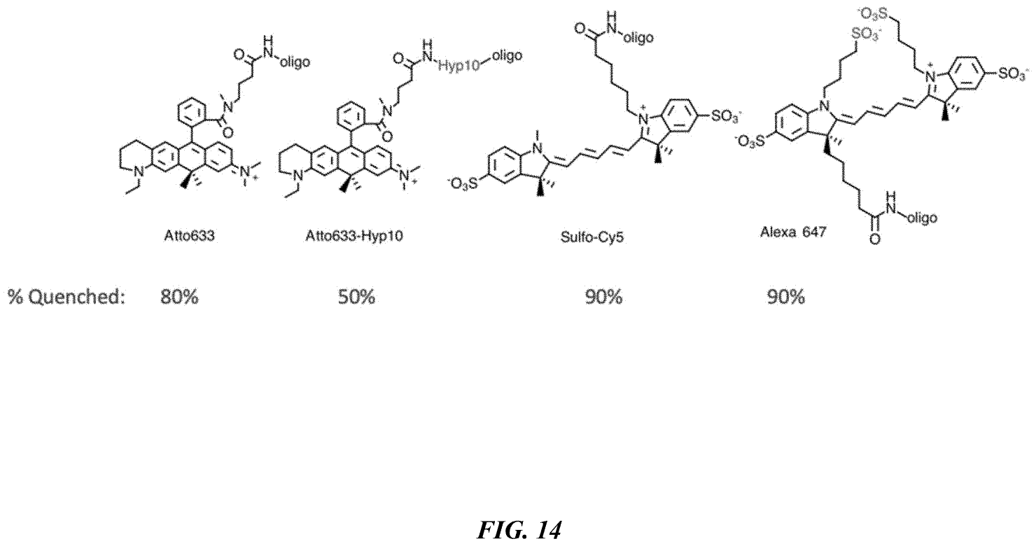

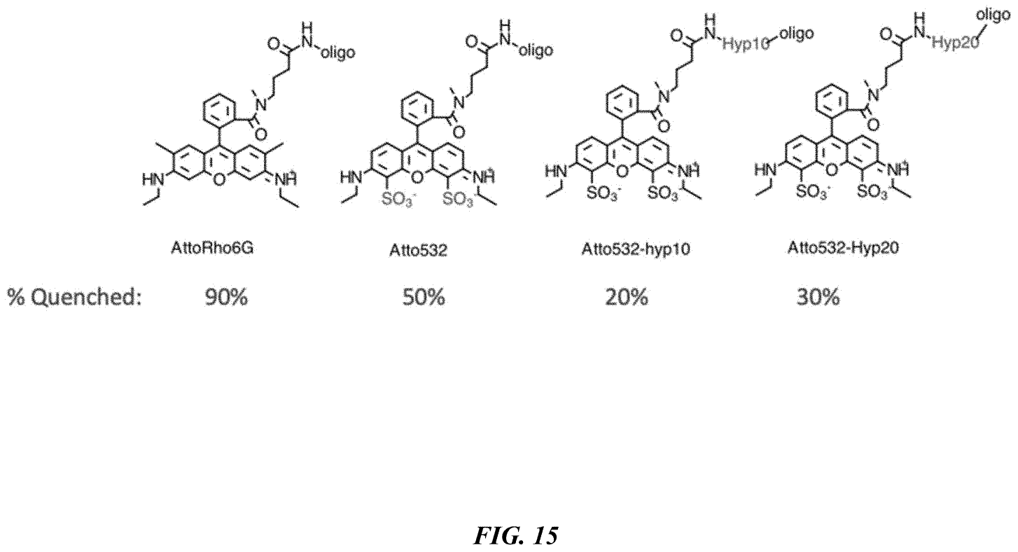

FIGS. 14 and 15 show quenching results for red dye linkers (FIG. 14) and green dye linkers (FIG. 15). The results show that the nature of the dye affects quenching. Negative charge (see Atto532 vs AttoRho6G) can improve quenching but if the dye is extremely large and flat (see Cy5, Alexa 647) quenching may not be improved. The hyp10 or hyp20 linkers improve quenching. As shown in FIG. 14, hyp10 improves quenching with Atto633, and cyanine dyes quench even with four sulfonic acid groups. As shown in FIG. 15, sulfonic acid groups on Atto532 improve quenching, and the combination of Atto532 and hyp10 also improves quenching.

### Example 9: Interrogation of Homopolymers

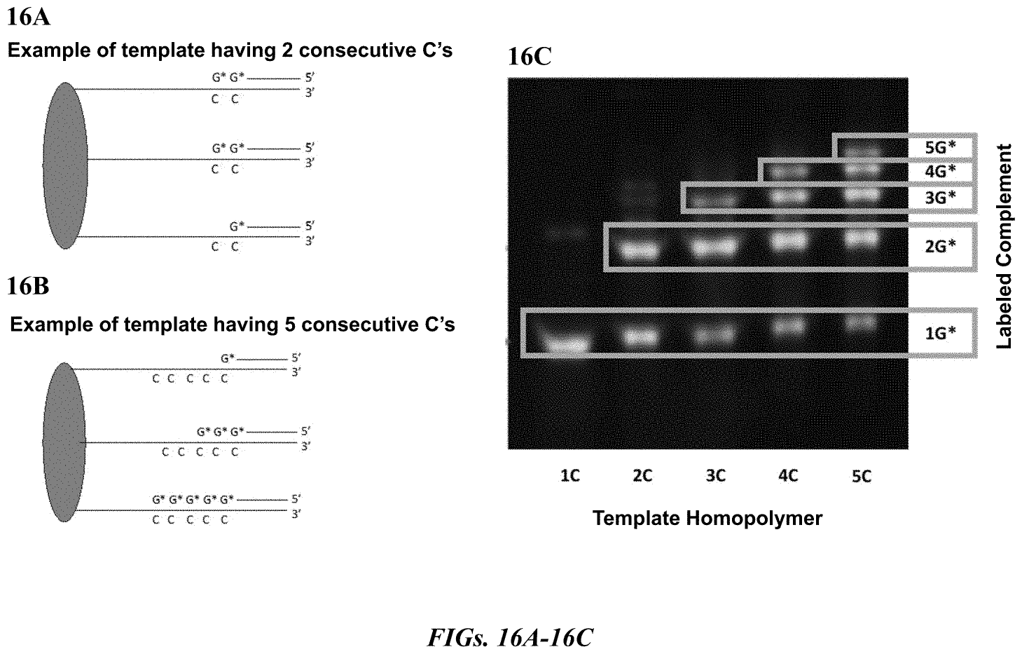

A nucleic acid template is provided that has various lengths of a homopolymer region comprising cytosines (1C, 2C, 3C, 4C, 5C). The template is contacted with guanosine-containing nucleotides labeled with Atto532 fluorophore (e.g., as described herein; denoted herein as G*). The labeled nucleotides may be provided in a solution as a nucleotide flow (e.g., as described herein). The nucleotide flow may include 100% labeled nucleotides (e.g., the nucleotide flow may include only labeled nucleotides and no unlabeled nucleotides) or may include both labeled and unlabeled nucleotides (e.g., as described herein). The labeled and, where present, unlabeled nucleotides may not be terminated so that multiple nucleotides can be incorporated into as many positions in succession as there appear cytosines in the template. An enzyme (e.g., a polymerase enzyme, such as Bst 3.0) may be used to incorporate labeled and/or unlabeled nucleotides into an extended primer using the nucleic acid having a polycytosine sequence as a template. A plurality of copies of the template may be immobilized to a bead or other support (e.g., as described herein). This procedure is schematically illustrated in FIGS. 16A and 16B.

In some cases, the labeled nucleotide incorporates into as many positions in succession as there appear cytosine in the template. In other cases, less than all potential G* are incorporated. Where unlabeled nucleotides are included in the nucleotide flow, both unlabeled and labeled nucleotides may be incorporated. For example, for a template including a homopolymeric region including three cytosines, the incorporated nucleotides may have the sequence GGG, GG*G, GGG*, G*GG, G*G*G, G*GG*, GG*G*, or G*G*G*, where G* indicates a labeled nucleotide and G indicates an unlabeled nucleotide. The sequence of the incorporated nucleotides may vary based on, for example, the labeling fraction of the nucleotide flow (e.g., the ratio of labeled to unlabeled nucleotides in the flow) and the optical (e.g., fluorescent) labeling reagent used to label the nucleotides.

Labeled polynucleotide products are separated on a Biorad denaturing acrylamide gel and imaged using blue and green LEDs to detect incorporated labeled nucleotides. As shown in FIGS. 16C, 1, 2, 3, 4, and 5 consecutive cytosines can be detected using this method.

### Example 10: Sequencing by Synthesis Using a High Fraction of Labeled Nucleotides

A template nucleic acid having a length of at least 30 nucleotides is sequenced using the procedures and labeled nucleotides described herein. The template to be sequenced may be immobilized to a support (e.g., as described herein). The template is subjected to a sequencing by synthesis reaction, in which the template is sequentially contacted with solutions (e.g., nucleotide flows) comprising PolD polymerase (New England Biolabs) and a plurality of nucleotides of a single canonical type (e.g., T, A, C, or G). In each nucleotide flow, approximately 20% of the nucleotide population is labeled with Atto633 as described herein above to provide a labeling fraction of about 20%. The remaining nucleotides are unlabeled. Nucleotides included in nucleotide flows are not terminated to allow efficient sequencing of homopolymeric regions of the template. After contacting the template with a first nucleotide flow including nucleotides of a first canonical type, the template is contacted with a wash flow to remove unincorporated nucleotides. A fluorescent image is collected. The linker of the fluorescent labeling reagent associated with incorporated labeled nucleotides is contacted with a cleavage flow comprising a cleavage reagent configured to cleave a cleavable group of the linker to separate the fluorescent dye (e.g., Atto633) of the fluorescent labeling reagent from the incorporated nucleotide. An additional wash flow may be used to remove the cleavage flow. In some cases, a chase flow including unlabeled nucleotides of the first canonical type may follow the initial nucleotide flow and precede or follow the imaging process. The process is repeated for the second, third, and fourth nucleotide types in succession, and then the entire cycle is repeated.

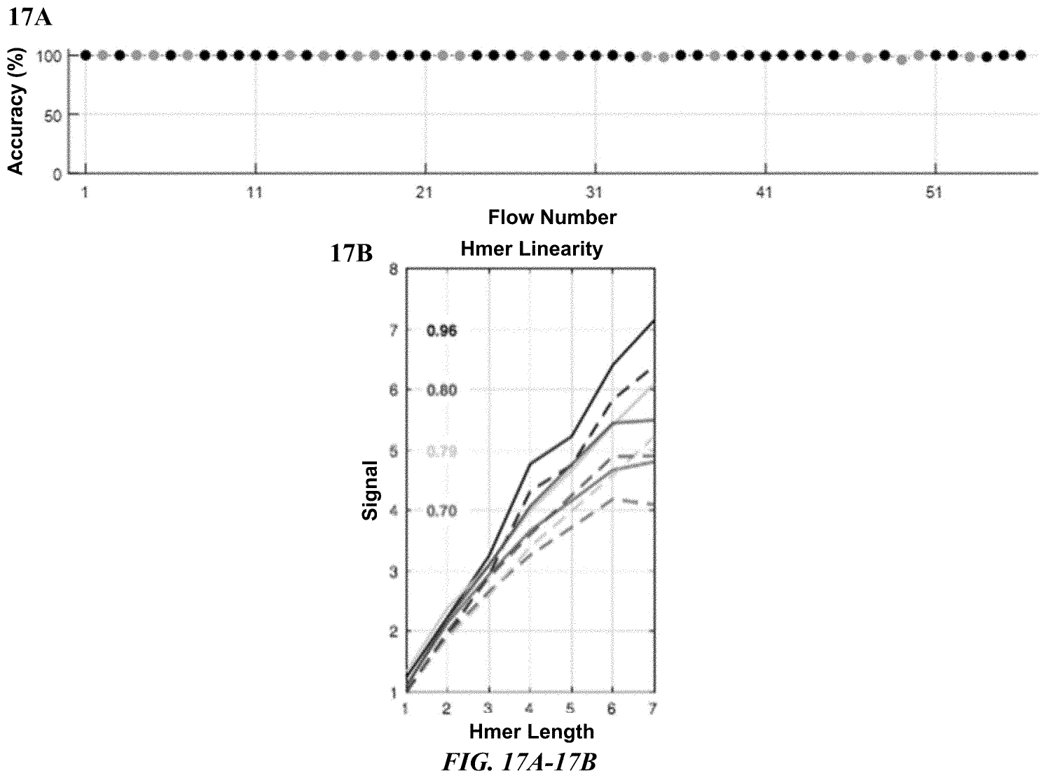

FIG. 17A shows the results of application of this method to a sample template. A black circle indicates that a nucleotide was incorporated and a gray circle indicates that no nucleotide was incorporated in a particular flow cycle. As shown in the figure, the incorporation of one or more nucleotides in a flow cycle can be determined with a high degree of accuracy. Furthermore, as is shown in FIG. 17B, the relationship between signal intensity and labeled nucleotide homopolymer length may be substantially linear across a plurality of templates (e.g., as described herein). For example, the signal intensity may be proportional to the length of a homopolymeric region of the template. This proportionality indicates that quenching effects have been substantially overcome. In FIG. 17B, the slope for G is 0.96, for C is 0.80, for A is 079, and for T is 0.70. The dotted line indicates the actual signal, while the solid line indicates the signal after correction for phasing.

### Example 11: Sequencing by Synthesis Using 100% Labeled Nucleotides

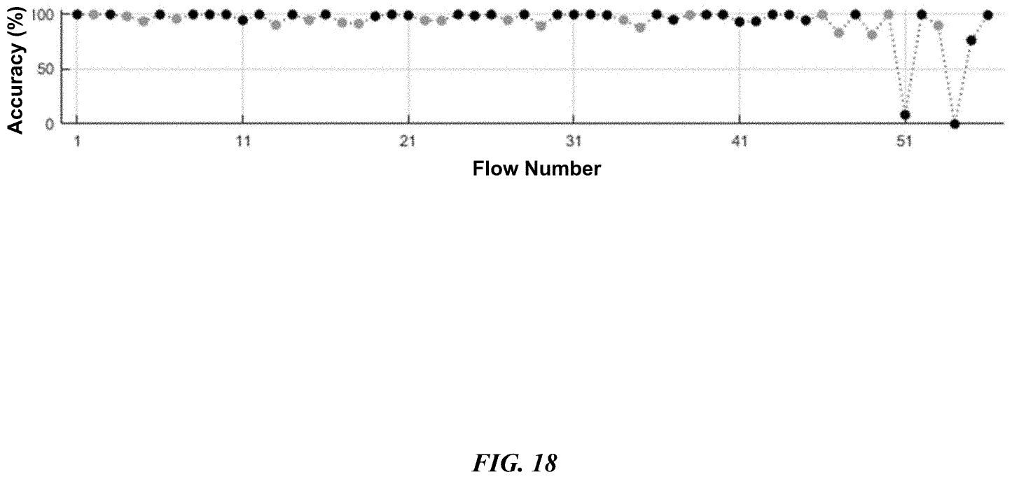

A template nucleic acid having a length of at least 30 nucleotides is sequenced as described in Example 13, but with solutions in which 100% of the nucleotides are labeled. In FIG. 18, black circles indicate that a base was incorporated in a given flow cycle, while gray circles indicate that a base was not incorporated in a given flow cycle. As can be seen from FIG. 18, the sequencing method can be used to detect base incorporation through 50 flow cycles.

### Example 12: Protein Labeling

A protein is labeled with a plurality of optical (e.g., fluorescent) labeling reagents (e.g., as described herein). For example, the protein may be labeled with three or more optical labeling reagents. The optical labeling reagents associated with the protein may all comprise a fluorescent dye of the same type. The optical labeling reagents associated with the protein may all comprise a linker of the same type. The protein may be an antibody, such as a monoclonal antibody.

The protein is used to label a cell. The cell may be a component of sample, which sample may comprise a plurality of cells. The cells of the sample may be analyzed and sorted using flow cytometry. Flow cytometric analysis may identify the cell as being labeled with the protein associated with the plurality of optical labeling reagents. In some cases, a plurality of cells of a sample may be labeled with optical labeling reagents (e.g., as described herein). For example, cells comprising a particular cell surface feature (e.g., an antigen) configured to associate with a protein (e.g., a protein labeled with a plurality of optical labeling reagents, such as an antibody labeled with a plurality of optical labeling reagents) may be labeled with labeled proteins and analyzed and/or sorted using flow cytometry. Analyzed and/or sorted cells may be subjected to further downstream analysis and processing, including, for example, nucleic acid sequencing, staining, imaging, function assays, immunoassays, isolation/expansion, additional labeling, immunoprecipitation, etc.

### Example 13: Effect of Separation of Dye and Substrate

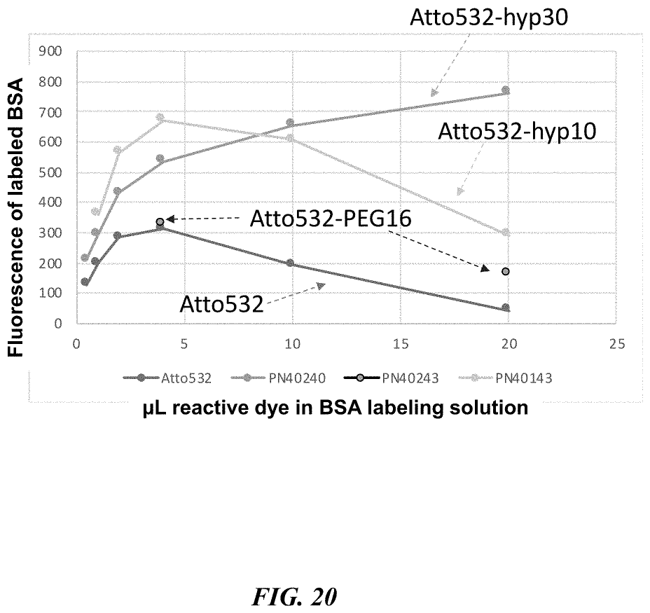

The effect of functional separation between an optically detectable moiety (e.g., fluorescent dye) and a substrate was investigated using bovine serum albumin (BSA). BSA was fluorescently labeled with Atto532 according to the following schemes: in the absence of a linker providing separation between the BSA and Atto532 moieties (“Atto532”), using PEG16 as a linker to provide separation between the BSA and Atto532 moieties (“Atto532-PEG16”), using a hyp10 moiety to provide separation between the BSA and Atto532 moieties (“Atto532-hyp10”), and using a hyp30 moiety to provide separation between the BSA and Atto532 moieties (“Atto532-hyp30”). Labeled BSA was purified from free dye using Millipore centrifugal filters. As shown in FIG. 20, the Atto532-hyp30 labeling scheme does not demonstrate self-quenching on the BSA protein. Atto532-hyp30 performed better than Atto532-hyp10, demonstrating that the added physical separation between the BSA and the dye moiety may be useful in reducing quenching. Atto532-PEG16 did not improve quenching over Atto532 alone.

### Example 14: Effect of Separation of Dye and Substrate

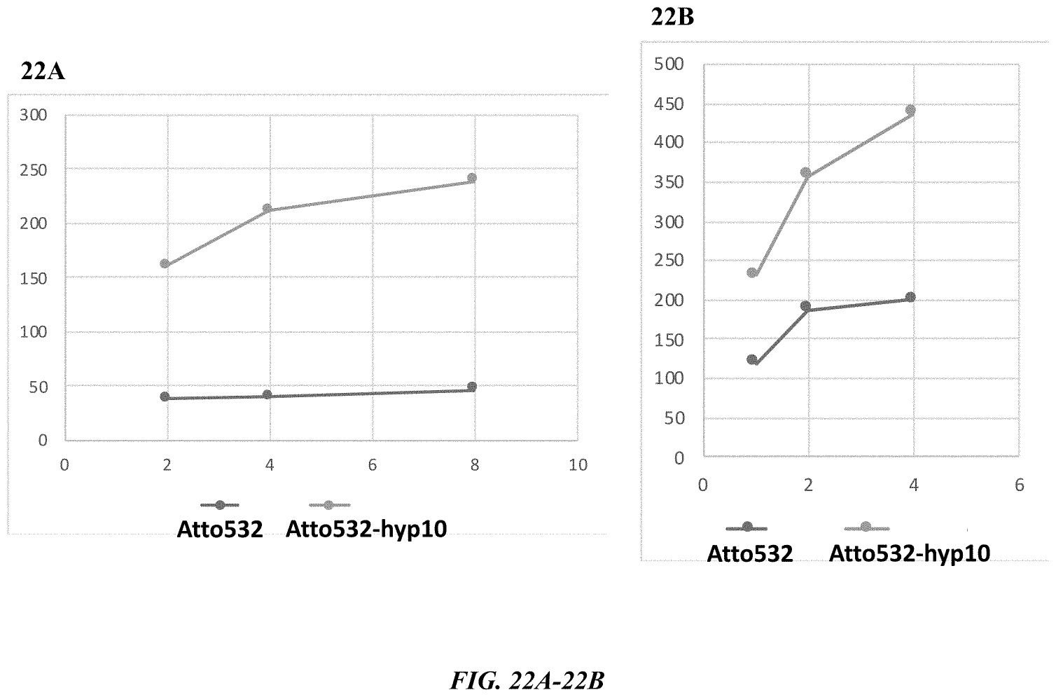

The effect of functional separation between an optically detectable moiety (e.g., fluorescent dye) and a substrate was investigated using streptavidin. Aliquots of streptavidin (0.8 milligrams/25 microliters (4), 0.1 Molar (M) bicarbonate) were combined with 2, 4, or 8 microliters of dye-PFP (pentafluorophenyl) at 12 millimolar (mM). After 1 hour at room temperature, samples were purified using centrifugal filter units with 30000 Daltons as a molecular weight cut off. Protein solutions were washed with TE and spun six times until the eluant was colorless. Absorbance spectra were measured using a Denovix UV/Visible spectrophotometer and absorbances at 280 and 534 nm were measured. The uncorrected protein and fluorophore concentrations were determined using extinction coefficients of 41,300 and 115,000 M⁻¹ centimeter (cm)⁻¹ for streptavidin and Atto532, respectively. Samples were diluted 20× to 200 μL and the green fluorescence was measured. FIG. 22A shows the brightness of streptavidin labeled with Atto532 that was physical separated by a hyp10 linker vs. streptavidin labeled with Atto532 in the absence of a linker providing physical separation between the streptavidin and the Atto532 moiety. The brightness of the Atto532-hyp10 labeled streptavidin was nearly five times the brightness of the streptavidin labeled with Atto532 alone. Table 1 summarizes relevant parameters.

TABLE 1
Parameters relevant to streptavidin labeling.

| | | Abs | Abs | | F | P | |
|---|---|---|---|---|---|---|---|
| | Volume | 280 | 534 | Fluor | conc | conc | F/P |
| Label | (μL) | nm | nm | (K) | (mM) | (mM) | (uncorrected) |
| Atto532- | 2 | 5.58 | 7.11 | 38 | 0.06 | 0.14 | 0.45 |
| PFP | | | | | | | |
| Atto532- | 4 | 6.02 | 10.45 | 40 | 0.09 | 0.15 | 0.62 |
| PFP | | | | | | | |
| Atto532- | 8 | 7.13 | 19.03 | 47 | 0.17 | 0.17 | 0.95 |
| PFP | | | | | | | |
| Atto532- | 2 | 5.55 | 5.09 | 161 | 0.04 | 0.14 | 0.33 |
| hyp10- | | | | | | | |
| PFP | | | | | | | |
| Atto532- | 4 | 5.83 | 7.33 | 212 | 0.06 | 0.14 | 0.45 |
| hyp10- | | | | | | | |
| PFP | | | | | | | |
| Atto532- | 8 | 5.74 | 9.17 | 239 | 0.08 | 0.14 | 0.57 |
| hyp10- | | | | | | | |
| PFP | | | | | | | |

The effect of functional separation between an optically detectable moiety (e.g., fluorescent dye) and a substrate was also investigated using a mouse antibody. Aliquots (0.2 mg) of Mouse IgG (polyclonal antibody, SigmaAldrich #PP54) in 25 microliters (4) of 0.1 Molar (M) bicarbonate were combined with 1, 2, and 4 microliters of dye-PFP (pentafluorophenyl) at 12 millimolar (mM). Samples were purified of free dye using centrifugal filter units with 30000 Daltons as a molecular weight cut off. Protein solutions were washed six times with 400 μL TE until the eluant was colorless. Absorbance spectra were measured using a Denovix UV/Visible spectrophotometer and absorbances at 280 and 534 nm were measured. The uncorrected protein and fluorophore concentrations were determined using extinction coefficients of 210,000 and 115,000 M⁻¹ cm⁻¹ for mouse IgG and Atto532, respectively. Samples were diluted 20× to 200 μL and the green fluorescence was measured. FIG. 22B shows the brightness of the mouse IgG labeled with Atto532 that was physical separated by a hyp10 linker vs. the mouse IgG labeled with Atto532 in the absence of a linker providing physical separation between the mouse IgG and the Atto532 moiety. The brightness of the Atto532-hyp10 labeled mouse IgG was more than double that of the mouse IgG labeled with Atto532 alone. Table 2 summarizes relevant parameters. Moreover, the fluorescence of the Atto532-hyp10 labeled mouse IgG has not leveled off at the measured concentration, indicating that potential higher brightness is possible.

TABLE 2
Parameters relevant to mouse IgG labeling.

| | | Abs | Abs | | F | P | |
|---|---|---|---|---|---|---|---|
| | Volume | 280 | 534 | Fluor | conc | conc | F/P |
| Label | (μL) | nm | nm | (K) | (mM) | (mM) | (uncorrected) |
| Atto532- | 1 | 2.21 | 3.82 | 119 | 0.033 | 0.011 | 3.2 |
| PFP | | | | | | | |
| Atto532- | 2 | 2.73 | 7.23 | 187 | 0.063 | 0.013 | 4.8 |
| PFP | | | | | | | |
| Atto532- | 4 | 3.6 | 13.45 | 200 | 0.117 | 0.017 | 6.8 |
| PFP | | | | | | | |
| Atto532- | 1 | 2.395 | 5.275 | 231 | 0.046 | 0.011 | 4.0 |
| hyp10- | | | | | | | |
| PFP | | | | | | | |
| Atto532- | 2 | 2.765 | 7.955 | 357 | 0.069 | 0.013 | 5.3 |
| hyp10- | | | | | | | |
| PFP | | | | | | | |
| Atto532- | 4 | 3.49 | 13.275 | 437 | 0.115 | 0.017 | 6.9 |
| hyp10- | | | | | | | |
| PFP | | | | | | | |

### Example 15: Labeling Reagents Including Multiple Optically Detectable Moieties

As described in the preceding sections, a labeling reagent may comprise multiple optically detectable moieties (e.g., fluorescent dye moieties). Multiple optically detectable moieties may be connected to a scaffold structure of a labeling reagent, which scaffold may comprise one or more lysines. FIGS. 21A, 21B, 21D, and 21E show examples of such structures. FIG. 21C shows relative quantum yields for selected labeling reagents. As quantum yield is difficult to obtain a priori, quantum yields were measured by comparison of compounds with similar excitation and emission wavelengths using the same instrument. Quantum yield ratios may be obtained by measuring a fluorescence ratio as follows:

$$\frac{{F}_{1}}{{F}_{2}} = \frac{{ε}_{1} {c}_{1} b {Φ}_{1} {kl}_{0}}{{ε}_{2} {c}_{2} b {Φ}_{2} {kl}_{0}} .$$

Substituting absorbance from Beer's law and matching the absorbance of two samples provides the following:

$$\frac{{F}_{1}}{{F}_{2}} = \frac{{Φ}_{1}}{{Φ}_{2}} .$$

Accordingly, the ratio of the fluorescence is the ratio of the quantum yields. Both absorbance and fluorescence were measured using the Denovix spectrophotometer. The optical densities at the absorbance maxima (534 nm) were measured and matched and the green fluorescence measured. The quantum yields were normalized to that of the free dye, Atto532, reported to be 0.9. As shown in FIG. 21C, quantum yield was higher for structures including hyp10 or hyp30 structures than for structures including dye moieties connected directly to the lysine backbone.

### Example 16: Cleavable Linker Moieties

As described herein, a labeling reagent may include a cleavable moiety comprising a cleavable group. The inclusion of a cleavable moiety in a labeling reagent may facilitate separation of the labeling reagent or a portion thereof from a substrate to which it is coupled.

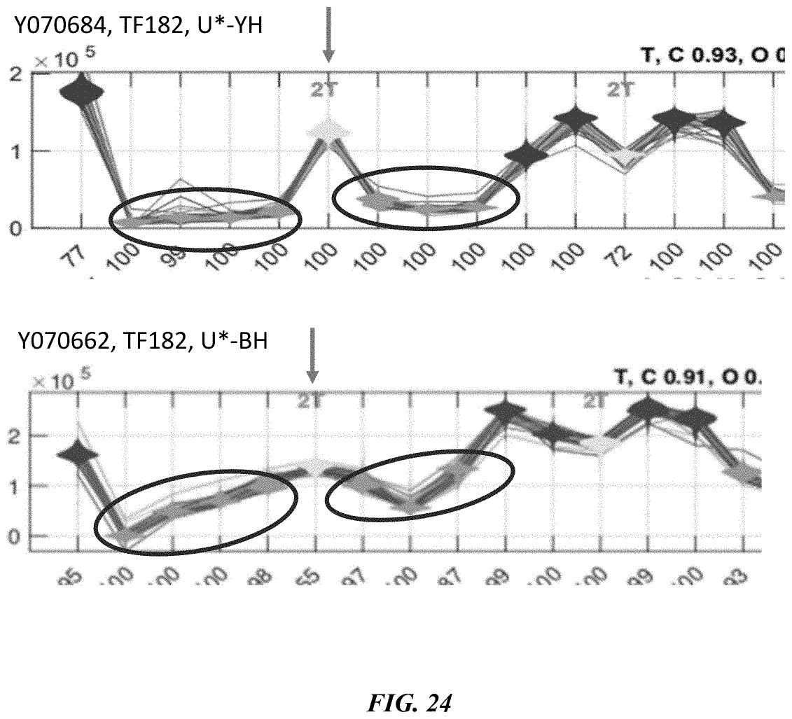

The performance of two labeled uracil-containing nucleotides was compared. Sequencing assays were performed using U*-YH (e.g., a uracil-containing nucleotide labeled with a labeling agent comprising a * dye, a Y cleavable linker, and a hyp10 moiety) or U*-BH (e.g., a uracil-containing nucleotide labeled with a labeling agent comprising a * dye, a B cleavable linker, and a hyp10 moiety). As shown in FIG. 24, U*-YH performed better in sequencing assays, providing low but constant signal for negative challenges (e.g., flows in which uracil was not intended to be incorporated into a template, circled), allowing positive signal (indicated by arrows) to be distinguished.

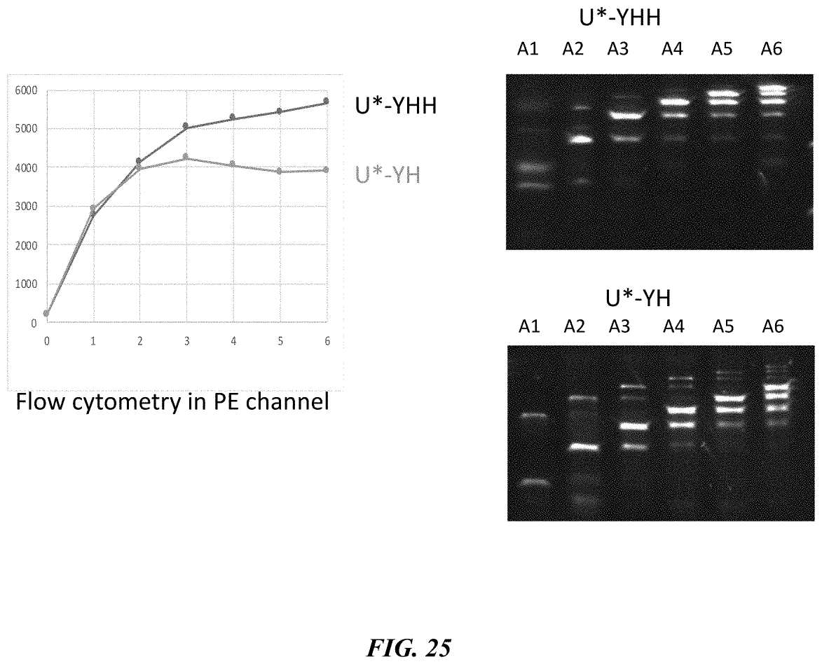

The performance of two labeled uracil-containing nucleotides including the same cleavable linker moieties and different semi-rigid portions was also compared. Sequencing assays were performed using U*-YH and U*-YHH (e.g., a uracil-containing nucleotide labeled with a labeling agent comprising a * dye, a Y cleavable linker, and two hyp10 moieties). Flow cytometry and gel-based analyses were used to evaluate the brightness of signal corresponding to each assay. As shown in FIG. 25, U*-YHH provided a brighter signal than U*-YH (left panel). As shown in the right panel of FIG. 25, for a template including six consecutive As (e.g., a homopolymeric region into which 6 uracils should incorporate), a range of products were measured using each labeled nucleotide. However, U*-YHH was less quenched than U*YH.

### Example 17: Dye Quenching

A labeling reagent of the present disclosure may comprise one or more different optically detectable moieties (e.g., dyes). An optically detectable moiety of a labeling reagent may fluoresce in the green region of the visible portion of the electromagnetic spectrum. A green-fluorescing dye may be, for example, Atto532. Alternatively or additionally, an optically detectable moiety of a labeling reagent may fluoresce in the red region of the visible portion of the electromagnetic spectrum. A red-fluorescing dye may be, for example, Atto633. The uses of red-fluorescing and green-fluorescing dyes were compared.

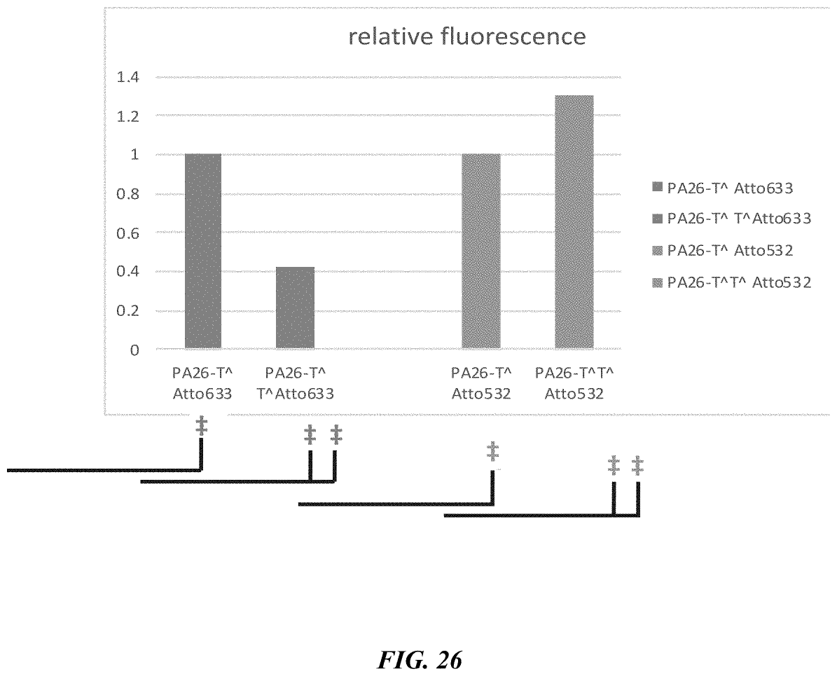

FIG. 26 shows relative fluorescence of Atto532 and Atto633 dyes coupled directly to oligos (e.g., in the absence of oligos). Fluorescence was measured with double-stranded DNA bound to beads using a flow cytometer. As shown in FIG. 26, two green dyes are 1.3× as bright as a single dye coupled to an oligo, while two red dyes are only 0.4× as bright as a single dye coupled to an oligo. Accordingly, green dyes may have an inherent advantage over red dyes. This difference may owe at least in part to Atto633 being hydrophobic while Atto532 is comparatively hydrophilic.

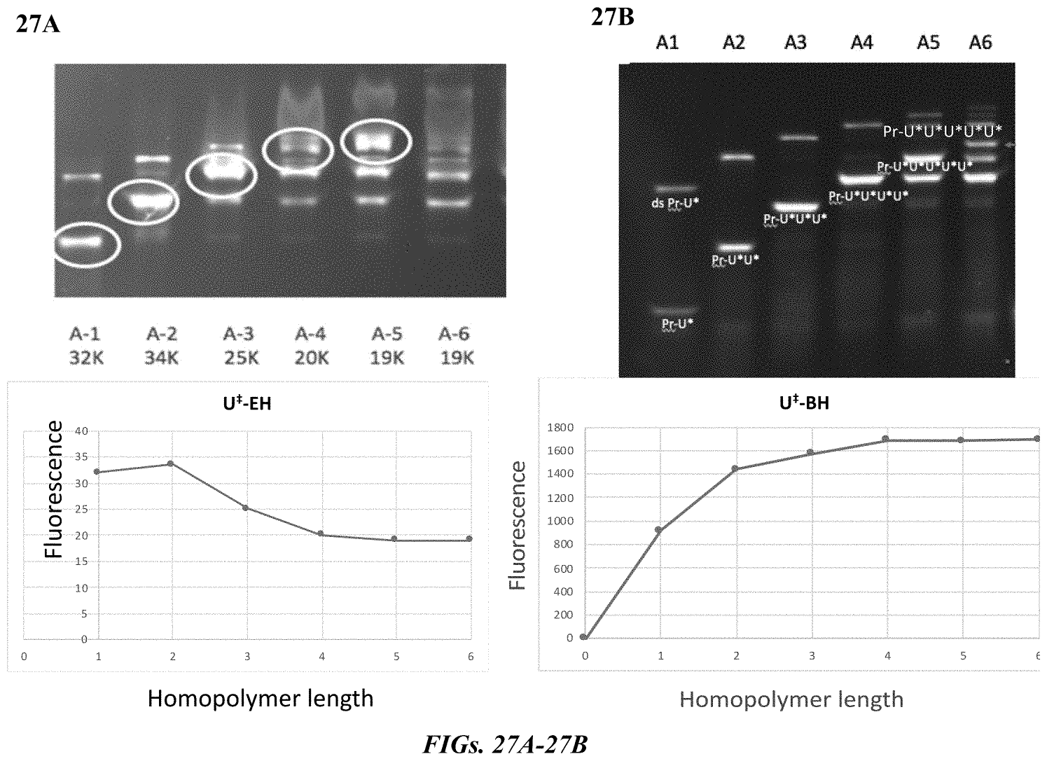

FIGS. 27A-27B show relative fluorescence as a function of homopolymer length. The data in FIG. 27A was prepared using a sequencing assay using dUTP-SS17-hyp10-Atto633 (e.g., a nucleotide labeled with a red-fluorescing dye) with a PolD polymerase. The labeled nucleotide is represented in FIG. 27A as U‡-EH where “‡” represents a fluorescent dye of any type. As shown in FIG. 27A, even with a hyp10 linker incorporated, two red dyes fluoresce only 1.1× as brightly as a single dye. This indicates that red dyes, even with a linker incorporated into a fluorescent labeling structure, may suffer from quenching effects. FIG. 27B shows relative fluorescence following a sequencing assay using dUTP-B-H-Atto532 (e.g., a nucleotide labeled with a green-fluorescing dye) with a Pol47 polymerase. The labeled nucleotide is represented in FIG. 27B as U‡-BH where “‡” represents a fluorescent dye of any type. As shown, with a hyp10 linker incorporated into a green dye system, two green dyes fluoresce 1.6× as brightly as a single dye. This indicates that green dyes may encounter fewer quenching effects than red dyes.

### Example 18: Varying Labeling Fractions

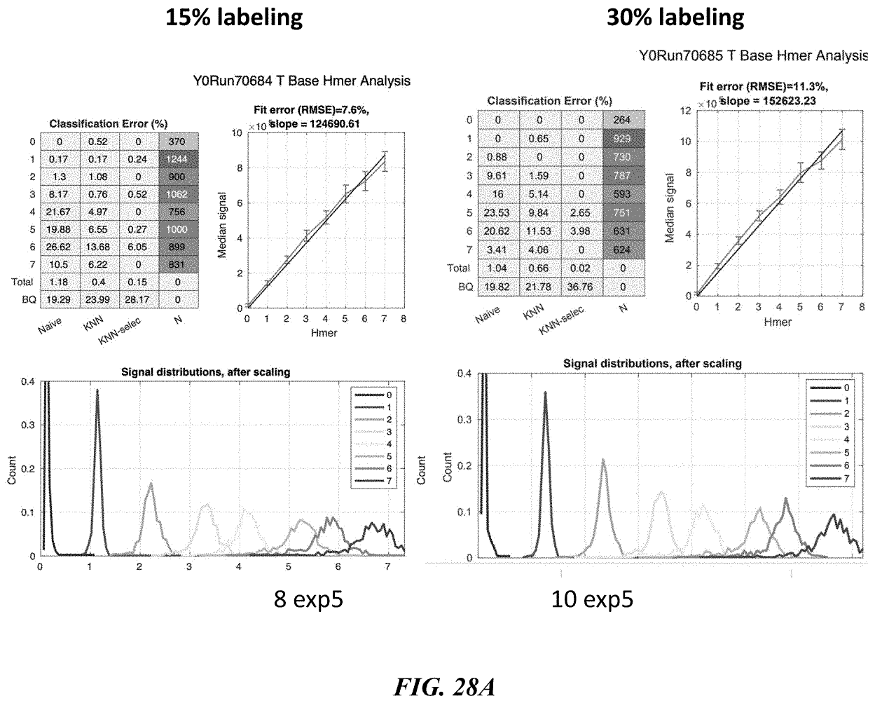

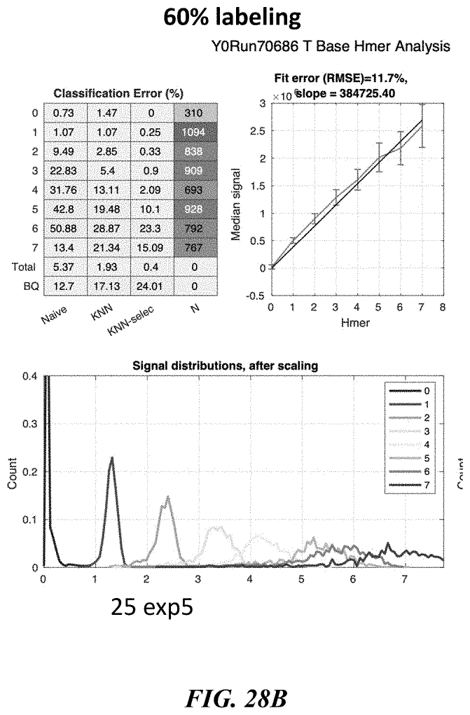

Labeled nucleotides were evaluated at different labeling fractions. The labeled nucleotide U*-EPH was used in a sequencing assay at 15%, 30%, and 60% labeling fractions. As shown in FIGS. 28A and 28B, labeling remained approximately linear for homopolymers through eight bases at 60% labeling fraction.

### Example 19: Optimization of Labeled Nucleotide Systems

A sequencing assay using labeled nucleotides may comprise the use of a polymerase. Table 3 below includes parameters corresponding to a sequencing assay performed with A*-EH, C*-YH, G*-EH, and U*-YH with a Pol19 polymerase enzyme and 110 mM sodium chloride.

TABLE 3
Parameters for sequencing assay performed with A*-EH, C*-
YH, G*-EH, and U*-YH using a Pol19 polymerase enzyme.

| | | Total | | |
|---|---|---|---|---|
| | | concentration of | Concentration of | 1mer |
| | Labeling | bright nucleotide | dark nucleotide | signal |
| Nucleotide | fraction | mixture (μM) | mixture (μM) | measured |
| A*-EH | 0.20 | 1 | 1 | 219899 |
| C*-YH | 0.20 | 1 | 2.5 | 234473 |
| G*-EH | 0.10 | 1 | 1 | 156905 |
| U*-YH | 0.40 | 1 | 2.5 | 250837 |

Measured lag and lead for this assay were 0.65 and 0.29, respectively.

A similar sequencing assay using the same nucleotides each at 20% labeling fraction using a Pol50 polymerase enzyme and 170 mM sodium chloride was also performed. Table 4 summarizes parameters corresponding to this sequencing assay.

TABLE 4
Parameters for sequencing assay performed with A*-EH, C*-
YH, G*-EH, and U*-YH using a Pol50 polymerase enzyme.

| | | Total | | |
|---|---|---|---|---|
| | | concentration of | Concentration of | 1mer |
| | Labeling | bright nucleotide | dark nucleotide | signal |
| Nucleotide | fraction | mixture (μM) | mixture (μM) | measured |
| A*-EH | 0.20 | 1 | 1 | 251515 |
| C*-YH | 0.20 | 1 | 2.5 | 323355 |
| G*-EH | 0.20 | 1 | 1 | 290579 |
| U*-YH | 0.20 | 2.5 | 2.5 | 145535 |

Measured lag and lead for this assay were 1.06 and 1.04, respectively. Accordingly, Pol50 performed more poorly than Pol19.

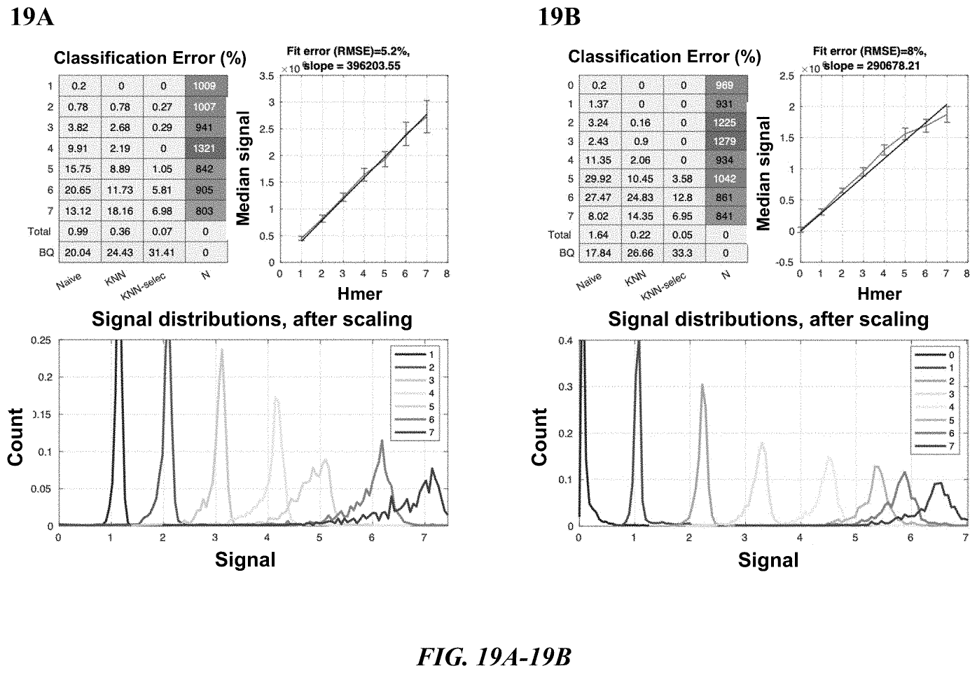

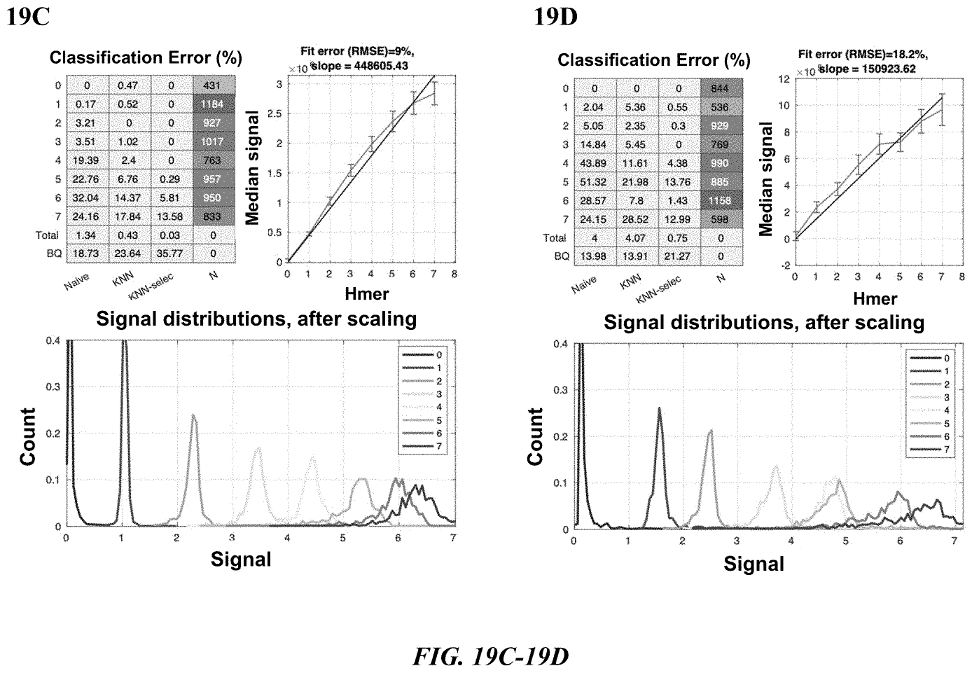

FIGS. 19A, 19B, 19C, and 19D show performance against homopolymeric regions for C-, A-, T-, and G-containing nucleotides, respectively, for the system described in Table 3 above. For each base, the context of the template prior to the homopolymeric sequence is identical, permitting assessment of performance against homopolymeric regions for different nucleotides. The upper right panel of each figure shows a plot of measured signal as a function of homopolymer length. In such figures, a linear fit indicates good response against a given homopolymer length with minimal overlap between homopolymers. Signal curves are relatively linear for each nucleotide for homopolymers (hmers) through n=7, particularly for C-containing nucleotides. The lower panel of each figure shows the signal distributions for each homopolymer length. As shown in these panels, signal peaks tend to broaden as homopolymer length increases. The tables included in the upper left panel of each figure summarize uncorrected data (leftmost column), raw counts (rightmost column), and error-corrected data (middle columns). FIGS. 19A-19D demonstrate that this system can effectively interrogate homopolymeric template sequences.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-263. (canceled)

264. A fluorescent labeling reagent comprising:
(a) a fluorescent dye moiety; and
(b) a linker that is connected to said fluorescent dye moiety and configured to couple to a substrate for fluorescently labelling said substrate,
wherein said linker comprises at least five non-proteinogenic amino acids.

265. The fluorescent labeling reagent of claim 264, further comprising a second fluorescent dye moiety, wherein said fluorescent dye moiety and said second fluorescent dye moiety are connected by said linker.

266. The fluorescent labeling reagent of claim 265, wherein said fluorescent dye moiety and said second fluorescent dye moiety are capable of energy transfer mediated via fluorescence resonance energy transfer (FRET).

267. The fluorescent labeling reagent of claim 264, wherein at least a subset of said at least five non-proteinogenic amino acids are hydroxyproline moieties.

268. The fluorescent labeling reagent of claim 267, wherein said linker comprises twenty or more hydroxyproline moieties.

269. The fluorescent labeling reagent of claim 268, wherein said linker comprises thirty or more hydroxyproline moieties.

270. The fluorescent labeling reagent of claim 264, wherein said linker further comprises one or more glycine moieties.

271. The fluorescent labeling reagent of claim 264, wherein said linker comprises a repeating unit, wherein said repeating unit comprises one or more of said at least five non-proteinogenic amino acid moieties.

272. The fluorescent labeling reagent of claim 271, wherein said repeating unit comprises a glycine moiety.

273. The fluorescent labeling reagent of claim 271, wherein said repeating unit is repeated at least three times.

274. The fluorescent labeling reagent of claim 264, wherein, when said fluorescent labeling reagent is coupled to said substrate, said linker provides an average physical separation between said fluorescent dye moiety and said substrate of at least about 30 Angstroms (Å).

275. The fluorescent labeling reagent of claim 274, wherein, when said fluorescent labeling reagent is coupled to said substrate, said linker provides said average physical separation between said fluorescent dye moiety and said substrate of at least about 60 Angstroms (Å).

276. The fluorescent labeling reagent of claim 264, wherein said fluorescent labeling reagent further comprises a cleavable group that is configured to be cleaved to separate said fluorescent labeling reagent or portion thereof from said substrate.

277. The fluorescent labeling reagent of claim 276, wherein said cleavable group is selected from the group consisting of an azidomethyl group, a disulfide bond, a hydrocarbyldithiomethyl group, and a 2-nitrobenzyloxy group.

278. The fluorescent labeling reagent of claim 277, wherein said cleavable group is said disulfide bond.

279. The fluorescent labeling reagent of claim 264, wherein said fluorescent labeling reagent comprises a moiety selected from the group consisting of

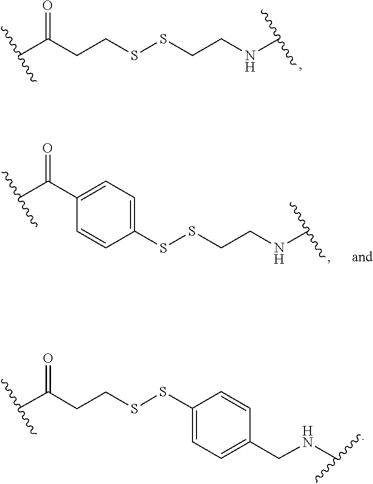 [SMILES: *NCCSSCCC(*)=O ; *NCCSSc1ccc(C(*)=O)cc1 ; *NCc1ccc(SSCCC(*)=O)cc1]

280. The fluorescent labeling reagent of claim 264, wherein said substrate is a nucleotide, polynucleotide, protein, lipid, cell, saccharide, polysaccharide, or antibody.

281. The fluorescent labeling reagent of claim 280, wherein said substrate is said nucleotide and said fluorescent labeling reagent is attached to said nucleotide via the nucleobase of said nucleotide.

282. The fluorescent labeling reagent of claim 280, wherein said substrate is said protein.

283. The fluorescent labeling reagent of claim 264, wherein said substrate is a fluorescence quencher, a fluorescence donor, or a fluorescence acceptor.
